(19) United States
(12) Patent Application Publication
(10) Pub. No.: US 2024/0342042 A1
(43) Pub. Date: Oct. 17, 2024

(54) DIGITAL APPARATUS AND APPLICATION FOR IMPROVING EYESIGHT

(71) Applicant: S-Alpha Therapeutics, Inc., Seoul (KR)

(72) Inventor: Seung Eun Choi, Seoul (KR)

(73) Assignee: S-Alpha Therapeutics, Inc., Seoul (KR)

(21) Appl. No.: 18/386,938

(22) Filed: Nov. 3, 2023

Related U.S. Application Data
(60) Provisional application No. 63/422,268, filed on Nov. 3, 2022.

(51) Int. Cl. A61H 5/00

(52) U.S. Cl. CPC A61H 5/00; A61H 2201/5012; A61H 2201/5046

(57) ABSTRACT

Systems and methods for improving an eyesight of a subject are provided. A system may include a digital apparatus, which may include a digital instruction generation unit configured to generate one or more digital therapeutic modules for improving the eyesight based on a mechanism of action (MOA) in and a therapeutic hypothesis for improving the eyesight, generate specified digital instructions based on the one or more digital therapeutic modules and provide the digital instructions to a first user, and an outcome collection unit configured to collect the first user's execution outcomes of the digital instructions. The system may also include a healthcare provider portal for a healthcare provider to manage their patients and/or an administrative portal.

The disclosure of U.S. application Ser. No. 16/747,980, filed Jan. 21, 2020, is incorporated herein by reference in its entirety.

### FIELD

The present disclosure relates to digital therapeutics (hereinafter referred to as DTx) intended for eyesight treatment improving an eyesight including myopia therapy, which includes inhibition of progression of myopia. The present disclosure also relates to systems that integrate digital therapeutics with one or both of a healthcare provider portal and an administrative portal to provide the eyesight treatment and treat myopia in a patient. In particular, embodiments of the present disclosure may comprise establishing a therapeutic hypothesis and a digital therapeutic hypothesis for improving the eyesight and inhibiting progression of axial myopia in the childhood/adolescence stages, providing the eyesight treatment and treating the axial myopia based on these findings. The present disclosure also relates to a rational design of an application for clinically verifying a digital therapeutic hypothesis for eyesight treatment and axial myopia in the childhood/adolescence stages and realizing the digital therapeutic hypothesis for digital therapeutics, and to the provision of a digital apparatus and an application for improving eyesight and inhibiting progression of axial myopia in childhood/adolescence stages, providing eyesight treatment and treating the axial myopia based on this rational design.

### BACKGROUND

AL (Axial Length) is the combination of anterior chamber depth, lens thickness and vitreous chamber depth, and it is the most significant contributor to refractive error. Myopia may result from an increase in AL outside of the normal rate expected for age. In children with rapidly progressing Myopia, AL will increase faster than the normal rate. In Korea, myopic patients have very high morbidity. The results of data analysis in the years 2008 to 2012 show that the morbidity of myopia (−0.75 diopters or higher) in 12- to 18-year-old adolescents in Korea is 80.4%, which is 4.35 times higher than the morbidity of myopia (18.5%) in the 60-year-old elderly in demographic aspects, and that the morbidity in high myopia (−6 diopter or higher) is 12%, which is 8 times higher than that of the 60-year-old elderly (1.5%), and also is three times higher than the morbidity of myopia of the adolescents in U.S.A, the United Kingdom, etc.

It is more serious that approximately 70% of the adolescent myopic patients in Korea were surveyed to be medium- and high-myopic patients. Also, the morbidity of myopia in the elementary school students was approximately 23% in 1980, but steadily increased from 38% in 1990 to 46.2% in 2000.

The World Health Organization (WHO) has recognized myopia as one of diseases, but there is no potent therapeutics against myopia around the world. In recent years, the myopia began again to receive academic attention as the morbidity of myopia has increased suddenly in China, Singapore, Korea, etc. Also, the myopia has emerged as an ophthalmologic disease that may also cause the loss of eyesight in the future.

Types of myopia are divided into axial myopia caused due to the extending axis of eyeball and refractive myopia (i.e., indexmyopia) caused due to an increased refractive index of the eye lens or the cornea, etc. Types of the axial myopia are divided into simple myopia having no influence on the retina or the choroid, and degenerative myopia causing deformation in the retina to induce the loss of eyesight. Except for nuclear sclerosis and keratoconum caused by the diabetes, most of the myopia corresponds to simple axial myopia whose progression is accelerated from the elementary school ages.

As this method for delaying progression of or treating myopia, a method of using a drug (atropine) and a special lens (for example, a dream lens) was known. However, atropine causes serious dazzling with the pupil dilation. Also, because the dream lens has a high risk of damage to the cornea, it has limited clinical applications, compared to glasses for vision corrosion.

Separately, although a variety of apparatuses for treating myopia, eye exercise methods, eye exercise applications, and the like have been developed and sold in the market, most of them have insufficient grounds for the clinical efficacy, and are sold without any additional permission. However, there is no highly reliable therapeutic method that the childhood/adolescent patients who have been diagnosed as myopia in the hospitals can use to inhibit progression of and treat myopia.

### SUMMARY OF THE DISCLOSURE

In some aspects, the present disclosure provides a method of improving an eyesight of a subject, the method comprising, providing, by a digital apparatus to the subject, a digital application comprising one or more digital therapeutic modules for improving the eyesight, each of the modules comprising one or more first instructions for the subject to follow, wherein the first instructions comprise a first eyeball exercise instruction for the subject to move at least one eyeball vertically. The digital therapeutic module for improving the eyesight may be generated based on a mechanism of action and a therapeutic hypothesis. In some embodiments, the digital therapeutic module for improving the eyesight may be generated based on neurohumoral factors. Each digital therapeutic module is a basic design unit of a digital therapeutic which can be implemented as a digital application or device. Each digital therapeutic module may include one or more instructions which are provided to the subject to follow.

In some aspects, the present disclosure provides a method of treating myopia in a subject in need thereof, the method comprising providing, by a digital apparatus to the subject, a digital application comprising modules for treating myopia, each of the modules comprising one or more first instructions for the subject to follow, wherein the first instructions comprise a first eyeball exercise instruction for the subject to move at least one eyeball vertically. In some aspects, the present disclosure provides anon-transitory computer readable medium having stored thereon software instructions for improving an eyesight of a subject that, when executed by a processor, cause the processor to display, by a digital apparatus to the subject, modules for improving an eyesight, each of the modules comprising one or more instructions for the subject to follow, the first instructions comprise an eyeball exercise instruction for the subject to move at least one eyeball vertically and sense, by a sensor in the digital apparatus, adherence by the subject to the instructions of the modules.

In some embodiments, the first eyeball exercise instruction is for the subject to move said at least one eyeball at least 50 out of 100 maximum vertical view of the subject. In some embodiments, the first eyeball exercise instruction is for the subject to move said at least one eyeball at least 70 out of 100 maximum vertical view of the subject. In some embodiments, the digital application comprises more instructions for vertical eye movements compared to instructions for horizontal eye movement. In some embodiments, the first eyeball exercise instruction is to move said at least one eyeball upward. In some embodiments, the first eyeball exercise instruction comprise more instruction to move said at least one eyeball upward compared to instruction to move said at least eyeball downward. In some embodiments, the first instructions exclude an instruction to move said at least one eyeball horizontally. In some embodiments, the method improves a growth rate of AL (Axial Length) of said at least one eyeball in the subject. In some embodiments, the method reduces a growth rate of AL (Axial Length) of said at least one eyeball in the subject. The method comprises further comprising measuring a maximum vertical view of the subject. In some embodiments, the measuring is performed by a sensor of the digital apparatus. In some embodiments, the subject is 10 years old or more. In some embodiments, the modules are selected based on a mechanism of action in and a therapeutic hypothesis, wherein the digital apparatus (i) comprises a sensor sensing adherence by the subject to the one or more first instructions of the modules, (ii) transmits adherence information, based on the adherence, to a server accessible by a healthcare provider through a healthcare provider portal, and (iii) receives one or more second instructions from the healthcare provider based on the adherence information. In some embodiments, said one or more second instructions comprise a second eyeball exercise instructions for an eyeball movement at a speed adjusted based on the adherence information.

In some embodiments, the digital application instructs a processor of the digital apparatus to execute operations comprising: generating digital therapeutic modules based on a mechanism of action and a therapeutic hypothesis. In some embodiments, the generating of the digital therapeutic modules comprises generating the digital therapeutic modules based on neurohumoral factors. In some embodiments, the operations further comprise generating a calibration module for calibrating one or more of an accuracy of measurement of the subject's eye position, and a light environment. In some embodiments, the calibration module is generated prior to generating the digital therapeutic modules. In some embodiments, wherein the accuracy of measurement of the subject's eye position is calibrated, and said calibrating the accuracy of measurement of the subject's eye position comprises one or more of instructing the subject to position their face to appear on a screen of the digital apparatus, detecting the subject's eyes for a given period of time, instructing the subject to blink their eyes, detecting if the subject blinked their eyes, instructing the subject to stare at the screen, instructing the subject to move their eyes in a given direction or rotate their eyes, and determining a threshold for detecting the subject's eyes. In some embodiments, the accuracy of measurement of the light environment is calibrated, and said calibrating the light environment comprises one or more of detecting light in the subject's environment using a light sensor of the digital apparatus, and instructing the subject to turn on one or more lights in their environment. In some embodiments, the digital apparatus comprises one or more sensors for tracking movement of the subject's eyeball. In some embodiments, the digital application instructs a processor of the digital apparatus to execute operations comprising: generating digital therapeutic modules based on a mechanism of action in and a therapeutic hypothesis; generating digital instructions based on the digital therapeutic modules; providing the digital instruction to a subject; and collecting the subject's execution outcomes of the digital instructions. In some embodiments, the generating of the digital instructions and the collecting of the subject's execution outcomes of the digital instructions are repeatedly executed several with multiple feedback loops, and the generating of the digital instructions comprises generating the subject's digital instructions for this cycle based on the subject's digital instructions in the previous cycle and the collected execution outcome data on the subject's digital instructions provided in the previous cycle. In some embodiments, the collecting the subject's execution outcomes of the digital instructions comprises determining one or both of an exercise intensity (EI) and an average exercise intensity (AEI). In some embodiments, AEI is determined as an averaged sum of differences between a final location of an eyeball of the subject and a starting location of the eyeball measured at a given interval. In some embodiments, the interval is between about 10 milliseconds (ms) and about 500 ms. In some embodiments, the EI is determined according the formula:

$$EI = \frac{AEI \times 100}{145}$$

In some embodiments, the AEI is determined as a sum of static AEI and dynamic AEI. In some embodiments, the generating of the digital therapeutic modules comprises generating the digital therapeutic modules by applying imaginary parameters about the subject's environments, behaviors, emotions, and cognition to the mechanism of action in and the therapeutic hypothesis. In some embodiments, the digital application instructs a processor of the digital apparatus to generate digital therapeutic modules comprising (i) an eye exercise module comprising the eyeball exercise instructions, and (ii) at least one of a relaxation module and a light therapy module. In some embodiments, the eye exercise module further comprises one or more of biofeedback control instructions and eyeball-related behavior control instructions; the relaxation module comprises one or more relaxation instructions for one or more of: physical exercise instructions, ego enhancement instructions, safety feeling instructions, comfort feeling instructions, and fun instructions; and the light therapy module comprises one or more light therapy instructions for controlling a light environment of the subject. In some embodiments, the one or more relaxation instructions comprise one or more of playing a sound or song, inducing blinking, and instructing the subject to perform gymnastics. In some embodiments, the digital therapeutic modules further comprise an accomplishment module comprising one or more accomplishment instructions for task accomplishment and for providing compensation for the subject's adherence to the instructions of the two or more first modules. In some embodiments, the digital therapeutic modules further comprise a fun module comprising one or more fun instructions for music, games, or videos. In some embodiments, the healthcare provider portal is configured to provide one or more options to the healthcare provider to perform one or more tasks to prescribe treatment in the subject based on the adherence information, wherein the one or more options provided to the healthcare provider are selected from the group consisting of adding or removing the subject, viewing or editing personal information for the subject, viewing adherence information for the subject, viewing a result of the subject for one or more at least partially completed digital therapeutic modules, prescribing one or more digital therapeutic modules to the subject, altering a prescription for one or more digital therapeutic modules, and communicating with the subject. In some embodiments, the one or more options comprise the viewing or editing personal information for the subject, and the personal information comprises one or more selected from the group consisting of an identification number for the subject, a name of the subject, a date of birth of the subject, an email of the subject, an email of the guardian of the subject, a contact phone number for the subject, a prescription for the subject, and one or more notes made by the healthcare provider about the subject. In some embodiments, the personal information comprises the prescription for the subject, and the prescription for the subject comprises one or more selected from the group consisting of a prescription identification number, a prescription type, a start date, a duration, a completion date, a number of scheduled or prescribed digital therapeutic modules to be performed by the subject, and a number of scheduled or prescribed digital therapeutic modules to be performed by the subject per day. In some embodiments, the one or more options comprise the viewing the adherence information, and the adherence information of the subject comprises one or more of a number of scheduled or prescribed digital therapeutic modules completed by the subject, and a calendar identifying one or more days on which the subject completed, partially completed, or did not complete one or more scheduled or prescribed digital therapeutic modules. In some embodiments, the one or more options comprise the viewing the result of the subject, and the result of the subject for one or more at least partially completed digital therapeutic modules comprises one or more selected from the group consisting of a time at which the subject started a scheduled or prescribed digital therapeutic module, a time at which the subject ended a scheduled or prescribed digital therapeutic module, an indicator of whether the scheduled or prescribed digital therapeutic module was fully or partially completed, and an exercise intensity (EI). In some embodiments, the server is accessible by an administrator through an administrative portal configured to provide one or more options to an administrator of the system to perform one or more tasks to manage access to the system by the healthcare provider, and wherein the one or more options provided to the administrator of the method are selected from the group consisting of adding or removing the healthcare provider, viewing or editing personal information for the healthcare provider, viewing or editing de-identified information of the subject, viewing adherence information for the subject, viewing a result of the subject for one or more at least partially completed digital therapeutic modules, and communicating with the healthcare provider. In some embodiments, the one or more options comprise the viewing or editing the personal information, and the personal information of the healthcare provider comprises one or more selected from the group consisting of an identification number for the healthcare provider, a name of the healthcare provider, an email of the healthcare provider, and a contact phone number for the healthcare provider. In some embodiments, the one or more options comprise the viewing or editing the de-identified information of the subject, and the de-identified information of the subject comprises one or more selected from the group consisting of an identification number for the subject, and the healthcare provider for the subject. In some embodiments, the one or more options comprise the viewing the adherence information for the subject, and the adherence information of the subject comprises one or more of a number of scheduled or prescribed digital therapeutic modules completed by the subject, and a calendar identifying one or more days on which the subject completed, partially completed, or did not complete one or more scheduled or prescribed digital therapeutic modules. In some embodiments, the one or more options comprise the viewing the result of the subject, and the result of the subject for one or more at least partially completed digital therapeutic modules comprises one or more selected from the group consisting of a time at which the subject started a scheduled or prescribed digital therapeutic module, a time at which the subject ended a scheduled or prescribed digital therapeutic module, an indicator of whether the scheduled or prescribed digital therapeutic module was fully or partially completed, and an exercise intensity (EI). In some embodiments, the digital application further comprises a push alarm for one or more of reminding the subject complete a digital therapeutic module and adjusting the light settings of the subject's environment. In some embodiments, the push alarm is activated to remind the subject to adjust the light settings such that the subject is exposed to sufficiently bright light at least 3 times per day. In some embodiments, the subject is a child. In some embodiments, the subject is less than about 15 years old. In some embodiments, the subject is assisted or supervised by an adult. In some embodiments, the digital apparatus comprises: a digital instruction generation unit configured to generate digital therapeutic modules based on a mechanism of action (MOA) in and a therapeutic hypothesis, generate digital instructions based on the digital therapeutic modules, and provide the digital instructions to the subject; and an outcome collection unit configured to collect the subject's execution outcomes of the digital instructions. In some embodiments, the digital instruction generation unit generates the digital therapeutic modules based on neurohumoral factors. In some embodiments, the neurohumoral factors comprise insulin-like growth factor (IGF), cortisol, and dopamine. In some embodiments, the digital instruction generation unit generates the digital therapeutic modules based on the inputs from the healthcare provider. In some embodiments, the digital instruction generation unit generates the digital therapeutic modules based on information received from the subject. In some embodiments, the information is received from the subject comprises at least one of basal factors, medical information, and digital therapeutics literacy of the subject, the basal factors including the subject's activity, heart rate, sleep, and diet (including nutrition and calories), the medical information including the subject's electronic medical record (EMR), family history, genetic vulnerability, and genetic susceptibility, and the digital therapeutics literacy including the subject's accessibility, and technology adoption to the digital therapeutics and the apparatus. In some embodiments, the digital instruction generation unit generates the digital therapeutic modules matching to imaginary parameters which correspond to the mechanism of action in and the therapeutic hypothesis. In some embodiments, the imaginary parameters are deduced in relation to the subject's environment, behaviors, emotions, and cognition. In some embodiments, the outcome collection unit collects the execution outcomes of the digital instructions by monitoring the subject's adherence to the digital instructions or allowing the subject to directly input the subject's adherence to the digital instructions. In some embodiments, the generation of the digital instructions at the digital instruction generation unit and the collection of the subject's execution outcomes of the digital instructions at the outcome collection unit are repeatedly executed several times with multiple feedback loops, and the digital instruction generation unit generates the subject's digital instructions for this cycle based on the subject's digital instructions in the previous cycle and the execution outcome data on the subject's digital instructions in the previous cycle collected at the outcome collection unit.

In some aspects, the present disclosure provides a system for improving an eyesight in a subject, the system comprising: a digital apparatus configured to execute a digital application for improving an eyesight in the subject depending the methods described above; a healthcare provider portal configured to provide one or more options to a healthcare provider to perform one or more tasks to prescribe treatment to improve the eyesight in the subject based on information received from the digital application; and an administrative portal configured to provide one or more options to an administrator of the system to perform one or more tasks to manage access to the system by the healthcare provider.

In some aspects, the present disclosure provides a system for treating myopia in a subject in need thereof, the system comprising: a digital apparatus configured to execute a digital application for treating myopia in the subject depending the methods describe above; a healthcare provider portal configured to provide one or more options to a healthcare provider to perform one or more tasks to prescribe treatment to treat myopia in the subject based on information received from the digital application; and an administrative portal configured to provide one or more options to an administrator of the system to perform one or more tasks to manage access to the system by the healthcare provider.

In some aspects, the present disclosure provides a non-transitory computer readable medium having stored thereon software instructions for treating myopia in a subject in need thereof that, when executed by a processor, cause the processor to display, by an digital apparatus to the subject, modules for treating myopia, each of the modules comprising one or more instructions for the subject to follow, the first instructions comprise an eyeball exercise instruction for the subject to move at least one eyeball vertically; sense, by a sensor in the digital apparatus, adherence by the subject to the instructions of the modules.

In some embodiments, the first eyeball exercise instruction is for the subject to move said at least one eyeball at least 50 out of 100 maximum vertical view of the subject. In some embodiments, the first eyeball exercise instruction is for the subject to move said at least one eyeball at least 70 out of 100 maximum vertical view of the subject. In some embodiments, the digital application comprises more instructions for vertical eye movements compared to instructions for horizontal eye movement. In some embodiments, the first eyeball exercise instruction is to move said at least one eyeball upward. In some embodiments, the first eyeball exercise instruction comprise more instruction to move said at least one eyeball upward compared to instruction to move said at least eyeball downward. In some embodiments, the first instructions exclude an instruction to move said at least one eyeball horizontally. In some embodiments, the method improves a growth rate of AL (Axial Length) of said at least one eyeball in the subject. In some embodiments, the method reduces a growth rate of AL (Axial Length) of said at least one eyeball in the subject. The method comprises further comprising measuring a maximum vertical view of the subject. In some embodiments, the measuring is performed by a sensor of the digital apparatus. In some embodiments, the subject is 10 years old or more. In some embodiments, the modules are selected based on a mechanism of action in and a therapeutic hypothesis, wherein the digital apparatus (i) comprises a sensor sensing adherence by the subject to the one or more first instructions of the modules, (ii) transmits adherence information, based on the adherence, to a server accessible by a healthcare provider through a healthcare provider portal, and (iii) receives one or more second instructions from the healthcare provider based on the adherence information. In some embodiments, said one or more second instructions comprise a second eyeball exercise instructions for an eyeball movement at a speed adjusted based on the adherence information.

In some embodiments, the digital application instructs a processor of the digital apparatus to execute operations comprising: generating digital therapeutic modules based on a mechanism of action and a therapeutic hypothesis. In some embodiments, the generating of the digital therapeutic modules comprises generating the digital therapeutic modules based on neurohumoral factors. In some embodiments, the operations further comprise generating a calibration module for calibrating one or more of an accuracy of measurement of the subject's eye position, and a light environment. In some embodiments, the calibration module is generated prior to generating the digital therapeutic modules. In some embodiments, wherein the accuracy of measurement of the subject's eye position is calibrated, and said calibrating the accuracy of measurement of the subject's eye position comprises one or more of instructing the subject to position their face to appear on a screen of the digital apparatus, detecting the subject's eyes for a given period of time, instructing the subject to blink their eyes, detecting if the subject blinked their eyes, instructing the subject to stare at the screen, instructing the subject to move their eyes in a given direction or rotate their eyes, and determining a threshold for detecting the subject's eyes. In some embodiments, the accuracy of measurement of the light environment is calibrated, and said calibrating the light environment comprises one or more of detecting light in the subject's environment using a light sensor of the digital apparatus, and instructing the subject to turn on one or more lights in their environment. In some embodiments, the digital apparatus comprises one or more sensors for tracking movement of the subject's eyeball. In some embodiments, the digital application instructs a processor of the digital apparatus to execute operations comprising: generating digital therapeutic modules based on a mechanism of action in and a therapeutic hypothesis; generating digital instructions based on the digital therapeutic modules; providing the digital instruction to a subject; and collecting the subject's execution outcomes of the digital instructions. In some embodiments, the generating of the digital instructions and the collecting of the subject's execution outcomes of the digital instructions are repeatedly executed several with multiple feedback loops, and the generating of the digital instructions comprises generating the subject's digital instructions for this cycle based on the subject's digital instructions in the previous cycle and the collected execution outcome data on the subject's digital instructions provided in the previous cycle. In some embodiments, the collecting the subject's execution outcomes of the digital instructions comprises determining one or both of an exercise intensity (EI) and an average exercise intensity (AEI). In some embodiments, AEI is determined as an averaged sum of differences between a final location of an eyeball of the subject and a starting location of the eyeball measured at a given interval. In some embodiments, the interval is between about 10 milliseconds (ms) and about 500 ms. In some embodiments, the EI is determined according the formula:

$$EI = \frac{AEI \times 100}{145}$$

In some embodiments, the AEI is determined as a sum of static AEI and dynamic AEI. In some embodiments, the generating of the digital therapeutic modules comprises generating the digital therapeutic modules by applying imaginary parameters about the subject's environments, behaviors, emotions, and cognition to the mechanism of action in and the therapeutic hypothesis. In some embodiments, the digital application instructs a processor of the digital apparatus to generate digital therapeutic modules comprising (i) an eye exercise module comprising the eyeball exercise instructions, and (ii) at least one of a relaxation module and a light therapy module. In some embodiments, the eye exercise module further comprises one or more of biofeedback control instructions and eyeball-related behavior control instructions; the relaxation module comprises one or more relaxation instructions for one or more of: physical exercise instructions, ego enhancement instructions, safety feeling instructions, comfort feeling instructions, and fun instructions; and the light therapy module comprises one or more light therapy instructions for controlling a light environment of the subject. In some embodiments, the one or more relaxation instructions comprise one or more of playing a sound or song, inducing blinking, and instructing the subject to perform gymnastics. In some embodiments, the digital therapeutic modules further comprise an accomplishment module comprising one or more accomplishment instructions for task accomplishment and for providing compensation for the subject's adherence to the instructions of the two or more first modules. In some embodiments, the digital therapeutic modules further comprise a fun module comprising one or more fun instructions for music, games, or videos. In some embodiments, the healthcare provider portal is configured to provide one or more options to the healthcare provider to perform one or more tasks to prescribe treatment in the subject based on the adherence information, wherein the one or more options provided to the healthcare provider are selected from the group consisting of adding or removing the subject, viewing or editing personal information for the subject, viewing adherence information for the subject, viewing a result of the subject for one or more at least partially completed digital therapeutic modules, prescribing one or more digital therapeutic modules to the subject, altering a prescription for one or more digital therapeutic modules, and communicating with the subject. In some embodiments, the one or more options comprise the viewing or editing personal information for the subject, and the personal information comprises one or more selected from the group consisting of an identification number for the subject, a name of the subject, a date of birth of the subject, an email of the subject, an email of the guardian of the subject, a contact phone number for the subject, a prescription for the subject, and one or more notes made by the healthcare provider about the subject. In some embodiments, the personal information comprises the prescription for the subject, and the prescription for the subject comprises one or more selected from the group consisting of a prescription identification number, a prescription type, a start date, a duration, a completion date, a number of scheduled or prescribed digital therapeutic modules to be performed by the subject, and a number of scheduled or prescribed digital therapeutic modules to be performed by the subject per day. In some embodiments, the one or more options comprise the viewing the adherence information, and the adherence information of the subject comprises one or more of a number of scheduled or prescribed digital therapeutic modules completed by the subject, and a calendar identifying one or more days on which the subject completed, partially completed, or did not complete one or more scheduled or prescribed digital therapeutic modules. In some embodiments, the one or more options comprise the viewing the result of the subject, and the result of the subject for one or more at least partially completed digital therapeutic modules comprises one or more selected from the group consisting of a time at which the subject started a scheduled or prescribed digital therapeutic module, a time at which the subject ended a scheduled or prescribed digital therapeutic module, an indicator of whether the scheduled or prescribed digital therapeutic module was fully or partially completed, and an exercise intensity (EI). In some embodiments, the server is accessible by an administrator through an administrative portal configured to provide one or more options to an administrator of the system to perform one or more tasks to manage access to the system by the healthcare provider, and wherein the one or more options provided to the administrator of the method are selected from the group consisting of adding or removing the healthcare provider, viewing or editing personal information for the healthcare provider, viewing or editing de-identified information of the subject, viewing adherence information for the subject, viewing a result of the subject for one or more at least partially completed digital therapeutic modules, and communicating with the healthcare provider. In some embodiments, the one or more options comprise the viewing or editing the personal information, and the personal information of the healthcare provider comprises one or more selected from the group consisting of an identification number for the healthcare provider, a name of the healthcare provider, an email of the healthcare provider, and a contact phone number for the healthcare provider. In some embodiments, the one or more options comprise the viewing or editing the de-identified information of the subject, and the de-identified information of the subject comprises one or more selected from the group consisting of an identification number for the subject, and the healthcare provider for the subject. In some embodiments, the one or more options comprise the viewing the adherence information for the subject, and the adherence information of the subject comprises one or more of a number of scheduled or prescribed digital therapeutic modules completed by the subject, and a calendar identifying one or more days on which the subject completed, partially completed, or did not complete one or more scheduled or prescribed digital therapeutic modules. In some embodiments, the one or more options comprise the viewing the result of the subject, and the result of the subject for one or more at least partially completed digital therapeutic modules comprises one or more selected from the group consisting of a time at which the subject started a scheduled or prescribed digital therapeutic module, a time at which the subject ended a scheduled or prescribed digital therapeutic module, an indicator of whether the scheduled or prescribed digital therapeutic module was fully or partially completed, and an exercise intensity (EI). In some embodiments, the digital application further comprises a push alarm for one or more of reminding the subject complete a digital therapeutic module and adjusting the light settings of the subject's environment. In some embodiments, the push alarm is activated to remind the subject to adjust the light settings such that the subject is exposed to sufficiently bright light at least 3 times per day. In some embodiments, the subject is a child. In some embodiments, the subject is less than about 15 years old. In some embodiments, the subject is assisted or supervised by an adult. In some embodiments, the digital apparatus comprises: a digital instruction generation unit configured to generate digital therapeutic modules based on a mechanism of action (MOA) in and a therapeutic hypothesis, generate digital instructions based on the digital therapeutic modules, and provide the digital instructions to the subject; and an outcome collection unit configured to collect the subject's execution outcomes of the digital instructions. In some embodiments, the digital instruction generation unit generates the digital therapeutic modules based on neurohumoral factors. In some embodiments, the neurohumoral factors comprise insulin-like growth factor (IGF), cortisol, and dopamine. In some embodiments, the digital instruction generation unit generates the digital therapeutic modules based on the inputs from the healthcare provider. In some embodiments, the digital instruction generation unit generates the digital therapeutic modules based on information received from the subject. In some embodiments, the information is received from the subject comprises at least one of basal factors, medical information, and digital therapeutics literacy of the subject, the basal factors including the subject's activity, heart rate, sleep, and diet (including nutrition and calories), the medical information including the subject's electronic medical record (EMR), family history, genetic vulnerability, and genetic susceptibility, and the digital therapeutics literacy including the subject's accessibility, and technology adoption to the digital therapeutics and the apparatus. In some embodiments, the digital instruction generation unit generates the digital therapeutic modules matching to imaginary parameters which correspond to the mechanism of action in and the therapeutic hypothesis. In some embodiments, the imaginary parameters are deduced in relation to the subject's environment, behaviors, emotions, and cognition. In some embodiments, the outcome collection unit collects the execution outcomes of the digital instructions by monitoring the subject's adherence to the digital instructions or allowing the subject to directly input the subject's adherence to the digital instructions. In some embodiments, the generation of the digital instructions at the digital instruction generation unit and the collection of the subject's execution outcomes of the digital instructions at the outcome collection unit are repeatedly executed several times with multiple feedback loops, and the digital instruction generation unit generates the subject's digital instructions for this cycle based on the subject's digital instructions in the previous cycle and the execution outcome data on the subject's digital instructions in the previous cycle collected at the outcome collection unit.

### BRIEF DESCRIPTION OF THE DRAWINGS

The above and other objects, features and advantages of the present disclosure will become more apparent to those of ordinary skill in the art by describing in detail exemplary embodiments thereof with reference to the attached drawings, in which:

FIG. 1A is a diagram showing a mechanism of action in axial myopia in the childhood/adolescence stages proposed in the present disclosure, FIG. 1B is a diagram showing a therapeutic hypothesis for the axial myopia proposed in the present disclosure, and

FIG. 1C is a diagram showing a digital therapeutic hypothesis for axial myopia proposed in the present disclosure;

FIG. 2 is a block diagram showing a configuration of a digital apparatus for treating myopia according to one embodiment of the present disclosure;

FIG. 3 is a diagram showing input and output loops of a digital application for treating myopia according to one embodiment of the present disclosure;

FIG. 4 is a diagram showing a feedback loop for a digital apparatus and an application for treating myopia according to one embodiment of the present disclosure;

FIG. 5A is a diagram showing a module design for realizing a digital therapy in the digital apparatus and the application for treating myopia according to one embodiment of the present disclosure, FIG. 5B is a diagram showing a background factors supporting the digital apparatus and the application for treating myopia according to one embodiment of the present disclosure;

FIG. 6 is a diagram showing a method of assigning a patient-customized digital prescription using the digital apparatus and the application for treating myopia according to one embodiment of the present disclosure;

FIG. 7A shows execution environment setups according to one embodiment of the present disclosure, and FIGS. 7B to 7G show examples of specific instructions for each module, and methods of collecting output data according to one embodiment of the present disclosure;

FIG. 8 is a flowchart illustrating operations in a digital application for treating myopia according to one embodiment of the present disclosure;

FIG. 9 is a flowchart illustrating a method of generating digital instructions in the digital application for treating myopia according to one embodiment of the present disclosure;

FIG. 10 is a flowchart illustrating a method of repeatedly executing the operations under the feedback control in the digital application for treating myopia according to one embodiment of the present disclosure; and

FIG. 11 is a diagram showing a hardware configuration of the digital apparatus for treating myopia according to one embodiment of the present disclosure.

FIG. 12 is a flow chart illustrating a system for treating myopia, the system comprising an administrative portal (e.g., Administrator's web), a healthcare provider portal (e.g., Doctor's web) and a digital apparatus configured to execute a digital application (e.g., an application or ‘app’) for treating myopia in a subject.

FIG. 13 is a flow chart illustrating an execution flow for a digital application of the present disclosure.

FIG. 14 is a flow chart illustrating an execution flow for a splash process at the starting of a digital application of the present disclosure.

FIG. 15 is a flow chart illustrating an execution flow for a login verification during a splash process at the starting of a digital application of the present disclosure.

FIG. 16 is a flow chart illustrating an execution flow for a prescription verification during a splash process at the starting of a digital application of the present disclosure.

FIG. 17 is a flow chart illustrating an execution flow for home entry during a prescription verification in a digital application of the present disclosure.

FIG. 18 is a flow chart illustrating an execution flow for sessions in a digital application of the present disclosure.

FIG. 19 is a flow chart illustrating an execution flow for a calibration module in a digital application of the present disclosure.

FIG. 20 is a flow chart illustrating an execution flow for a session in a digital application of the present disclosure, wherein the session comprises 2 or more digital therapeutic modules.

FIG. 21 depicts a splash screen of a digital application of the present disclosure, wherein the splash screen comprises a company logo, loading icon, and/or information on the version of the digital application.

FIG. 22 depicts TrueDepth Camera Notification Screens of a digital application of the present disclosure.

FIG. 23 depicts Home screens of a digital application of the present disclosure, wherein the Home screen indicates the availability of sessions for a subject to complete.

FIG. 24 depicts a Bright Environment Requirement Notification screen of a light therapy module of a digital application of the present disclosure, wherein the Bright Environment Requirement Notification screen indicates the amount of light detected by the digital apparatus.

FIG. 25 depicts a Calibration Notification screen of a digital application of the present disclosure, wherein the Calibration Notification screen indicates whether a subject's eye and/or movement of the eye are detectable by the camera.

FIG. 26A depicts a screenshot of an eye exercise digital therapeutic module of the present disclosure, and FIG. 26B depicts a flow chart illustrating an execution flow for an eye exercise digital therapeutic module.

FIG. 27A depicts a screenshot of a rest with relaxation digital therapeutic module of the present disclosure, and FIG. 27B depicts a flow chart illustrating an execution flow for a rest with relaxation digital therapeutic module.

FIG. 28A depicts a screenshot of an eye exercise digital therapeutic module of the present disclosure, and FIG. 28B depicts a flow chart illustrating an execution flow for an eye exercise digital therapeutic module.

FIG. 29A depicts a screenshot of a rest with relaxation using sounds digital therapeutic module of the present disclosure, and FIG. 29B depicts a flow chart illustrating an execution flow for a rest with relaxation using sounds digital therapeutic module.

FIG. 30A depicts a screenshot of an eye exercise digital therapeutic module of the present disclosure, and FIG. 30B depicts a flow chart illustrating an execution flow for an eye exercise digital therapeutic module.

FIG. 31A depicts a screenshot of a deep breathing digital therapeutic module of the present disclosure, and FIG. 31B depicts a flow chart illustrating an execution flow for a deep breathing digital therapeutic module.

FIG. 32A depicts a screenshot of an eye exercise digital therapeutic module of the present disclosure, and FIG. 32B depicts a flow chart illustrating an execution flow for an eye exercise digital therapeutic module.

FIG. 33A depicts a screenshot of a deep breathing digital therapeutic module of the present disclosure, FIG. 33B depicts screenshots of a deep breathing digital therapeutic module when instructing the subject to inhale (left) and exhale (right), and FIG. 33C depicts a flow chart illustrating an execution flow for a deep breathing digital therapeutic module.

FIG. 34A depicts a screenshot of an eye exercise digital therapeutic module of the present disclosure, and FIG. 34B depicts a flow chart illustrating an execution flow for an eye exercise digital therapeutic module.

FIG. 35A depicts a screenshot of a rest with relaxation digital therapeutic module of the present disclosure, and FIG. 35B depicts a flow chart illustrating an execution flow for a rest with relaxation digital therapeutic module.

FIG. 36 depicts screenshots shown at the completion of a single session, at the completion of all daily sessions, and at stop/start verification in a digital application of the present disclosure.

FIG. 37A depicts a screenshot of a Room Decoration Board in an accomplishment module in a digital application of the present disclosure, and FIG. 37B depicts a timeline showing the days on which a subject may acquire a given Room Decoration item.

FIG. 38 depicts a screenshot of a Parent Section in a digital application of the present disclosure.

FIG. 39 depicts a screenshot of a Change Password Section in a digital application of the present disclosure.

FIG. 40 is a table showing push messages, a time when a given push message is sent to a subject, and an outcome when a given push message is opened.

FIG. 41 depicts the layout for an exemplary healthcare provider portal and/or an administrative portal of the present disclosure. The full screen may be used in login screen, etc., and there may be no headers or side bar menus. The default screen may be used in almost all screens after logging in such as dashboard, patient list, etc. The modal pop-up may be used in situations where the user's click is needed such as checking before deleting a patient from the patient list. The toast pop-up may be used to provide adequate notifications to the users and may use different colors for each situation such as success or failure in order for the users to easily check.

FIG. 42 depicts the layout for a healthcare provider portal and/or an administrative portal of the present disclosure.

FIG. 43 depicts the layout for a healthcare provider portal and/or an administrative portal of the present disclosure.

FIG. 44 is a flow chart illustrating an execution flow for a healthcare provider portal in a system of the present disclosure.

FIG. 45A depicts a dashboard of a healthcare provider portal, FIG. 45B depicts a patient tab in a healthcare provider portal, the patient tab displaying a list of patients, FIG. 45C depicts a patient tab in a healthcare provider portal, the patient tab displaying detailed information on a given patient, (D) a patient tab in a healthcare provider portal for adding a new patient, (E) a patient tab in a healthcare provider portal for editing information of an existing patient, (F) a patient tab in a healthcare provider portal that displays detailed prescription information for a given patient, (G-H) a patient tab in a healthcare provider portal for editing prescription information for a given patient, and (I) a patient tab in a healthcare provider portal for viewing details (e.g., date, status, duration, results) of a given session for a given patient.

FIG. 46 is a flow chart illustrating an execution flow for an administrative portal in a system of the present disclosure.

FIG. 47A depicts a dashboard of an administrative portal, FIG. 47B depicts a doctor tab in an administrative portal, the doctor tab displaying a list of doctors, FIG. 47C depicts a doctor tab in an administrative portal, the doctor tab displaying a list of patients being cared for by a given doctor, with patient-identifying information redacted (*), FIG. 47D depicts a doctor tab in an administrative portal for adding a new doctor, FIG. 47E depicts a doctor tab in an administrative portal for editing information of an existing doctor, FIG. 47F depicts a patient tab in an administrative portal that displays information for one or more patients, wherein sensitive information is redacted, FIG. 47G depicts a patient tab in an administrative portal that displays detailed patient or prescription information for a given patient, FIG. 47H depicts a patient tab in an administrative portal that displays detailed prescription information for a given patient, and FIG. 47I depicts a patient tab in an administrative portal for viewing details (e.g., date, status, duration, results) of a given session for a given patient.

FIG. 48 is a table showing privileges for the doctors using the healthcare provider portal and the administrators using the administrative portal.

FIG. 49 depicts graphs representing an AL (Axial Length) changes of subject groups, including an experimental group and a control group, between visits (V1, V4 and V5) and a correlation with age.

FIG. 50 depicts graphs representing an AL (Axial Length) change rate and correlation values of experimental group between V1 and V5 with age.

FIG. 51 depicts AL (Axial Length) growth rates in mm/year for ALOS (Oculus Sinister) of subject groups.

FIG. 52 depicts AL (Axial Length) growth rates in mm/year for ALOD (Oculus Dexter) of subject groups.

FIG. 53 depicts normalized AL (Axial Length) growth rate for ALOS of subject groups.

FIG. 54 depicts normalized AL (Axial Length) growth rate for ALOD of subject groups.

FIG. 55 depicts effect of an age on AL (Axial Length) growth rate of OS and OD in experimental groups.

FIG. 56 depicts a correlation between AL (Axial Length) of a first prescription (e.g., V4-V1) and AL (Axial Length) of a second prescription (e.g., V5-V4) in a control group and an experimental group, respectively.

FIG. 57 depicts a correlation between adherence of a subject to follow the eyeball exercise instruction and a growth rate of a CR (Cycloplegic refraction test).

FIG. 58 depicts a correlation between a speed of eyeball movement oculus sinister of a subject following the eyeball exercise instruction and a growth rate of AL (Axial Length).

FIG. 59 depicts a correlation between the speed of eyeball movement oculus dexter of a subject following the eyeball exercise instruction and a growth rate of AL (axial length).

FIG. 60 depicts a correlation between the grow rate of AL and the average distance of eye movements that the subject performs in each game.

FIG. 61 depicts a correlation between the grow rate of AL and the average of the maximum distance of eye movements that the subject performs in each game.

FIG. 62 depicts a correlation between the grow rate of the CR and the average of the maximum distance of eye movements that the subject performs in each game.

FIG. 63 depicts a correlation between the grow rate of the CR and a normalized game count. The normalized game count represents a number of movements per a game or an attending day to play the corresponding game.

FIG. 64 depicts a correlation between the grow rate of AL and the speed of eye movements.

FIG. 65 depicts graphs showing correlations between a growth rate and an average distance or average of maximum distance of eye movements that the subject performs the first eyeball exercise instruction.

FIG. 66 depicts graphs showing a total count, an average distance, an average distance of maximum distances and a maximum distance for up-down movements, up-side movements and down-side movements, respectively.

FIG. 67 depicts correlations between CROD growth rate, and the average distance, the average distance of maximum distances and the maximum distance for up-side movements and down-side movements, respectively.

FIGS. 68-71 depicts correlations between a growth rate of AL (Axial Length), and the average distance, the average distance of maximum distances and the maximum distance for up-side movements and down-side movements, respectively.

FIG. 72A depicts an example of a session provided in a digital application of the present disclosure.

FIG. 72B depicts one or more digital therapeutic modules in a session.

FIG. 73 depicts a flow chart illustrating an execution flow for a session in a digital application of the present disclosure.

FIG. 74 depicts a flow chart illustrating an execution flow for a session in a digital application of the present disclosure.

FIGS. 75-76 depict examples of user interfaces provided by a digital application of the present disclosure.

FIGS. 77-78 depict an example of a user interface for an eyeball exercise instruction displayed in the digital apparatus.

FIG. 79 is a flow chart illustrating an execution flow for the eyeball exercise instructions in FIGS. 77-78.

FIG. 80 depicts an example of a user interface for an eyeball exercise instruction displayed in the digital apparatus.

FIG. 81 is a flow chart illustrating an execution flow for the eyeball exercise instructions in FIG. 80.

FIG. 82 depicts an example of a user interface for an eyeball exercise instruction displayed in the digital apparatus.

FIG. 83 is a flow chart illustrating an execution flow for the eyeball exercise instructions in FIG. 82.

FIGS. 84-85 depict an example of a user interface for an eyeball exercise instruction displayed in the digital apparatus.

FIG. 86 is a flow chart illustrating an execution flow for the eyeball exercise instructions in FIGS. 84-85.

FIG. 87 depicts an example of a user interface for an eyeball exercise instruction displayed in the digital apparatus.

FIG. 88 is a flow chart illustrating an execution flow for the eyeball exercise instructions in FIG. 87.

FIG. 89A depicts a screenshot of an eye exercise digital therapeutic module in FIGS. 77-78 of the present disclosure and FIG. 89B depicts a flow chart illustrating an execution flow for the eye exercise digital therapeutic module in FIGS. 77-78 of the present disclosure.

FIG. 90A depicts a screenshot of an eye exercise digital therapeutic module in FIGS. 77-78 of the present disclosure and FIG. 90B depicts a flow chart illustrating an execution flow for the eye exercise digital therapeutic module in FIGS. 77-78 of the present disclosure.

FIG. 91A depicts a screenshot of an eye exercise digital therapeutic module in FIGS. 77-78 of the present disclosure and FIG. 91B depicts a flow chart illustrating an execution flow for the eye exercise digital therapeutic module in FIGS. 77-78 of the present disclosure.

FIG. 92A depicts a screenshot of an eye exercise digital therapeutic module in FIGS. 77-78 of the present disclosure and FIG. 92B depicts a flow chart illustrating an execution flow for the eye exercise digital therapeutic module in FIGS. 77-78 of the present disclosure.

FIG. 93A depicts a screenshot of an eye exercise digital therapeutic module in FIGS. 84-85 of the present disclosure and FIG. 93B depicts a flow chart illustrating an execution flow for the eye exercise digital therapeutic module in FIGS. 84-85 of the present disclosure.

FIG. 94A depicts a screenshot of an eye exercise digital therapeutic module in FIGS. 84-85 of the present disclosure and FIG. 94B depicts a flow chart illustrating an execution flow for the eye exercise digital therapeutic module in FIGS. 84-85 of the present disclosure.

FIG. 95A depicts a screenshot of an eye exercise digital therapeutic module in FIGS. 84-85 of the present disclosure and FIG. 95B depicts a flow chart illustrating an execution flow for the eye exercise digital therapeutic module in FIGS. 84-85 of the present disclosure.

FIG. 96A depicts a screenshot of an eye exercise digital therapeutic module in FIGS. 84-85 of the present disclosure and FIG. 96B depicts a flow chart illustrating an execution flow for the eye exercise digital therapeutic module in FIGS. 84-85 of the present disclosure.

FIG. 97A depicts a screenshot of an eye exercise digital therapeutic module in FIG. 82 of the present disclosure and FIG. 97B depicts a flow chart illustrating an execution flow for the eye exercise digital therapeutic module in FIG. 82 of the present disclosure.

FIG. 98A depicts a screenshot of an eye exercise digital therapeutic module in FIG. 82 of the present disclosure and FIG. 98B depicts a flow chart illustrating an execution flow for the eye exercise digital therapeutic module in FIG. 82 of the present disclosure.

FIG. 99A depicts a screenshot of an eye exercise digital therapeutic module in FIG. 82 of the present disclosure and FIG. 99B depicts a flow chart illustrating an execution flow for the eye exercise digital therapeutic module in FIG. 82 of the present disclosure.

FIG. 100A depicts a screenshot of an eye exercise digital therapeutic module in FIG. 82 of the present disclosure and FIG. 100B depicts a flow chart illustrating an execution flow for the eye exercise digital therapeutic module in FIG. 82 of the present disclosure.

FIG. 101A depicts a screenshot of an eye exercise digital therapeutic module in FIG. 87 of the present disclosure and FIG. 101B depicts a flow chart illustrating an execution flow for the eye exercise digital therapeutic module in FIG. 87 of the present disclosure.

FIG. 102A depicts a screenshot of an eye exercise digital therapeutic module in FIG. 87 of the present disclosure and FIG. 102B depicts a flow chart illustrating an execution flow for the eye exercise digital therapeutic module in FIG. 87 of the present disclosure.

FIG. 103A depicts a screenshot of an eye exercise digital therapeutic module in FIG. 87 of the present disclosure and FIG. 103B depicts a flow chart illustrating an execution flow for the eye exercise digital therapeutic module in FIG. 87 of the present disclosure.

FIG. 104A depicts a screenshot of an eye exercise digital therapeutic module in FIG. 87 of the present disclosure and FIG. 104B depicts a flow chart illustrating an execution flow for the eye exercise digital therapeutic module in FIG. 87 of the present disclosure.

FIG. 105A depicts a screenshot of an eye exercise digital therapeutic module in FIG. 80 of the present disclosure and FIG. 105B depicts a flow chart illustrating an execution flow for the eye exercise digital therapeutic module in FIG. 80 of the present disclosure.

FIG. 106A depicts a screenshot of an eye exercise digital therapeutic module in FIG. 80 of the present disclosure and FIG. 106B depicts a flow chart illustrating an execution flow for the eye exercise digital therapeutic module in FIG. 80 of the present disclosure.

FIG. 107A depicts a screenshot of an eye exercise digital therapeutic module in FIG. 80 of the present disclosure and FIG. 107B depicts a flow chart illustrating an execution flow for the eye exercise digital therapeutic module in FIG. 80 of the present disclosure.

FIG. 108A depicts a screenshot of an eye exercise digital therapeutic module in FIG. 80 of the present disclosure and FIG. 108B depicts a flow chart illustrating an execution flow for the eye exercise digital therapeutic module in FIG. 80 of the present disclosure.

FIGS. 109-112 are flow charts illustrating an execution flow for a relaxation module.

FIG. 113 is a flow chart illustrating an execution flow for a deep breathing module.

FIGS. 114A-C depict screenshots of a series of behavioral instructions of a relaxation module.

FIGS. 115A-B depict screenshots of a series of behavioral instructions of a deep breathing module.

FIG. 116A depicts a flow chart illustrating an execution flow for a customization process of eyeball exercises, FIG. 116B depicts a flow chart illustrating six execution steps of the customization process of eyeball exercises, and FIG. 116C depicts a flow chart illustrating an execution flow for each execution step of the customization process of eyeball exercises.

FIGS. 117A-B depict examples of user interfaces for a customization process of eyeball exercises provided by a digital application of the present disclosure.

FIG. 118 is a flow chart illustrating an execution flow for a customization process of eyeball exercises when at least one eyeball movement is not recognized.

FIG. 119A-B depict examples of user interfaces for a customization process of eyeball exercises provided by a digital application of the present disclosure.

FIGS. 120A-E show execution screens according to a different type of game instructing the subject to perform different eye movements.

FIG. 121A is a photograph illustrating operating state of the subject's eyes due to a game that induces vertical or horizontal movement of the eyes.

FIG. 121B and FIG. 121C illustrate graphs showing the average and maximum transactions of a subject's effective eye movements according to the guidance of the game illustrated in FIGS. 120A-E.

FIGS. 122A-B, FIGS. 123A-B, FIGS. 124A-B and FIGS. 125A-B are graphs showing the results of a clinical trial to confirm the correlation between the motor performance pattern and myopia progression of a subject or group of subjects of the digital therapy in accordance with the present disclosure.

While the above-identified drawings set forth presently disclosed embodiments, other embodiments are also contemplated, as noted in the discussion. This disclosure presents illustrative embodiments by way of representation and not limitation. Numerous other modifications and embodiments may be devised by those skilled in the art which fall within the scope and spirit of the principles of the presently disclosed embodiments.

### DETAILED DESCRIPTION

Hereinafter, exemplary embodiments of the present disclosure will be described in detail. However, the present disclosure is not limited to the embodiments disclosed below, but may be implemented in various forms. The following embodiments are described in order to enable those of ordinary skill in the art to embody and practice embodiments of the present disclosure.

### Definitions

Although the terms first, second, etc. may be used to describe various elements, these elements are not limited by these terms. These terms are only used to distinguish one element from another. For example, a first element could be termed a second element, and, similarly, a second element could be termed a first element, without departing from the scope of exemplary embodiments. The term “and/or” includes any and all combinations of one or more of the associated listed items.

The terminology used herein is for the purpose of describing particular embodiments only and is not intended to be limiting of exemplary embodiments. The singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements, components and/or groups thereof, but do not preclude the presence or addition of one or more other features, integers, steps, operations, elements, components and/or groups thereof.

As used herein, the term “about” generally refers to a particular numeric value that is within an acceptable error range as determined by one of ordinary skill in the art, which will depend in part on how the numeric value is measured or determined, i.e., the limitations of the measurement system. For example, “about” may mean a range of +20%, +10%, or +5% of a given numeric value.

### Overview

With reference to the appended drawings, exemplary embodiments of the present disclosure will be described in detail below. To aid in understanding the present disclosure, like numbers refer to like elements throughout the description of the figures, and the description of the same elements will be not reiterated.

In the prior art, the development of new drugs starts with confirming a medial demand in situ, proposing a mechanism of action based on the expert reviews and meta-analysis on the corresponding disease, and deducing a therapeutic hypothesis based on the expert reviews and the meta-analysis. Also, after a library of drugs whose therapeutic effects are expected is prepared based on the therapeutic hypothesis, a candidate material is found through screening, and the corresponding candidate material is subjected to optimization and preclinical trials to check its effectiveness and safety from a preclinical stage, thereby deciding the candidate material as a final candidate drug. To mass-produce the corresponding candidate drug, a CMC (chemistry, manufacturing, and control) process is also established, a clinical trial is carried out on the corresponding candidate drug to verify a mechanism of action and a therapeutic hypothesis of the candidate drug, thereby ensuring the clinical effectiveness and safety of the candidate drug.

From the point of view of this patent, drug targeting and signaling, which fall upstream of the development of new drugs, have many uncertainties. In many cases, because the drug targeting and signaling take a methodology of putting together the outcomes, which have been reported in the art, and interpreting the outcomes, it may be difficult to guarantee the novelty of disclosure. On the contrary, the disclosure of drugs capable of regulating the drug targeting and signaling to treat a disease requires the highest level of creativity except for the field of some antibody or nucleic acid (DNA, RNA) therapeutics in spite of the development of research methodology for research and development of numerous new drugs. As a result, the molecular structures of the drugs are the most critical factors that constitute the most potent substance patent in the field of new drugs.

Unlike the drugs whose rights are strongly protected through this substance patent, digital therapeutics are basically realized using software. Due to the nature of the digital therapeutics, the rational design of digital therapeutics against the corresponding disease, and the software realization of the digital therapeutics based on the rational design may be considered to be a very creative process of disclosure to be protected as a patent when considering the clinical verification and approval processes as the therapeutics.

That is, the core of the digital therapeutics as in the present disclosure depends on the rational design of digital therapeutics suitable for treatment of the corresponding disease, and the development of specific software capable of clinically verifying the digital therapeutics based on the rational design. Hereinafter, a digital apparatus and an application for treating myopia according to the present disclosure realized in this aspect will be described in detail.

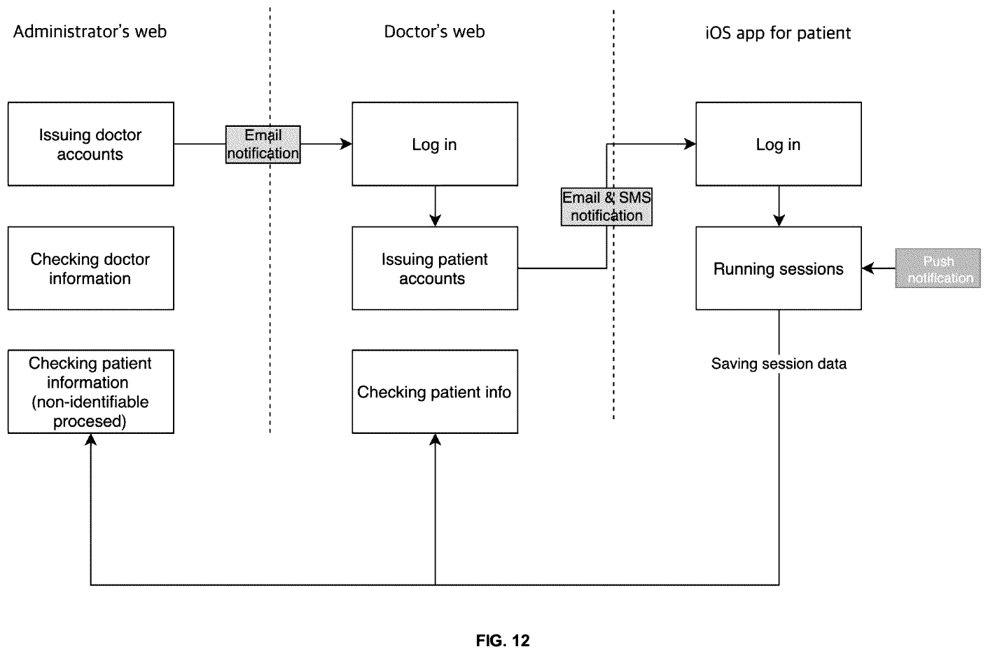

In certain aspects, the present disclosure provides a system for treating myopia. In some embodiments, the system comprises a digital apparatus configured to execute a digital application for treating myopia in a subject. In some embodiments, the system comprises a healthcare provider portal configured to provide one or more options to a healthcare provider to perform one or more tasks to prescribe treatment for the myopia in the subject based on information received from the digital application. In some embodiments, the system comprises an administrative portal configured to provide one or more options to an administrator of the system to perform one or more tasks to manage access to the system by the healthcare provider. FIG. 12 depicts a flow chart illustrating a system for treating myopia, the system comprising an administrative portal (e.g., Administrator's web), a healthcare provider portal (e.g., Doctor's web) and a digital apparatus configured to execute a digital application (e.g., an application or ‘app’) for treating myopia in a subject. Among other things, the Administrator's portal allows an administrator to issue doctor accounts, review doctor information, and review de-identified patient information. Among other things, the Healthcare Provider's portal allows a healthcare provider (e.g., a doctor) to issue patient accounts, and review patient information (e.g., age, prescription information, and status for having completed one or more digital therapeutic modules or sessions). Among other things, the digital application allows a patent access to complete one or more digital therapeutic modules or sessions.

The healthcare provider portal may be accessible via a client device (e.g., a personal device) such as a laptop computer, a smart phone, a tablet, or other computing device. The administrator's portal is configured to provide services related to the digital application and the healthcare provider portal (e.g., front end and/or back end services), and may be in communication with one or more databases for storing information related to the digital application and the healthcare provider portal, such as patient profile information and/or patient behavior information obtained via the one or more sensors of the digital apparatus.

In some implementations, the system for treating myopia is implemented by a network, which transmits the encrypted information to the terminals of the digital application, the healthcare provider portal, and the administrator's portal.

The software configuration of the system for treating myopia according to some implementations of the present invention can be implemented as an integrated application connecting the digital application, the healthcare provider portal, and the administrative portal through a network. This integrated application provides compatibility for input/output with various external sensors from a system perspective, an environment required for the operation of interfaces in various computers or mobiles of the patient and the doctor, and security solutions for legal management of related information.

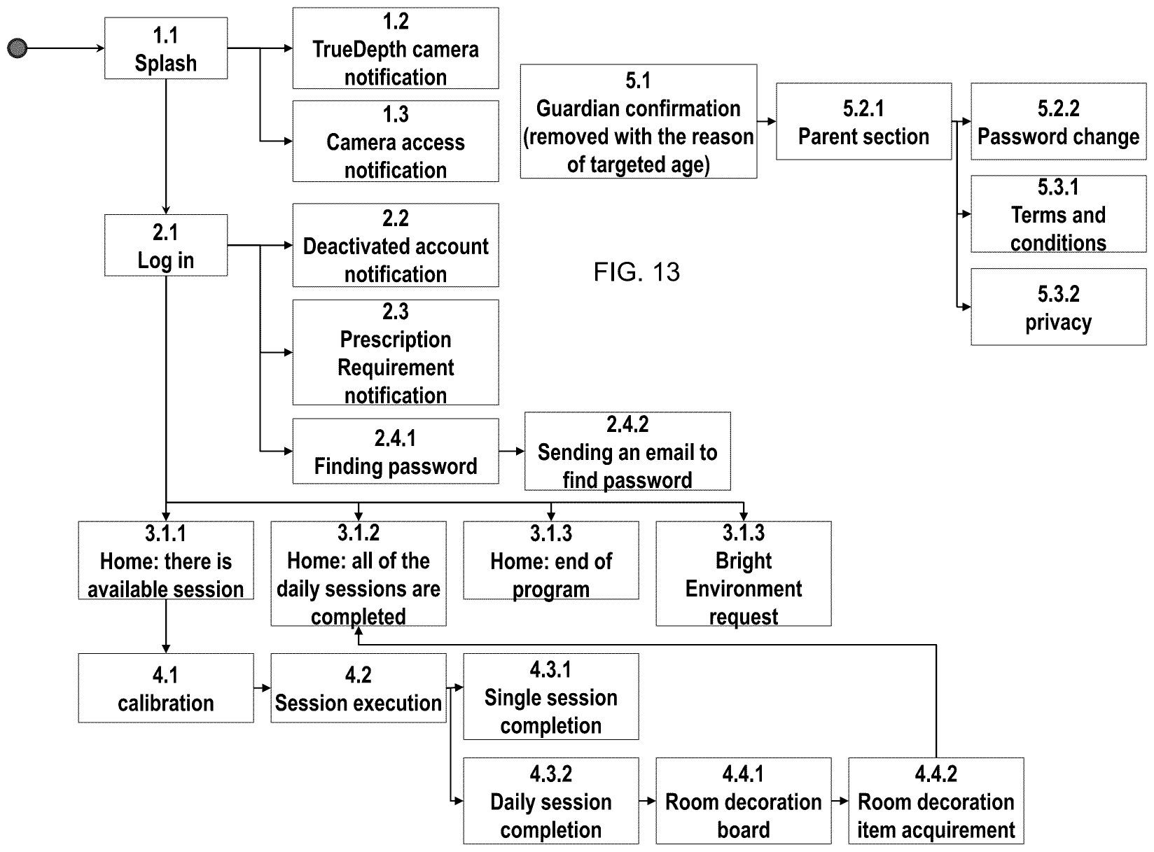

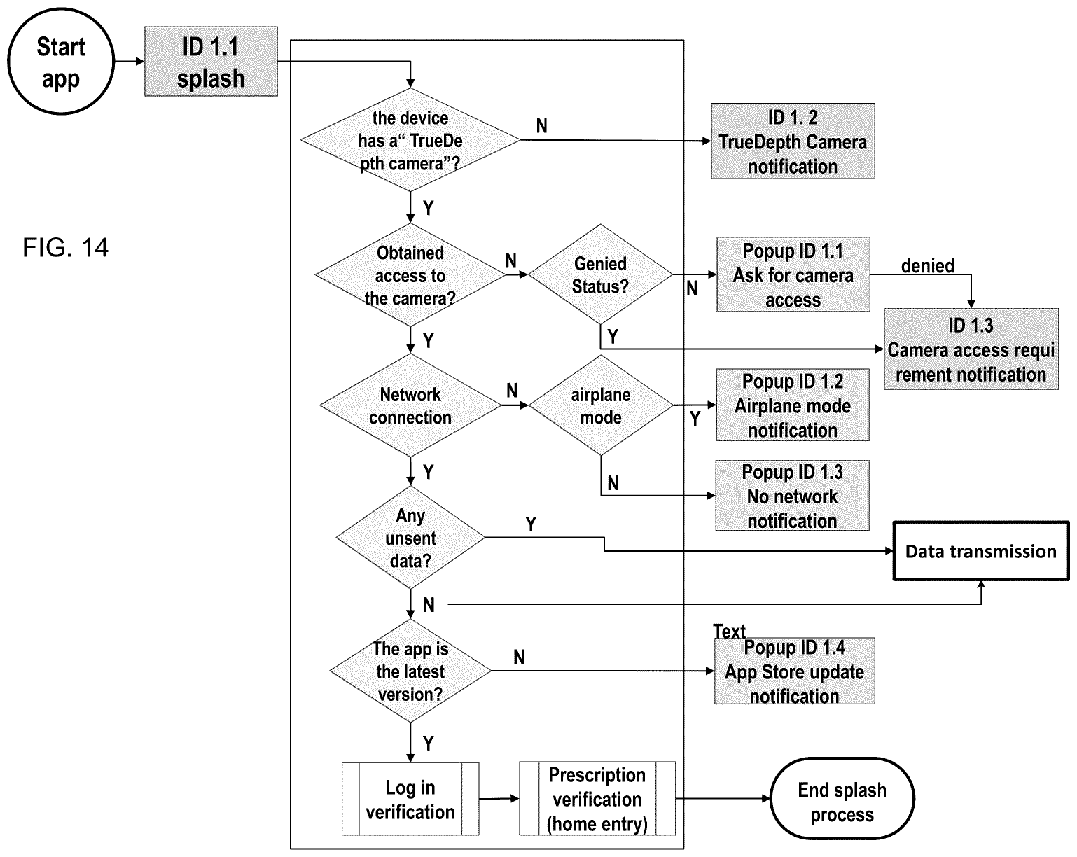

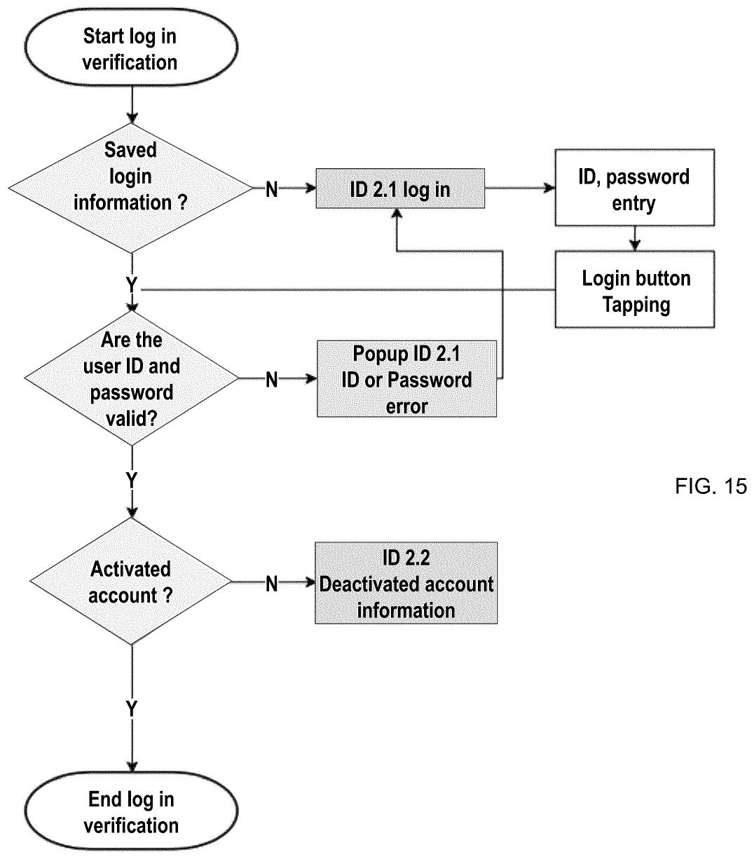

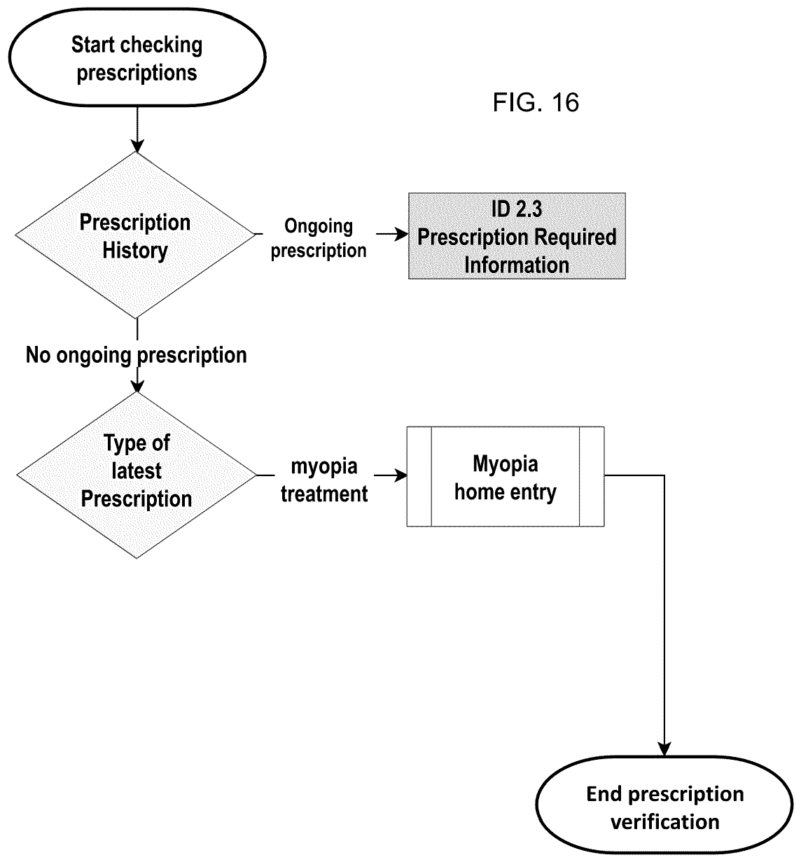

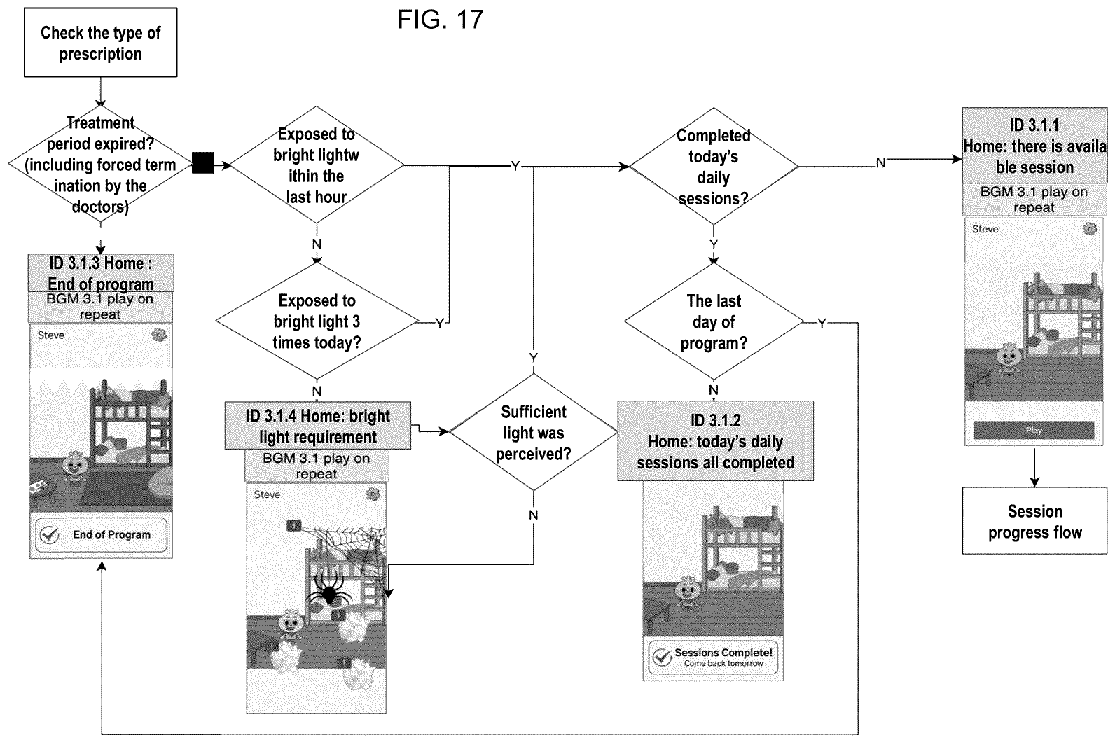

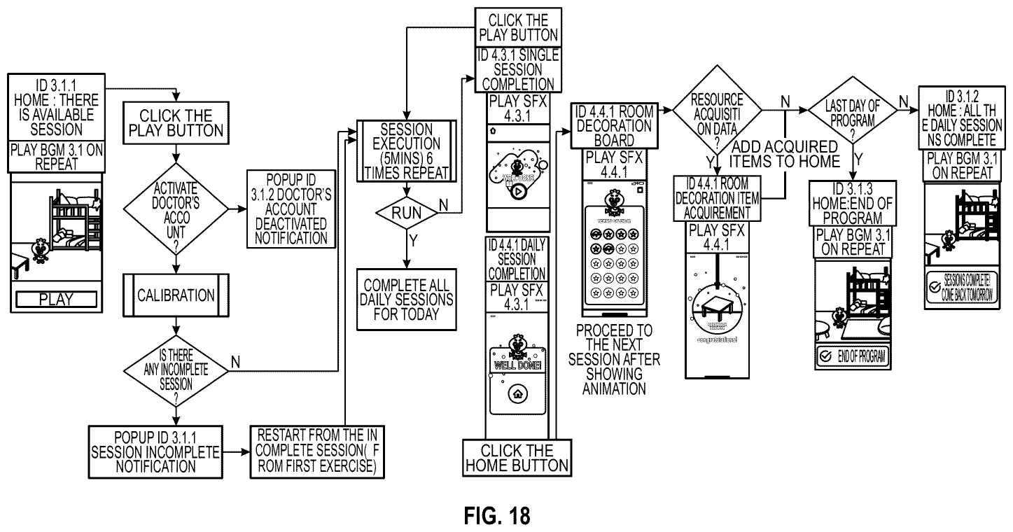

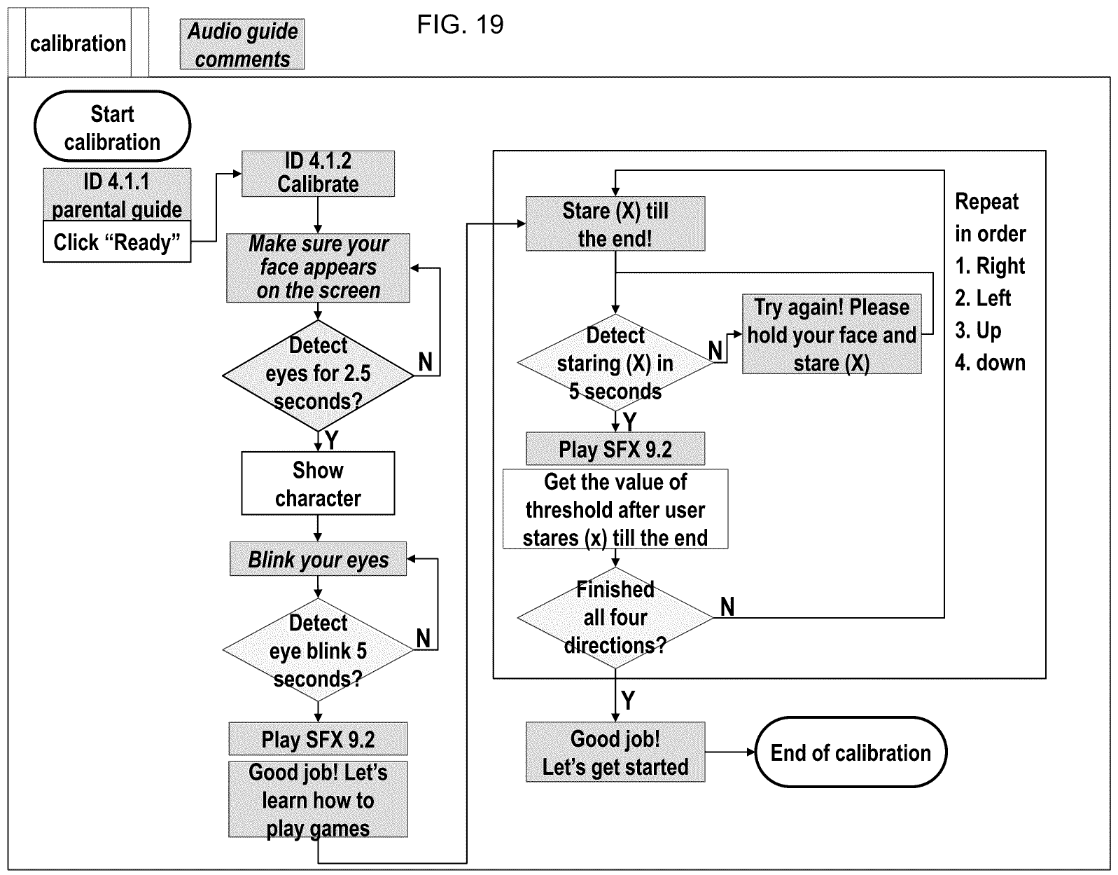

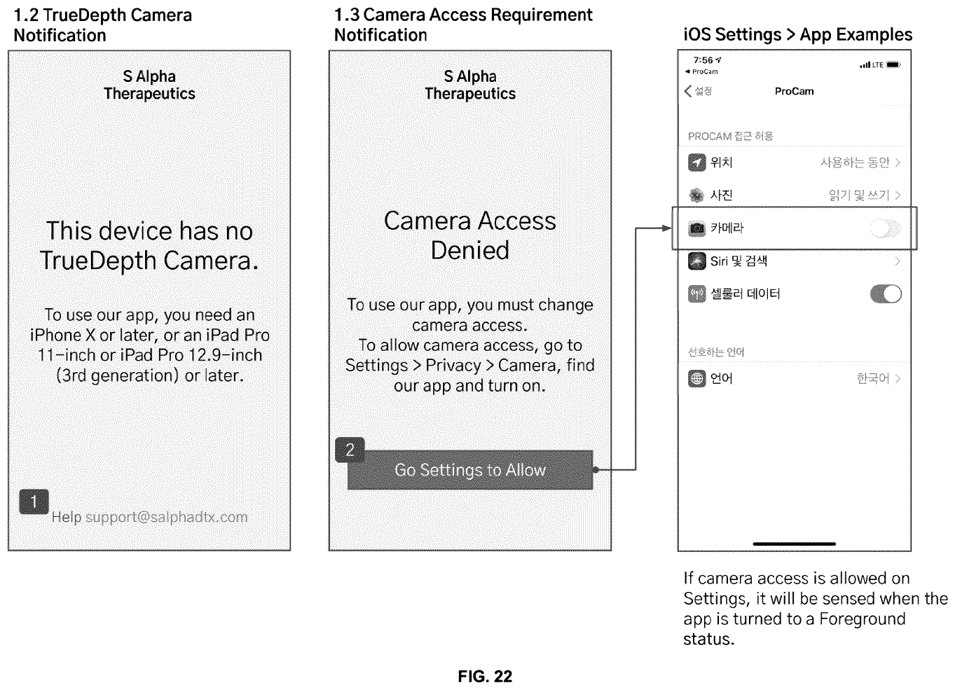

FIG. 13 depicts a flow chart illustrating an execution flow for the digital application. Upon opening the digital application, a splash screen is displayed, followed by a request for log in information, as well as a verification of prescription information for the subject. FIG. 14 depicts a flow chart illustrating an execution flow for a splash process at the starting of the digital application. The splash process may comprise detecting whether the digital apparatus comprises a TrueDepth camera, detecting whether the digital application has access to the camera, detecting whether the digital application has a network connection, whether the digital application has been updated to the latest version, login verification, and prescription verification. FIG. 15 depicts a flow chart illustrating an execution flow for login verification during a splash process at the starting of the digital application. Similarly, FIG. 16 depicts a flow chart illustrating an execution flow for prescription verification during a splash process at the starting of the digital application. The prescription verification process may comprise, for example, determining if the treatment period has expired, determining whether the subject has been recently (e.g., within the last hour) been exposed to bright light), determining if, based on the prescription, the subject's sessions for the day have been completed (e.g., the subject is compliant with the prescription). In such instances, the digital apparatus may notify the subject that there are no sessions available to be completed, and/or expose the subject to a light therapy module prior to beginning any digital therapeutic modules. FIG. 21 depicts a splash screen of a digital application of the present disclosure, wherein the splash screen comprises a logo (labeled 1), loading icon (labeled 2), and/or information on the version of the digital application (labeled 3). The splash entry process checks the network, version, login verification and others based on the execution flow. If there are data which had not been sent due to forced app termination, network errors, etc., the data are checked and sent during splash entry. During the splash entry process, the loading icon is shown when the process takes too long. In certain embodiments, appropriate pop-ups are shown for different situations of application execution flow. The splash entry process may also include camera detection. FIG. 22 depicts TrueDepth Camera Notification Screens of a digital application of the present disclosure. In devices that do not support TrueDepth Camera, eye exercises cannot be conducted, and thus prevent further application execution from appearing on the screen. The camera (also referred to as a sensor, a depth sensor, or a range sensor) can generate depth data indicating distance to points in a surrounding. In some embodiments, the digital apparatus includes (i) a camera that faces a user (e.g., to obtain facial data about the user's face, such as the location of the eyeball, hand data about the user's hand, or other data about other parts of the user's body) and/or (i) a camera that faces a user's environment (e.g., to obtain location data about the user's physical environment, such as the location of a light). In certain embodiments, the camera comprises a three dimensional camera system, such as the TrueDepth® camera system manufactured by Apple, Inc., Cupertino, Calif. (USA). In another embodiment, the camera comprises a time-of-flight (ToF) camera that measures the time-of-flight of a light signal between the ToF camera and a target in an environment (e.g., the eyeball of the subject). In yet another embodiment, the camera comprises a structured-light 3D scanner (e.g., an infrared emitter and infrared camera) to implement a structured-light technique that projects a known pattern (e.g., structured light) onto a surface and captures image(s). A person of skill in the art will appreciate that the camera can implement other techniques, such as sheet of light triangulation, stereo triangulation, interferometry, and the like. As non-limiting examples, the camera can implement techniques and/or components used by a RealSense® camera from Intel®, a Hololens® from Microsoft®, a TrueDepth® camera from Apple®, a Tango® system from Google®, a Kinect® system from Microsoft®, and the like. In some embodiments, the camera is configured to capture an image. In some embodiments, the camera is configured to generate depth data, such as a range image, depth map, or the like. The depth data may indicate one or more distances to one or more points (e.g., the distance moved by an eyeball over a given interval of time) represented in the depth data, respectively. In some examples, depth data may be used to identify distances to points in an environment, identify an object or surface in the environment, determine distance travelled by an object over a given interval, and/or position and/or maintain a representation of user or other content in relation to the object or the surface as the digital apparatus moves within the environment (e.g., in an AR or VR implementation).

A Mailto link (labeled 1) may be included to help the subject send mail to a support team. Camera access may be allowed/disallowed (e.g., turned on/off) by the user whenever he/she wants. Camera access status is checked every time the app launches, and if the access is disallowed (off), a screen indicating that camera access is denied is displayed and the application is prevented from further execution. A button (labeled 2) may also be displayed to assist the subject in jumping to the settings panel of the digital apparatus to adjust the settings (e.g., allow camera access).

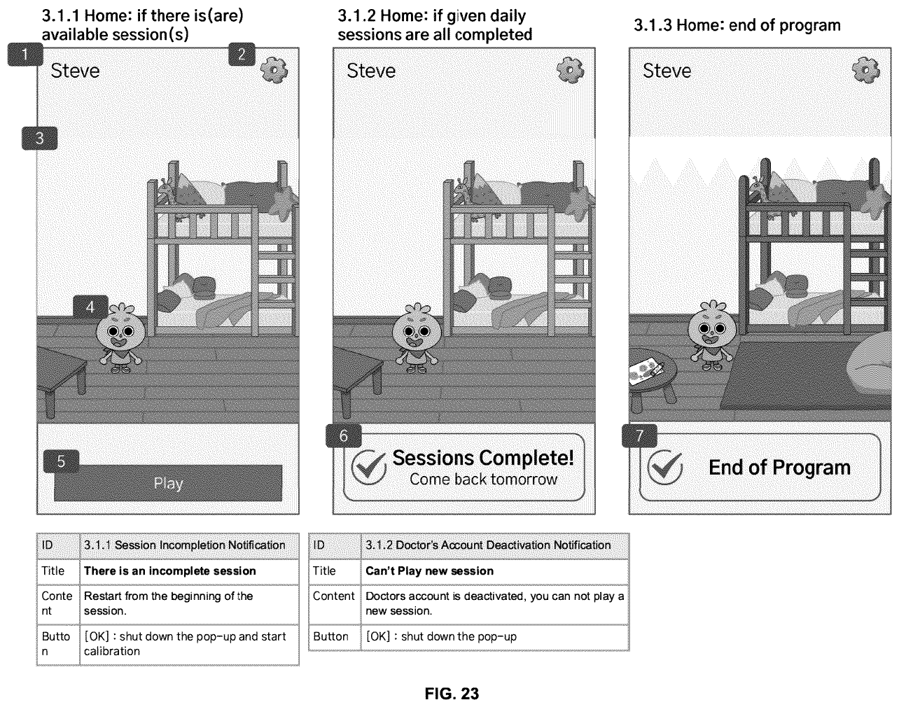

As described above, the availability of sessions may be determined during the prescription verification process. FIG. 23 depicts Home screens of the digital application of the present disclosure, wherein the Home screen indicates the availability of sessions for a subject to complete. As shown in FIG. 23, (1) Patient name: no tapping required, (2) Guardian mode entry button, (3) Room, which gets decorated as the treatment proceeds (decorations are automatically added or upgraded as the sessions are completed, and the decorations may display simple movements), (4) a character, which appears in the middle of the room, undergoes no changes during the treatment, and jumps up when tapped, (5) a Play button, (6) Notifications that the given daily sessions are completed, and (7) Notification of the end of the treatment program.

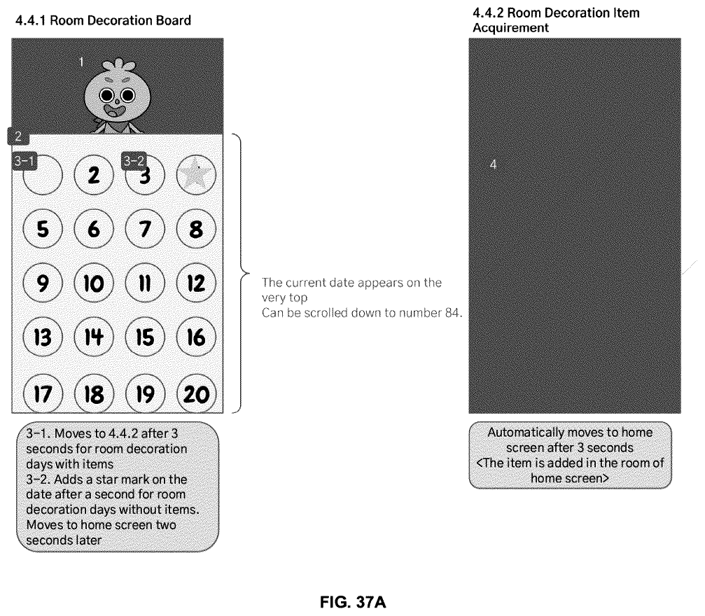

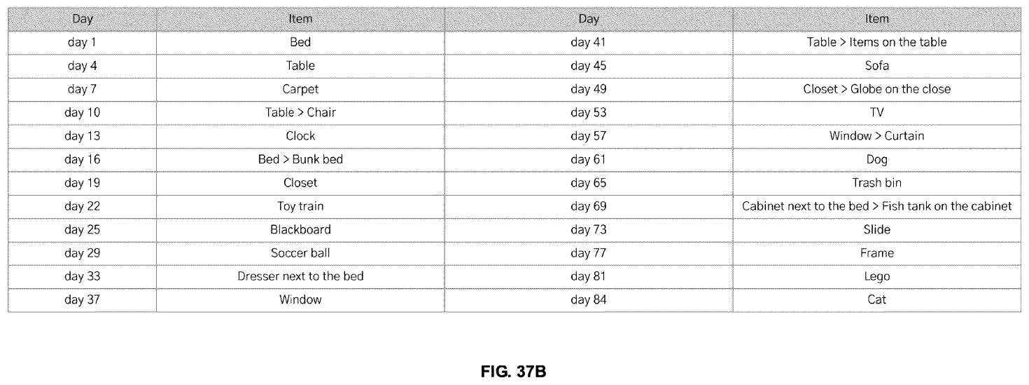

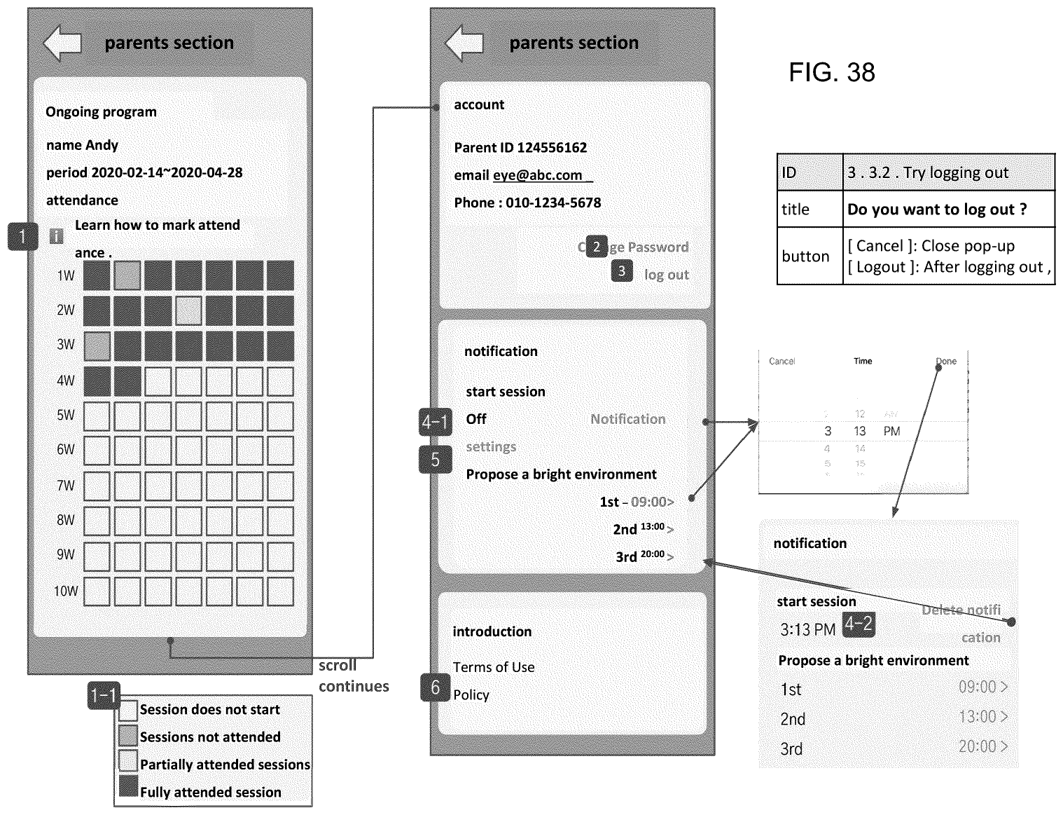

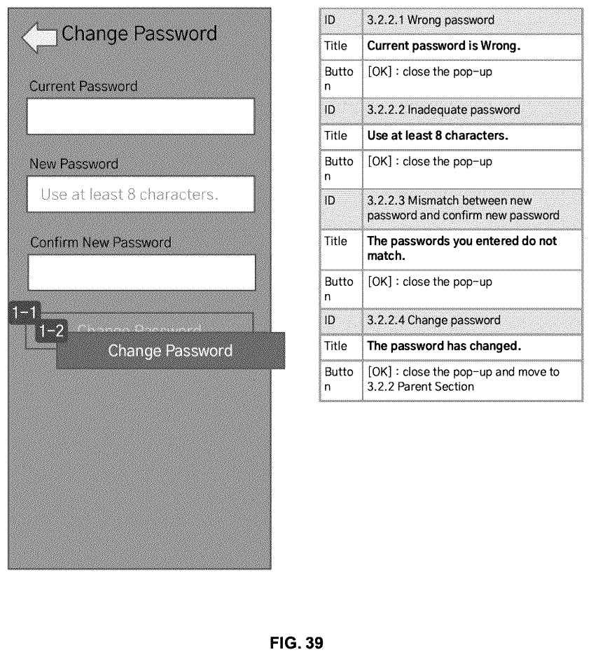

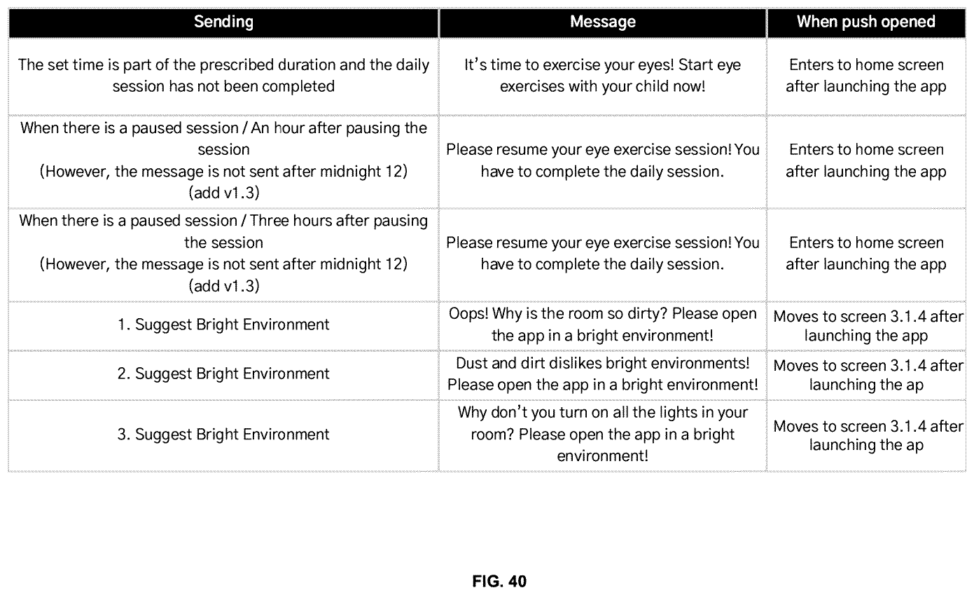

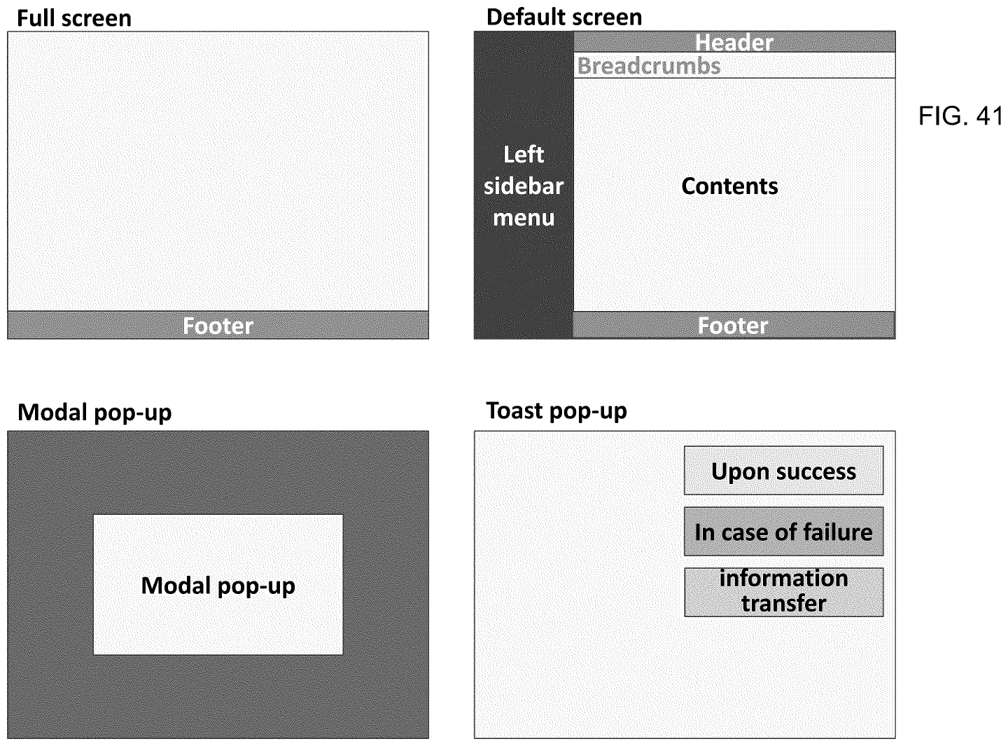

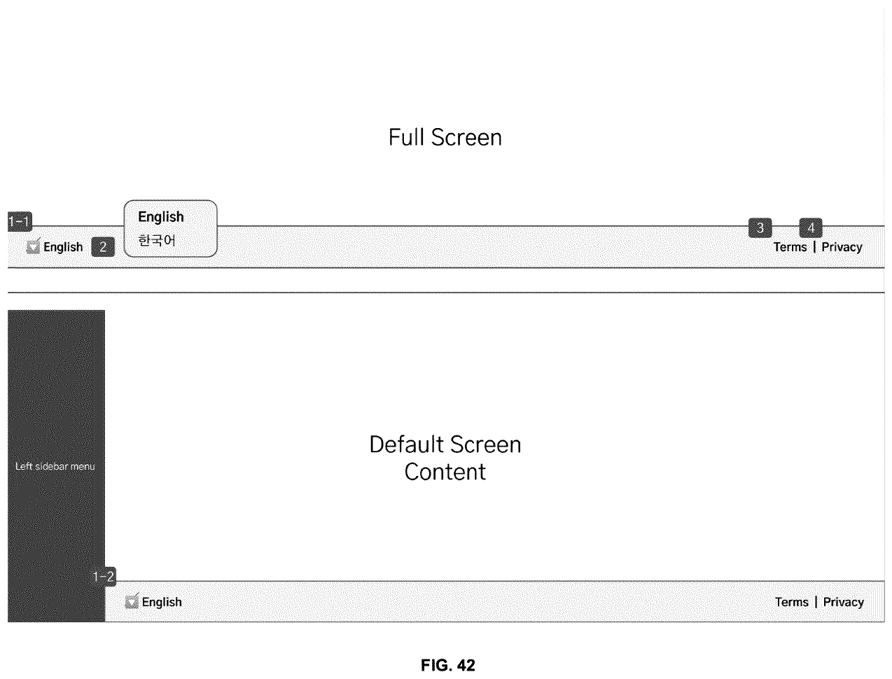

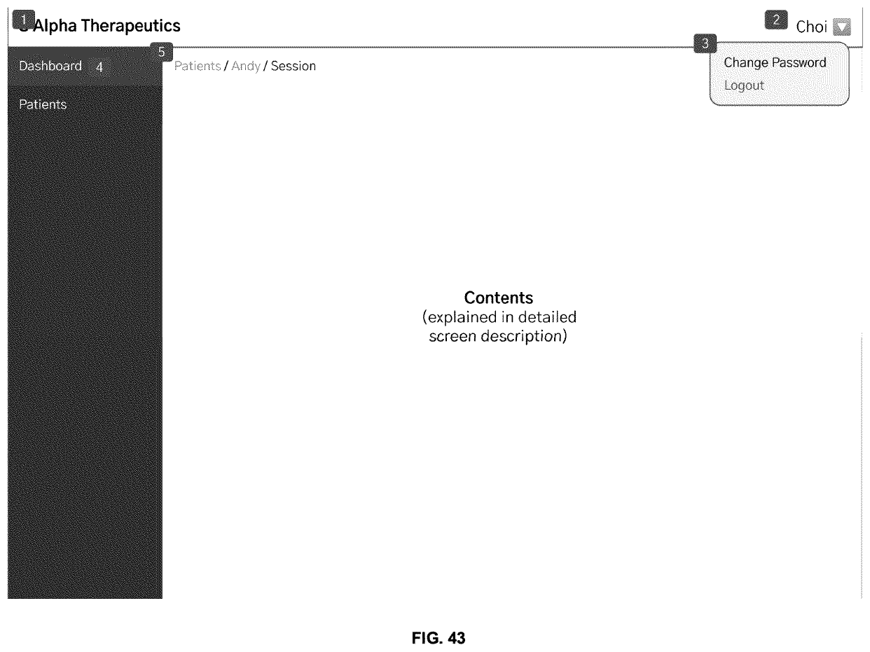

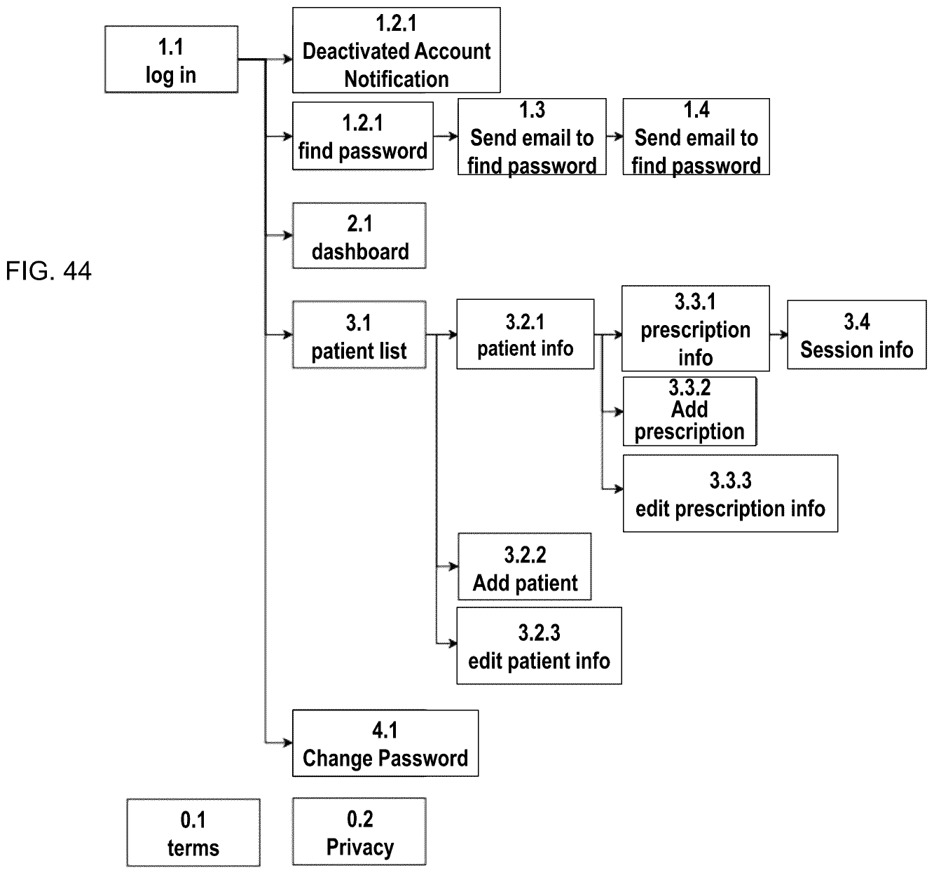

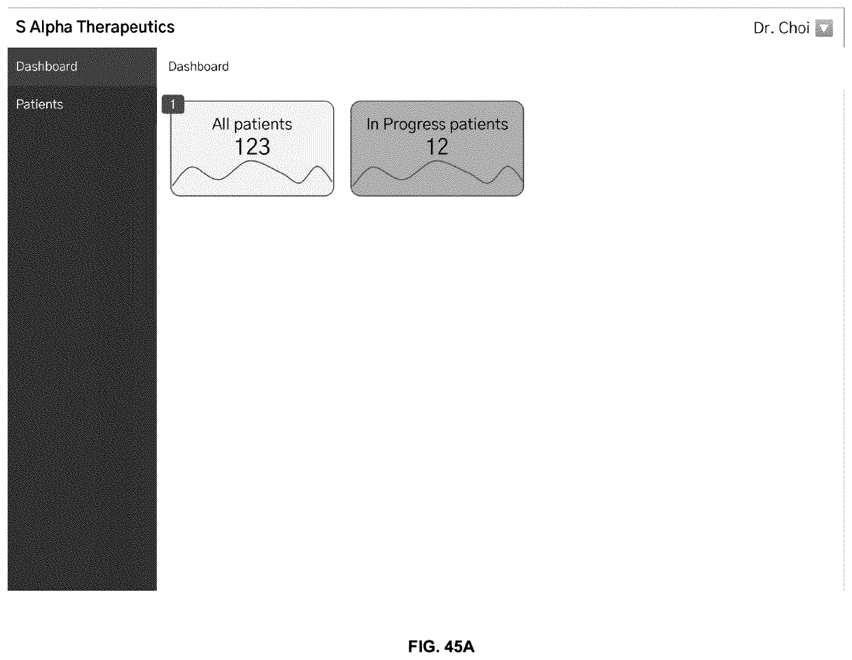

FIGS. 37A-B depict (A) a screenshot of a Room Decoration Board in an accomplishment module in a digital application of the present disclosure, and (B) a timeline showing the days on which a subject may acquire a given Room Decoration item. As shown, (1) a character, (2) a Room Decoration Board, where the numbers are presented like a calendar, and the Decoration Board maps a number or room decoration item for each date; the user cannot acquire the item on days with only numbers (3-2.) and vice versa on days with decoration items (3-1), and (4) shows items of 3-1 (room decoration items received) in close-up (e.g., a magnified view of the room decoration item(s) obtained).

In some embodiments, the digital application for treating myopia instructs a processor of the digital apparatus to execute operations comprising generating digital therapeutic modules for treating myopia based on a mechanism of action in and a therapeutic hypothesis for the myopia. In some embodiments, the digital therapeutic modules comprises generating the digital therapeutic modules based on neurohumoral factors related to the myopia onset.

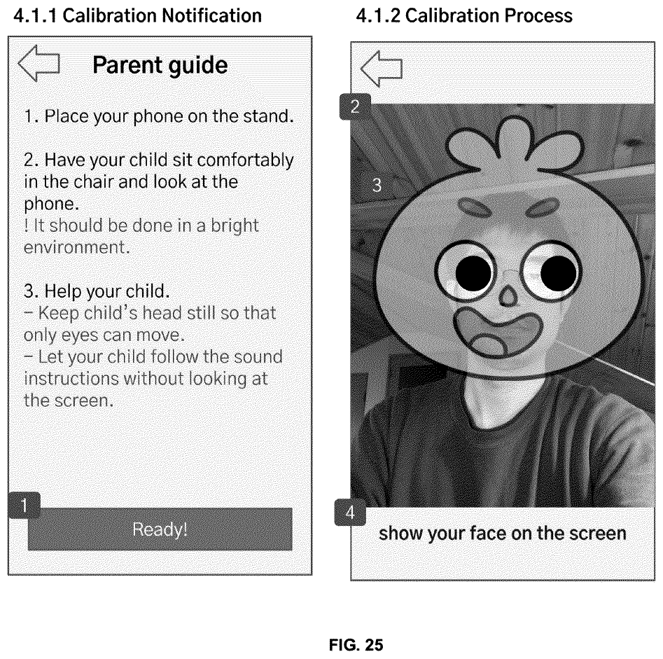

In some embodiments, the operations further comprise generating a calibration module for calibrating one or more of an accuracy of measurement of the subject's eye position, and a light environment. In some embodiments, the calibration module may be generated prior to generating the digital therapeutic modules. In some embodiments, the calibration module may not performed, and calibration settings from a previous session are used. Calibration may be performed at any time before, during, or after a session comprising two or more digital therapeutic modules. For example, calibration may precede the session. In another example, if the results from a digital therapeutic module exhibit large variability, the digital application may stop the session, and initiate calibration to confirm that the results of the digital therapeutic module are true, and not a result of poor calibration. FIG. 19 depicts a flow chart illustrating an execution flow for a calibration module in the digital application. Calibration may proceed at the start of the session each day for accuracy of eye measurement. Calibration may take between 35 and 60 seconds depending on the execution of outcomes. As shown in FIG. 19, the calibration module may comprise a series of instructions from the digital application to the subject such as orienting the subjects face in a particular direction (e.g., for better detection of the subject's eye(s)), or blinking the subject's eyes. FIG. 25 depicts a Calibration Notification screen of the digital application, wherein the Calibration Notification screen indicates whether a subject's eye and/or movement of the eye are detectable by the camera. As shown in FIG. 25, (1) a ready button, (2) a display of the Front Camera view on screen, (3) a character that is only shown when the digital application perceives the pupil (the character may be a 2D character with big eyes, which copy the user's eye movements, and may appear in a semi-transparent manner so that the user's face may be seen), and (4) a notice that provides action guides for user's guardian.

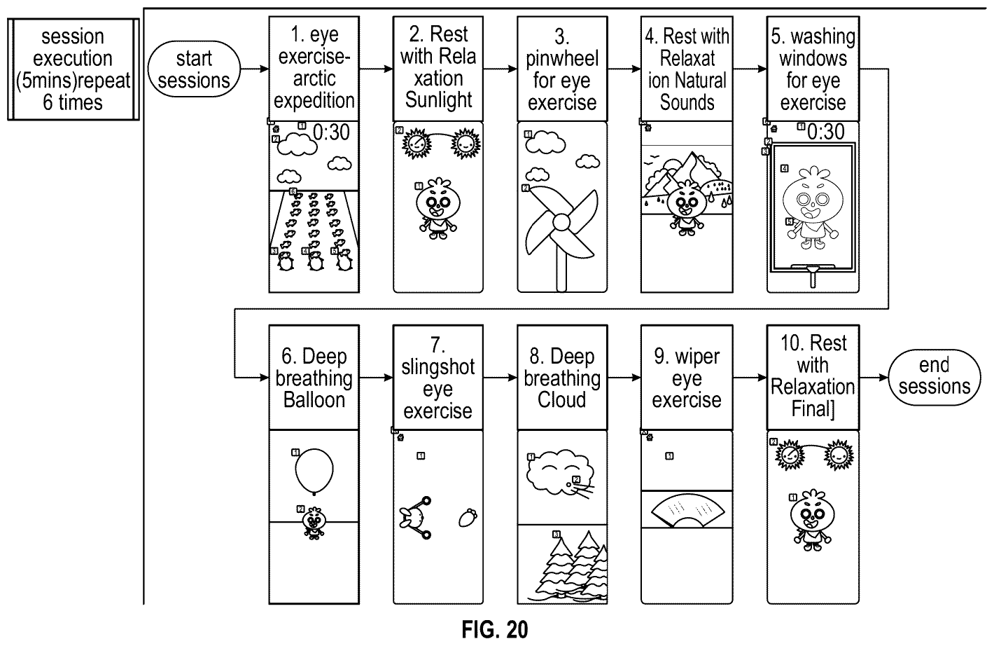

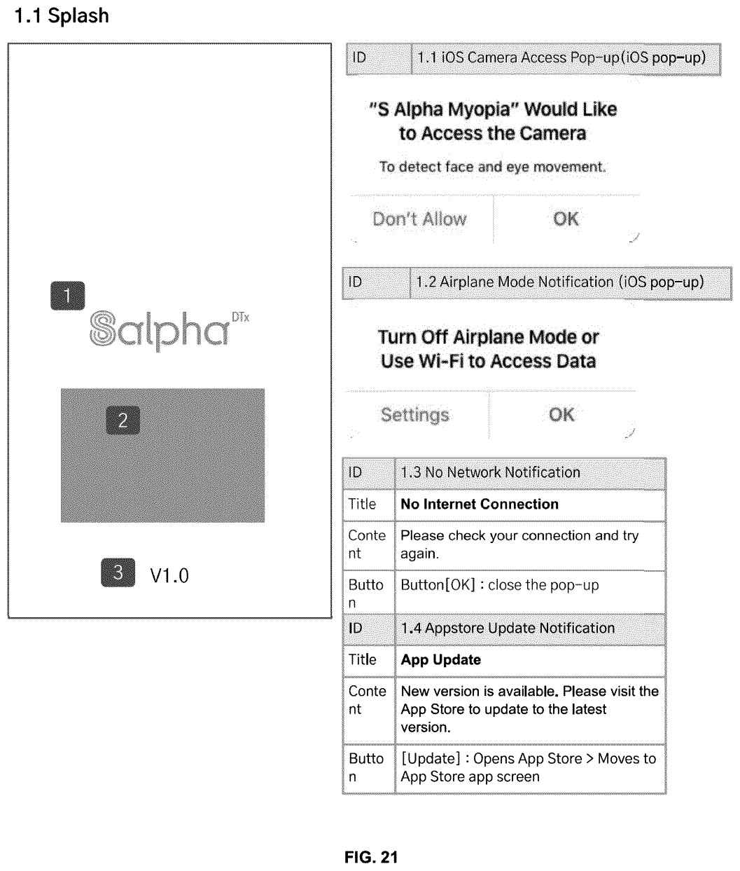

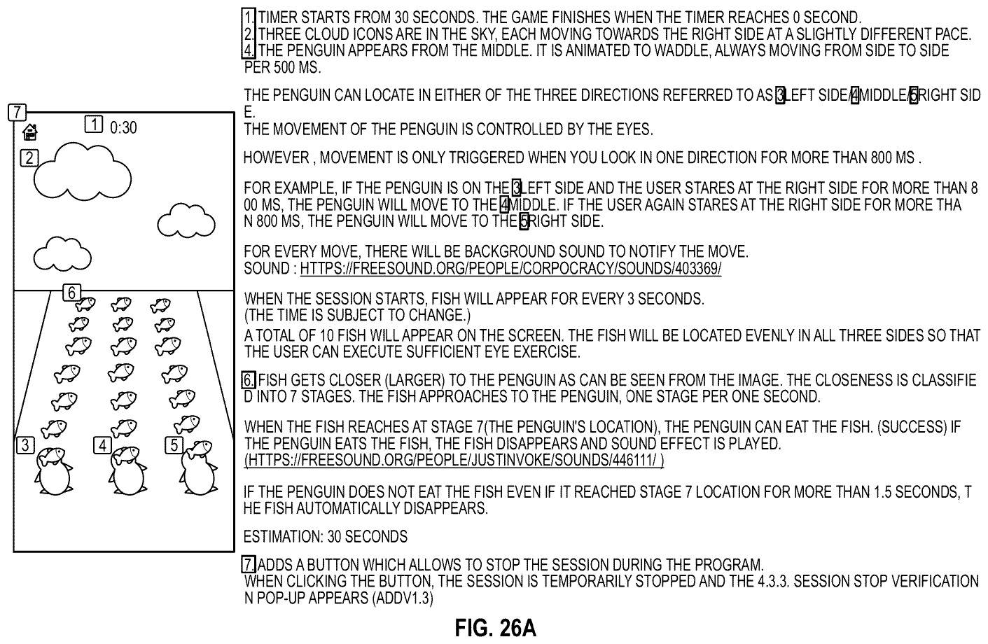

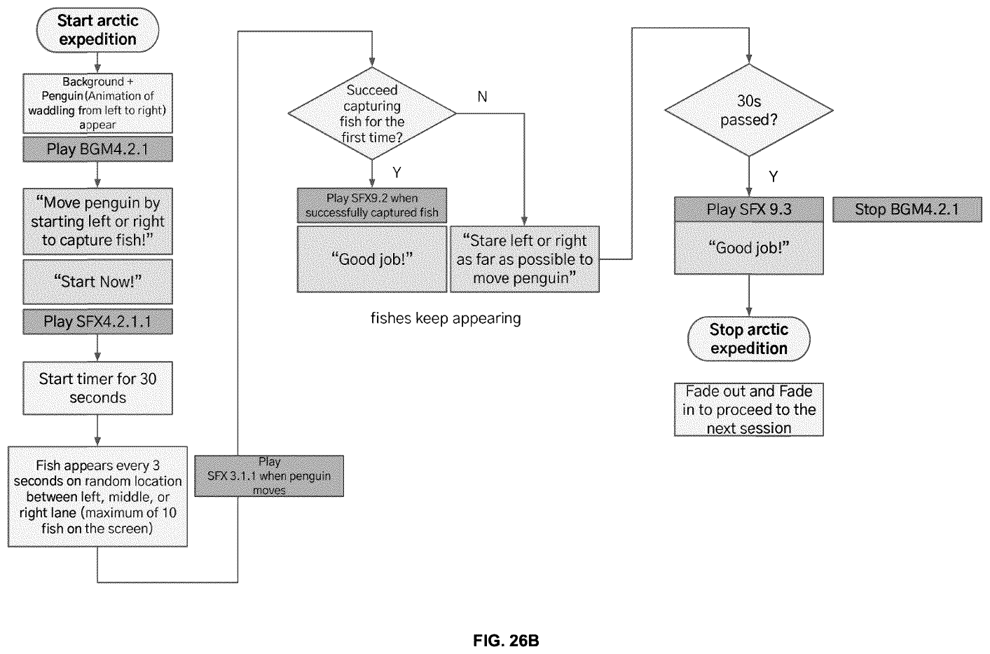

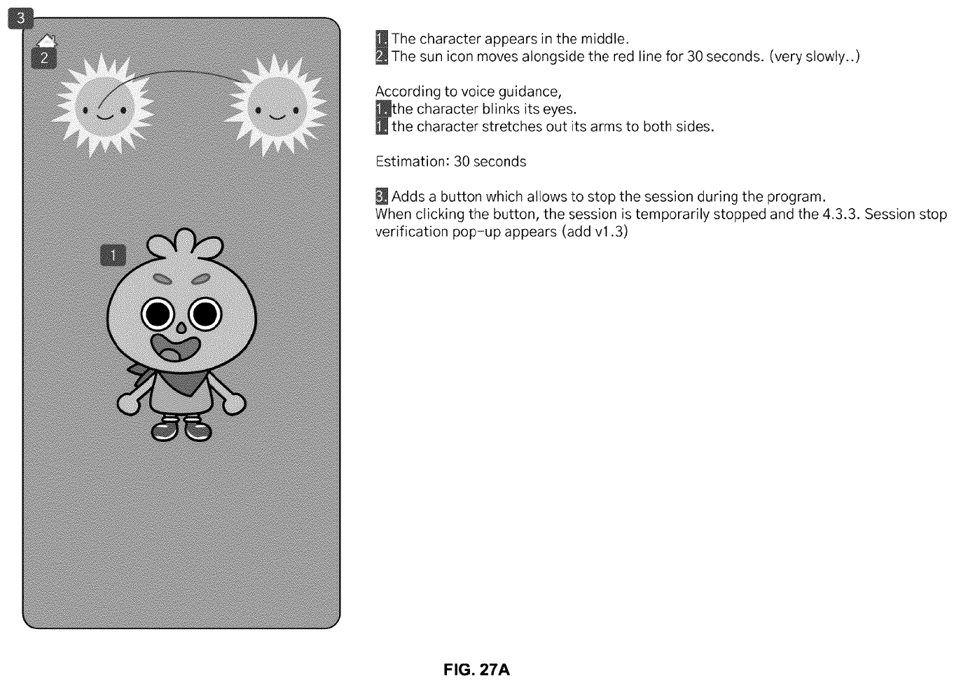

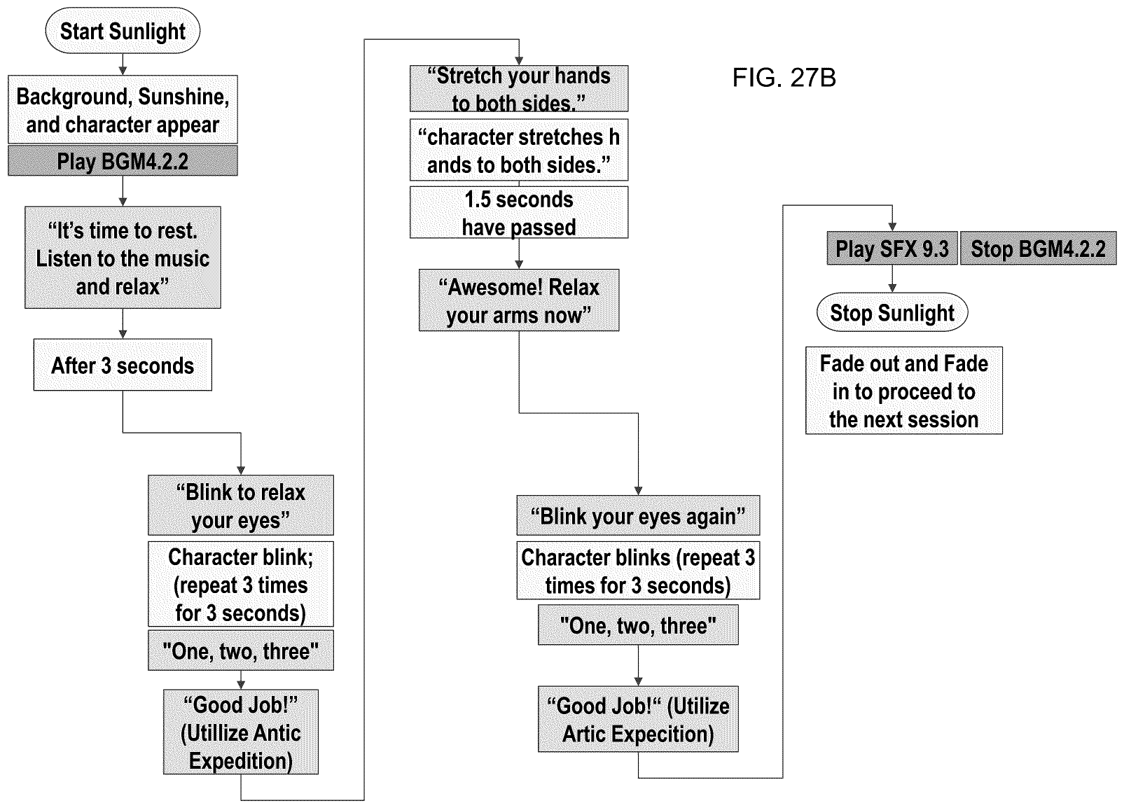

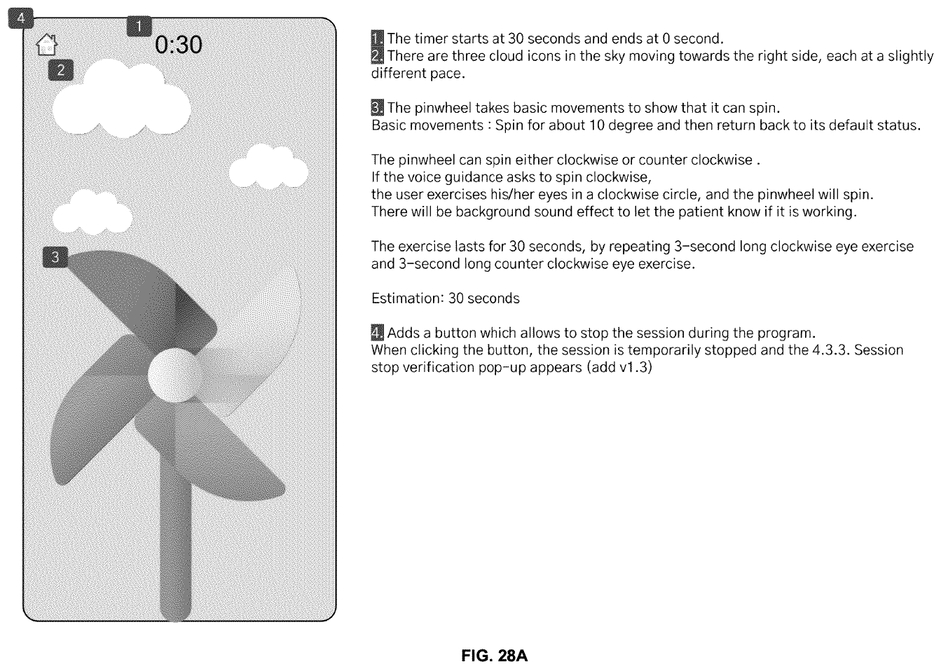

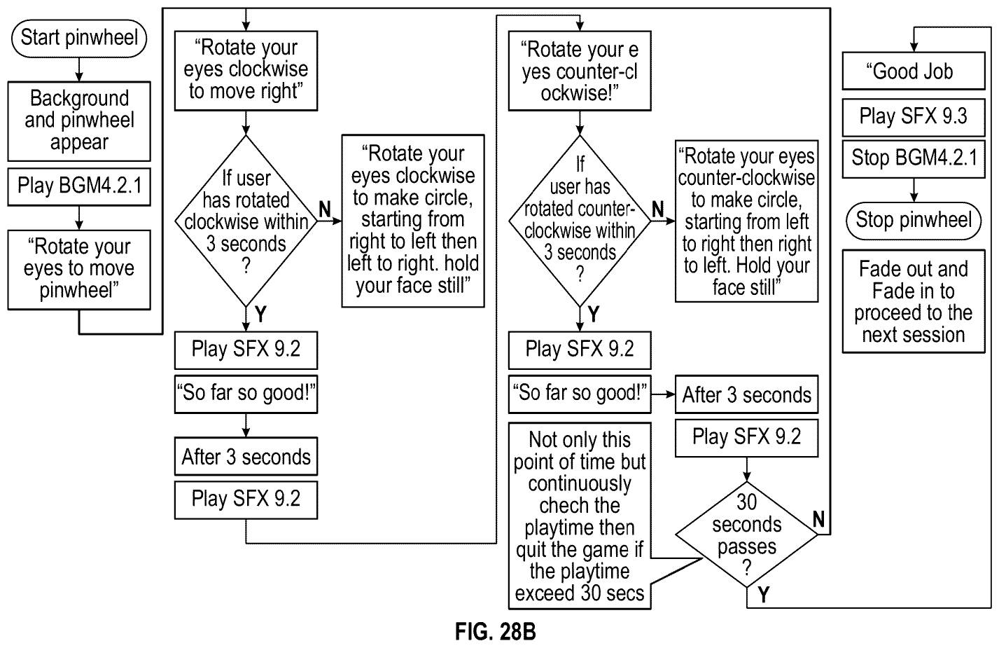

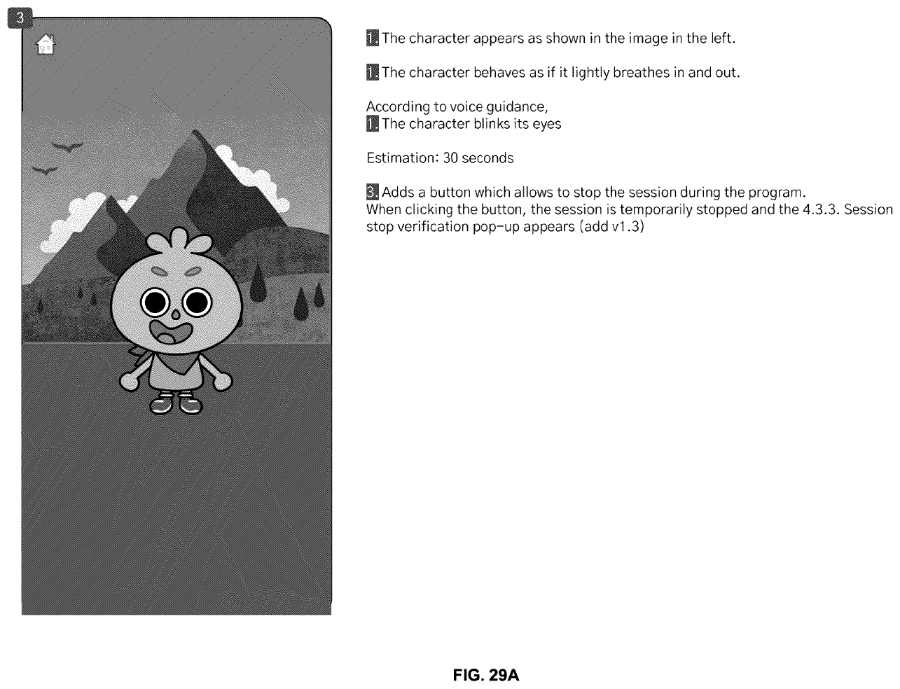

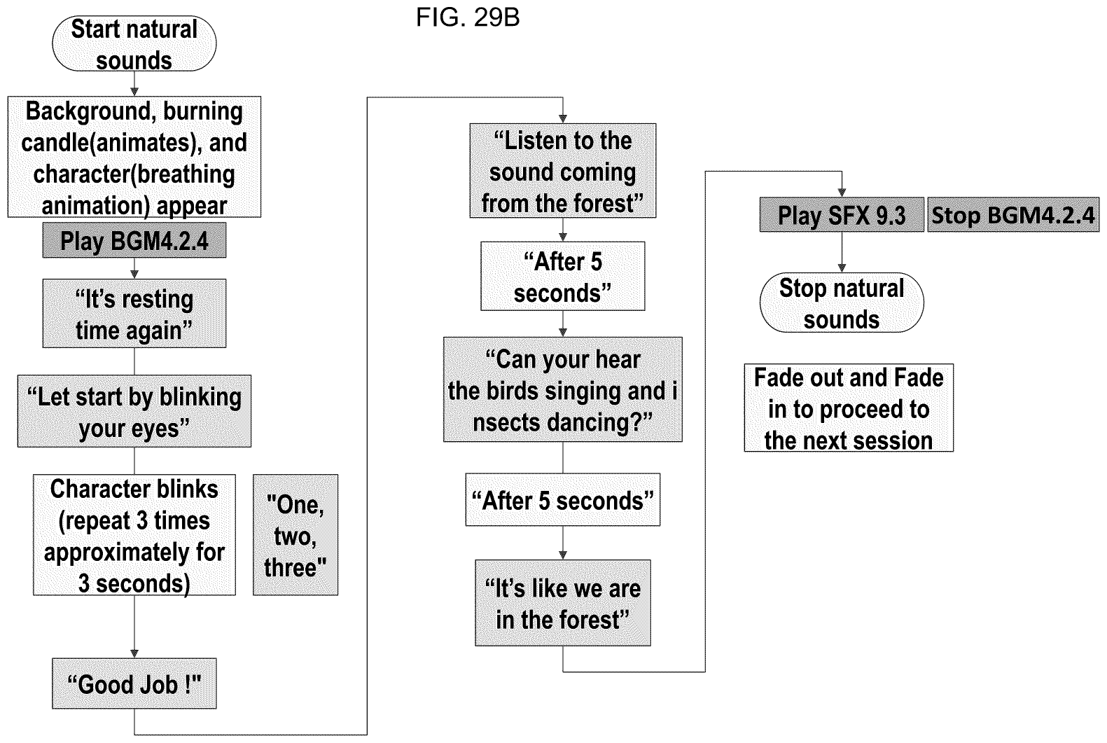

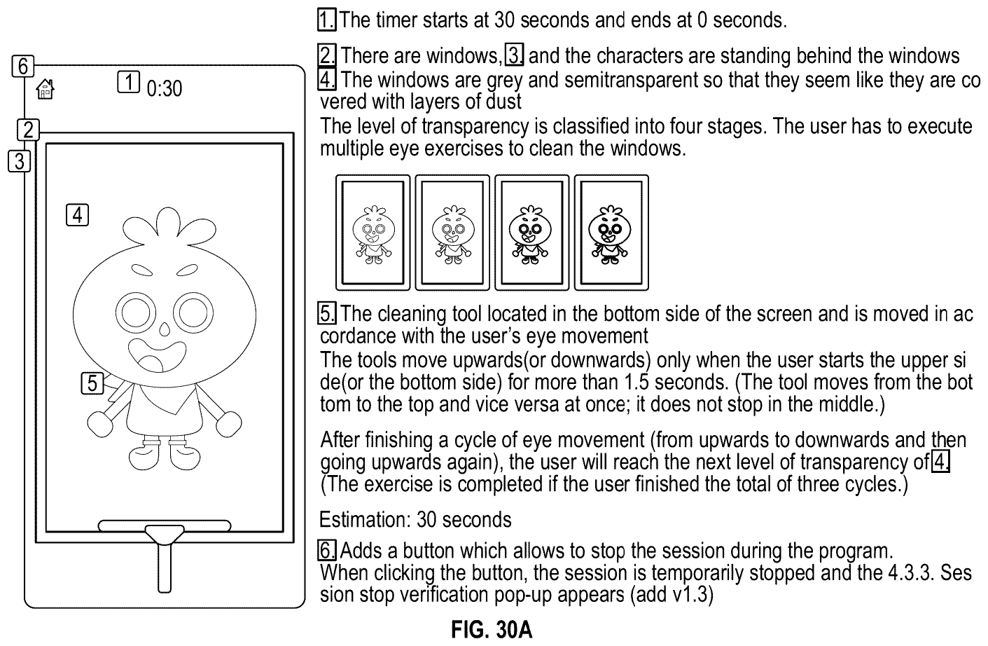

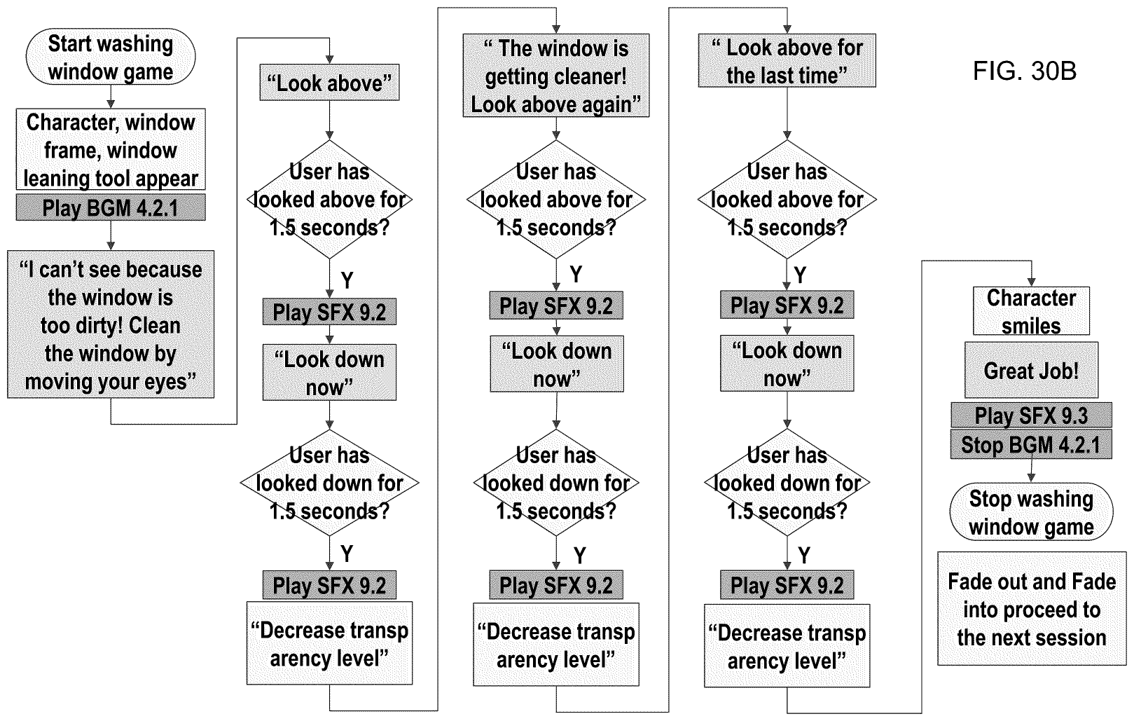

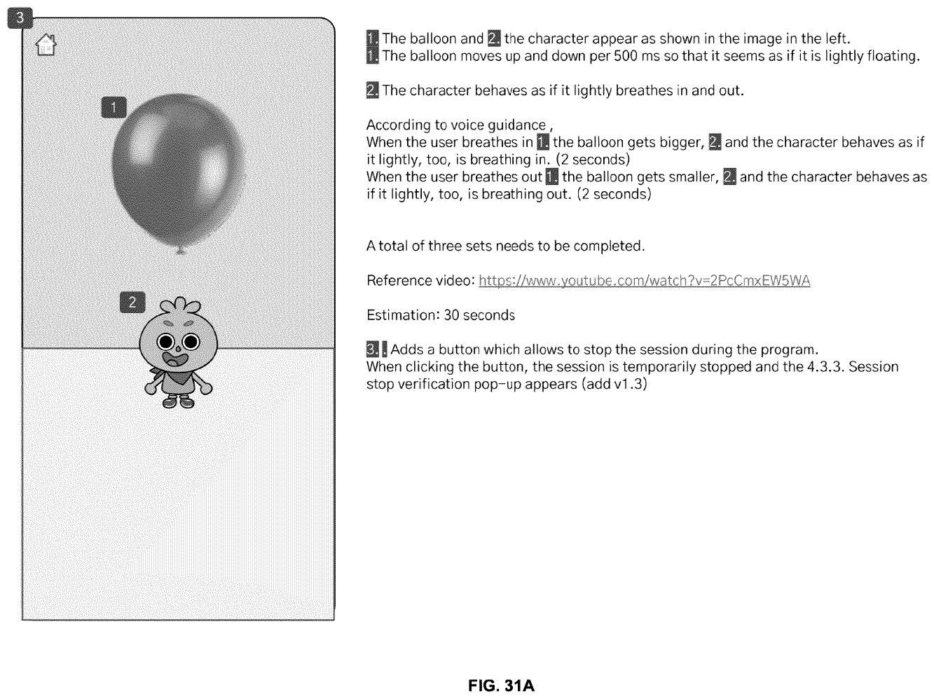

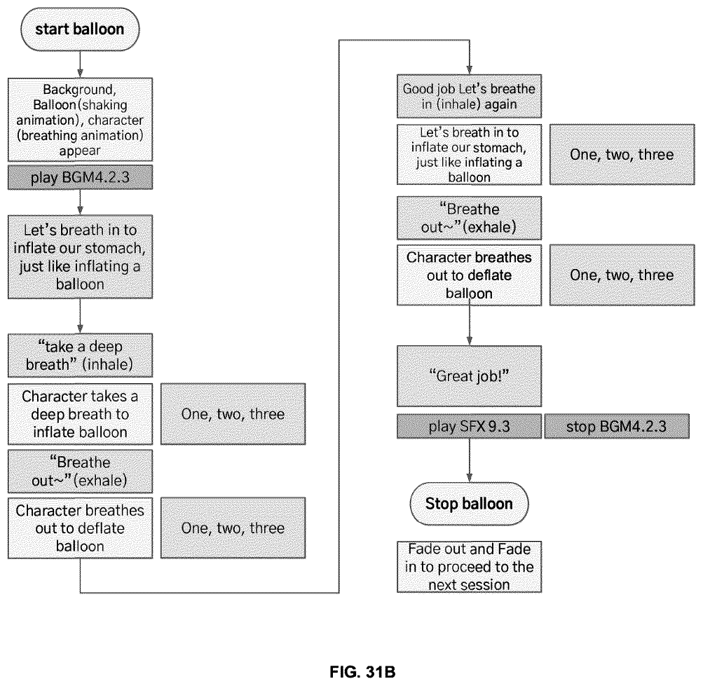

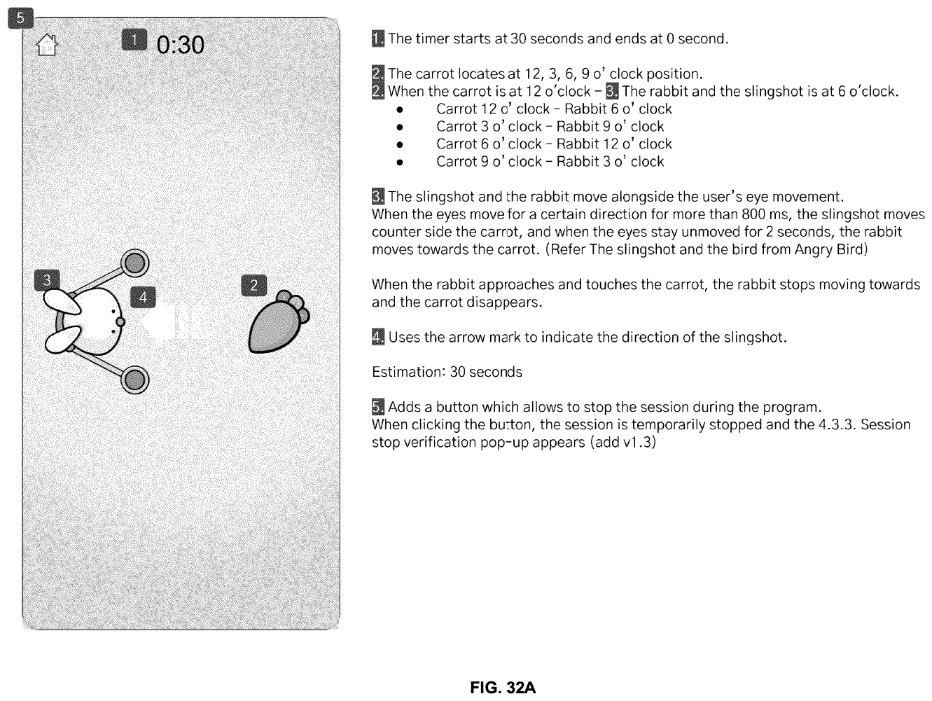

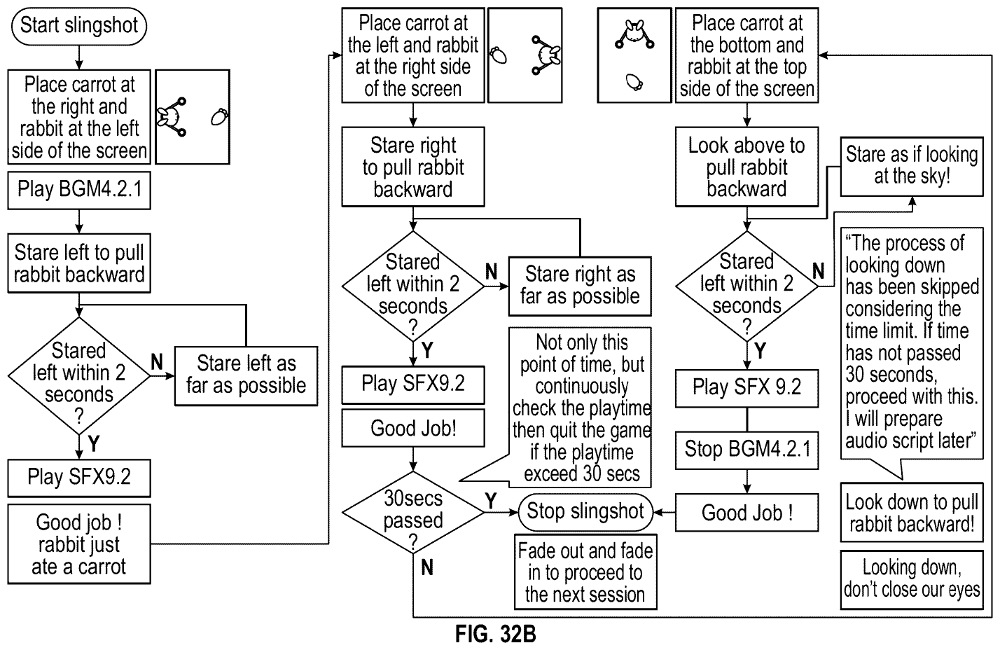

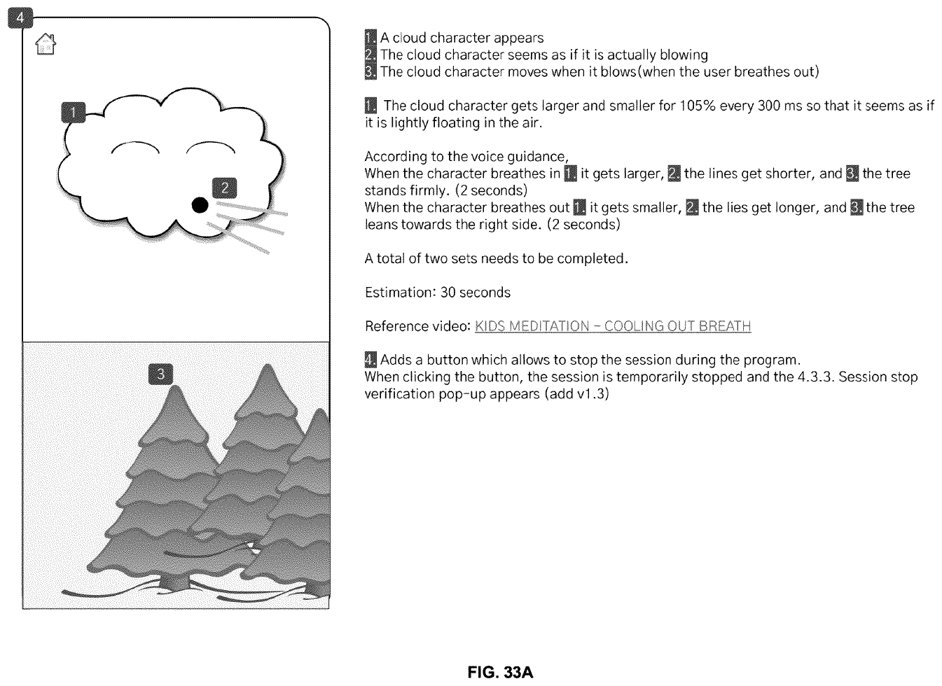

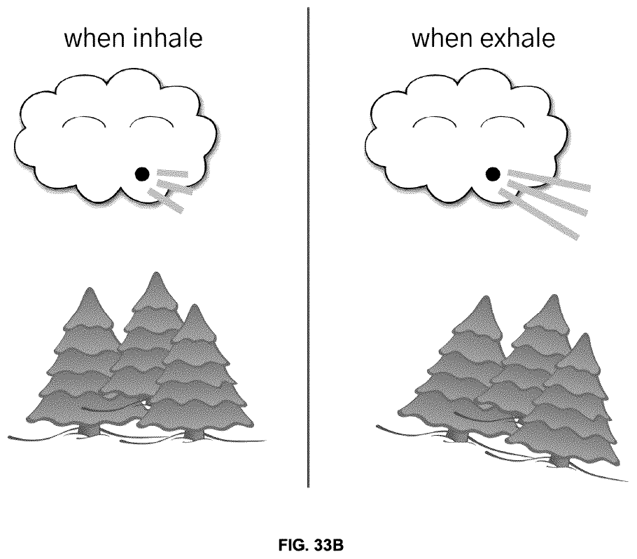

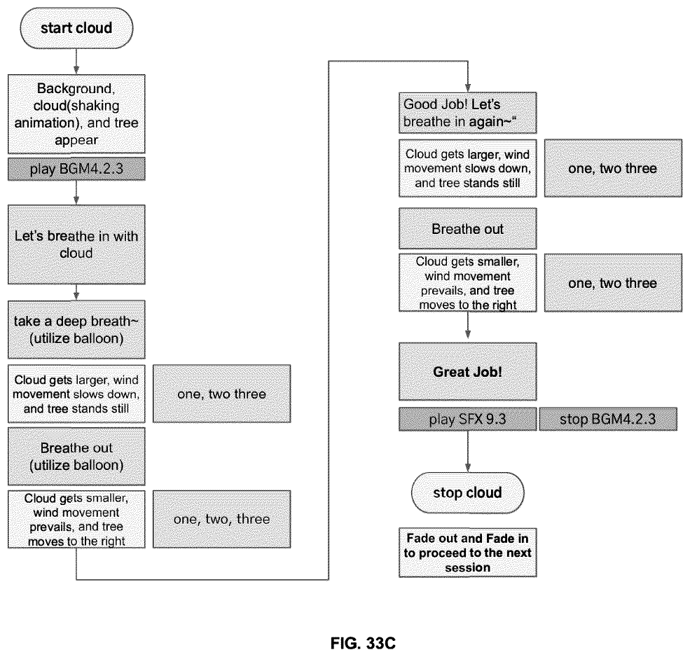

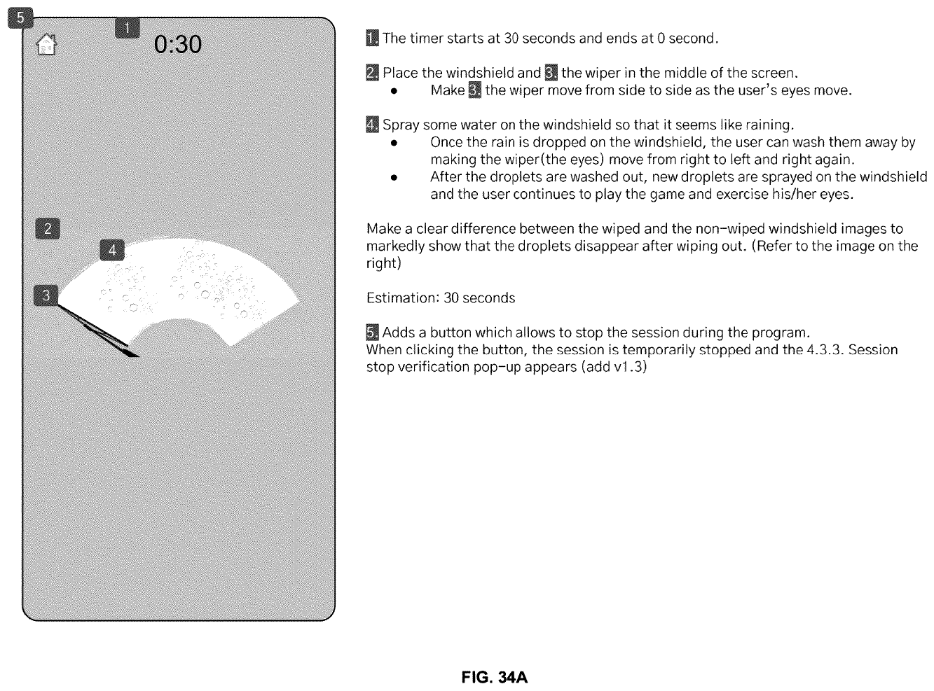

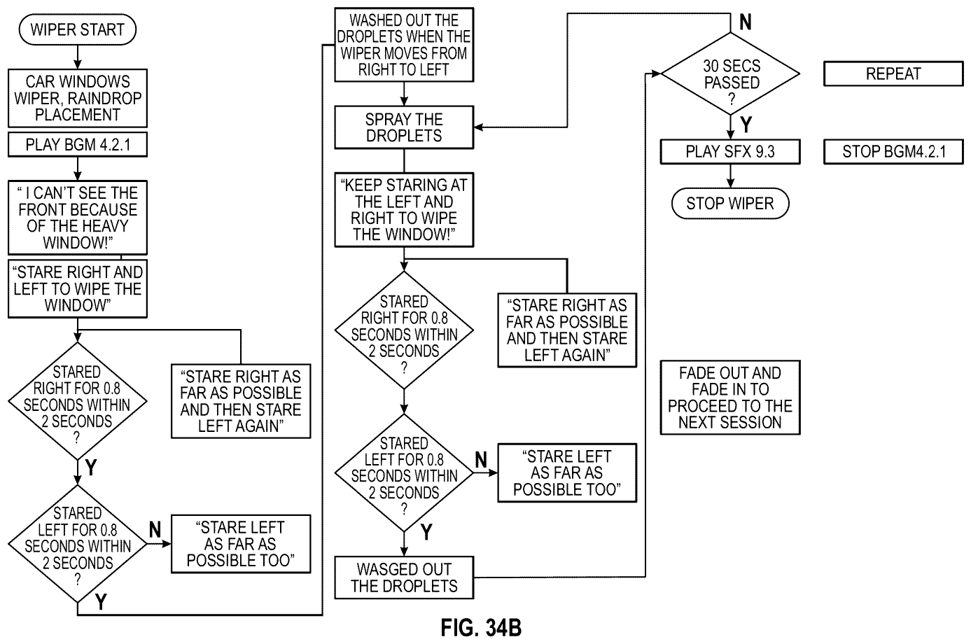

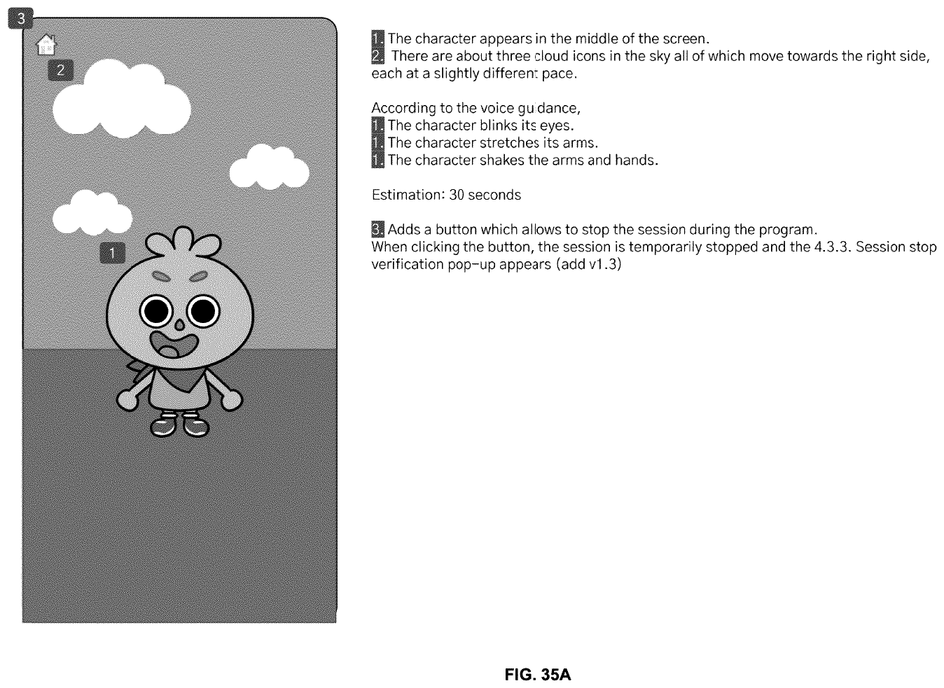

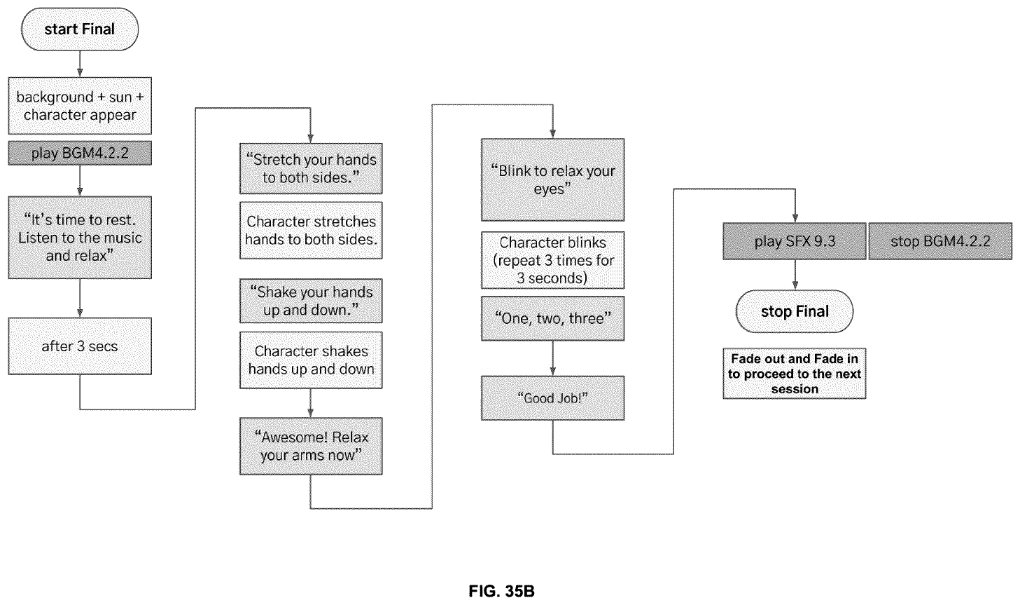

A session may comprise any number of digital therapeutic modules. In some embodiments, a session may comprise two or more digital therapeutic modules. In some embodiments, a session may comprise 3 or more, 4 or more, 5 or more, 6 or more, 7 or more, 8 or more, 9 or more, 10 or more, 11 or more, 12 or more, 13 or more, 14 or more, 15 or more, 20 or more, or 25 or more digital therapeutic modules. A session may comprise any number of digital therapeutic modules, and the digital therapeutic modules may be independently selected from an eye exercise module, a relaxation module, a deep breathing module, and a light therapy module. FIG. 20 depicts a flow chart illustrating an execution flow for a session in a digital application, wherein the session comprises 10 digital therapeutic modules. In some embodiments, a session may consist of 10 digital therapeutic modules, and the digital therapeutic modules comprise 5 eye exercise modules, 3 relaxation modules, and 2 deep breathing modules. A person of skill in the art will appreciate that there are a vast number of combinations for the number and type(s) of digital therapeutic modules that may go into a particular session. FIG. 26A through FIG. 35B depict various types of digital therapeutic modules (e.g., eye exercise, relaxation, and deep breathing).

In some embodiments, the accuracy of measurement of the subject's eye position may be calibrated, and said calibrating comprises determining threshold for detecting the subject's eyes. In additional embodiments, said calibrating the accuracy of measurement of the subject's eye position comprises one or more of instructing the subject to position their face to appear on a screen of the digital apparatus, detecting the subject's eyes for a given period of time, instructing the subject to blink their eyes, detecting if the subject blinked their eyes, instructing the subject to stare at the screen, instructing the subject to move their eyes in a given direction or rotate their eyes, and determining a threshold for detecting the subject's eyes. In some embodiments, the digital apparatus comprises one or more sensors for tracking movement of the subject's eyeball. The threshold for detecting the subject's eyes may be determined in a variety of ways. For example, the movement of eyes left to right may be scaled to 100 out of a maximum horizontal view. The movement of eyes from the top to bottom may also be scaled to 100 out of a maximum vertical view. Movement of the eyes for an average person is about 70 based on the scale of 100. For children and patients having myopia, movement of the eyes is less than 70. In one example, a threshold may be 70% of 70 (e.g., about 49). In another example, a threshold may be 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% of a predetermined value based on the scale of 100. The predetermined value may be 70. In other embodiments, the threshold may be about 40, about 45, about 50, about 55, about 60, about 65, about 70, about 75, about 80, about 85, or about 90. In other embodiments, the threshold may be 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99 based on the scale of 100.

In one aspect, the digital therapeutic modules is generated based on the threshold. For example, an eye exercise module is generated based on the threshold. The eye exercise may include moving an object within 10, 9, 8, 7, 6, 5, 4, 3, 2, or 1 from a boundary of the threshold of the subject based on the scale of 100 in order to increase the threshold of the subject. The eye exercise may include moving an object within 10, 9, 8, 7, 6, 5, 4, 3, 2, or 1 from a boundary of the threshold of the subject after a sensor detects a gaze of eyes within 10, 9, 8, 7, 6, 5, 4, 3, 2, or 1 from a boundary of the threshold.

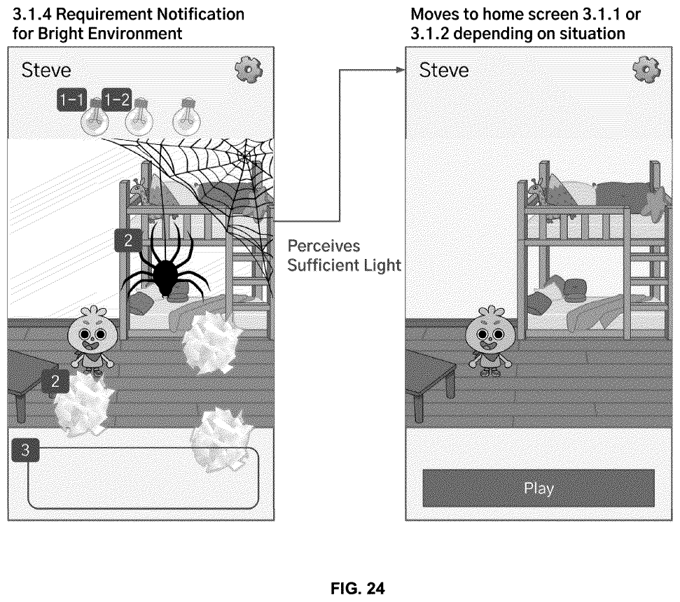

In some embodiments, the accuracy of measurement of the light environment may be calibrated, and said calibrating the light environment comprises one or more of detecting light in the subject's environment using a light sensor of the digital apparatus, and instructing the subject to turn on one or more lights in their environment. FIG. 24 depicts a Bright Environment Requirement Notification screen of a light therapy module of a digital application of the present disclosure, wherein the Bright Environment Requirement Notification screen indicates the amount of light detected by the digital apparatus. It is important to be exposed to bright light while performing eye exercise. The room starts at a very messy state, and becomes clean as the digital apparatus perceives the light. The digital application exposes the patient to bright light at least 3 times a day. After launching the app, the camera sensor perceives bright light and the lights that are off turn on. As shown in FIG. 24, (1) the light bulb helps induce the user to be exposed to bright light 3 times a day. All lights are off on the very first launch of the digital application (1-1), and the light bulbs start to go on after perceiving sufficient light (1-2), (2) an inducement element help induce the user to be exposed to bright light (e.g., dark background, spider, spider web, dust, etc.), (3) skipping the bottom of home element (e.g., play button, and completion notifications are left out on this page).

In some embodiments, the digital application for treating myopia instructs a processor of the digital apparatus to execute operations. In some embodiments, the executed operations comprise generating digital therapeutic modules for treating myopia based on a mechanism of action in and a therapeutic hypothesis for the myopia. In some embodiments, the executed operations comprise generating digital instructions based on the digital therapeutic modules. In some embodiments, the executed operations comprise providing the digital instruction to a subject. In some embodiments, the executed operations comprise collecting the subject's execution outcomes of the digital instructions. In some embodiments, the generating of the digital instructions and the collecting of the subject's execution outcomes of the digital instructions are repeatedly executed several with multiple feedback loops. In some embodiments, the generating of the digital instructions comprises generating the subject's digital instructions for this cycle based on the subject's digital instructions in the previous cycle and the collected execution outcome data on the subject's digital instructions provided in the previous cycle.

In some embodiments, the given interval at which a location of the eyeball is measured is about 10 milliseconds (ms), 25 ms, 50 ms, 60 ms, 70 ms, 80 ms, 90 ms, 100 ms, 110 ms, 120 ms, 130 ms, 140 ms, 150 ms, 175 ms, 200 ms, 250 ms, 300 ms, 350 ms, 400 ms, or a range of two values therebetween. In some embodiments, the given interval at which a location of the eyeball is measured is between about 10 ms, and about 500 ms, about 50 ms and about 250 ms, about 75 and about 150 ms, or about 90 ms and about 110 ms.

In some embodiments, the generating of the digital therapeutic modules comprises generating the digital therapeutic modules by applying imaginary parameters about the subject's environments, behaviors, emotions, and cognition to the mechanism of action in and the therapeutic hypothesis for the myopia.

In some embodiments, the digital application for treating myopia instructs a processor of the digital apparatus to generate digital therapeutic modules comprising two or more modules selected from the group consisting of an eye exercise module, a relaxation module, and a light therapy module.

In some embodiments, the eye exercise module comprises one or more exercise instructions for one or more of: eyeball exercise instructions, biofeedback control instructions, and eyeball-related behavior control instructions.

In some embodiments, the relaxation module comprises one or more relaxation instructions for one or more of: physical exercise instructions, ego enhancement instructions, safety feeling instructions, comfort feeling instructions, and fun instructions. In some embodiments, the light therapy module comprises one or more light therapy instructions for controlling a light environment of the subject. In some embodiments, the one or more relaxation instructions comprise one or more of playing a sound or song, inducing blinking, and instructing the subject to perform gymnastics.

In some embodiments, the digital therapeutic modules further comprise an accomplishment module comprising one or more accomplishment instructions for task accomplishment and for providing compensation for the subject's adherence to the instructions of the two or more first modules. In some embodiments, the digital therapeutic modules further comprise a fun module comprising one or more fun instructions for music, games, or videos.

In some embodiments, the healthcare provider portal provides a healthcare provider with one or more options, and the one or more options provided to the healthcare provider are selected from the group consisting of adding or removing the subject, viewing or editing personal information for the subject, viewing adherence information for the subject, viewing a result of the subject for one or more at least partially completed digital therapeutic modules, prescribing one or more digital therapeutic modules to the subject, altering a prescription for one or more digital therapeutic modules, and communicating with the subject. In some embodiments, the one or more options comprise the viewing or editing personal information for the subject, and the personal information comprises one or more selected from the group consisting of an identification number for the subject, a name of the subject, a date of birth of the subject, an email of the subject, an email of the guardian of the subject, a contact phone number for the subject, a prescription for the subject, and one or more notes made by the healthcare provider about the subject. In some embodiments, the personal information comprises the prescription for the subject, and the prescription for the subject comprises one or more selected from the group consisting of a prescription identification number, a prescription type, a start date, a duration, a completion date, a number of scheduled or prescribed digital therapeutic modules to be performed by the subject, and a number of scheduled or prescribed digital therapeutic modules to be performed by the subject per day. In some embodiments, the one or more options comprise the viewing the adherence information, and the adherence information of the subject comprises one or more of a number of scheduled or prescribed digital therapeutic modules completed by the subject, and a calendar identifying one or more days on which the subject completed, partially completed, or did not complete one or more scheduled or prescribed digital therapeutic modules. In some embodiments, the one or more options comprise the viewing the result of the subject, and the result of the subject for one or more at least partially completed digital therapeutic modules comprises one or more selected from the group consisting of a time at which the subject started a scheduled or prescribed digital therapeutic module, a time at which the subject ended a scheduled or prescribed digital therapeutic module, an indicator of whether the scheduled or prescribed digital therapeutic module was fully or partially completed, and an exercise intensity (EI).

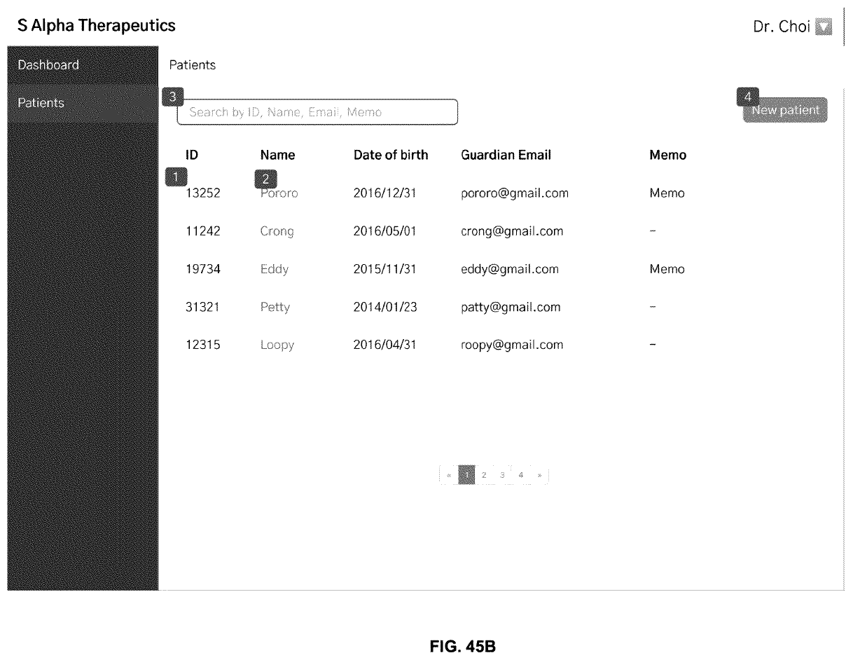

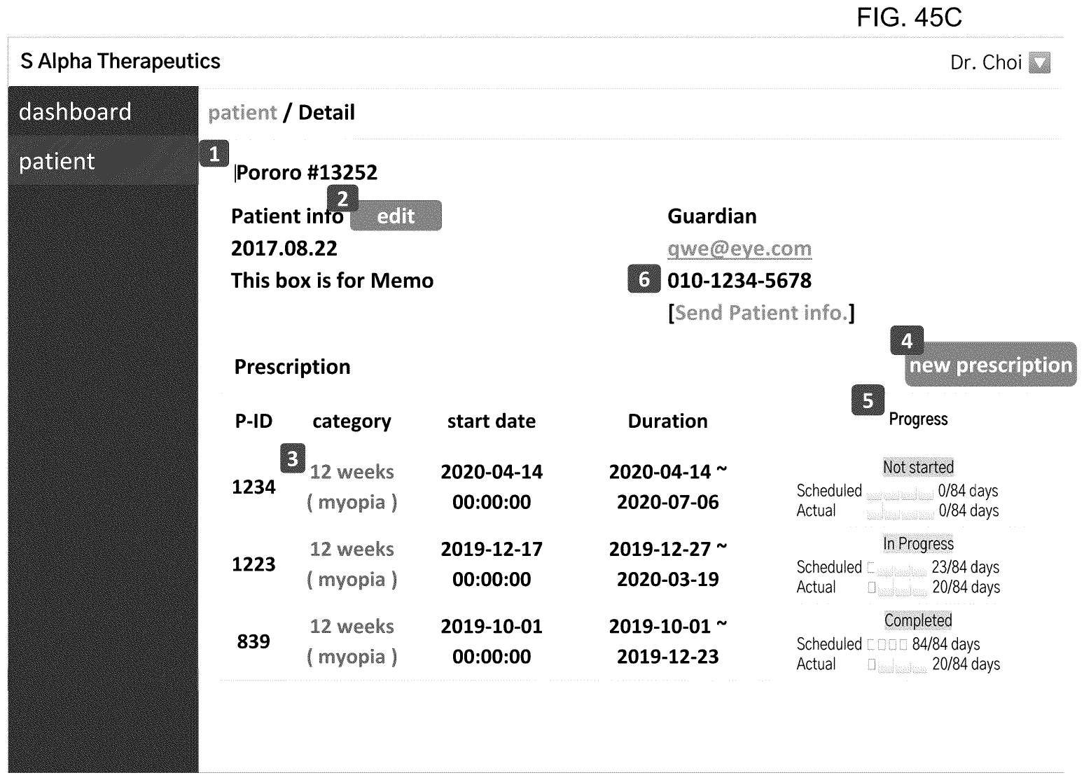

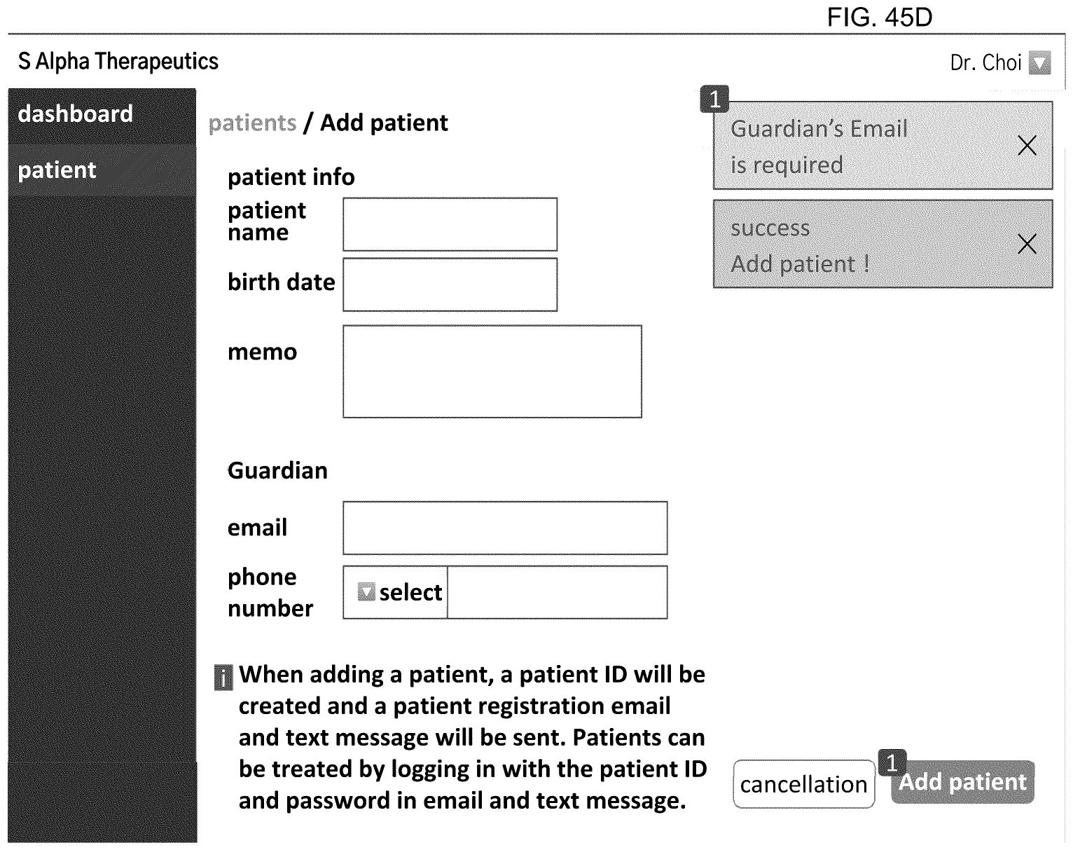

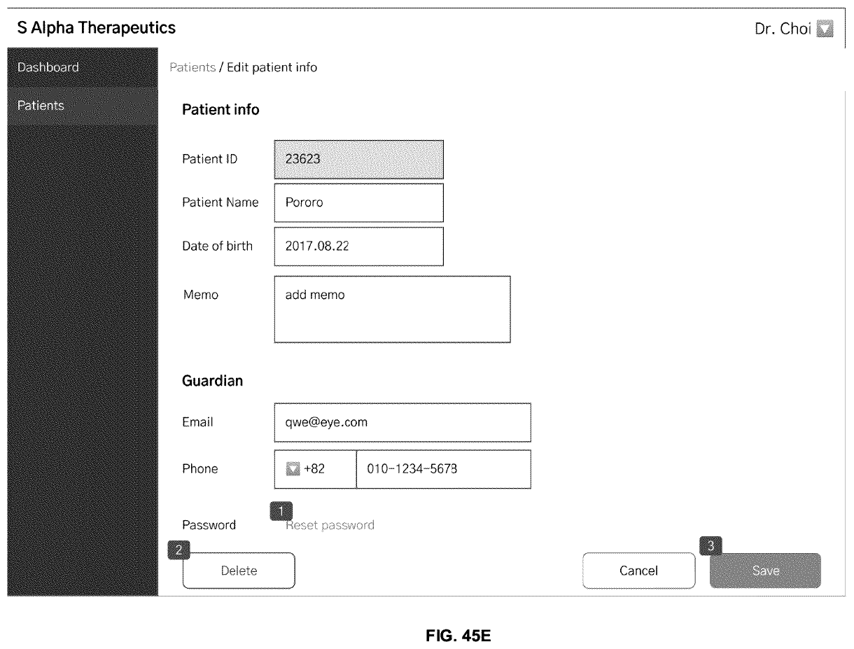

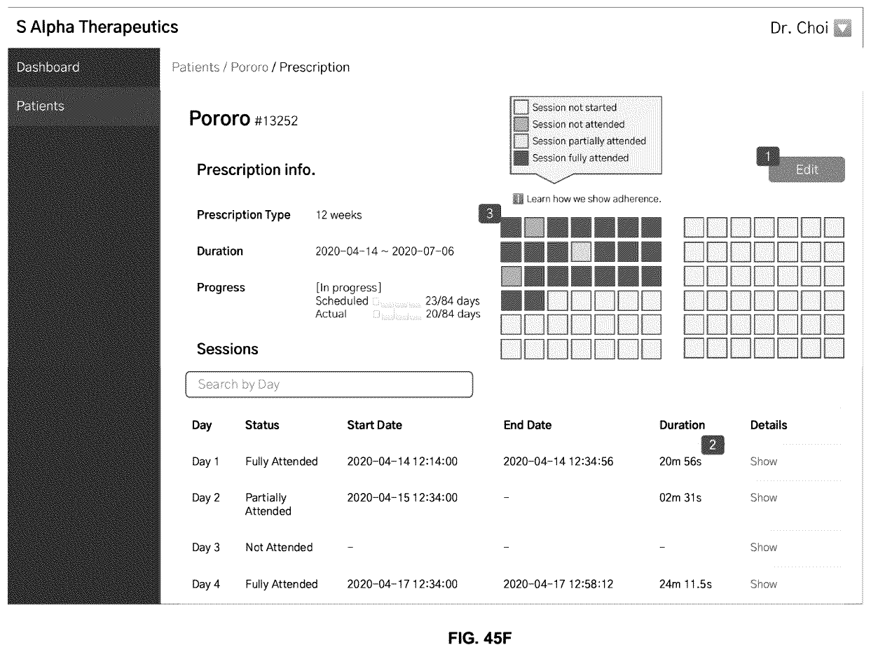

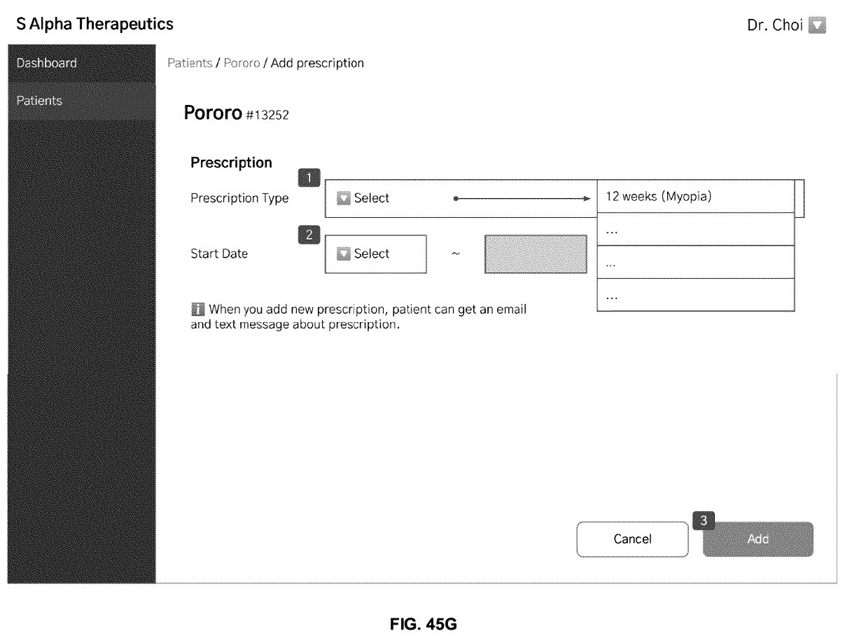

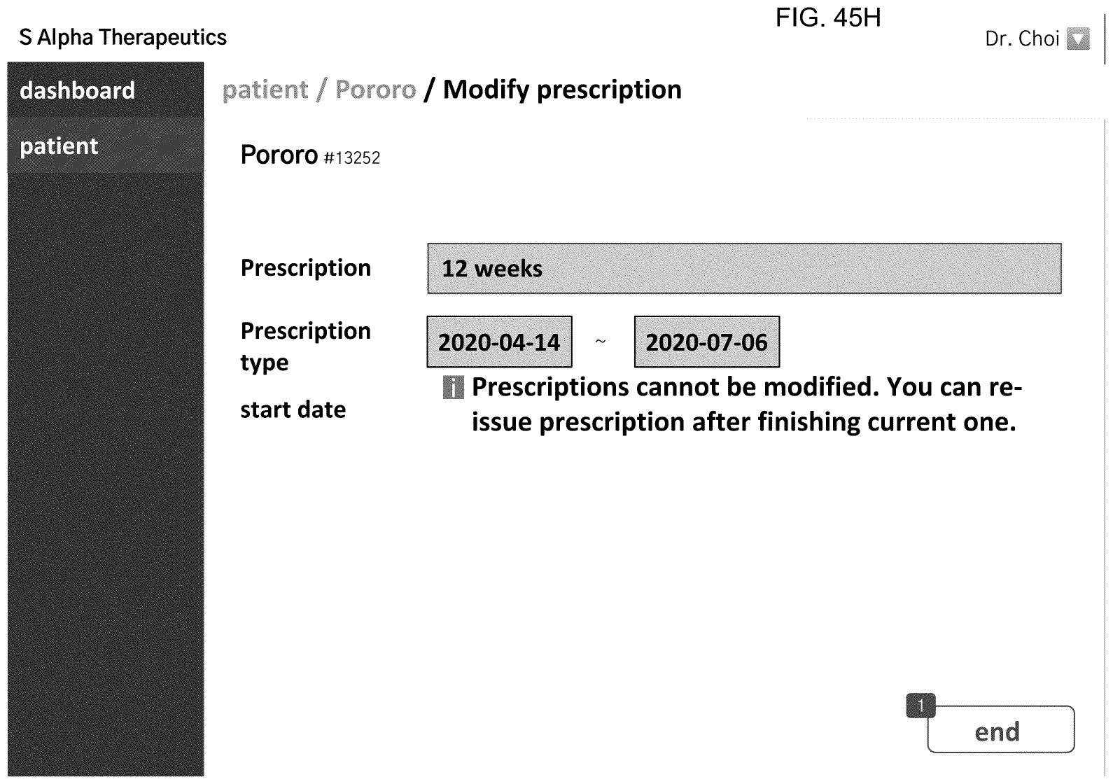

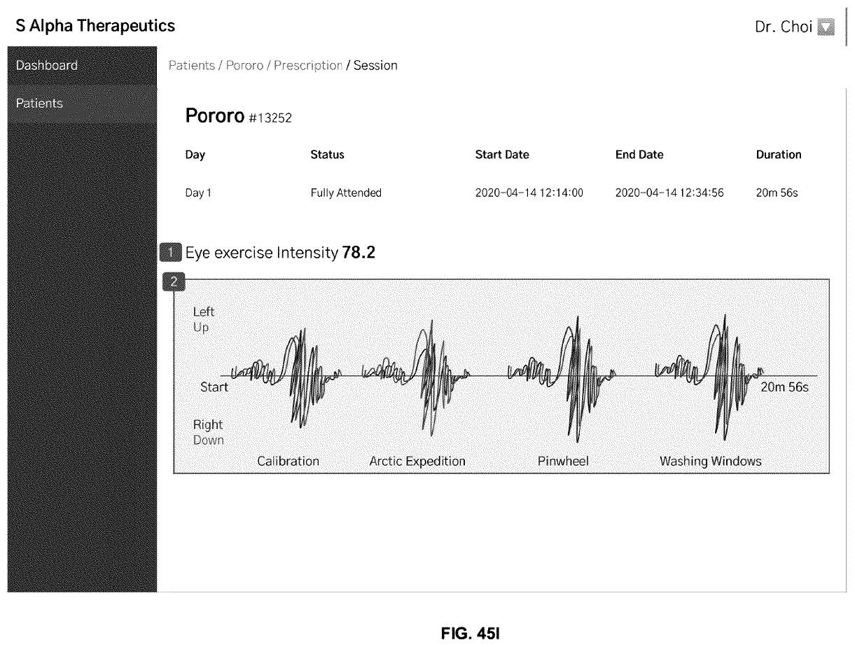

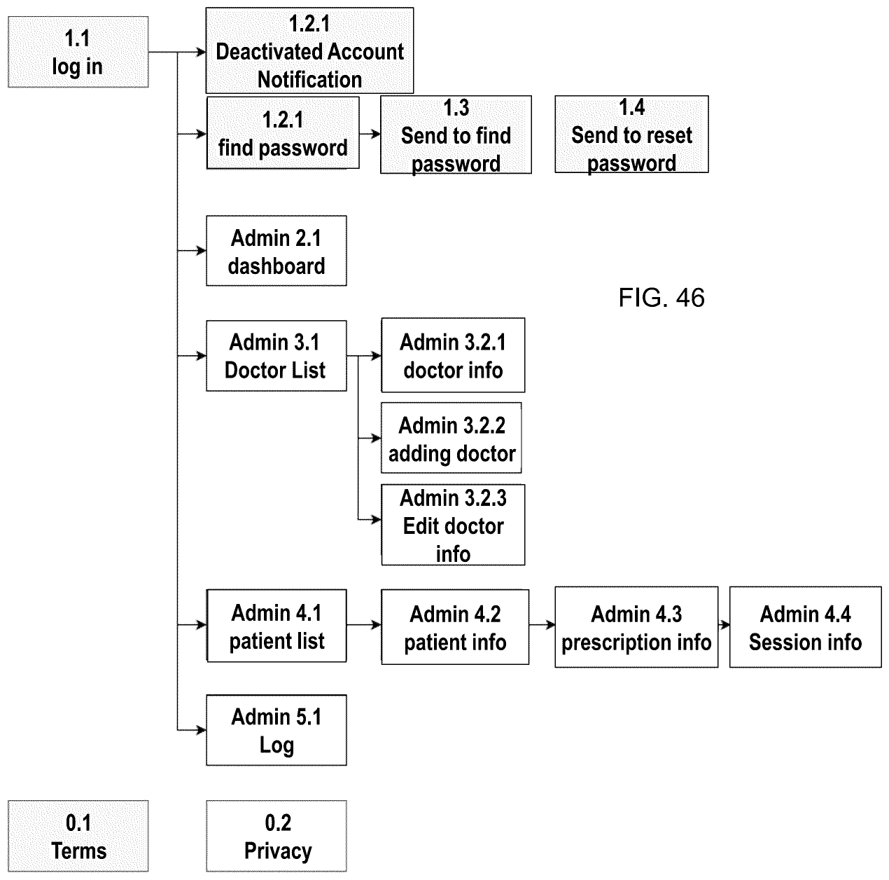

FIG. 45A depicts a dashboard of a healthcare provider portal. (1) The number of all patients associated with the present doctor's account. A graph may be used to show the number of patients who have opened the digital application for patient per day in the most recent 90 days. The number of patients in progress may also be viewed. A graph may be used to show the number of patients who have completed the daily sessions per day in the most recent 90 days. FIG. 45B depicts a patient tab in a healthcare provider portal, the patient tab displaying a list of patients. As shown, (1) Patient ID (the unique identification number temporarily given to each patient when adding them on the list), (2) Patient Name, (3) Search bar for searching by ID, Name, Email, Memo, etc., and (4) Add New Patient button for adding new patients. FIG. 45C depicts a patient tab in a healthcare provider portal, the patient tab displaying detailed information on a given patient. As shown, (1) detailed patient information, (2) a button for editing patient information, (3) prescription information, (4) a button for adding a new prescription, (5) displays a progress status for different each prescription, and (6) a button or link for sending an email to the patient. FIG. 45D depicts a patient tab in a healthcare provider portal for adding a new patient. As shown, (1) shows a button for adding a new patient, and (3) shows an error message displayed when required patient information has not been provided. FIG. 45E depicts a patient tab in a healthcare provider portal for editing information of an existing patient. As shown, (1) is a button or link for resetting a password, (2) is a button for deleting a given patient, and (3) is a button for saving changes. FIG. 45F depicts a patient tab in a healthcare provider portal that displays detailed prescription information for a given patient. As shown, (1) is a button for editing prescription information, (2) displays the duration of the sessions attended by the patient or subject, and (3) shows an overview the treatment progress. Seven days are represented as a line or row of 7 squares. For 12 weeks, each 6 weeks may be presented separately. Different colors may be used to discern session statuses (e.g., grey for sessions not started, red for sessions not attended, yellow for sessions partially attended, and green for sessions fully attended). FIGS. 45G-H depict a patient tab in a healthcare provider portal for editing prescription information for a given patient. FIG. 451 depicts a patient tab in a healthcare provider portal for viewing details (e.g., date, status, duration, results such as EI or AEI) of a given session for a given patient. As shown, (1) is the eye exercise intensity, and (2) is a graph of the exercise intensity. Different colors may be used in the graph to differentiate between up/down or right/left movement of the eye. In the graph, the larger the amplitude, the more the eye has been exercised.

In some embodiments, collecting the subject's execution outcomes of the digital instructions comprises determining one or both of an exercise intensity (EI) and an average exercise intensity (AEI). In some embodiments, AEI may be determined as an averaged sum of differences between a final location of an eyeball of the subject and a starting location of the eyeball measured at a given interval. The EI may be determined according the formula:

$$EI = \frac{AEI \times 100}{145}$$

In certain embodiments, total AEI can be determined as a sum of dynamic AEI and static AEI. Dynamic AEI may be determined based on the eyeball movement over a given time, and static AEI may be determined based on holding a stretched position for an eyeball over a given time. For example, while dynamic AEI can be determined as the averaged sum of differences between a final location of an eyeball of the subject and a starting location of the eyeball measured over given interval (e.g., corresponding to a measure of the how much an eyeball is moving), static AEI can be determined as the averaged sum of distances of the eyeball from a resting position (e.g., looking straight ahead) measured over given interval as the eyeball is fixed in place (e.g., not moving). With respect to dynamic AEI, when the eyeball tracking is started with the eye in a resting position (time=0), AEI is calculated by measuring changes in distance travelled by the eyeball (d) over a given interval (e.g., 10 to 500 msec). That is, if d is large, a lot of eye movement is measured, resulting in a high AEI. Small changes in d (e.g., when the eyeball moves less or not at all) result in a low AEI. However, dynamic AEI does not account for exercise of the eye muscles when the eye is fixed at a location that is not the resting position. In other words, over a given interval, the subject's eye can be held at a position that is not the resting state (e.g., d=0), however eye muscles are still being exercised in order to hold the eye at that position. Static AEI accounts exercise of the eye that is not related to eye movement.

In some embodiments, the administrative portal provides an administrator with one or more options, and the one or more options provided to the administrator of the system are selected from the group consisting of adding or removing the healthcare provider, viewing or editing personal information for the healthcare provider, viewing or editing de-identified information of the subject, viewing adherence information for the subject, viewing a result of the subject for one or more at least partially completed digital therapeutic modules, and communicating with the healthcare provider. In some embodiments, the one or more options comprise the viewing or editing the personal information, and the personal information of the healthcare provider comprises one or more selected from the group consisting of an identification number for the healthcare provider, a name of the healthcare provider, an email of the healthcare provider, and a contact phone number for the healthcare provider. In some embodiments, the one or more options comprise the viewing or editing the de-identified information of the subject, and the de-identified information of the subject comprises one or more selected from the group consisting of an identification number for the subject, and the healthcare provider for the subject. In some embodiments, the one or more options comprise the viewing the adherence information for the subject, and the adherence information of the subject comprises one or more of a number of scheduled or prescribed digital therapeutic modules completed by the subject, and a calendar identifying one or more days on which the subject completed, partially completed, or did not complete one or more scheduled or prescribed digital therapeutic modules. In some embodiments, the one or more options comprise the viewing the result of the subject, and the result of the subject for one or more at least partially completed digital therapeutic modules comprises one or more selected from the group consisting of a time at which the subject started a scheduled or prescribed digital therapeutic module, a time at which the subject ended a scheduled or prescribed digital therapeutic module, an indicator of whether the scheduled or prescribed digital therapeutic module was fully or partially completed, and an exercise intensity (EI).

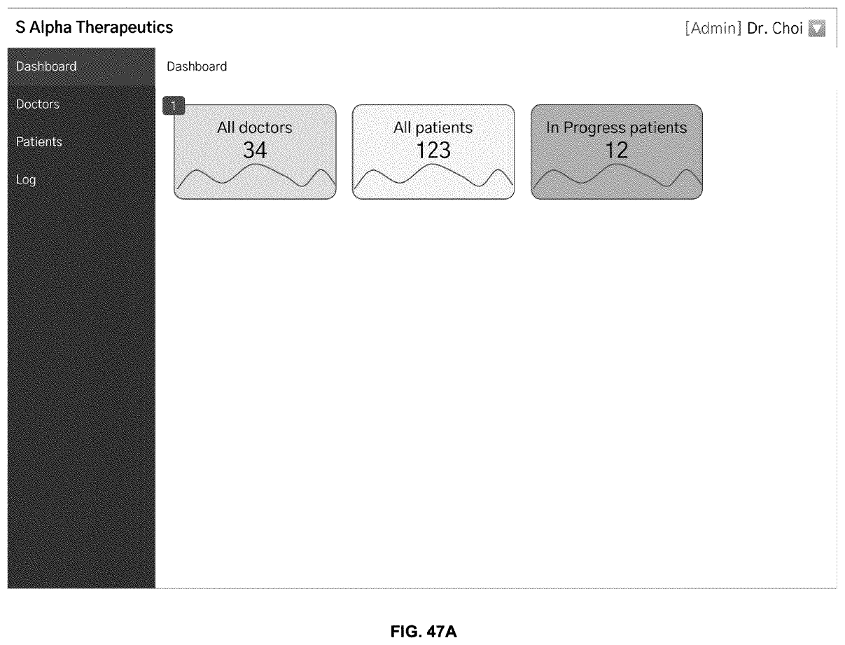

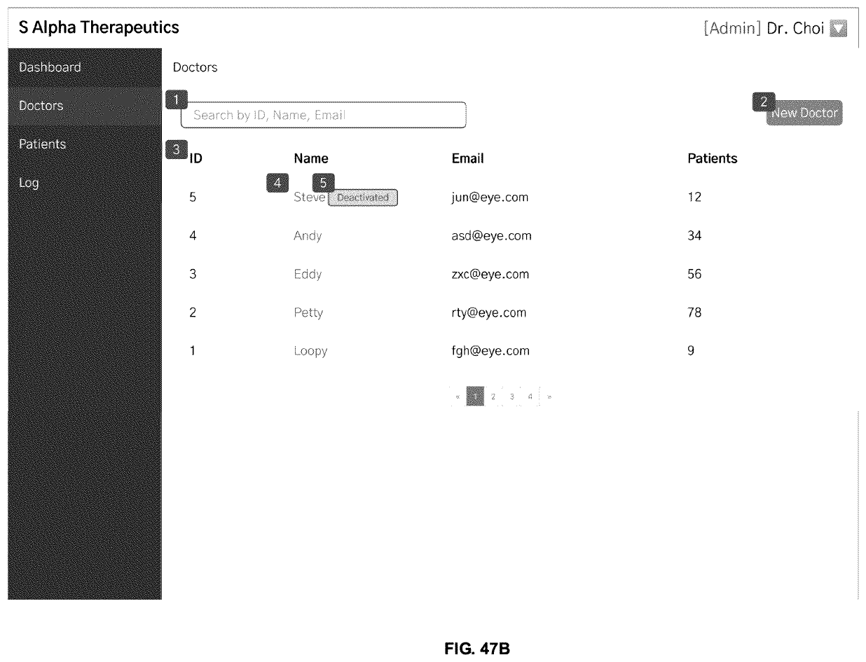

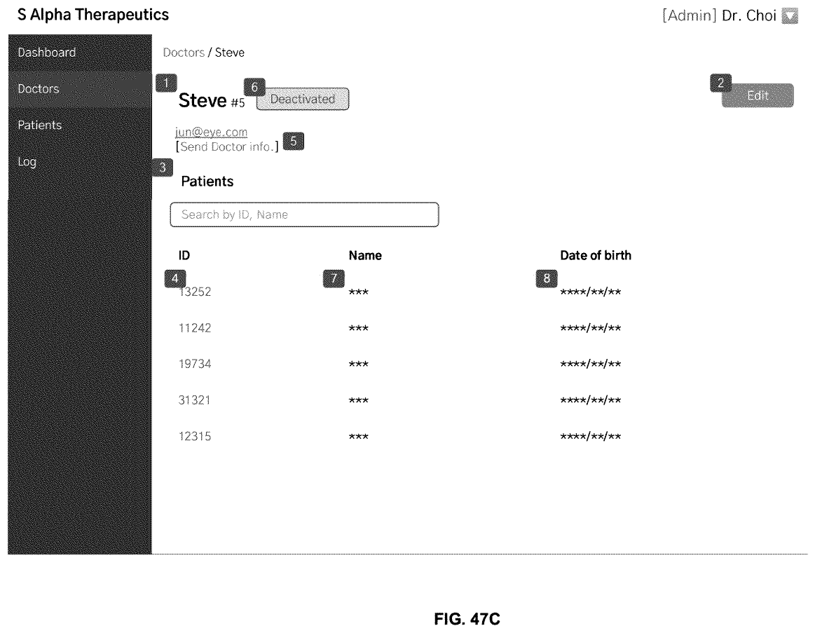

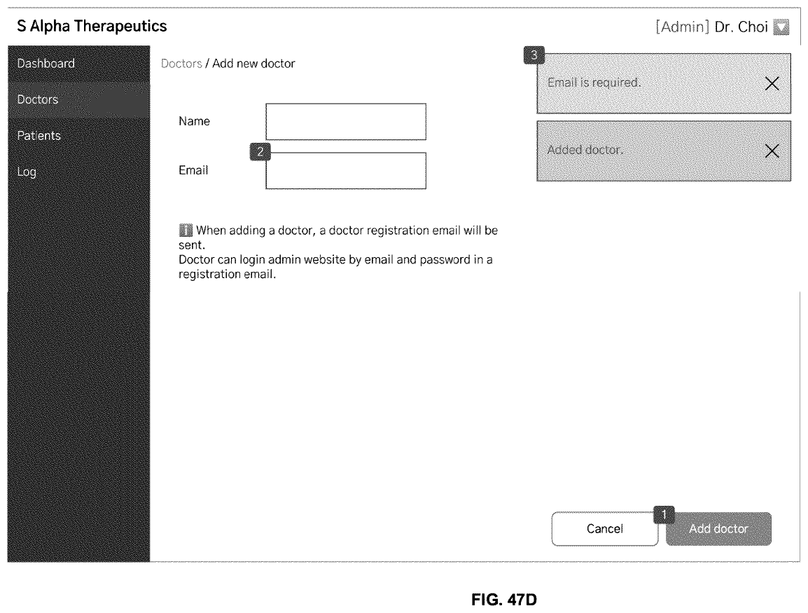

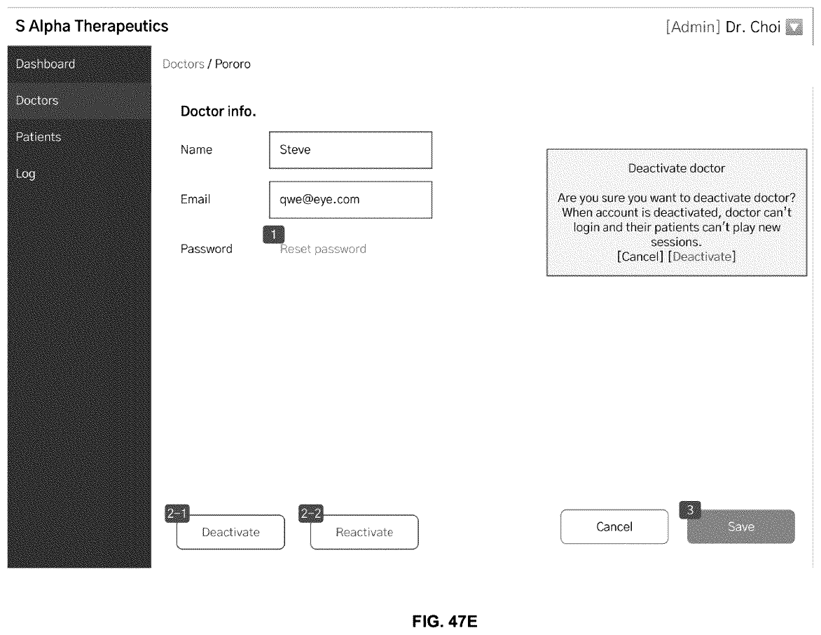

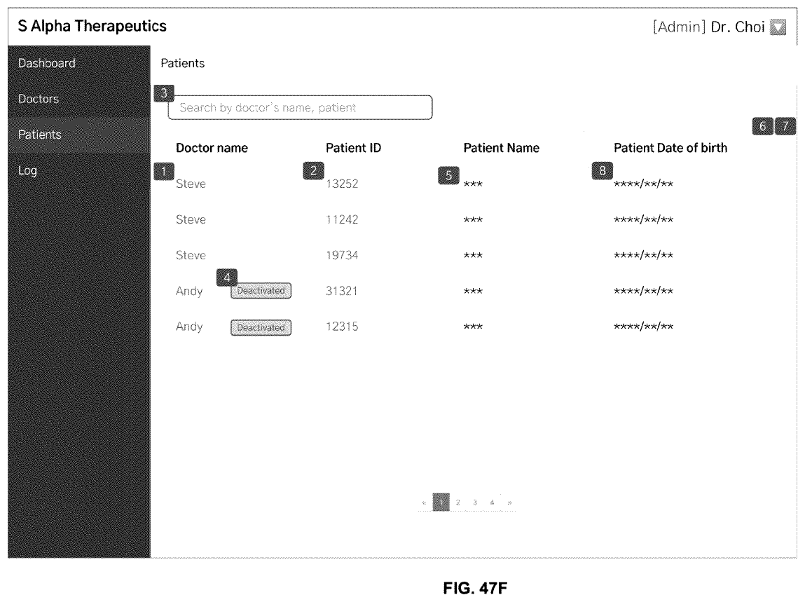

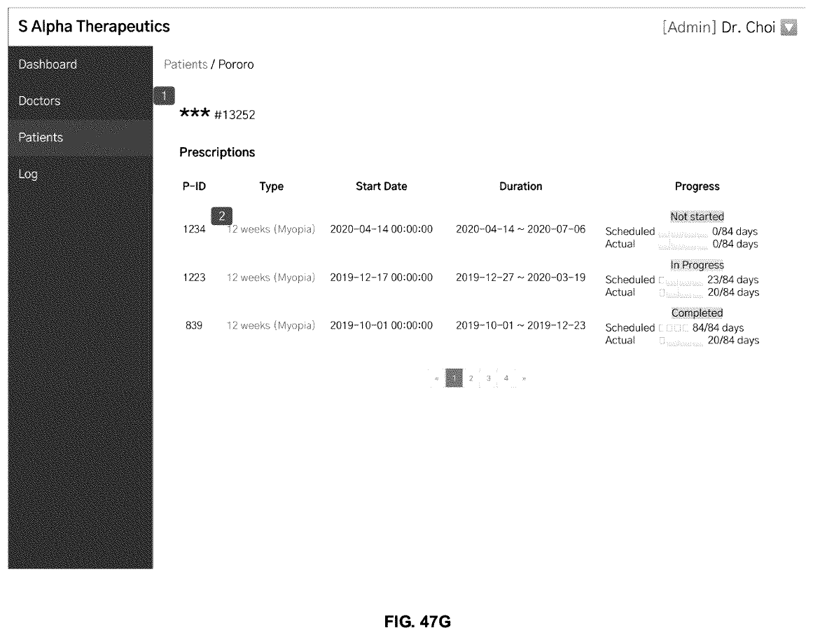

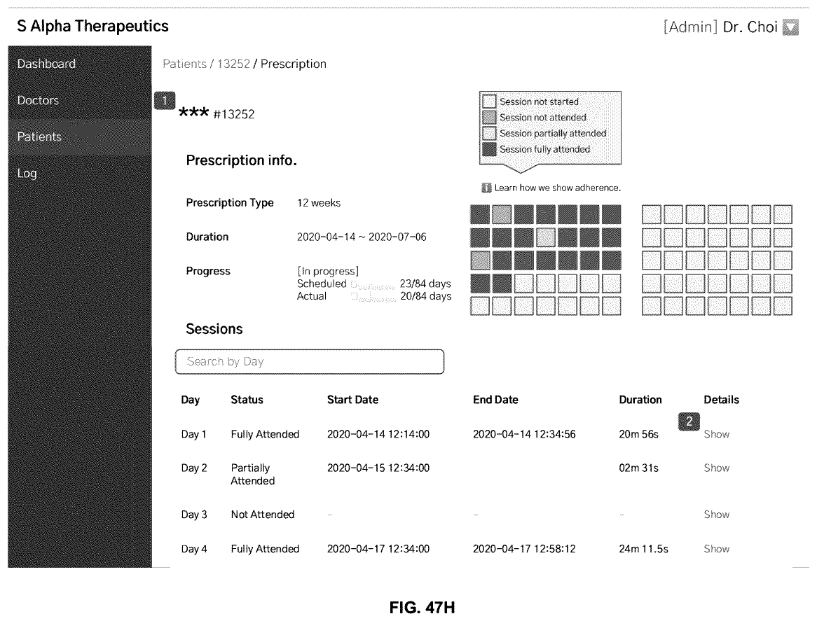

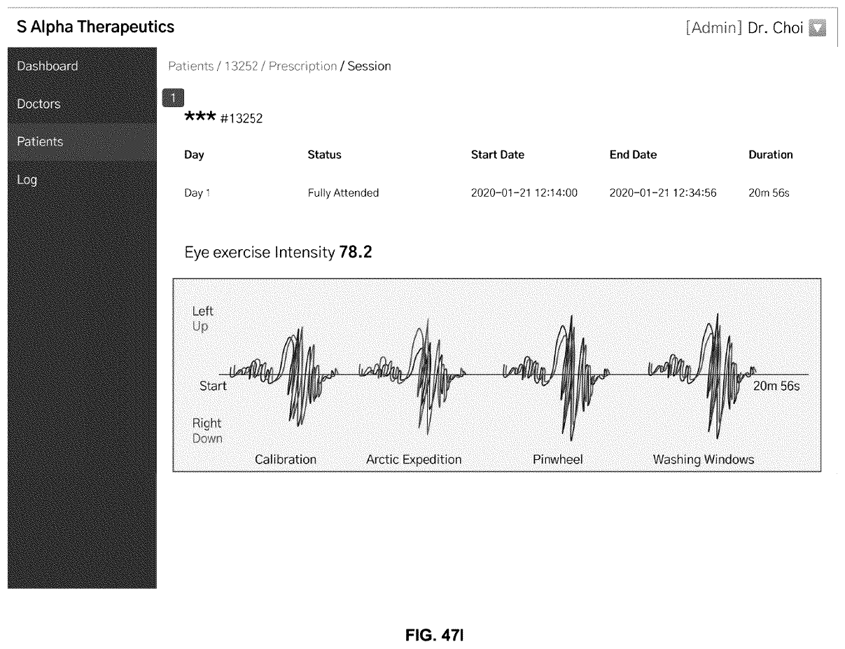

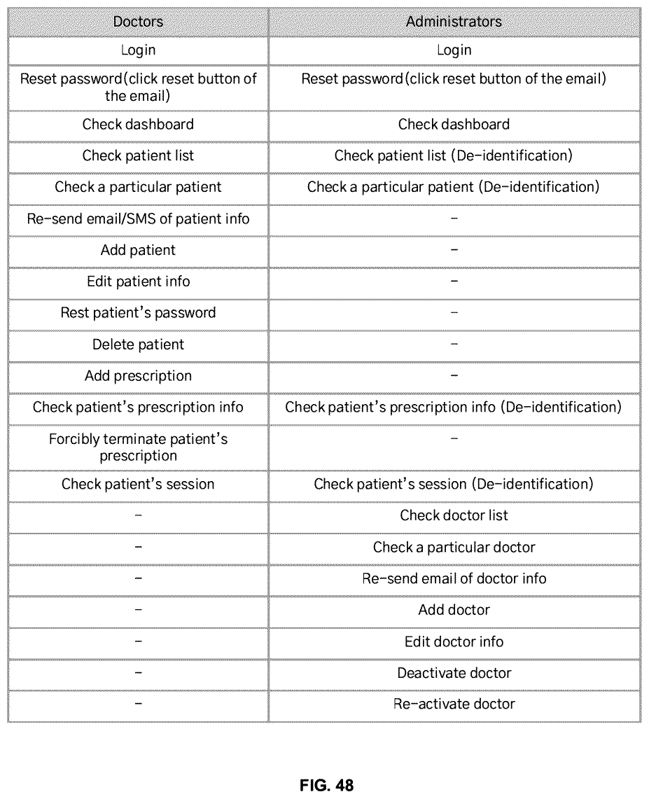

FIG. 47A depicts (A) a dashboard of an administrative portal. As shown, (1) shows the number of doctors. A graph may be used to show the number of doctors that have visited the digital application per day in the most recent 90 days. A graph may be used to show the number of patients who have opened the digital application for patient per day in the most recent 90 days. The number of patients in progress may also be viewed. A graph may be used to show the number of patients who have completed the daily sessions per day in the most recent 90 days. FIG. 47B depicts a doctor tab in an administrative portal, the doctor tab displaying a list of doctors. As shown, (1) is a search bar for searching for various doctors by name, email, etc., (2) shows a button for adding a new doctor, (3) is the doctor's ID, (4) is a button for viewing detailed doctor information, and (5) shows deactivated doctor accounts. FIG. 47C depicts a doctor tab in an administrative portal, the doctor tab displaying a list of patients being cared for by a given doctor, with patient-identifying information redacted (*). As shown, (1) is the doctor's account information, (2) is a button for editing the doctor's account information, (3) is a list of patients being cared for by the doctor, (4) is a list of patient ID numbers, (5) a link or button for sending the doctor a registration email, (6) a notification that the doctor's account has been deactivated, which only appears for deactivated accounts, and (7 and 8) redacted or de-identified patient information. FIG. 47D depicts a doctor tab in an administrative portal for adding a new doctor. FIG. 47E depicts a doctor tab in an administrative portal for editing information of an existing doctor, including activating or deactivating a doctor's account. FIG. 47F depicts a patient tab in an administrative portal that displays information for one or more patients, wherein sensitive information is redacted. FIG. 47G depicts a patient tab in an administrative portal that displays detailed patient or prescription information for a given patient. FIG. 47H depicts a patient tab in an administrative portal that displays detailed prescription information for a given patient. FIG. 47I depicts a patient tab in an administrative portal for viewing details (e.g., date, status, duration, results) of a given session for a given patient. FIG. 48 provides a table showing privileges for the doctors using the healthcare provider portal and the administrators using the administrative portal.

In some embodiments, the digital application further comprises a push alarm and/or push notifications for one or more of reminding the subject complete a digital therapeutic module and adjusting the light settings of the subject's environment. In some embodiments, the push alarm and/or push notification is activated to remind the subject to adjust the light settings such that the subject is exposed to sufficiently bright light at least 3 times per day.

A patient or subject treated by any of the methods, systems, or digital applications described herein may be of any age and may be an adult, infant or child, however the methods and systems of the present disclosure are particularly suitable for children. In some cases, the patient or subject is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 years old, or within a range therein (e.g., between 2 and 20 years old, between 20 and 40 years old, or between 40 and 90 years old). In some embodiments, the patient or subject is a child. In some embodiments, the patient or subject is a child, and is supervised by an adult when using the methods, systems, or digital applications of the present disclosure. In some embodiments, the patient or subject is less than about 20 years old, less than about 15 years old, less than about 10 years old, or less than about 5 years old.

In some embodiments, the digital apparatus comprises a digital instruction generation unit configured to generate digital therapeutic modules for treating myopia based on a mechanism of action (MOA) in and a therapeutic hypothesis for the myopia, generate digital instructions based on the digital therapeutic modules, and provide the digital instructions to the subject. In some embodiments, the digital apparats comprises an outcome collection unit configured to collect the subject's execution outcomes of the digital instructions. In some embodiments, the digital instruction generation unit generates the digital therapeutic modules based on neurohumoral factors related to the myopia onset. In some embodiments, the neurohumoral factors comprise insulin-like growth factor (IGF), cortisol, and dopamine.

In some embodiments, the digital instruction generation unit generates the digital therapeutic modules based on the inputs from the healthcare provider. In some embodiments, the digital instruction generation unit generates the digital therapeutic modules based on information received from the subject.

In some embodiments, the information is received from the subject comprises at least one of basal factors, medical information, and digital therapeutics literacy of the subject. In some embodiments, the basal factors including the subject's activity, heart rate, sleep, and diet (including nutrition and calories). In some embodiments, the medical information including the subject's electronic medical record (EMR), family history, genetic vulnerability, and genetic susceptibility. In some embodiments, the digital therapeutics literacy including the subject's accessibility, and technology adoption to the digital therapeutics and the apparatus.

In some embodiments, the digital instruction generation unit generates the digital therapeutic modules matching to imaginary parameters which correspond to the mechanism of action in and the therapeutic hypothesis for the myopia. In some embodiments, the imaginary parameters are deduced in relation to the subject's environment, behaviors, emotions, and cognition.

In some embodiments, the digital apparats comprises an outcome collection unit configured to collect the subject's execution outcomes of the digital instructions, and the outcome collection unit collects the execution outcomes of the digital instructions by monitoring the subject's adherence to the digital instructions or allowing the subject to directly input the subject's adherence to the digital instructions. In some embodiments, the generation of the digital instructions at the digital instruction generation unit and the collection of the subject's execution outcomes of the digital instructions at the outcome collection unit are repeatedly executed several times with multiple feedback loops. In some embodiments, the digital instruction generation unit generates the subject's digital instructions for this cycle based on the subject's digital instructions in the previous cycle and the execution outcome data on the subject's digital instructions in the previous cycle collected at the outcome collection unit.

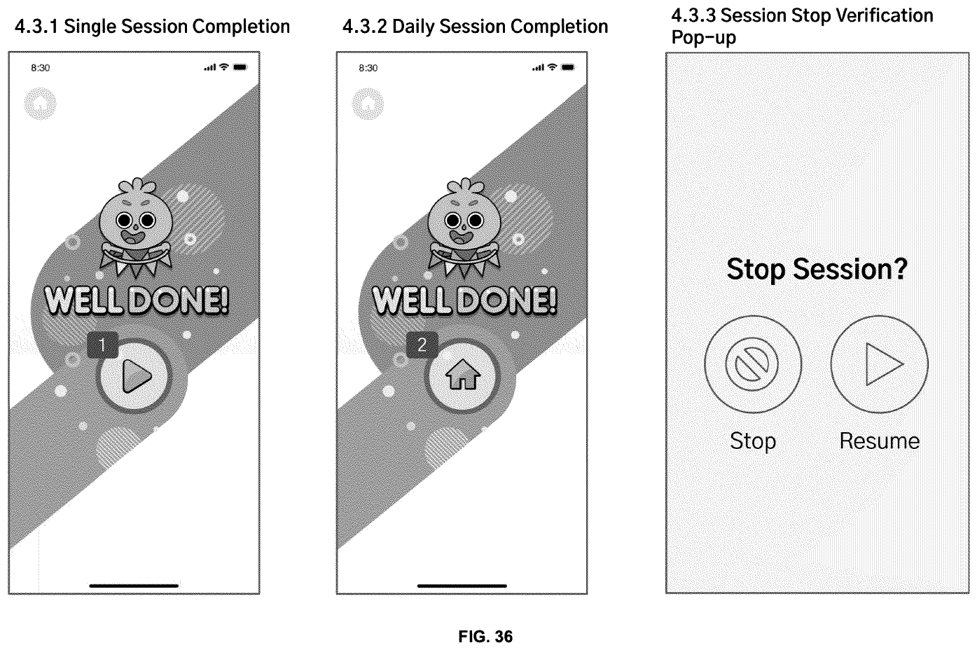

FIG. 36 depicts screenshots shown at the completion of a single session, at the completion of all daily sessions, and at stop/start verification in a digital application of the present disclosure. As shown, (1) a button to continue on the next session, (2) a button to move onto the home screen. If it is the last day of the prescribed duration move to screen 3.1.3, if not, 3.1.2 (see, e.g., FIG. 23). Session Stop Verification Pop-up appears when taps the Home button on the upper left while executing the session.

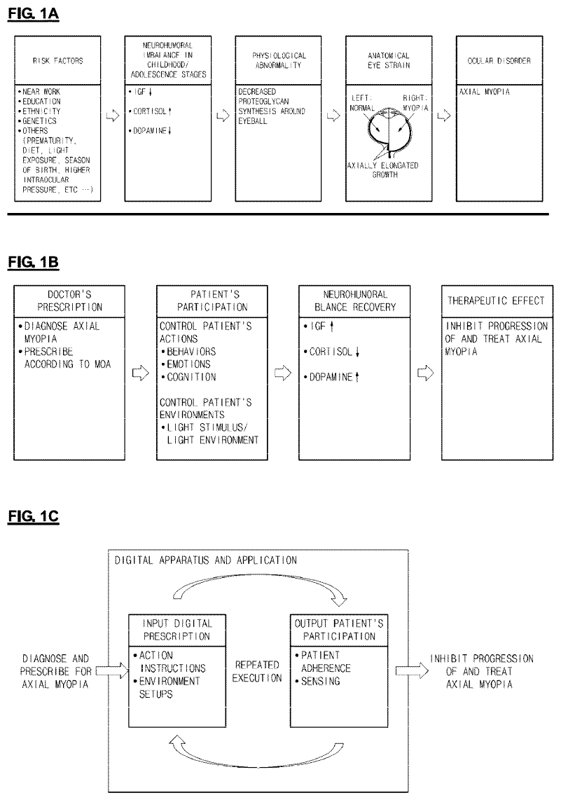

FIG. 1A is a diagram showing a mechanism of action in axial myopia in the childhood/adolescence stages proposed in the present disclosure, FIG. 1B is a diagram showing a therapeutic hypothesis for the axial myopia proposed in the present disclosure, and FIG. 1C is a diagram showing a digital therapeutic hypothesis for axial myopia proposed in the present disclosure.

A digital apparatus and an application for inhibiting progression of and treating myopia according to the present disclosure as will be described below are realized based on the mechanism of action and therapeutic hypothesis deduced through the literature search and expert reviews of clinical trial articles on axial myopia in childhood/adolescence stages.

Generally speaking, disease therapy is carried out by analyzing a certain disease in terms of pathophysiological functions and dispositions in order to determine a start point, a progression point, and an end point for the disease. Also, an indication of the disease is defined by characterization of the corresponding disease and statistical analysis of the disease. Also, patient's physiological factors, especially neurohumoral factors, which correspond to the verified indications, are analyzed, and the patient's neurohumoral factors are restricted a narrow extent associated with the disease to deduce a mechanism of action.

Next, a therapeutic hypothesis, in which the corresponding disease is treated by controlling actions and environments directly associated with regulation of the corresponding neurohumoral factors associated with the disease, is deduced. To realize this therapeutic hypothesis into digital therapeutics, a digital therapeutic hypothesis for achieving a therapeutic effect through repeated digital instruction and execution, which are associated with the “control of patient's action/environment→regulation of neurohumoral factors, is proposed. The digital therapeutic hypothesis of the present disclosure is realized as a digital apparatus and an application is realized as a digital apparatus and an application configured to present changes in patient's actions (including behavioral, emotional, and cognitive areas), improvement of patient's environment, and patient's participation in the form of specific instructions and collect and analyze execution of the specific instructions.

Literature search for the clinical trials as described above may be executed through meta-analysis and data mining, and the clinical specialist's feedbacks and deep reviews may be applied in each analysis step. Basically, the present disclosure encompasses extracting a mechanism of action in and a therapeutic hypothesis for axial myopia using the procedure as described above, and regulating the neurohumoral factors based on these results to provide a digital apparatus and an application as digital therapeutics for inhibiting progression of and treating axial myopia.

However, a method of extracting a mechanism of action in and a therapeutic hypothesis for axial myopia according to the present disclosure is not limited to the methods as described above. In addition, mechanisms of action in and therapeutic hypotheses for diseases may be extracted using various methods.

Referring to FIG. 1A, various risk factors in the childhood/adolescence stages, for example, near working, education, race, genetics, and other factors (premature birth, diet, light exposure, birth season, increased intraocular pressure, etc.) may cause the imbalance of neurohumoral factors (which are related to the myopia onset) in the childhood/adolescence stages. As a result, proteoglycans are abnormally produced in the sclera around the eye due to IGF, cortisol, and dopamine dysregulation, which results in axial myopia developing due to abnormal growth of the optical axis.

Referring to FIG. 1B, the therapeutic hypothesis for axial myopia according to the present disclosure includes inhibition of progression of and treatment of axial myopia by restoring the balance of neurohumoral factors through the patient's actions (including behavioral, emotional, and cognitive areas) and environment, and the patient's participation.

Referring to FIG. 1C, the digital therapeutic hypothesis for axial myopia is realized as a digital apparatus and an application configured to present changes in patient's actions, improvement of patient's environment, and patient's participation in the form of specific instructions and collect and analyze execution of the specific instructions. When the digital therapeutics of the present disclosure are used, the imbalance of neurohumoral factors for axial myopic patients in the childhood/adolescence stages may be corrected through the digital inputs (instructions) and outputs (execution) to achieve inhibition of progression of and treatment of axial myopia.

Meanwhile, the mechanism of action in and the therapeutic hypothesis for axial myopia are described with reference to FIGS. 1A and 1B, but the present disclosure is not limited thereto. For example, the methodology of the present disclosure may be applied to all types of myopia, and any other diseases.

Also, although the insulin-like growth factor (IGF), cortisol, and dopamine are described as the neurohumoral factors as shown in FIGS. 1A and 1B, it should be understood that the description of the neurohumoral factors is given by way of illustration only, and are not intended to be limiting in all aspects of the mechanism of action in and the therapeutic hypothesis for myopia according to the present disclosure. Accordingly, all the neurohumoral factors having an influence on the myopia may be considered.

FIG. 2 is a block diagram showing a configuration of the digital apparatus for treating myopia according to one embodiment of the present disclosure.

Referring to FIG. 2, a digital system 000 for treating myopia according to one embodiment of the present disclosure may include a digital instruction generation unit 010, a sensing data collection unit 020, an execution input unit 030, an outcome analysis unit 040, a database 050, and a security unit 060.

Based on the mechanism of action in and the therapeutic hypothesis and digital therapeutic hypothesis for axial myopia in the childhood/adolescence stages, a doctor (a second user) may prescribe digital therapeutics, which are realized in a digital apparatus and an application for treating myopia, for the corresponding patient. In this case, the digital instruction generation unit 010 is a device configured to provide a prescription of the digital therapeutics to a patient as a specific behavioral instruction that the patient may execute based on the interaction between the neurohumoral factors for myopia and the patient's behaviors/environments. For example, the neurohumoral factors may include IGF, cortisol, dopamine, and the like, but the present disclosure is not limited thereto. For example, all types of neurohumoral factors that may cause myopia may be considered.

The digital instruction generation unit 010 may generate digital instructions based on the inputs from the doctor. In this case, the digital instruction generation unit 010 may generate digital instructions based on information collected by the doctor when diagnosing a patient. Also, the digital instruction generation unit 010 may generate digital instructions based on the information received from the patient. For example, the information received from the patient may include the patient's basal factors, medical information, and digital therapeutics literacy. In this case, the basal factors may include amount of the patient's activity, heart rates, sleep, meals (nutrition and calories), and the like. The medical information may include the patient's electronic medical record (EMR), family history, genetic vulnerability, genetic susceptibility, and the like. The digital therapeutics literacy may include the patient's accessibility and an acceptance posture to the digital therapy instructions and the apparatus, and the like.

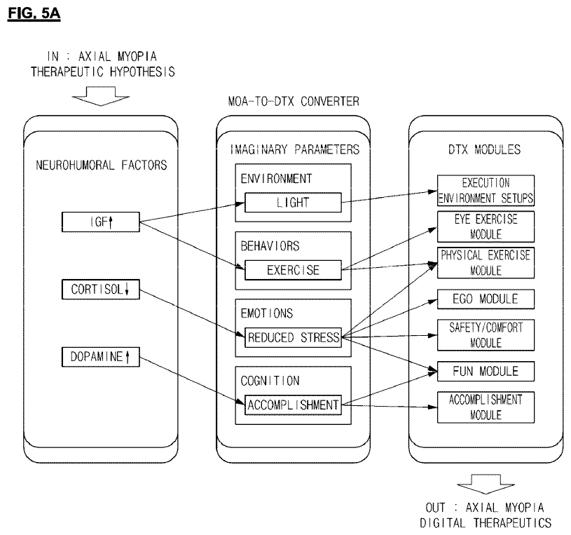

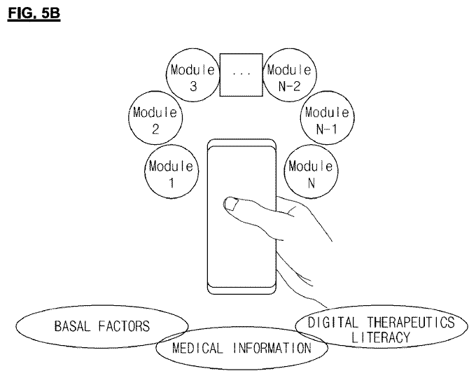

The digital instruction generation unit 010 may reflect the mechanism of action in and the therapeutic hypothesis for myopia in order to utilize imaginary parameters and generate a digital module. In this case, the imaginary parameters may be deduced in term of the patient's environments, behaviors, emotions, and cognition. In this regard, the imaginary parameters will be described in detail as shown in FIG. 5.

The digital instruction generation unit 010 generates digital instructions particularly designed to allow a patient to have a therapeutic effect, and provides the instructions to the patient. For example, the digital instruction generation unit 010 may provide light stimuli under a bright light environment, and simultaneously generate specific digital instructions in each of digital therapeutic modules.

The sensing data collection unit 020 and the execution input unit 030 may collect the patient's execution outcomes of the digital instructions provided at the digital instruction generation unit 010. In some implementations, the sensing data collection unit 020 is an output unit of various sensor devices. Specifically, the sensing data collection unit 020 configured to sense the patient's adherence to the digital instructions and the execution input unit 030 configured to allow a patient to directly input the execution outcomes of the digital instructions are included, and thus serve to output the patient's execution outcomes of the digital instructions. The execution input unit 030 may receive an input regarding a result of performing a digital action.

The outcome analysis unit 040 may collect the patient's behavior adherence or participation in predetermined periods and report the patient's behavior adherence or participation to external systems. Therefore, a doctor may continue to monitor an execution course of the digital instructions through the application even when a patient does not directly visit a hospital.

The database 050 may store the mechanism of action in myopia, the therapeutic hypothesis for myopia, the digital instructions provided to the user, and the user's execution outcome data. FIG. 2 shows that the database 050 is included in the digital apparatus 000 for treating myopia. However, the database 050 may be provided in an external server.

Meanwhile, a series of loops including inputting the digital instructions at the digital instruction generation unit 010, outputting the patient's execution outcomes of the digital instructions at the sensing data collection unit 020/execution input unit 030, and evaluating the execution outcomes at the outcome analysis unit 040 may be repeatedly executed several times. In this case, the digital instruction generation unit 010 may generate patient-customized digital instructions for this cycle by reflecting the patient's digital instructions provided in the previous cycle and output values, and the evaluation.

As described above, according to the digital therapy apparatus for inhibiting progression of and treating axial myopia according to the present disclosure, the myopia therapy whose reliability may be ensured is possible by deducing the mechanism of action in axial myopia and the therapeutic hypothesis and digital therapeutic hypothesis for axial myopia in consideration of the neurohumoral factors for axial myopia, presenting the setups of light stimulus environments suitable for the patient and digital instructions for treating axial myopia based on the mechanism of action and the therapeutic hypotheses, and collecting and analyzing execution of specific instructions.

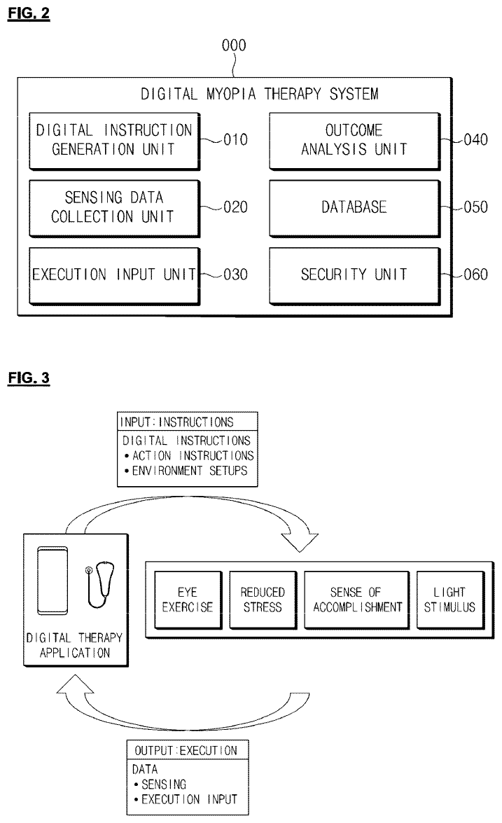

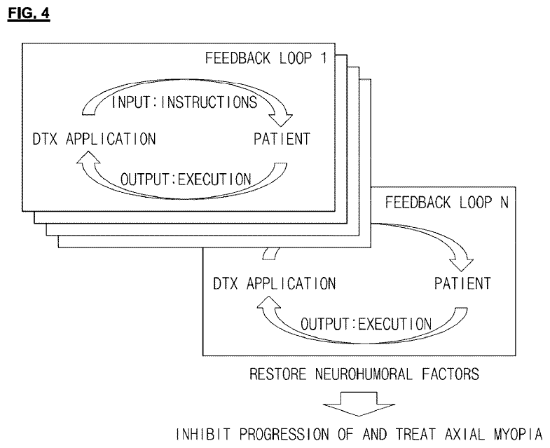

FIG. 3 is a diagram showing input and output loops of the digital application for treating myopia according to one embodiment of the present disclosure.

Referring to FIG. 3, the digital application for treating myopia according to one embodiment of the present disclosure may input the corresponding digital prescription for a patient in the form of instructions, and may output execution outcomes of the corresponding digital instructions.

The digital instructions provided to the patient may include specific action instructions for behaviors, emotions, cognition, and the like, and control of the patient's light environments. As shown in FIG. 3, the digital instructions may include eye exercise, reduced stress, a sense of accomplishment, light stimulus, and the like. However, the digital instructions are given by way of illustration only, and are not intended to be limiting to the digital instruction according to the present disclosure.

The patient's execution outcomes of the digital instructions consist of 1) log-in/log-out information for instructions and execution, 2) adherence information sensed as passive data such as eye exercise, heart rates associated with the stress, a change in oxygen saturation, and the like, and 3) directly input information on the patient's execution outcomes.

FIG. 4 is a diagram showing a feedback loop for the digital apparatus and the application for treating myopia according to one embodiment of the present disclosure.

Referring to FIG. 4, the inhibition of the progression of and the treatment of axial myopia are shown to be achieved by repeatedly executing the aforementioned single feedback loop of FIG. 3 several times to regulate the neurohumoral factors.

In the case of the axial myopia, the digital therapy and observation take a short period of 10 weeks to the whole period of the childhood/adolescence stages to treat the axial myopia due to the pathological characteristics of the axial myopia. Due to these characteristics, inhibitory and therapeutic effects on progression of the axial myopia may be more effectively achieved by gradual improvement of an instruction-execution cycle in the feedback loop, compared to the simply repeated instruction-execution cycle during the corresponding course of therapy.

For example, the digital instructions and the execution outcomes for the first cycle are given as input values and output values in a single loop, but new digital instructions may be generated by reflecting input values and output values generated in this loop using a feedback process of the loop to adjust the input for the next loop when the feedback loop is executed N times. This feedback loop may be repeated to deduce patient-customized digital instructions and maximize a therapeutic effect at the same time.

As such, in the digital apparatus and the application for treating myopia according to one embodiment of the present disclosure, the patient's digital instructions provided in the previous cycle (for example, a N−1^{st} cycle), and the data on instruction execution outcomes may be used to calculate the patient's digital instructions and execution outcomes in this cycle (for example, a N^{th} cycle). That is, the digital instructions in the next loop may be generated based on the patient's digital instructions and execution outcomes of the digital instructions calculated in the previous loop. In this case, various algorithms and statistical models may be used for the feedback process, when necessary.

As described above, in the digital apparatus and the application for treating myopia according to one embodiment of the present disclosure, it is possible to optimize the patient-customized digital instructions suitable for the patient through the rapid feedback loop.

FIG. 5A is a diagram showing a module design for realizing a digital therapy in the digital apparatus and the application for treating myopia according to one embodiment of the present disclosure, FIG. 5B is a diagram showing a background factors supporting the digital apparatus and the application for treating myopia according to one embodiment of the present disclosure

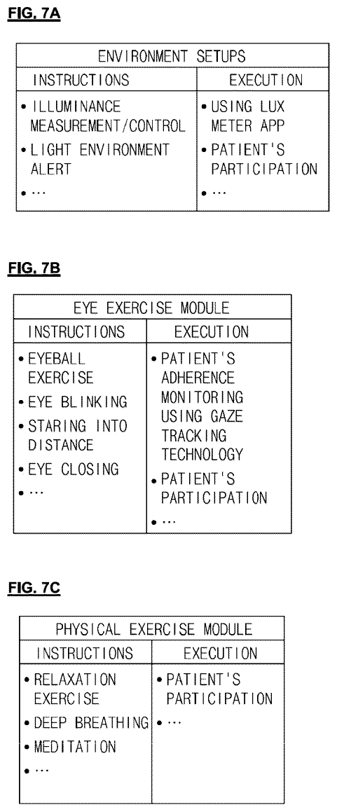

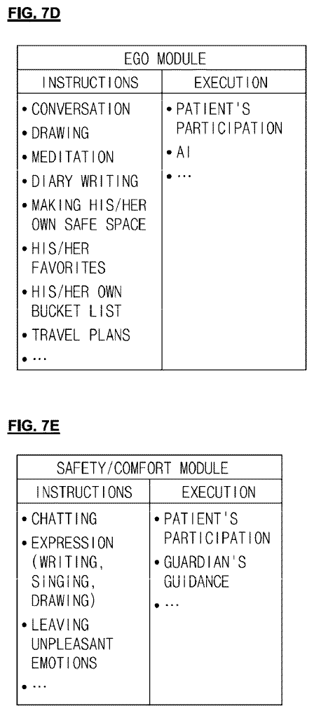

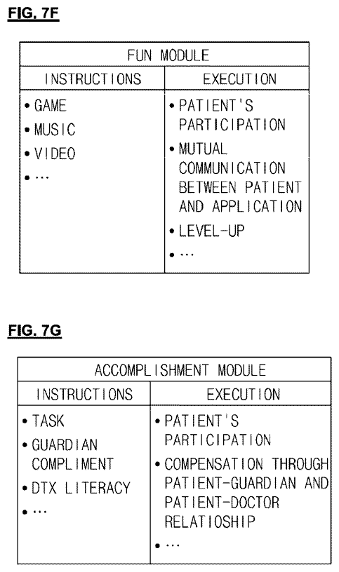

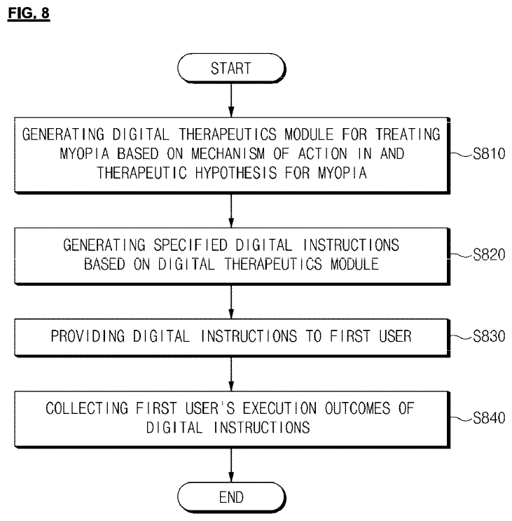

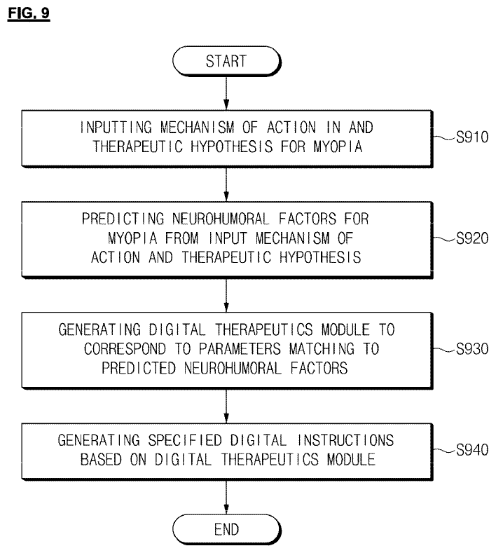

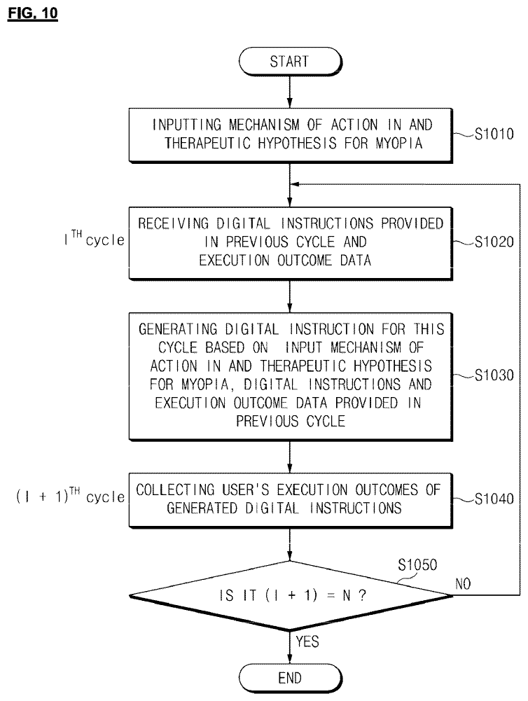

As shown in FIG. 5A, when the therapeutic hypothesis based on the mechanism of action in myopia is created, targeted neurohumoral factors (for example, IGF, cortisol, dopamine, etc.) may be deduced. Imaginary parameters may be utilized to allow specific instructions to correspond to the regulation of these neurohumoral factors. Modules required to treat myopia was deduced using the “neurohumoral factor-imaginary parameter-module” interrelation. Each of the modules will be described in the form of modular instructions in further detail with reference to FIG. 7 as will be described below. In this case, each of the modules is in fact a basic design unit for digital therapeutics realized in the digital apparatus or the application, and is a collection of specific instructions.

Specifically, referring to FIG. 5A, the neurohumoral factors deduced based on the mechanism of action in and the therapeutic hypothesis for axial myopia may be IGF, cortisol (or TGF-beta influenced by the cortisol), and dopamine (or GABA agonist/antagonists, glucagon). To treat myopia, the neurohumoral factors should be regulated in the corresponding age groups to promote secretion of IGF and dopamine having an influence on the eye development and inhibit secretion of cortisol.

The control of each of the neurohumoral factors corresponds to the digital therapeutics module using environments (light), behaviors (exercise), emotions (reduced stress), and cognition (a sense of accomplishment) as imaginary parameters. Specific digital instructions for each module are generated based on the converted modules. In this case, the digital instructions may include execution environment setups and modules (e.g., eye exercise, gymnastics, ego, safety/comfort, fun, and accomplishment modules), which may be output by monitoring. However, the modules are given by way of illustration only, and are not intended to be limiting to the modules according to the present disclosure.

Meanwhile, referring to FIG. 5B, the background factors may be considered together in the design of the modules in the digital apparatus and the application for treating myopia according to one embodiment of the present disclosure.

In this case, the background factors are elements necessary for correction of clinical trial outcomes during verification of the clinical effectiveness of digital myopia therapy according to the present disclosure. Specifically, in the background factors shown in FIG. 5B, the basal factors may include activity, heart rates, sleep, meals (nutrition and calories), and the like, the medical information may include EMR, family history, genetic vulnerability, and susceptibility, and the like, which have been written when a patient visited a hospital, and the digital therapeutics literacy may include the patient's accessibility to the digital therapy instructions and the apparatus, and an acceptance posture.

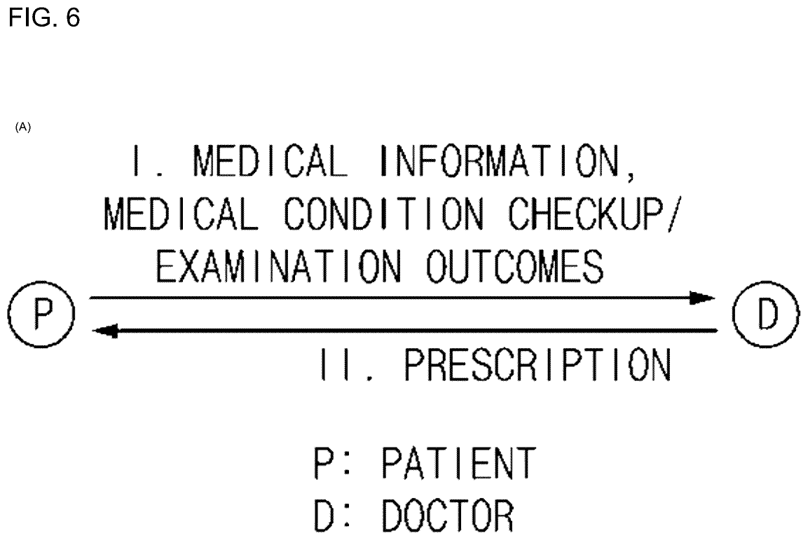

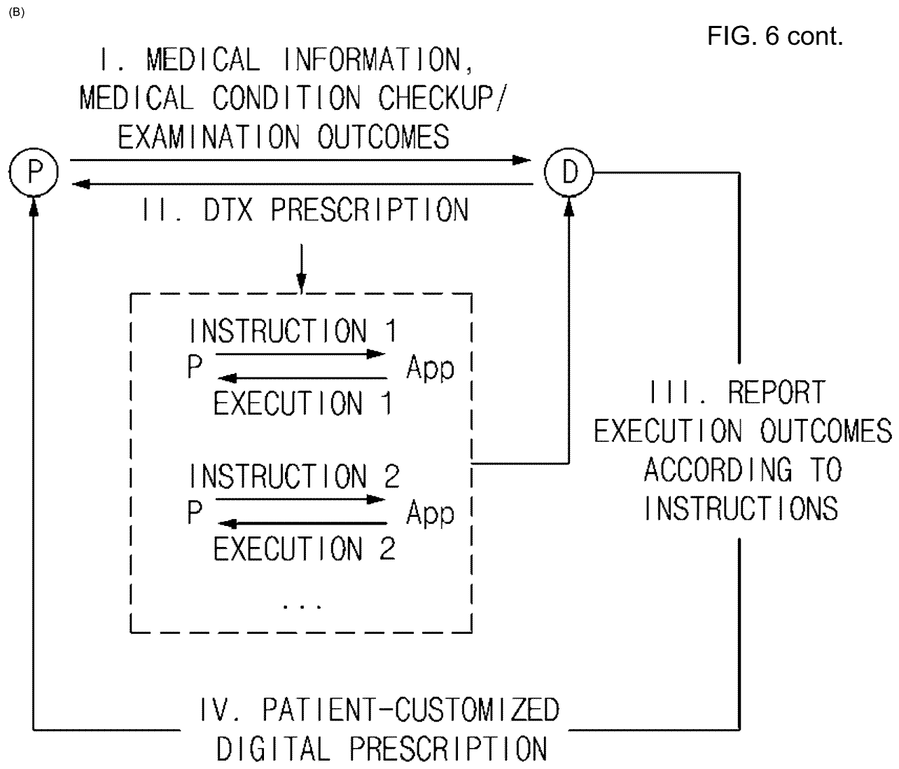

FIG. 6 is a diagram showing a method of assigning a patient-customized digital prescription using the digital apparatus and the application for treating myopia according to one embodiment of the present disclosure.

FIG. 6(A) show a prescription procedure for routine medical condition checkup of a patient by a doctor, and FIG. 6(B) show a method of allowing a doctor to assign a patient-customized digital prescription based on the analysis of a plurality of digital instructions and execution outcomes of the digital instructions.

In this way, when the digital apparatus and the application for treating myopia according to one embodiment of the present disclosure are used, the doctor may check the patient's instructions and execution outcomes for a given period and adjust the types of modules for treating myopia, and the instructions for each module in a patient-customized manner, as shown in FIG. 6(B).

FIG. 7A shows execution environment setups according to one embodiment of the present disclosure, and FIGS. 7B to 7G show examples of specific instructions for each module, and methods of collecting output data according to one embodiment of the present disclosure.

For digital therapy of axial myopia, because the patient's persistent participation is generally essential for 10 weeks or more, it is far more important that adolescent children have fun in digital therapy and voluntarily participate in the digital therapy. In this context, the modules may be configured by adding game elements to each module. In the digital apparatus and the application for treating myopia, which have been realized to relieve and treat axial myopia, as will be described below, each module is a basic design unit and is a collection of specific instructions.

Referring to FIG. 7A, specific examples of instructions for execution environment setups, and a method of collecting output data are shown. In this case, the execution environment setups may be included as part of the configuration of the digital instruction generation unit 010 shown in FIG. 2.

Specifically, the execution environment setups include setup of brightness of an execution environment using an illuminance sensor, and other modules are executed under a set light environment.

In general, sunlight is closely related to the eye health. The same strong light stimulation as in exposure to direct sunlight acts in nerve cells of the retina to promote secretion of dopamine, thereby inducing synthesis of proteoglycans. This is a factor essential for normally adjusting an axial length of the eye.

As described above, the illuminance under a current environment may be measured using an illuminance sensor to provide light stimuli to a patient, an alarm of a current light environment may be provided to brightly control an environment at which a patient participates in digital therapy.

Referring to FIG. 7B, specific examples of instructions for an eye exercise module, and a method of collecting output data are shown. In this case, the may be included as part of the configuration of the digital instruction generation unit 010 shown in FIG. 2.

The digital instructions for eye exercise include controlling the patient's eye exercise, biofeedbacks, eyeball-related behaviors, and the like, and promote secretion of IGF in oculomotor muscles. Specifically, behavioral instructions for the eye exercise module may monitor the patient's adherence using eye tracking technology such as eye exercise, eye blinking, remote staring, eye closing, and the like. However, a collection of the execution outcomes for the eye exercise module is not limited to the eye tracking technology, and include directly inputting the execution outcomes of the instructions by the patient.

Referring to FIG. 7C, specific examples of instructions for a physical exercise module, and a method of collecting output data are shown. In this case, the physical exercise module may be included as part of the configuration of the digital instruction generation unit 010 shown in FIG. 2. The physical exercise module includes slow and comfortable physical exercise, and abdominal exercise, and may be composed of a series of behavioral instructions configured to reduce stress through breaks, relaxation, deep breathing, and the like in order to inhibit secretion of cortisol.

Specifically, the behavioral instructions for the physical exercise module include behavioral instructions such as relaxation exercise, deep breathing, meditation, eye massage, and the like. Also, the behavioral instructions include a method of collecting the execution outcomes of the behavioral instructions at the sensing data collection unit 020 using a biofeedback apparatus (for measuring EEG, ECG, EMG, EDG, etc.) or a general-purpose sensor (for measuring activity, HR, etc.), or a method of allowing a patient to directly input the execution outcomes using the execution input unit 030. The behavioral instructions of the present disclosure are composed based on a behavior therapy method which is widely used to relieve stress of children in the child psychiatry.

In general, the progression of myopia is closely related to the course of adolescence. In particular, there might be a great deal of variation among the adolescent children in this stage, depending on the age, gender, character, and preference of the children. To cover these deviations, it is desirable that the digital instructions for each module are presented in a customized manner according to the individual characteristics of each patient. In particular, instructions requiring the mutual communication (for example, conversation) with an application may be developed in combination with big data analysis and artificial intelligence analysis.

Referring to FIG. 7D, specific examples of instructions for an ego module, and a method of collecting output data are shown. In this case, the ego module may be included as part of the configuration of the digital instruction generation unit 010 shown in FIG. 2.

Specifically, the instructions for the ego module aim to serve to increase the adolescents' self-esteem and relive the stress. To do this, the instructions for the ego module may, for example, include instructions such as conversation, drawing, meditation, diary writing, making his/her own safe space (safety place instructions), his/her favorites (places, time, seasons, colors, foods, humans, etc.), his/her own bucket lists, choosing places to travel and planning for travel, and the like. These instructions are composed based on a psychotherapy which has been widely used in the child psychiatry to increase the self-esteem and relive the stress of children or adolescents.

Referring to FIG. 7E, specific examples of instructions for a safety/comfort module, and a method of collecting output data are shown. In this case, the safety/comfort module may be included as part of the configuration of the digital instruction generation unit 010 shown in FIG. 2.

Specifically, the instructions for the safety/comfort module aim to serve as ventilation to reduce the adolescents' stress. To do this, the instructions for the safety/comfort module may, for example, include instructions such as chatting, expression (writing, singing, drawing), leaving unpleasant emotions in an animational aspect (trash may instructions), and the like. These instructions are composed based on a psychotherapy which has been widely used in the child psychiatry to increase the self-esteem and relive the stress of children or adolescents.

Referring to FIG. 7F, specific examples of instructions for a fun module, and a method of collecting output data are shown. In this case, the fun module may be included as part of the configuration of the digital instruction generation unit 010 shown in FIG. 2.

Specifically, the instructions for the fun module are instructions that allow a patient to use an application and have fun, may be compose of various contents such as music, games, or videos, depending on the adolescent characteristics. Also, fun instructions in the fun module also aim to improve the patient's persistent participation in the digital therapy.

Referring to FIG. 7G, specific examples of instructions for an accomplishment module, and a method of collecting output data are shown. In this case, the accomplishment module may be included as part of the configuration of the digital instruction generation unit 010 shown in FIG. 2.

Specifically, the instructions for the accomplishment module may include instructions that promote secretion of dopamine through senses of accomplishment such as the patient's task execution and completion. Here, the task accomplishment instructions are instructions that allow a patient to feel a sense of accomplishment when a given task is accomplished, and thus may include games whose tasks may be updated over a patient's participation duration and which may induce the patient's voluntary participation. For example, a specific format of the game may be composed of various times such as learning, hidden or difference pictorial puzzles, quizzes, and the like.

In particular, some instructions realized in the form of quizzes at the accomplishment module may be expected to have an additional effect of improving the patient's health information literacy and digital therapeutics literacy. Such improvement of the health information and digital therapeutics literacy is an element essential for the patient's persistent participation and execution in the therapy.

As mentioned above, the digital therapy according to the present disclosure requires not less than 10 weeks of the patient's participation. During this period, sincerely executing the instructions for the aforementioned modules makes it possible to form compliment instructions in the accomplishment module so that a patient feels a sense of accomplishment. For the compliment instructions, the patient's active participation in the therapy may be fed back as a sense of accomplishment based on the reliance and compensation between the patient and a guardian and between the patient and the doctor.

The digital instruction shown above in FIG. 7B to FIG. 7G are given by way of illustration only, and are not intended to limit the present disclosure. For example, the digital instructions provided to the patient may be set in various manners, when necessary.

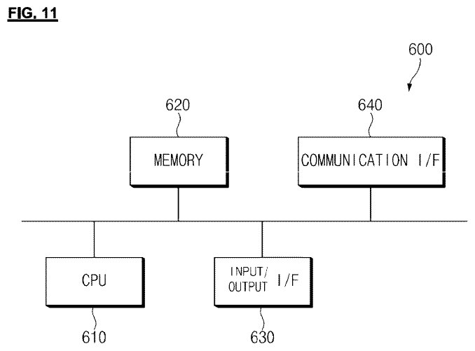

FIG. 8 is a flowchart illustrating operations in the digital application for treating myopia according to one embodiment of the present disclosure.

Referring to FIG. 8, the digital application for treating myopia according to one embodiment of the present disclosure may first generate a digital therapeutics module for treating myopia based on the mechanism of action in and the therapeutic hypothesis for myopia (S810). In this case, in S810, the digital therapeutics module may be generated based on the neurohumoral factors (for example, IGF, cortisol, dopamine, etc.) for myopia.

Meanwhile, in S810, the digital therapeutics module may be generated based on the inputs from the doctor. In this case, a digital therapeutics module may be generated based on the information collected by the doctor when diagnosing a patient, and the prescription outcomes recorded based on the information. Also, in S810, the digital therapeutics module may be generated based on the information (for example, basal factors, medical information, digital therapeutics literacy, etc.) received from the patient.

Next, in S820, specified digital instructions may be generated based on the digital therapeutics module. S820 may generate a digital therapeutics module by applying imaginary parameters about the patient's environments, behaviors, emotions, and cognition to the mechanism of action in and the therapeutic hypothesis for myopia. This digital therapeutics module is described with reference to FIG. 5, and thus description thereof will be omitted.

In this case, the digital instructions may be generated for at least one of light environment setup, eye exercise, physical exercise, ego, safety/comfort, fun, and accomplishment modules. Description of the execution environment setups and the specific digital instructions for each of the modules is as described in FIGS. 7A to 7G.

Then, the digital instructions may be provided to a patient (S830). In this case, the digital instructions may be provided in the form of digital instructions which are associated with behaviors, emotions, cognition and in which the patient's instruction adherence such as eye exercise/physical exercise may be monitored using a sensor, or provided in the form of digital instructions in which a patient is allowed to directly input the execution outcomes.

After the patient executes the presented digital instructions, the patient's execution outcomes of the digital instructions may be collected (S840). In S840, the execution outcomes of the digital instructions may be collected by monitoring the patient's adherence to the digital instructions as described above, or allowing the patient to input the execution outcomes of the digital instructions.

Meanwhile, the digital application for treating myopia according to one embodiment of the present disclosure may repeatedly execute operations several times, wherein the operations include generating the digital instruction and collecting the patient's execution outcomes of the digital instructions. In this case, the generating of the digital instruction may include generating the patient's digital instructions for this cycle based on the patient's digital instructions provided in the previous cycle and the execution outcome data on the patient's collected digital instructions provided in the previous cycle.

As described above, according to the digital application for treating myopia according to one embodiment of the present disclosure, the reliability of the inhibition of progression of and treatment of myopia may be ensured by deducing the mechanism of action in and the therapeutic hypothesis for myopia in consideration of the neurohumoral factors for myopia, presenting the digital instructions to a patient based on the mechanism of action in and the therapeutic hypothesis for myopia, executing the digital instructions under a suitable light stimulus environment, and collecting and analyzing the outcomes of the digital instructions.

Although the digital apparatus and the application for treating myopia according to one embodiment of the present disclosure have been described in terms of myopia therapy, the present disclosure is not limited thereto. For the other diseases other than the myopia, the digital therapy may be executed substantially in the same manner as described above.

FIG. 9 is a flowchart illustrating a method of generating digital instructions in the digital application for treating myopia according to one embodiment of the present disclosure.

Referring to FIG. 9, operations of the method of generating digital instructions are as described above in the process of generating the module for treating myopia and the specified digital instructions based on the mechanism of action in and the therapeutic hypothesis for myopia (S810 and S820 shown in FIG. 8) and the processes shown in FIG. 5.

In S910, first of all, the mechanism of action in and the therapeutic hypothesis for myopia may be input. In this case, the mechanism of action in and the therapeutic hypothesis for myopia may be previously deduced through the literature search and expert reviews on the systematic related clinical trials on myopia, as described above.

Next, neurohumoral factors for myopia may be predicted from the input mechanism of action and therapeutic hypothesis (S920). In this case, the neurohumoral factors for myopia predicted in S920 may be deduced in the form of IGF, cortisol, dopamine, and the like. These neurohumoral factors have been described in detail with reference to FIG. 5, and thus description thereof will be omitted.

In S930, a digital therapeutics module may be generated so that the imaginary parameters may correspond to the predicted neurohumoral factors. Here, the imaginary parameters may serve as converters that convert the neurohumoral factors for myopia into a digital therapeutics module, and this procedure is to set the physiological interrelation between the neurohumoral factors and the environmental, behavioral, emotional and cognition factors, as shown in FIG. 5.

Then, specified digital instructions may be generated based on the generated digital therapeutics module (S940). In this case, the specific digital instructions may be generated at the aforementioned light environment setup, eye exercise, physical exercise, ego, safety/comfort, fun, and accomplishment modules with reference to FIGS. 7A to 7G.

FIG. 10 is a flowchart illustrating a method of repeatedly executing the operations under the feedback control in the digital application for treating myopia according to one embodiment of the present disclosure.

In FIG. 10, it is explained that generation of the digital instructions and collection of the execution outcomes at the digital application for treating myopia are executed N times. In this case, the mechanism of action in and the therapeutic hypothesis fir myopia may be first input (S1010). Also, the digital instructions provided in the previous cycle, and the execution outcome data may be received (S1020). When the first cycle of execution is now in progress, S1020 may be omitted because there are no previous data.

Next, digital instructions for this cycle may be generated based on the input mechanism of action and therapeutic hypothesis, the digital instruction provided in the previous cycle, and the execution outcome data (S1030). Then, the user's execution outcomes of the generated digital instructions may be collected (S1040).

In S1050, it is judged whether this cycle is greater than N^{th} cycle. When this cycle is less than the N^{th} cycle (NO), this may return again to S1020, thus repeatedly executing S1020 to S1040. On the other hand, when this cycle is greater than the N^{th} cycle (YES), that is, when the generation of the digital instructions and the collection of the execution outcomes are executed N times, a feedback operation may be terminated.

FIG. 11 is a diagram showing a hardware configuration of the digital apparatus for treating myopia according to one embodiment of the present disclosure. Various examples of the digital apparatus include a desktop computer, a laptop computer, a tablet computer, a server computer, a server system, a wearable device such as a smart watch, and other computing devices. The digital apparatus may be a data server that hosts one or more databases (e.g., database of images or videos), models, or modules, or may provide various executable applications or modules.

Referring to FIG. 11, hardware 600 of the digital apparatus for treating myopia according to one embodiment of the present disclosure may include a CPU 610, a memory 620, an input/output I/F 630, and a communication I/F 640.

The CPU 610 may include a processor configured to execute a digital program for treating myopia stored in the memory 620, process various data for treating digital myopia and execute functions associated with the digital myopia therapy. That is, the CPU 610 may act to execute functions for each of the configurations shown in FIG. 2 by executing the digital program for treating myopia stored in the memory 620.

The memory 620 may have a digital program for treating myopia stored therein. Also, the memory 620 may include the data used for the digital myopia therapy included in the aforementioned database 050, for example, the patient's digital instructions and instruction execution outcomes, the patient's medical information, and the like.

A plurality of such memories 620 may be provided, when necessary. The memory 620 may be a volatile memory or a non-volatile memory. When the memory 620 is a volatile memory, RAM, DRAM, SRAM, and the like may be used as the memory 620. When the memory 620 is a non-volatile memory, ROM, PROM, EAROM, EPROM, EEPROM, a flash memory, and the like may be used as the memory 620. Examples of the memories 620 as listed above are given by way of illustration only, and are not intended to limit the present disclosure.

In some implementations, the memory 620 or the computer-readable storage medium of the memory 620 store the following programs, modules, and data structures, or a subset or superset thereof.
an operating system, which includes procedures for handling various basic system services and for performing hardware dependent tasks;
a communications module, which is used for connecting the computing device 200 to other computers and devices via the one or more communication network interfaces 204 (wired or wireless), such as the Internet, other wide area networks, local area networks, metropolitan area networks, and so on;
a web browser (or other application capable of displaying web pages), which enables a user to communicate over a network with remote computers or devices;
an audio input module (e.g., a microphone module) for processing audio captured by the audio input device. The captured audio may be sent to a remote server and/or processed by an application executing on the digital device;
a healthcare application, which includes a graphical user interface that allows a user to navigate the healthcare application, such as accessing a patient profile, viewing patient information for the patient profile, and selecting digital therapeutic modules. In some implementations, the healthcare application may utilize a healthcare provider communication module to send patient information, such as adherence information or sensor information to a healthcare provider. The healthcare application may also utilize the healthcare provider communication module to receive instructions from a healthcare provider to update or modify one or more computer programs (or one or more digital therapeutic modules). In some implementations, the healthcare application may include a sensor module that stores information regarding sensor configurations for tracking user activity or user adherence to the computer programs (digital therapeutic modules);
digital therapeutic modules configured to generate instructions for a subject to follow and/or modify instructions to generate customized digital therapeutic modules that are customized for a specific patient based on the patient's patient profile; and
a database, which stores information, such as patient profiles, healthcare provider data, and digital therapeutic modules. Patient profile may include sensor information, such as user adherence information and/or use progress information, and patient information, such as age, gender, weight height, diagnosis, and health care provider.

The input/output I/F 630 may provide an interface in which input apparatuses (not shown) such as a display device (e.g., a screen or monitor), a keyboard, a mouse, a touch panel, and the like, and output apparatuses such as a display (not shown), and the like may transmit and receive data (e.g., wirelessly or by hardline) to the CPU 610. The display device includes a touch-sensitive surface in which case the display device is a touch-sensitive display. In some implementations, the touch-sensitive surface is configured to detect various swipe gestures (e.g., continuous gestures in vertical and/or horizontal directions) and/or other gestures (e.g., single/double tap). The input/output I/F 630 also includes an audio output device, such as speakers or an audio output connection connected to speakers, earphones, or headphones. Furthermore, some digital apparatus 600 may use a microphone and voice recognition software to supplement or replace the keyboard. An audio input device (e.g., a microphone) captures audio (e.g., speech from a user).

The communication I/F 640 is configured to transmit and receive various types of data to/from a server, and may be one of various apparatuses capable of supporting wire or wireless communication. For example, the types of data on the aforementioned digital behavior-based therapy may be received from a separately available external server through the communication I/F 640.

Each of the above identified executable modules, applications, or sets of procedures may be stored in one or more of the previously mentioned memory 620, and corresponds to a set of instructions for performing a function described above. The above identified modules or programs (i.e., sets of instructions) need not be implemented as separate software programs, procedures, or modules, and thus various subsets of these modules may be combined or otherwise re-arranged in various implementations. In some implementations, the memory 620 stores a subset of the modules and data structures identified above. Furthermore, the memories 620 may store additional modules or data structures not described above.

As described above, the computer program according to one embodiment of the present disclosure may be recorded in the memory 620 and processed at the CPU 610, for example, so that the computer program may be realized as a module configured to execute each of functional blocks shown in FIG. 2.

The processor according to an exemplary embodiment of the present disclosure may be a hardware device implemented by various electronic circuits (e.g., computer, microprocessor, CPU, ASIC, circuitry, logic circuits, etc.). The processor may be implemented by a non-transitory memory storⁱng, e.g., a program(s), software instructions reproducing algorithms, etc., which, when executed, performs various functions described hereinafter, and a processor configured to execute the program(s), software instructions reproducing algorithms, etc. Herein, the memory and the processor may be implemented as separate semiconductor circuits. Alternatively, the memory and the processor may be implemented as a single integrated semiconductor circuit. The processor may embody one or more processor(s).

According to the digital apparatus and the application for treating axial myopia according to the present disclosure, a reliable digital apparatus and application capable of inhibiting progression of and treating myopia may be provided by deducing a mechanism of action in myopia and a therapeutic hypothesis and a digital therapeutic hypothesis for myopia in consideration of neurohumoral factors for progression of axial myopia, presenting digital instructions to a patient under suitable light stimulus environment setups based on the mechanism of action, the therapeutic hypothesis, and the digital therapeutic hypothesis, and collecting and analyzing execution outcomes of the digital instructions.

While the disclosure has been shown and described with reference to certain exemplary embodiments thereof, it will be understood by those skilled in the art that various changes in form and details may be made therein without departing from the spirit and scope of the disclosure as defined by the appended claims.

AL (Axial Length) is the combination of anterior chamber depth, lens thickness and vitreous chamber depth, and it is the most significant contributor to refractive error. Myopia results from an increase in AL outside of the normal rate expected for age. In children with rapidly progressing Myopia, AL will increase faster than the normal rate.

Therefore, a method of improving an eyesight of a subject is provide in the present disclosure. The method of improving an eyesight of a subject according to the present disclosure comprises: providing, by a digital apparatus to the subject, a digital application comprising one or more digital therapeutic modules for improving the eyesight. The method according to the present disclosure may improve a growth rate of AL (Axial Length) of said at least one eyeball in the subject. The method according to the present disclosure may reduce a growth rate of AL (Axial Length) of said at least one eyeball in the subject.

In some embodiments, a method of treating myopia in a subject in need thereof, comprises: providing, by a digital apparatus to the subject, a digital application comprising one or more digital therapeutic modules for treating myopia, each of the modules comprising one or more first instructions for the subject to follow, wherein the first instructions comprise a first eyeball exercise instruction for the subject to move at least one eyeball vertically.

In some embodiments, a system for improving an eyesight in a subject, the system comprises a digital apparatus configured to execute a digital application for improving an eyesight in the subject depending methods described above and below; a healthcare provider portal configured to provide one or more options to a healthcare provider to perform one or more tasks to prescribe treatment to improve the eyesight in the subject based on information received from the digital application; and an administrative portal configured to provide one or more options to an administrator of the system to perform one or more tasks to manage access to the system by the healthcare provider.

In some embodiments, a system for treating myopia in a subject in need thereof comprises: a digital apparatus configured to execute a digital application for treating myopia in the subject depending methods described above and below; a healthcare provider portal configured to provide one or more options to a healthcare provider to perform one or more tasks to prescribe treatment to treat myopia in the subject based on information received from the digital application; and an administrative portal configured to provide one or more options to an administrator of the system to perform one or more tasks to manage access to the system by the healthcare provider.

In some embodiments, a non-transitory computer readable medium has stored thereon software instructions for improving an eyesight of a subject that, when executed by a processor, cause the processor to: display, by a digital apparatus to the subject, modules for improving an eyesight, each of the modules comprising one or more instructions for the subject to follow, wherein the first instructions comprise an eyeball exercise instruction for the subject to move at least one eyeball vertically; and sense, by a sensor in the digital apparatus, adherence by the subject to the instructions of the modules.

In some embodiments, a non-transitory computer readable medium having stored thereon software instructions for treating myopia in a subject in need thereof that, when executed by a processor, cause the processor to: display, by a digital apparatus to the subject, modules for treating myopia, each of the modules comprising one or more instructions for the subject to follow, wherein the first instructions comprise an eyeball exercise instruction for the subject to move at least one eyeball vertically; and sense, by a sensor in the digital apparatus, adherence by the subject to the instructions of the modules.

The method of improving the eyesight of the subject according to the present disclosure may be applied to a subject in the growth state from 5 to 12 years of age, preferably, a subject is 10 years old or more.

In some embodiments the modules are selected based on a mechanism of action in and a therapeutic hypothesis, wherein the digital apparatus (i) comprises a sensor sensing adherence by the subject to the one or more first instructions of the modules, (ii) transmits adherence information, based on the adherence, to a server accessible by a healthcare provider through a healthcare provider portal, and (iii) receives one or more second instructions from the healthcare provider based on the adherence information.

In some embodiments, one or more second instructions comprise a second eyeball exercise instructions for an eyeball movement at a speed adjusted based on the adherence information.

In some embodiments, the digital application instructs a processor of the digital apparatus to execute operations comprising: generating digital therapeutic modules based on a mechanism of action and a therapeutic hypothesis.

In some embodiments, the generating of the digital therapeutic modules comprises generating the digital therapeutic modules based on neurohumoral factors.

In some embodiments, the operations further comprise generating a calibration module for calibrating one or more of an accuracy of measurement of the subject's eye position, and a light environment.

In some embodiments, the calibration module is generated prior to generating the digital therapeutic modules.

In some embodiments, the accuracy of measurement of the subject's eye position is calibrated, and said calibrating the accuracy of measurement of the subject's eye position comprises one or more of instructing the subject to position their face to appear on a screen of the digital apparatus, detecting the subject's eyes for a given period of time, instructing the subject to blink their eyes, detecting if the subject blinked their eyes, instructing the subject to stare at the screen, instructing the subject to move their eyes in a given direction or rotate their eyes, and determining a threshold for detecting the subject's eyes

In some embodiments, the accuracy of measurement of the light environment is calibrated, and said calibrating the light environment comprises one or more of detecting light in the subject's environment using a light sensor of the digital apparatus, and instructing the subject to turn on one or more lights in their environment.

In some embodiments, the digital apparatus comprises one or more sensors for tracking movement of the subject's eyeball.

In some embodiments, the digital application instructs a processor of the digital apparatus to execute operations comprising: generating digital therapeutic modules based on a mechanism of action in and a therapeutic hypothesis; generating digital instructions based on the digital therapeutic modules; providing the digital instruction to a subject; and collecting the subject's execution outcomes of the digital instructions.

In some embodiments, generating of the digital instructions and the collecting of the subject's execution outcomes of the digital instructions are repeatedly executed several with multiple feedback loops, and the generating of the digital instructions comprises generating the subject's digital instructions for this cycle based on the subject's digital instructions in the previous cycle and the collected execution outcome data on the subject's digital instructions provided in the previous cycle.

In some embodiments, the collecting the subject's execution outcomes of the digital instructions comprises determining one or both of an exercise intensity (EI) and an average exercise intensity (AEI).

In some embodiments, AEI is determined as an averaged sum of differences between a final location of an eyeball of the subject and a starting location of the eyeball measured at a given interval.

In some embodiments, the interval is between about 10 milliseconds (ms) and about 500 ms.

In some embodiments, the EI is determined according the formula:

$$EI = \frac{AEI \times 100}{145}$$

In some embodiments, the AEI is determined as a sum of static AEI and dynamic AEI.

In some embodiments, the generating of the digital therapeutic modules comprises generating the digital therapeutic modules by applying imaginary parameters about the subject's environments, behaviors, emotions, and cognition to the mechanism of action in and the therapeutic hypothesis.

In some embodiments, the digital application instructs a processor of the digital apparatus to generate digital therapeutic modules comprises: (i) an eye exercise module comprising the eyeball exercise instructions, and (ii) at least one of a relaxation module and a light therapy module.

In some embodiments, the eye exercise module further comprises one or more of biofeedback control instructions and eyeball-related behavior control instructions; the relaxation module comprises one or more relaxation instructions for one or more of: physical exercise instructions, ego enhancement instructions, safety feeling instructions, comfort feeling instructions, and fun instructions; and the light therapy module comprises one or more light therapy instructions for controlling a light environment of the subject.

In some embodiments, the one or more relaxation instructions comprise one or more of playing a sound or song, inducing blinking, and instructing the subject to perform gymnastics.

In some embodiments, the digital therapeutic modules further comprise an accomplishment module comprising: one or more accomplishment instructions for task accomplishment and for providing compensation for the subject's adherence to the instructions of the two or more first modules.

In some embodiments, the digital therapeutic modules further comprise a fun module comprising one or more fun instructions for music, games, or videos.

In some embodiments, the healthcare provider portal is configured to provide one or more options to the healthcare provider to perform one or more tasks to prescribe treatment in the subject based on the adherence information, wherein the one or more options provided to the healthcare provider are selected from the group consisting of adding or removing the subject, viewing or editing personal information for the subject, viewing adherence information for the subject, viewing a result of the subject for one or more at least partially completed digital therapeutic modules, prescribing one or more digital therapeutic modules to the subject, altering a prescription for one or more digital therapeutic modules, and communicating with the subject.

In some embodiments, the one or more options comprise the viewing or editing personal information for the subject, and the personal information comprises one or more selected from the group consisting of an identification number for the subject, a name of the subject, a date of birth of the subject, an email of the subject, an email of the guardian of the subject, a contact phone number for the subject, a prescription for the subject, and one or more notes made by the healthcare provider about the subject.

In some embodiments, the personal information comprises the prescription for the subject, and the prescription for the subject comprises one or more selected from the group consisting of a prescription identification number, a prescription type, a start date, a duration, a completion date, a number of scheduled or prescribed digital therapeutic modules to be performed by the subject, and a number of scheduled or prescribed digital therapeutic modules to be performed by the subject per day.

In some embodiments, the one or more options comprise the viewing the adherence information, and the adherence information of the subject comprises one or more of a number of scheduled or prescribed digital therapeutic modules completed by the subject, and a calendar identifying one or more days on which the subject completed, partially completed, or did not complete one or more scheduled or prescribed digital therapeutic modules.

In some embodiments, the one or more options comprise the viewing the result of the subject, and the result of the subject for one or more at least partially completed digital therapeutic modules comprises one or more selected from the group consisting of a time at which the subject started a scheduled or prescribed digital therapeutic module, a time at which the subject ended a scheduled or prescribed digital therapeutic module, an indicator of whether the scheduled or prescribed digital therapeutic module was fully or partially completed, and an exercise intensity (EI).

In some embodiments, the server is accessible by an administrator through an administrative portal configured to provide one or more options to an administrator of the system to perform one or more tasks to manage access to the system by the healthcare provider, and wherein the one or more options provided to the administrator of the method are selected from the group consisting of adding or removing the healthcare provider, viewing or editing personal information for the healthcare provider, viewing or editing de-identified information of the subject, viewing adherence information for the subject, viewing a result of the subject for one or more at least partially completed digital therapeutic modules, and communicating with the healthcare provider.

In some embodiments, the one or more options comprise the viewing or editing the personal information, and the personal information of the healthcare provider comprises one or more selected from the group consisting of an identification number for the healthcare provider, a name of the healthcare provider, an email of the healthcare provider, and a contact phone number for the healthcare provider.

In some embodiments, the one or more options comprise the viewing or editing the de-identified information of the subject, and the de-identified information of the subject comprises one or more selected from the group consisting of an identification number for the subject, and the healthcare provider for the subject.

In some embodiments, the one or more options comprise the viewing the adherence information for the subject, and the adherence information of the subject comprises one or more of a number of scheduled or prescribed digital therapeutic modules completed by the subject, and a calendar identifying one or more days on which the subject completed, partially completed, or did not complete one or more scheduled or prescribed digital therapeutic modules.

In some embodiments, the one or more options comprise the viewing the result of the subject, and the result of the subject for one or more at least partially completed digital therapeutic modules comprises one or more selected from the group consisting of a time at which the subject started a scheduled or prescribed digital therapeutic module, a time at which the subject ended a scheduled or prescribed digital therapeutic module, an indicator of whether the scheduled or prescribed digital therapeutic module was fully or partially completed, and an exercise intensity (EI).

In some embodiments, the digital application further comprises a push alarm for one or more of reminding the subject complete a digital therapeutic module and adjusting the light settings of the subject's environment.

In some embodiments, the push alarm is activated to remind the subject to adjust the light settings such that the subject is exposed to sufficiently bright light at least 3 times per day.

In some embodiments, the digital apparatus comprises: a digital instruction generation unit configured to generate digital therapeutic modules based on a mechanism of action (MOA) in and a therapeutic hypothesis, generate digital instructions based on the digital therapeutic modules, and provide the digital instructions to the subject; and an outcome collection unit configured to collect the subject's execution outcomes of the digital instructions.

In some embodiments, the digital instruction generation unit generates the digital therapeutic modules based on neurohumoral factors.

In some embodiments, the neurohumoral factors comprise insulin-like growth factor (IGF), cortisol, and dopamine.

In some embodiments, the digital instruction generation unit generates the digital therapeutic modules based on the inputs from the healthcare provider.

In some embodiments, the digital instruction generation unit generates the digital therapeutic modules based on information received from the subject.

In some embodiments, the information is received from the subject comprises at least one of basal factors, medical information, and digital therapeutics literacy of the subject, the basal factors including the subject's activity, heart rate, sleep, and diet (including nutrition and calories), the medical information including the subject's electronic medical record (EMR), family history, genetic vulnerability, and genetic susceptibility, and the digital therapeutics literacy including the subject's accessibility, and technology adoption to the digital therapeutics and the apparatus.

In some embodiments, the digital instruction generation unit generates the digital therapeutic modules matching to imaginary parameters which correspond to the mechanism of action in and the therapeutic hypothesis.

In some embodiments, the imaginary parameters are deduced in relation to the subject's environment, behaviors, emotions, and cognition.

In some embodiments, the outcome collection unit collects the execution outcomes of the digital instructions by monitoring the subject's adherence to the digital instructions or allowing the subject to directly input the subject's adherence to the digital instructions.

In some embodiments, the generation of the digital instructions at the digital instruction generation unit and the collection of the subject's execution outcomes of the digital instructions at the outcome collection unit are repeatedly executed several times with multiple feedback loops, and the digital instruction generation unit generates the subject's digital instructions for this cycle based on the subject's digital instructions in the previous cycle and the execution outcome data on the subject's digital instructions in the previous cycle collected at the outcome collection unit.

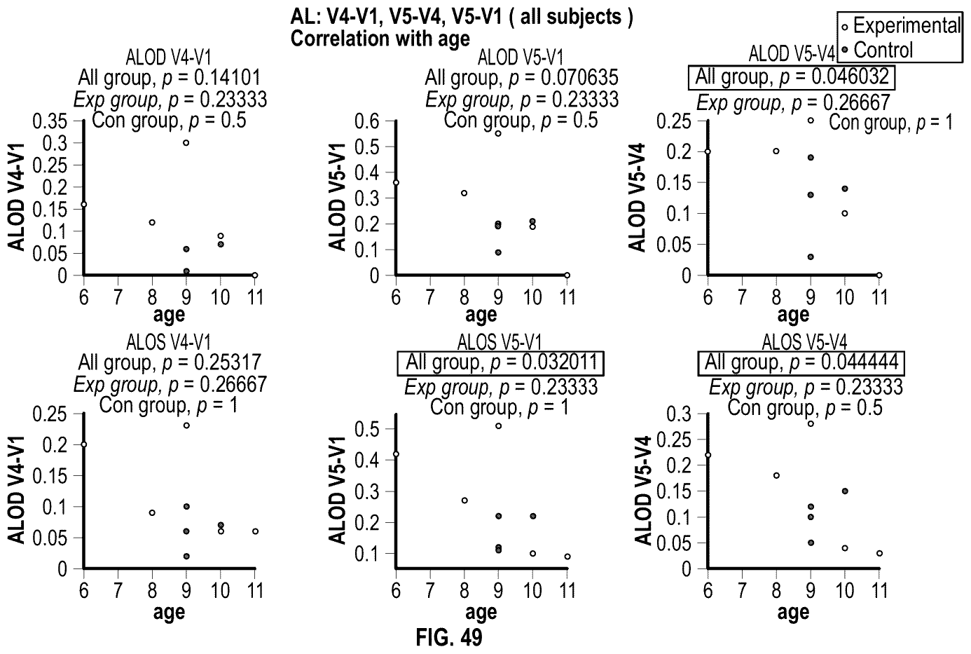

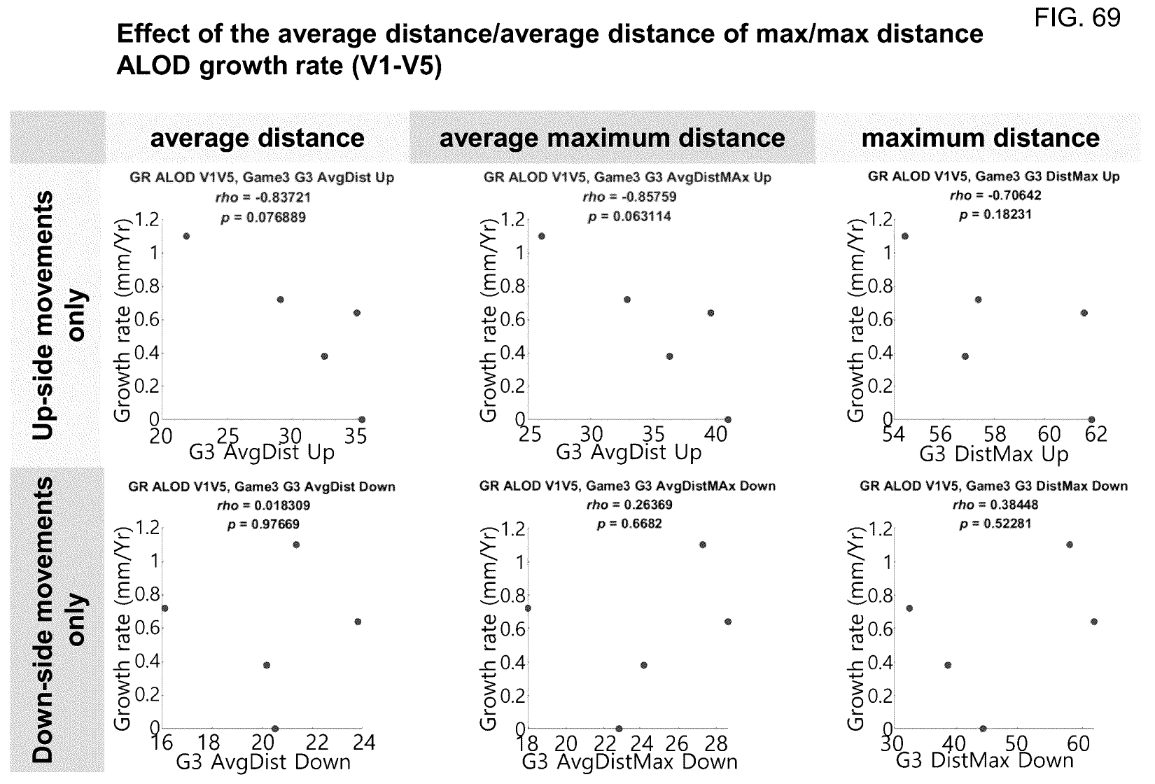

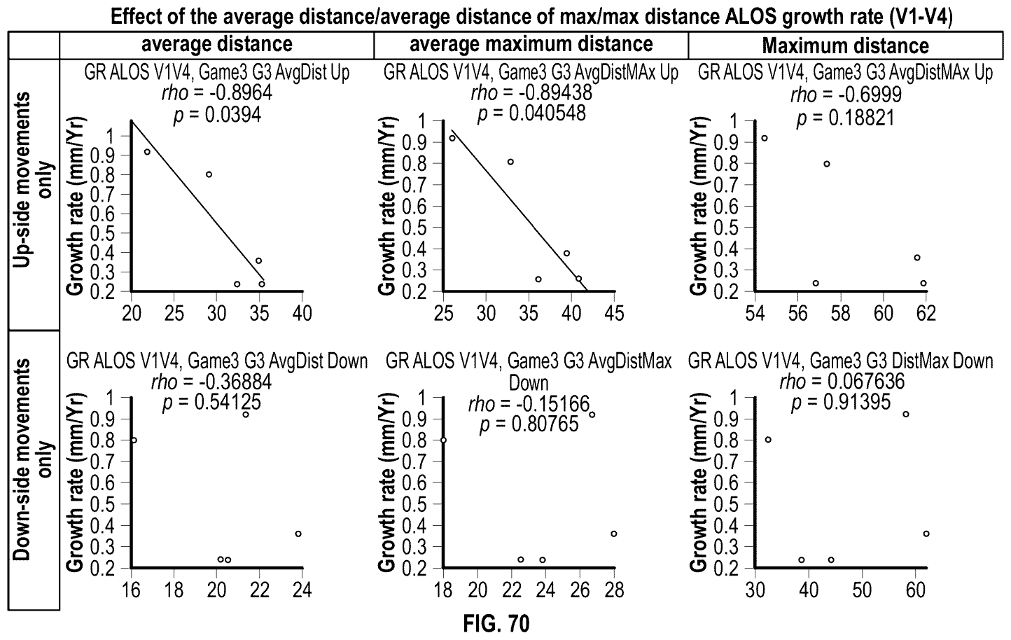

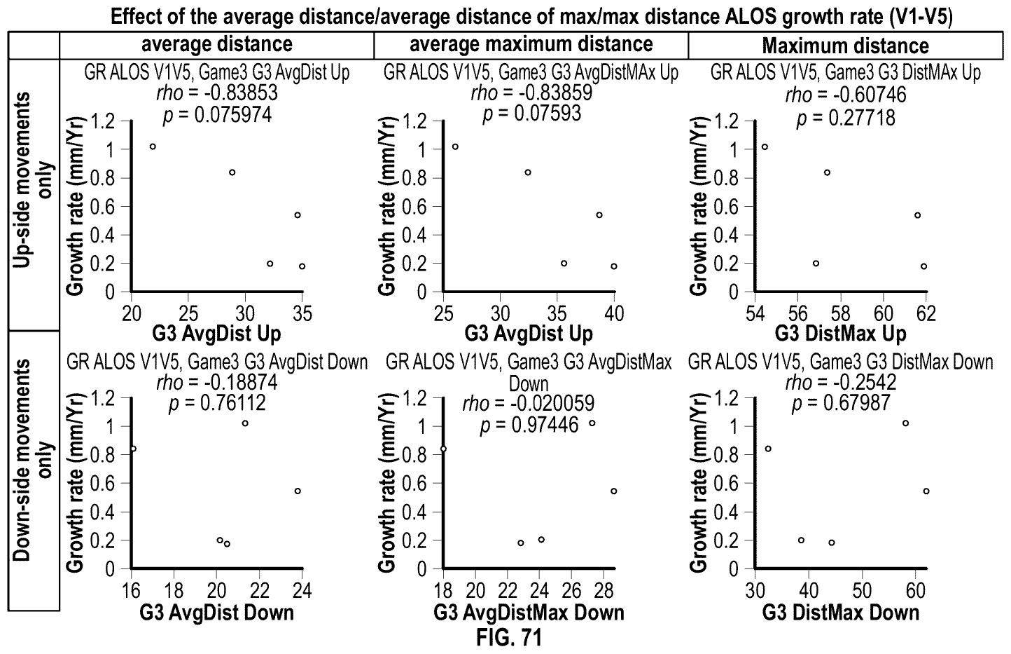

FIGS. 49-71 are data results by analyzing data from eye movement of subjects or a subject group in real time.

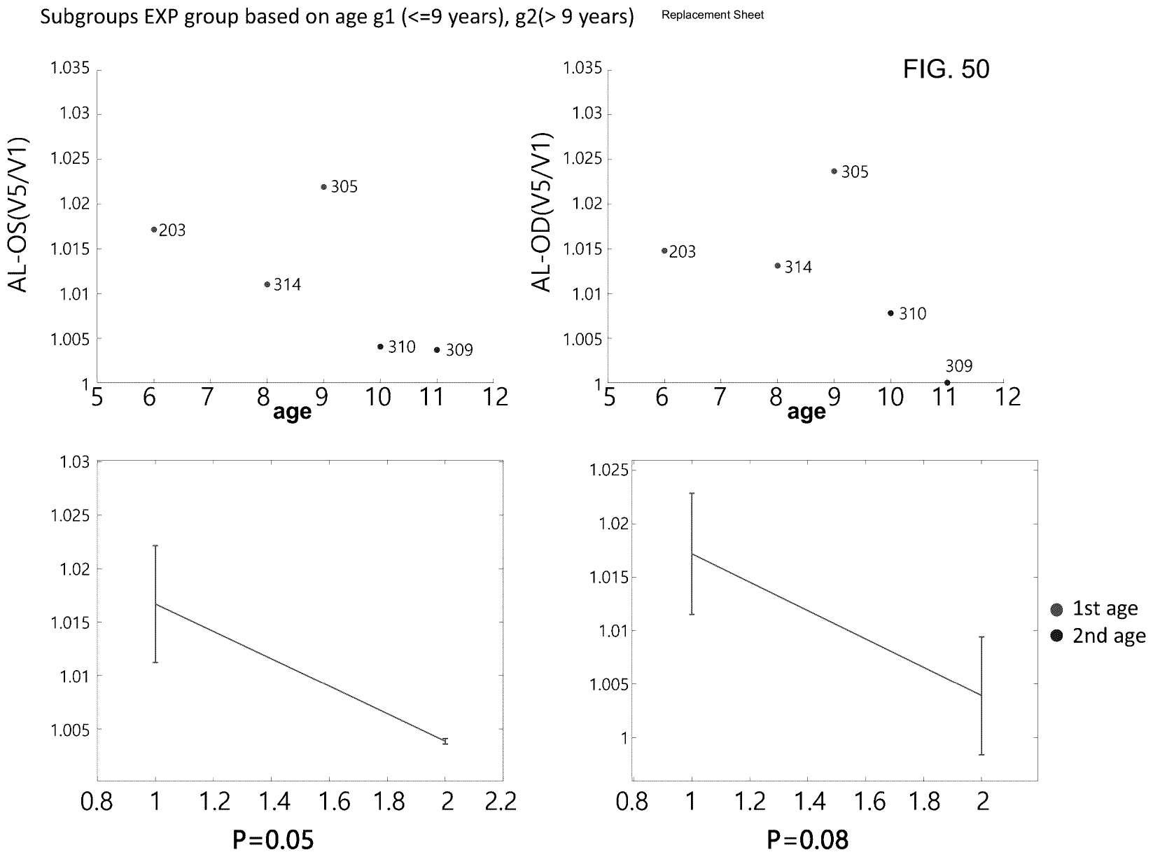

FIGS. 49-50 represents a correlation between ALOD or ALOS and age of subjects in a subject group.

FIG. 49 depicts graphs representing an ALOD and ALOS difference and a correlation between ALOD and ALOS, and age of subjects among visits V1, V4 and V5 are verified using a p-value. In some embodiments, a subject is from 5 years old to 12 years old. In some embodiments, the subject is a child. In some embodiments, the subject is less than about 15 years old. In some embodiments, the subject is assisted or supervised by an adult.

An ALOD represents Axial Length of Oculus Dexter (right eye) and is measured from a right eye of each subject. An ALOS represents Axial Length of Oculus Sinister (left eye) and is measured from a left eye of each subject. The axial length represents a normalized axial length.

The subject group may include an experimental group to which the method according to the present disclosure is applied and a control group to be compared with a corresponding experimental group.

Each Graph depicted in FIG. 49 represents a p-value of the total subject group, a p-value for the experimental group and a p-value for the control group which are based on measurement of the axial length of the right or left eye of the subject in the subject group, In the graph, x axis represents an age of a subject in the subject group and y axis represents the difference of measured axial length of the right or left eye of the subject in the subject group.

In the graphs, the letter V represents a visit of the subject group to a doctor and the number placed after the letter V represents a time offset from the first visit to specify the specific visit of the subject group. For example, a V1 may represent the first visit of the subject group, a V3 may represent the visit of the subject group after 3 weeks from the first visit, a V4 may represent the visit of the subject group after 12 weeks from the first visit, and a V5 may represent the visit of the subject group after 24 weeks from the first visit.

In some embodiments, a prescription may be provided to the subject group at each visit or a specific visit. For example, a prescription may be provided to the experimental group at the first visit corresponding to the V1, and a prescription may be provided at the visit corresponding to V4. In some embodiments, the prescription provided at the first visit may be referred to as a first prescription, the prescription provided at the visit corresponding to V4 may be referred to as a second prescription.

In some embodiments, ALOD V4-V1 may represent that the first prescription has been provided to the subject group at the first visit and a difference of measurements of the axial length of the right eye of one or more subjects between the first visit and the visit after 12 weeks from the first visit. ALOD V5-V4 may represent that the second prescription has been provided to the subject group at the visit after 12 weeks from the first visit and a difference of measurements of the axial length of the right eye of one or more subjects between the 12 weeks visit and the 24 weeks visit. ALOD V5-V1 may represent that the first prescription has been provided to the subject group at the first visit and a difference of measurements of the axial length of the right eye of one or more subjects in the subject group between the first visit and the visit after 24 weeks from the first visit.

In some embodiments, ALOS V4-V1 may represent that the first prescription has been provided to the subject group at the first visit and a difference of measurements of the axial length of the left eye of one or more subjects in the subject group between the first visit and the visit after 12 weeks from the first visit. ALOS V5-V4 may represent that the second prescription has been provided to the subject group at the visit after 12 weeks from the first visit and a difference of measurements of the axial length of the left eye of one or more subjects in the subject group between the 12 weeks visit and the 24 weeks visit. ALOS V5-V1 may represent that the first prescription has been provided to the subject group at the first visit and a difference of measurements of the axial length of the left eye of one or more subjects in the subject group between the first visit and the visit after 24 weeks from the first visit. The method of improving the eyesight of the present disclosure significantly reduces the AL growth rate in a subject who is 10 years old or more especially.

FIG. 50 depicts ALOS and ALOD change ratio of experimental groups (e.g., ALOD V5/V1 and ALOS V5/V1). A correlation between ALOS and ALOD change ratio, and age factor (age-subgroups) are verified with a p-value. The age-subgroups are categorized by predetermined age including a first age and a second age. The first age is 9 years old or under and the second age is over 9 years old.

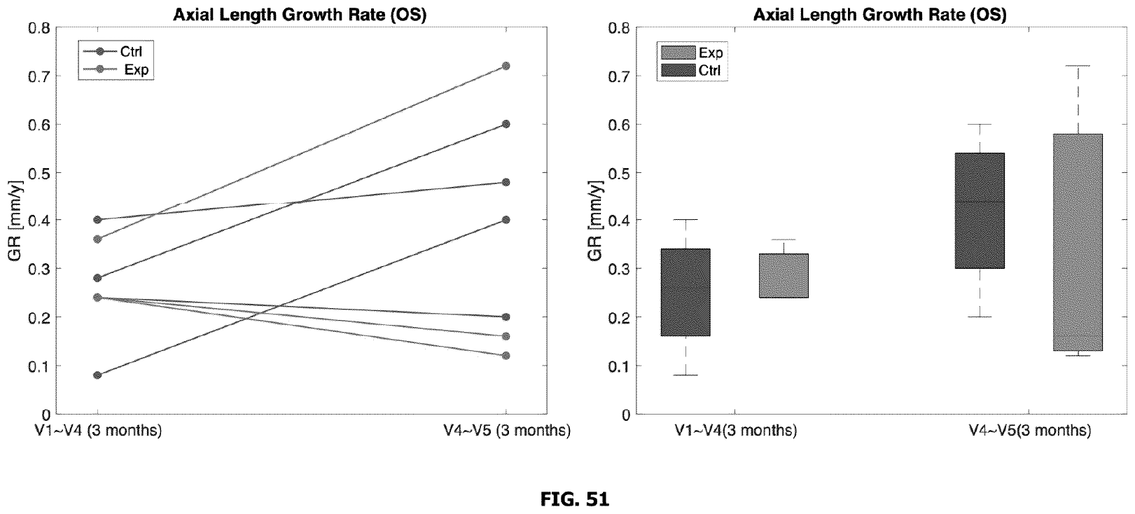

The method of improving an eyesight of a subject according to the present disclosure may lower a growth rate of AL (Axial Length) of a subject. FIGS. 51-54 represent the growth rate of the AL (Axial Length) slowed according to the method of improving eyesight of the present disclosure.

FIG. 51 depicts ALOS growth rates in mm/year for each of an experimental group and a control group in a period from V1 to V4 (V1-V4) and in a period from V4 to V5 (V4-V5). As shown in the graph on the right side, a change (and/or change ratio) of the ALOS growth rate of the experimental group between V1-V4 to V4-V5, is less than a change (and/or change ratio) of the ALOS growth rate of the control group.

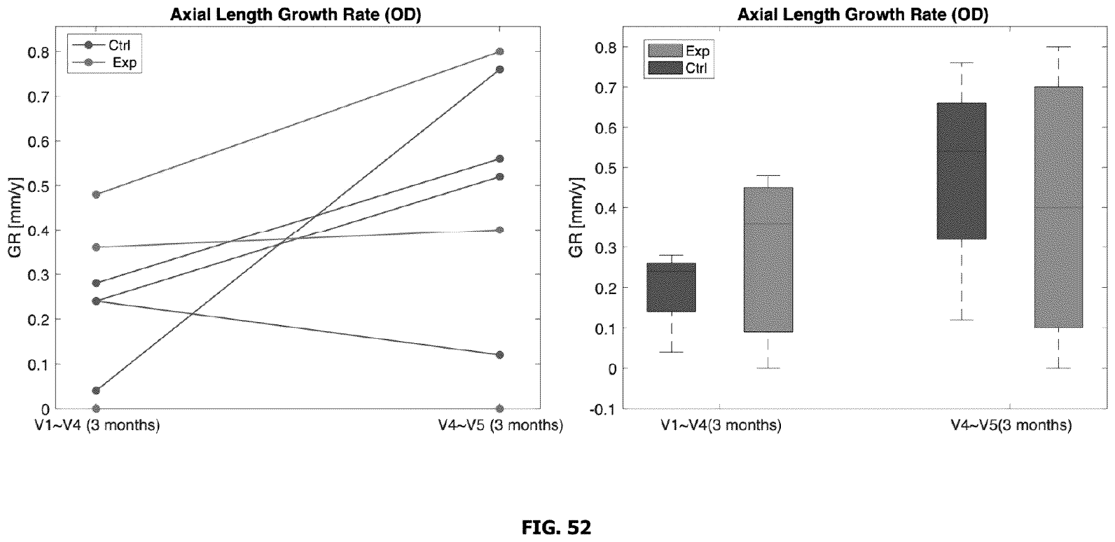

FIG. 52 depicts ALOD growth rates in mm/year for each of an experimental group and a control group in a period from V1 to V4 (V1-V4) and in a period from V4 to V5 (V4-V5). As shown in the graph on the right side, a change (and/or change ratio) of the ALOD growth rate of the experimental group between V1-V4 to V4-V5, is less than a change (and/or change ratio) of the ALOD growth rate of the control group.

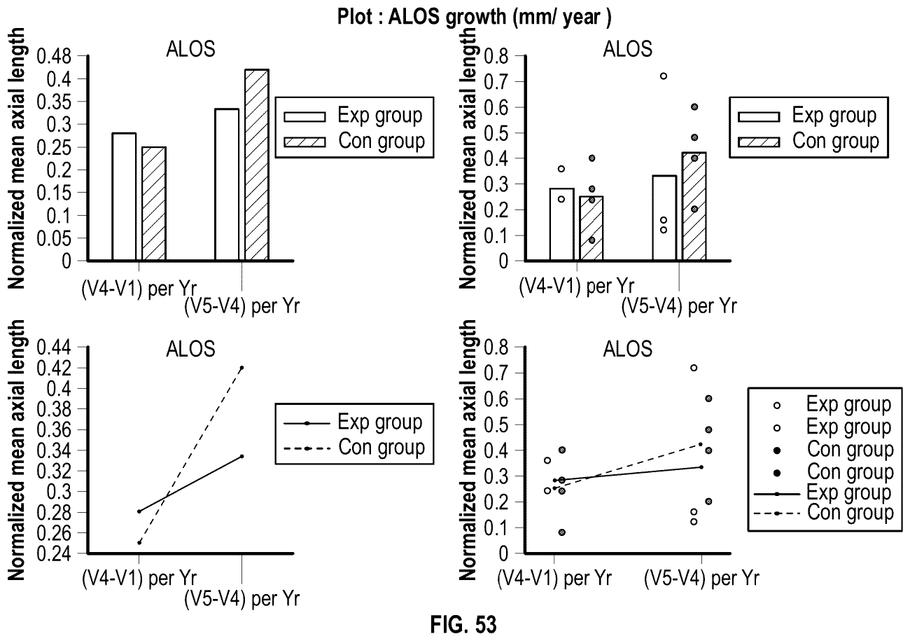

FIG. 53 depicts normalized ALOS of an experimental group (in red color) and control group (in blue color) in a period from V1 to V4 (V4-V1) and in a period from V4 to V5 (V5-V4), respectively. As shown in graphs, the change ratio (or change) of the normalized AL of experimental group is less than that of the control group.

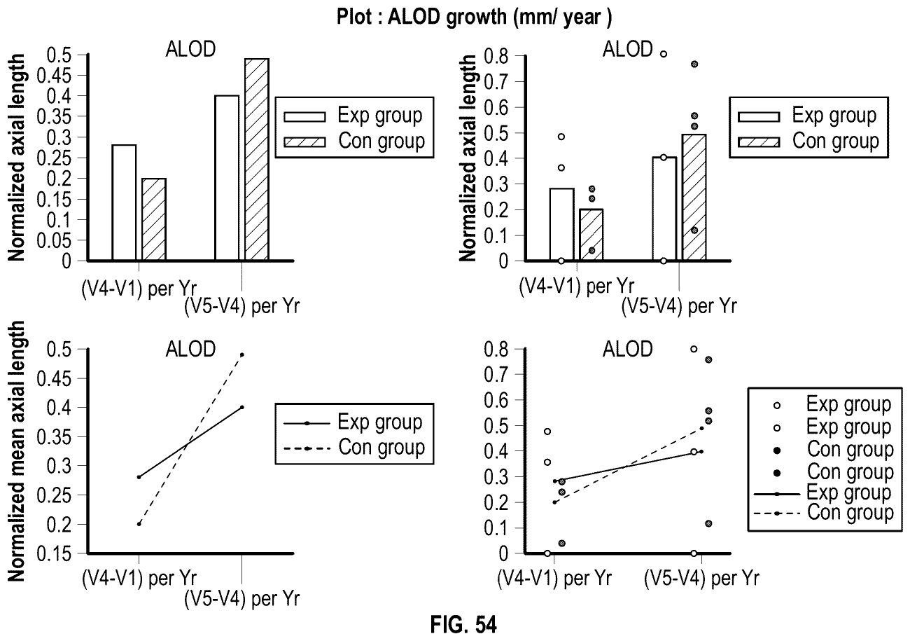

FIG. 54 depicts normalized ALOD of an experimental group (in red color) and control group (in blue color) in a period from V1 to V4 (V4-V1) and in a period from V4 to V5 (V5-V4), respectively. As shown in graphs, the change ratio of the normalized AL of experimental group is less than that of the control group.

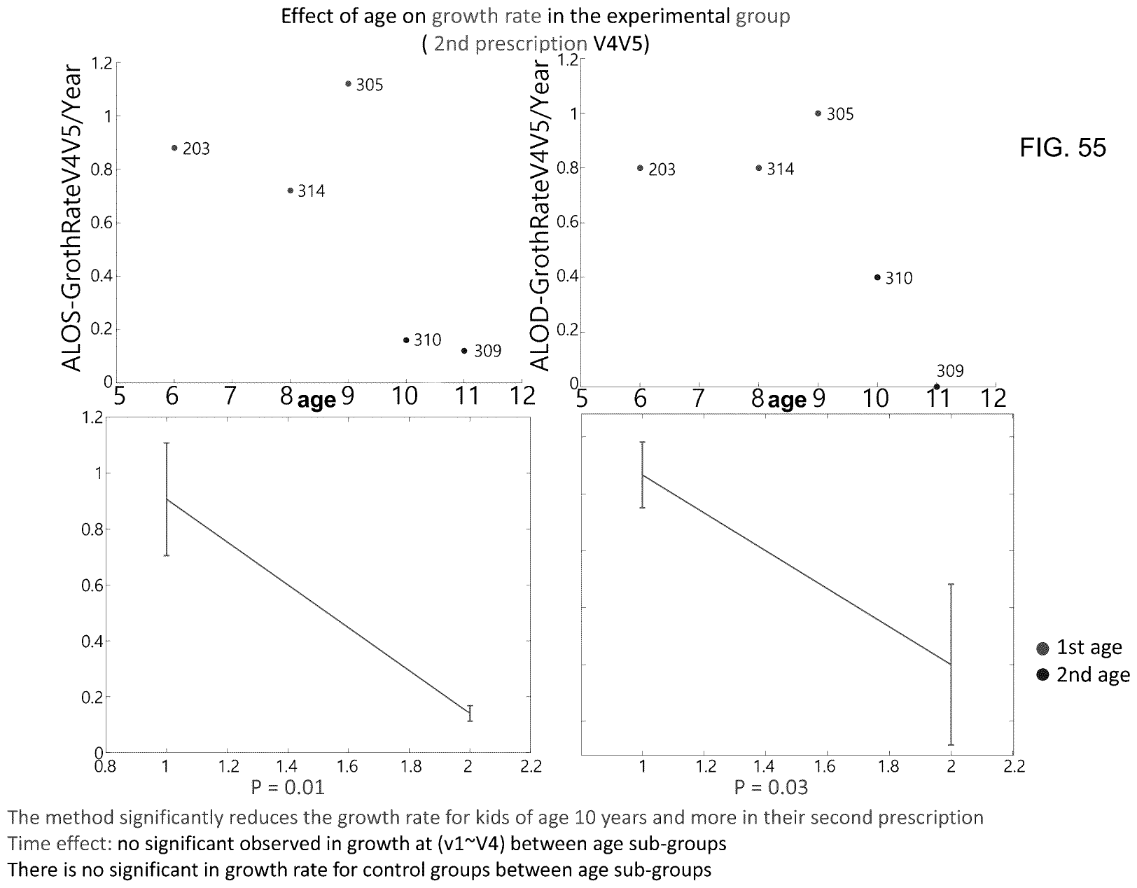

The method of improving an eyesight of a subject according to the present disclosure may control a growth rate of AL (Axial Length) of a subject. FIGS. 55-57 represent the growth rate of the AL regulated according to the method of improving eyesight of the present disclosure.

FIG. 55 depicts effect of an age on ALOS and ALOD growth rates in an experimental group (e.g., ALOS V5/V4 and ALOD V5/V4). The age-subgroups are categorized by predetermined age including a first age and a second age. The first age is 9 years old or under and the second age is over 9 years old. The method of improving the eyesight of the present disclosure significantly reduces the AL growth rate in a subject who is 10 years old or more especially at their second prescription (see FIG. 49). Here, time may not affect the AL growth rate at V1-V4 between age-subgroups.

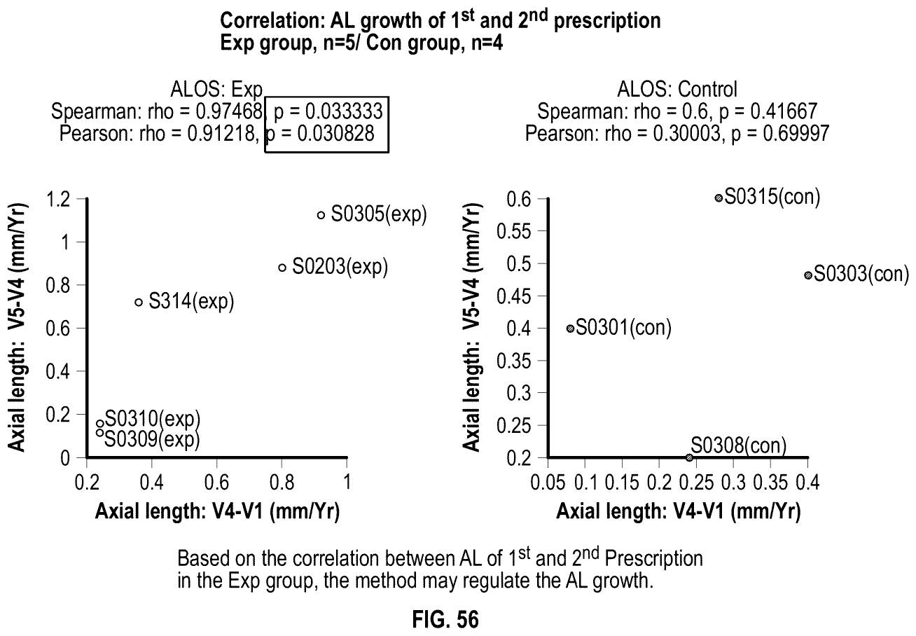

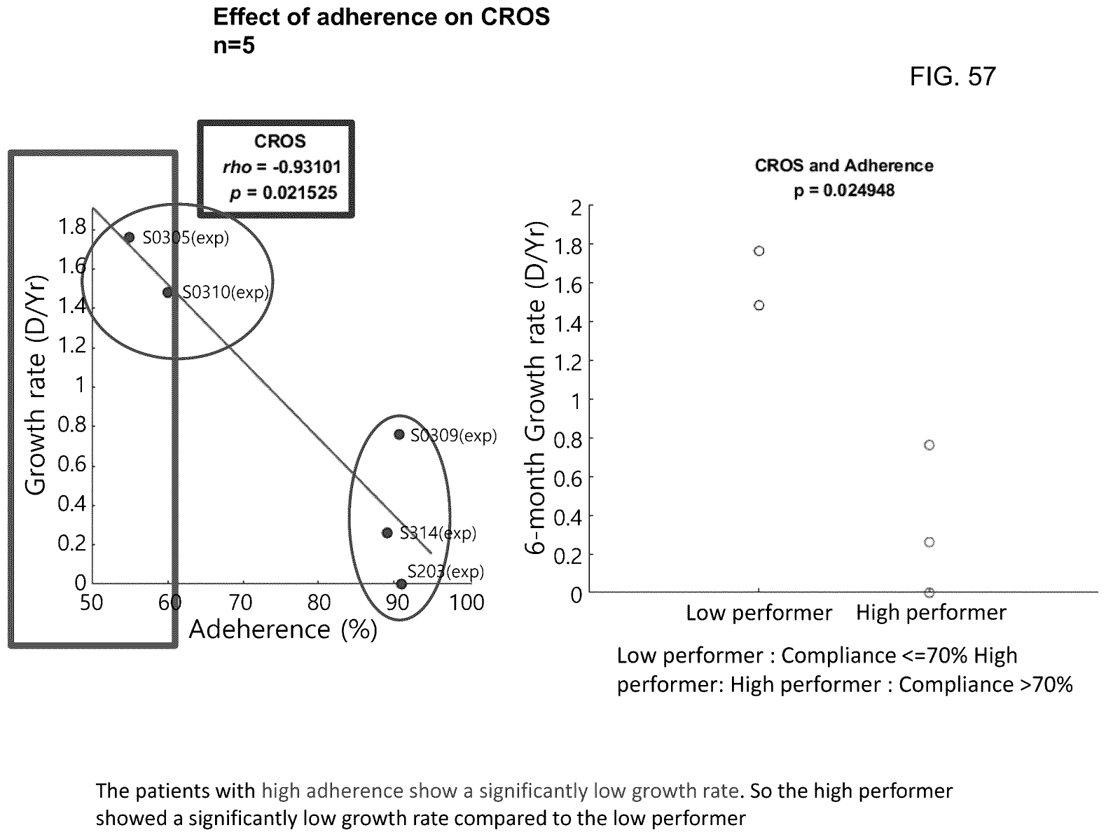

FIG. 56 depicts a correlation between AL (Axial Length) growth rate by a first prescription (e.g., V4-V1) and AL (Axial Length) growth rate by a second prescription (e.g., V5-V4) in a control group and an experimental group, respectively. A graph on the left side of FIG. 56 represents a correlation between AL of a first prescription (e.g., V4-V1) and AL of a second prescription (e.g., V5-V4) The method of improving the eyesight of the present disclosure may regulate the AL based on the correlation depicted in FIG. 57.

FIG. 57-64 represent correlations between a growth rate of AL (Axial Length) and several factors in performance of a subject following an eyeball exercise instruction provided by the method of improving an eyesight of a subject according to the present disclosure.

In some embodiments, the method according to the present disclosure may provide a digital application comprising one or more digital therapeutic modules for improving the eyesight, each of the modules comprises one or more first instructions comprising a first eyeball exercise instruction for the subject to follow. In some embodiments, the first instructions comprise a first eyeball exercise instruction for the subject to move at least one eyeball vertically. In some embodiments, the first eyeball exercise instruction is for the subject to move the at least one eyeball at least 50 out of 100 maximum vertical view of the subject. In some embodiments, the first eyeball exercise instruction is for the subject to move the at least one eyeball at least 70 out of 100 maximum vertical view of the subject. In some embodiments, the method according to the present disclosure may measure a maximum vertical view by a sensor of the digital apparatus.

In some embodiments, the digital application comprises more instructions for vertical eye movements compared to instructions for horizontal eye movement. In some embodiments, the first eyeball exercise instruction is to move the at least one eyeball upward. The first instructions exclude an instruction to move said at least one eyeball horizontally.

FIG. 57 depicts a correlation between adherence of a subject to follow the eyeball exercise instruction and a growth rate of a CR (Cycloplegic refraction test). As shown in FIG. 57, the subjects with high adherence have a significantly low growth rate compared to subjects with lower adherence. The high adherence may be greater than 70%, the low adherence may be less than or equal to 70%.

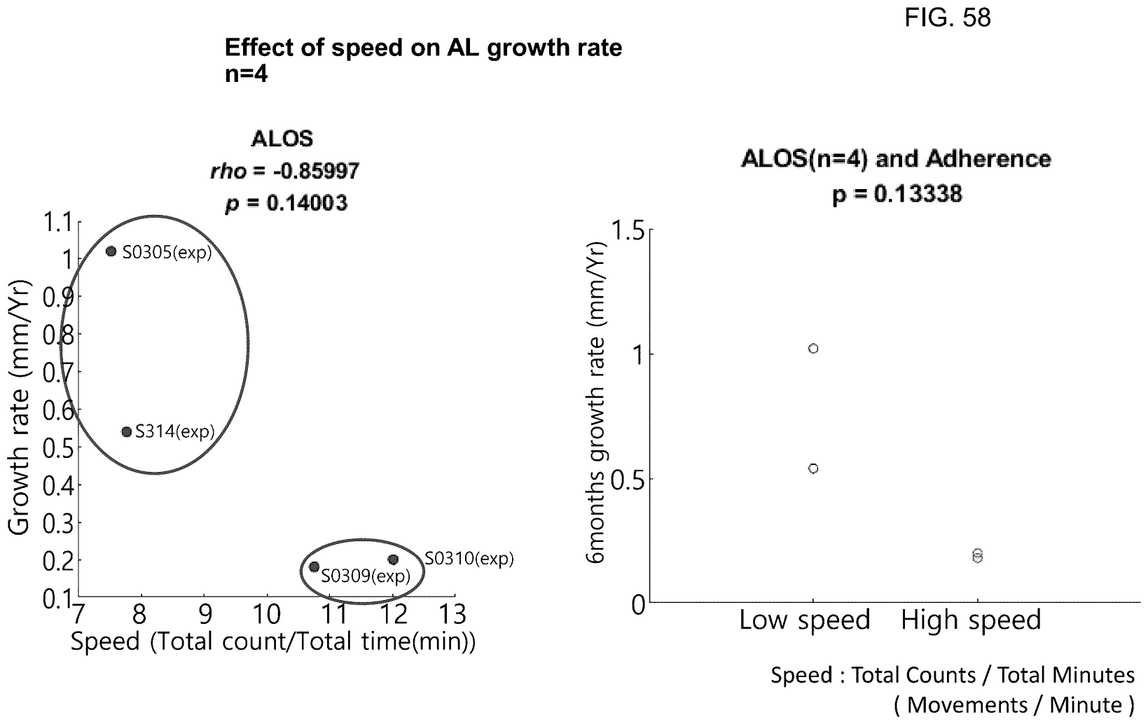

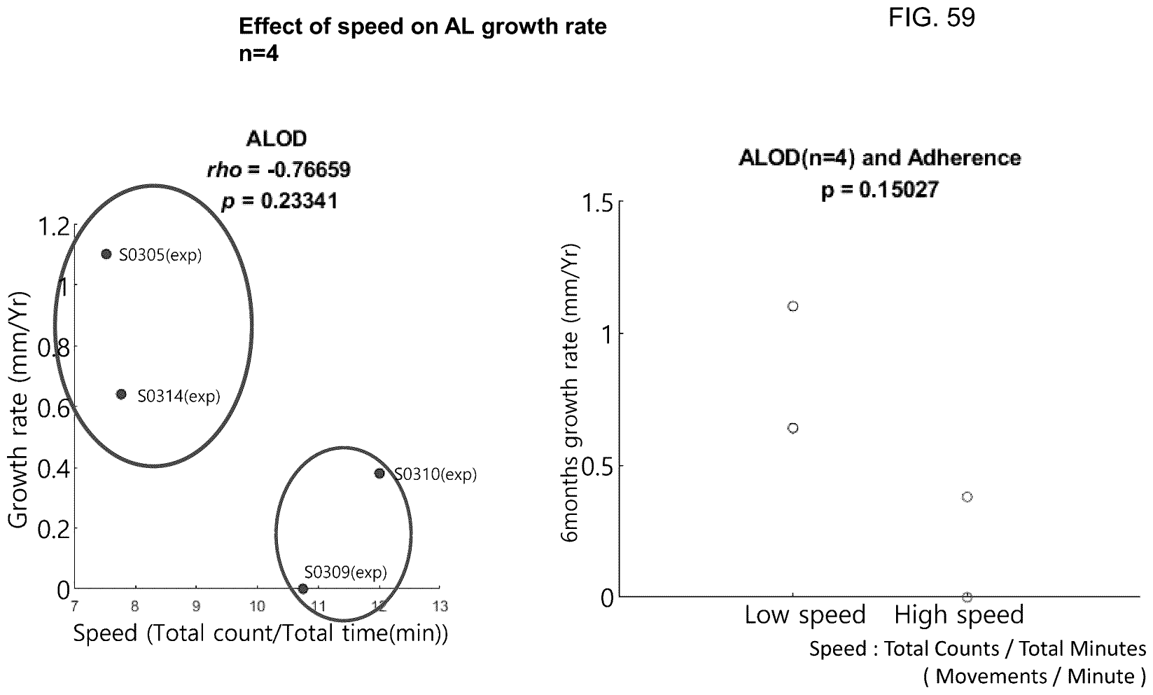

FIG. 58 depicts a correlation between a speed of movement of eyeball of a subject in an ALOS group following the eyeball exercise instruction and a growth rate of AL (Axial Length) and FIG. 59 depicts a correlation between the speed of movement of eyeball of a subject in an ALOD group following the eyeball exercise instruction and a growth rate of axial length. The speed may be represented as total count/total number of minutes or movements/minutes. As shown in FIGS. 58-59, the subjects with a high speed have a significantly low growth rate compared to subjects with a low speed.

According to the present disclosure, the first eye exercise module may be implemented as a game so that the subject enjoys the game while following the first eyeball exercise instruction provided in the game. In some embodiments, various eyeball exercise instructions for vertical eye movements, horizontal eye movements, or combination thereof may be provided in the game.

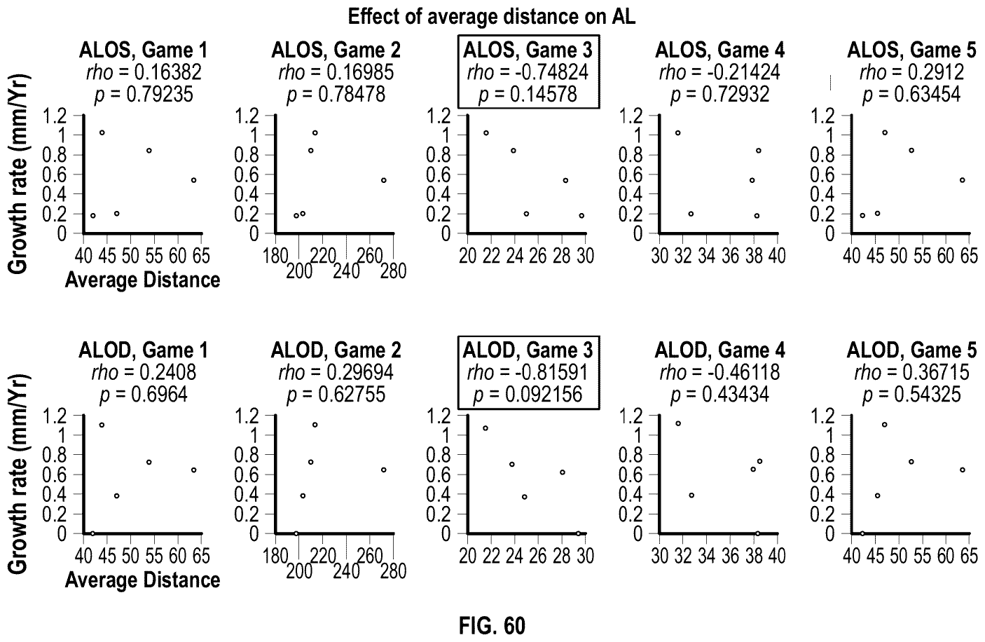

In the first eyeball exercise instruction for the subject to move at least one eyeball vertically, a growth rate of AL (Axial Length) is significantly related to an average distance of eye movements. FIG. 60 depicts a correlation between the grow rate of AL and the average distance of eye movements that the subject performs in each game.

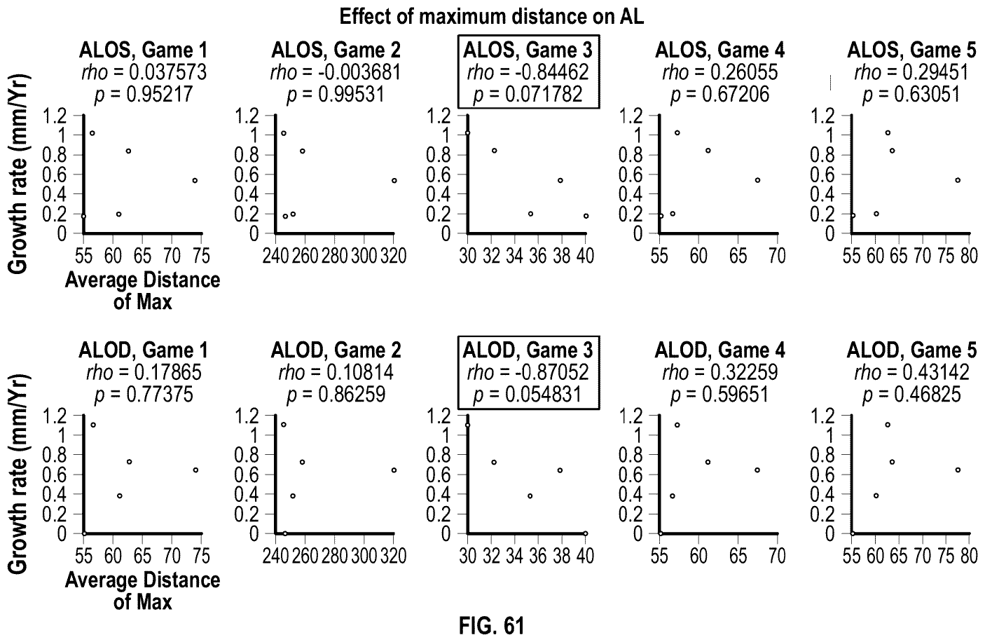

In the first eyeball exercise instruction for the subject to move at least one eyeball vertically, a growth rate of AL (Axial Length) is significantly related to a maximum distance of eye movements. FIG. 61 depicts a correlation between the grow rate of AL and the maximum distance of eye movements that the subject performs in each game.

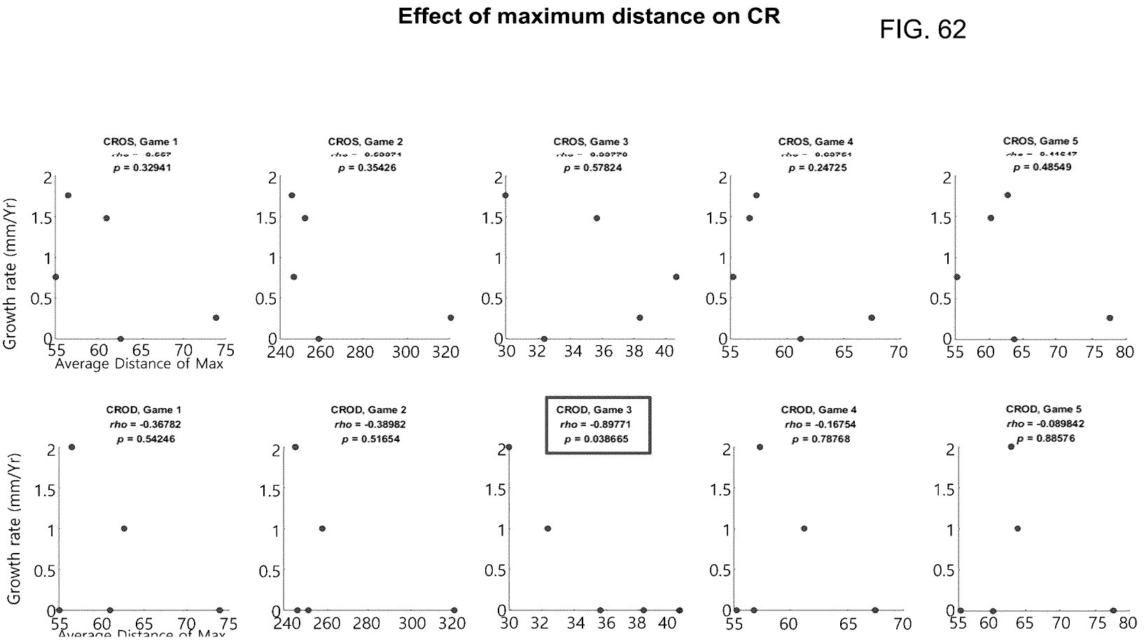

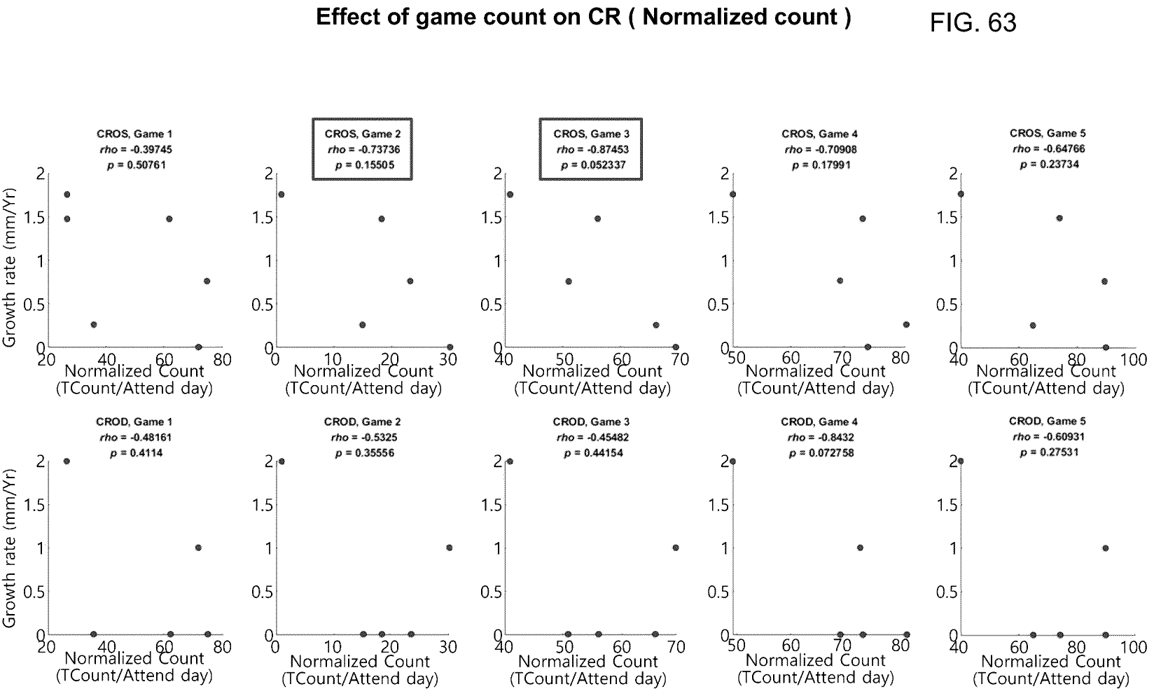

In the first eyeball exercise instruction for the subject to move at least one eyeball vertically, a growth rate of a CR (Cycloplegic refraction test) is significantly related to a maximum distance of eye movements. FIG. 62 depicts a correlation between the grow rate of the CR and the maximum distance of eye movements that the subject performs in each game.

In the first eyeball exercise instruction for the subject to move at least one eyeball vertically, a growth rate of a CR (Cycloplegic refraction test) is significantly related to a game count that the subject performs in each game. FIG. 63 depicts a correlation between the grow rate of the CR and a normalized game count. The normalized game count represents a number of movements per a game or an attending day to play the corresponding game.

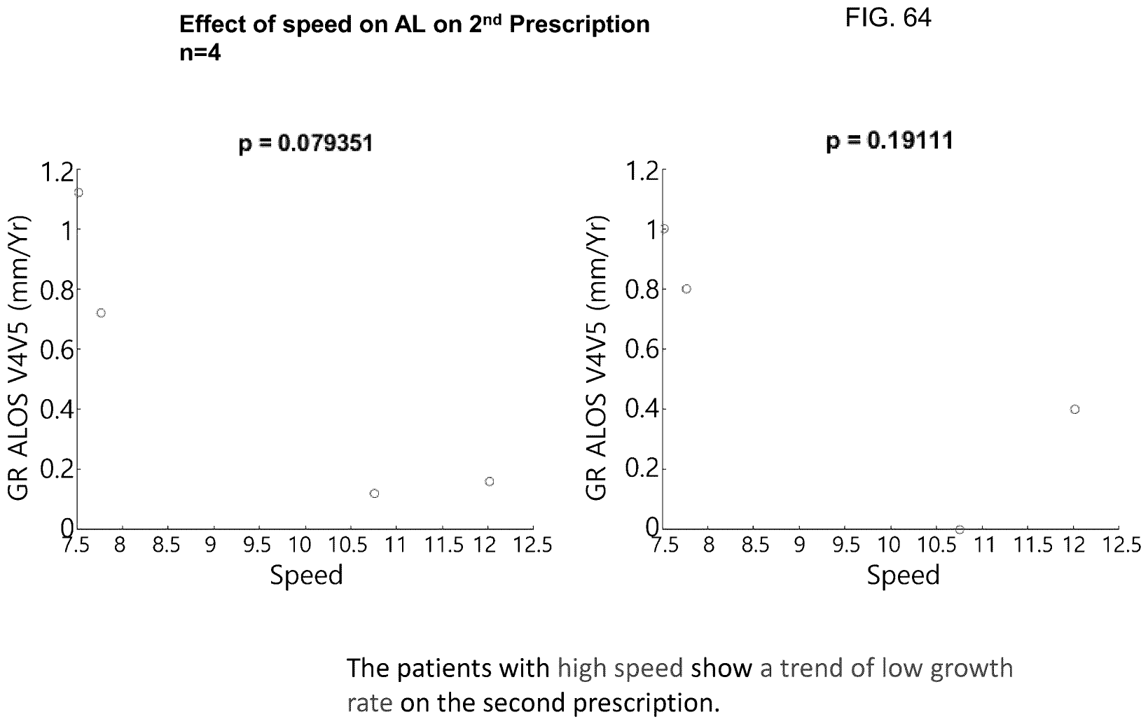

In the first eyeball exercise instruction for the subject to move at least one eyeball vertically, a growth rate of AL (Axial Length) is related to a speed of eye movements. FIG. 64 depicts a correlation between the grow rate of AL and the speed of eye movements. As shown in FIG. 64, the subjects with a high speed have a low growth rate compared to subjects with a low speed.

The vertical eye movements according to the present disclosure may include up-down movements, up-side movements and down-side movements.

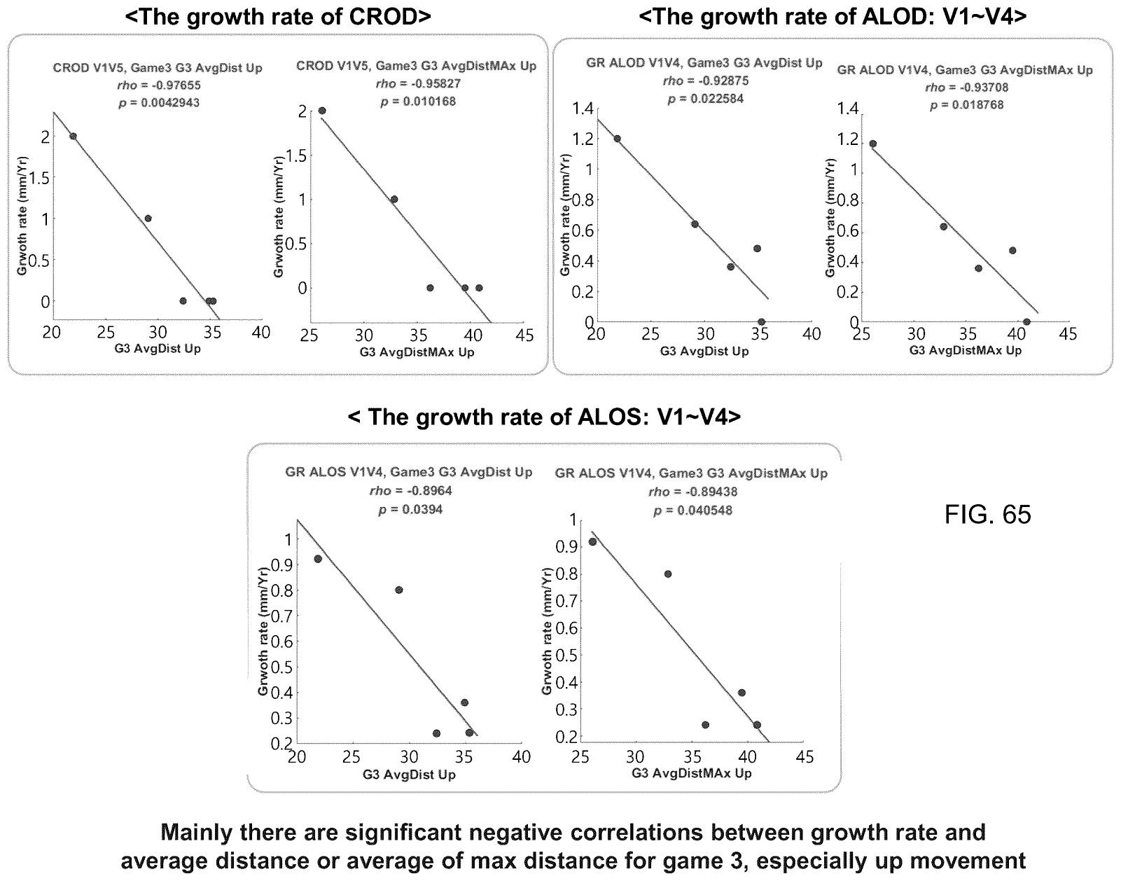

FIG. 65 depicts graphs showing correlations between a growth rate and an average distance or average of max distance of eye movements that the subject performs the first eyeball exercise instruction. The first eyeball exercise instruction is for the subject to move at least one eyeball vertically and may be implemented as a game. The game implementing the first eyeball exercise may be the third game which is explained in FIGS. 60-63. As shown in FIG. 65, the growth rate and the average distance or average of max distance have significant negative correlations.

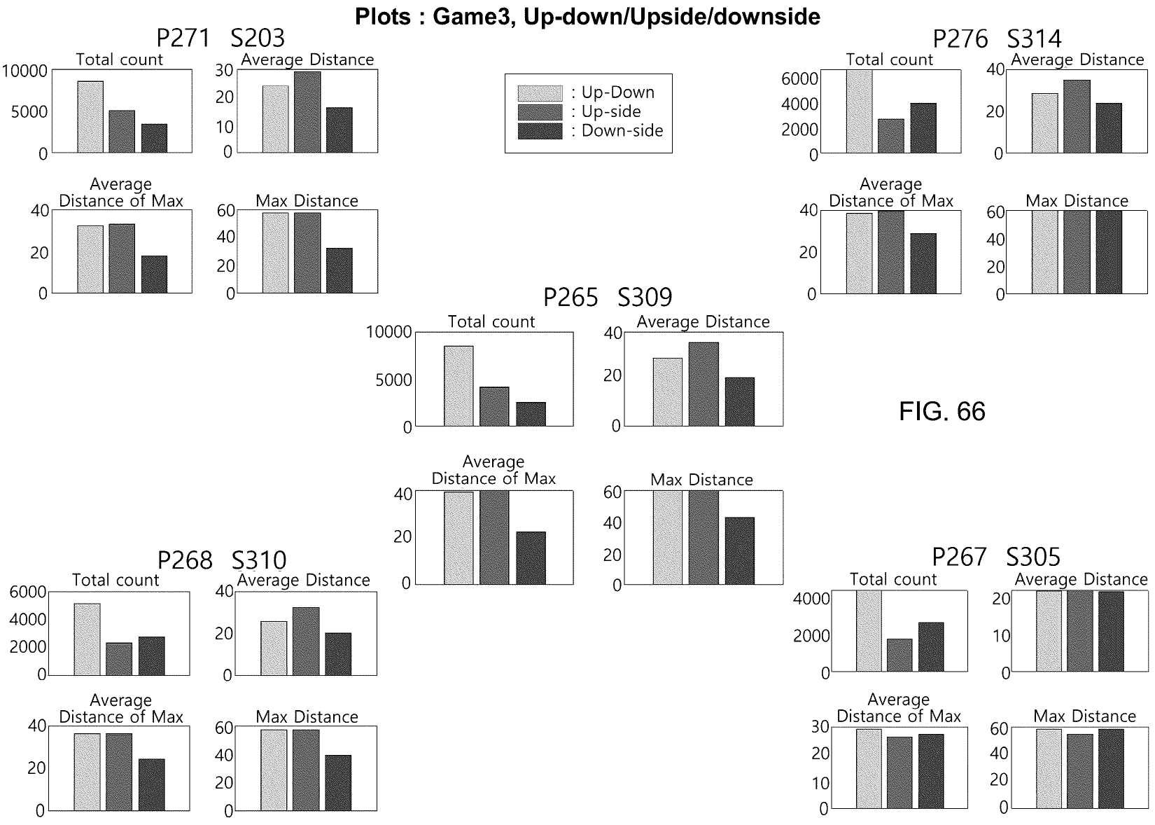

FIG. 66 depicts graphs showing a total count, an average distance, an average distance of maximum distances and a maximum distance for up-down movements, up-side movements and down-side movements, respectively. FIG. 66 depicts five groups, each group including four graphs representing a total count, an average distance, an average distance of maximum distances and a maximum distance for up-down movements, up-side movements and down-side movements, respectively. Each group represents performance of each patient when each patient performs the first eyeball exercise (or the game 3).

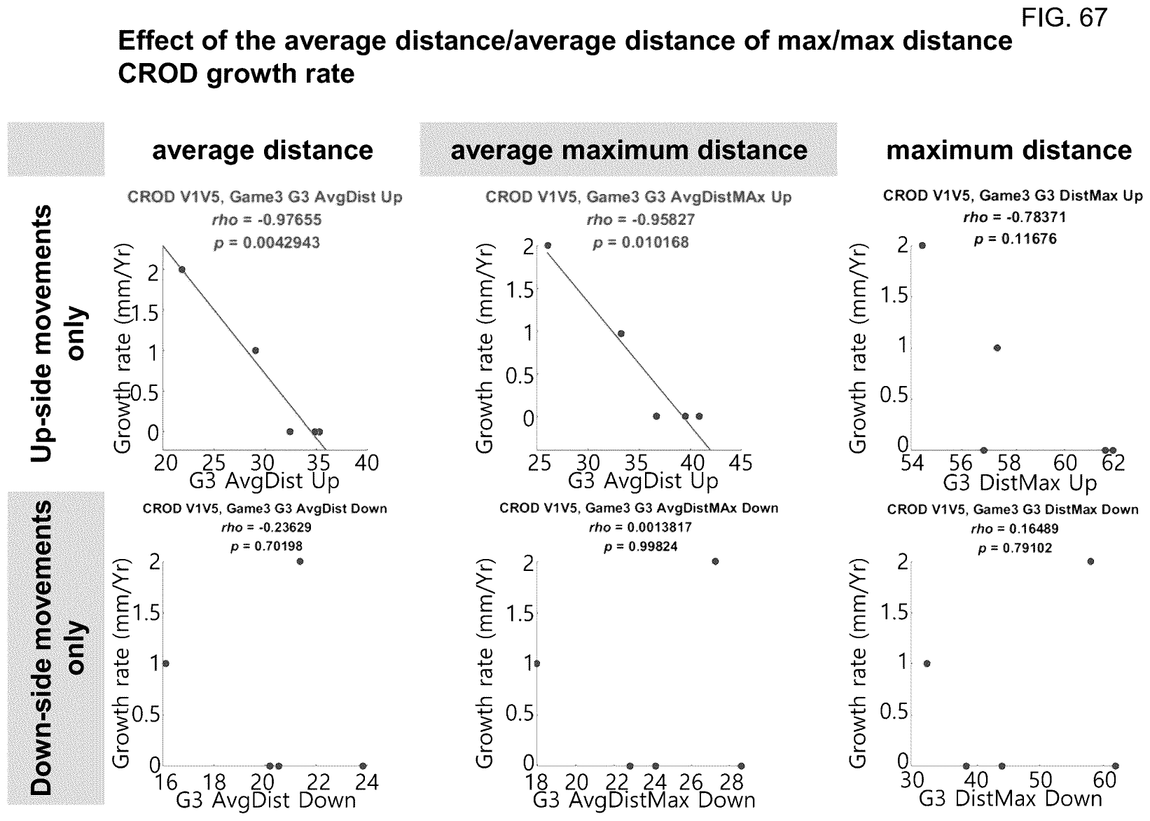

FIG. 67 depicts correlations between CROD growth rate, and the average distance, the average distance of maximum distances and the maximum distance for up-side movements and down-side movements, respectively. FIG. 67 discloses graphs representing correlations when the patient performs the first eyeball exercise.

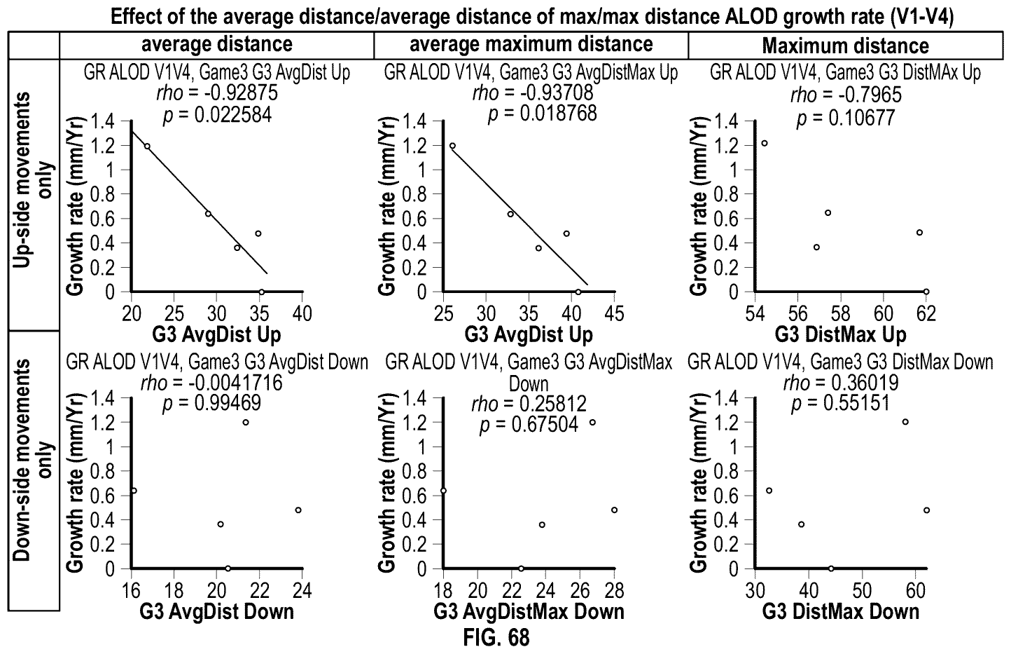

FIGS. 68-71 depicts correlations between a growth rate of AL (Axial Length), and the average distance, the average distance of maximum distances and the maximum distance for up-side movements and down-side movements, respectively. FIGS. 68-71 disclose graphs representing correlations when the patient performs the first eyeball exercise.

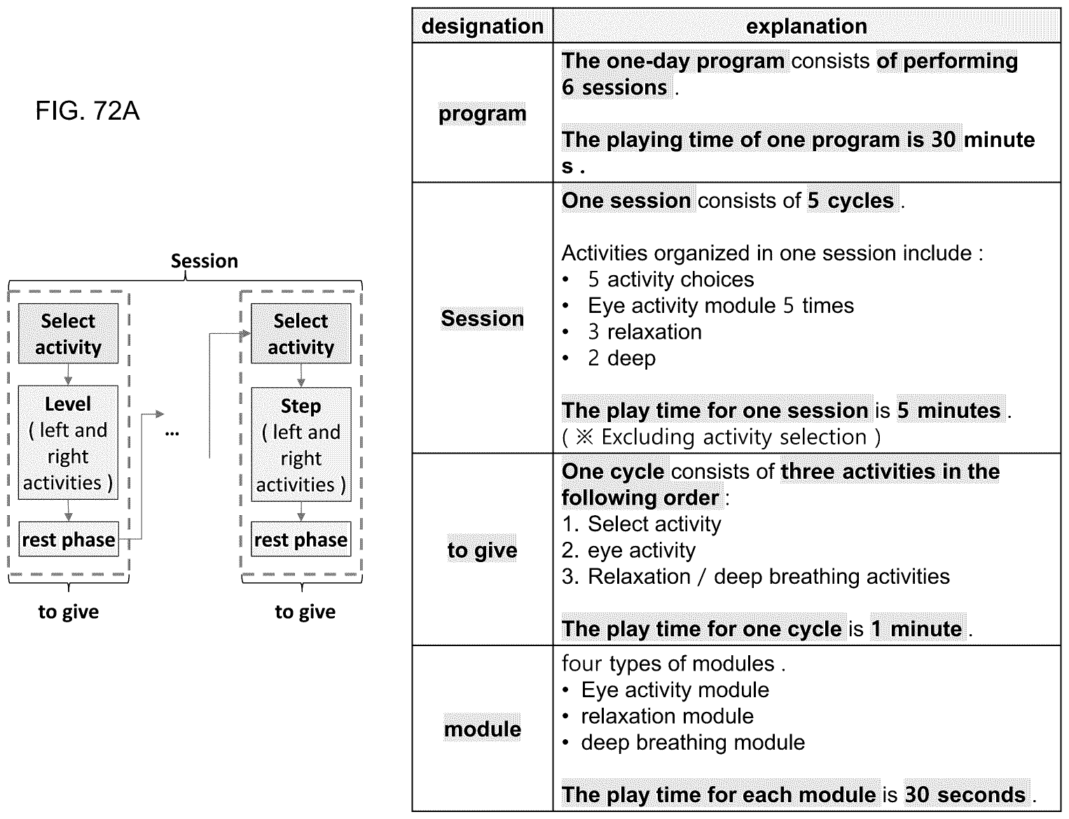

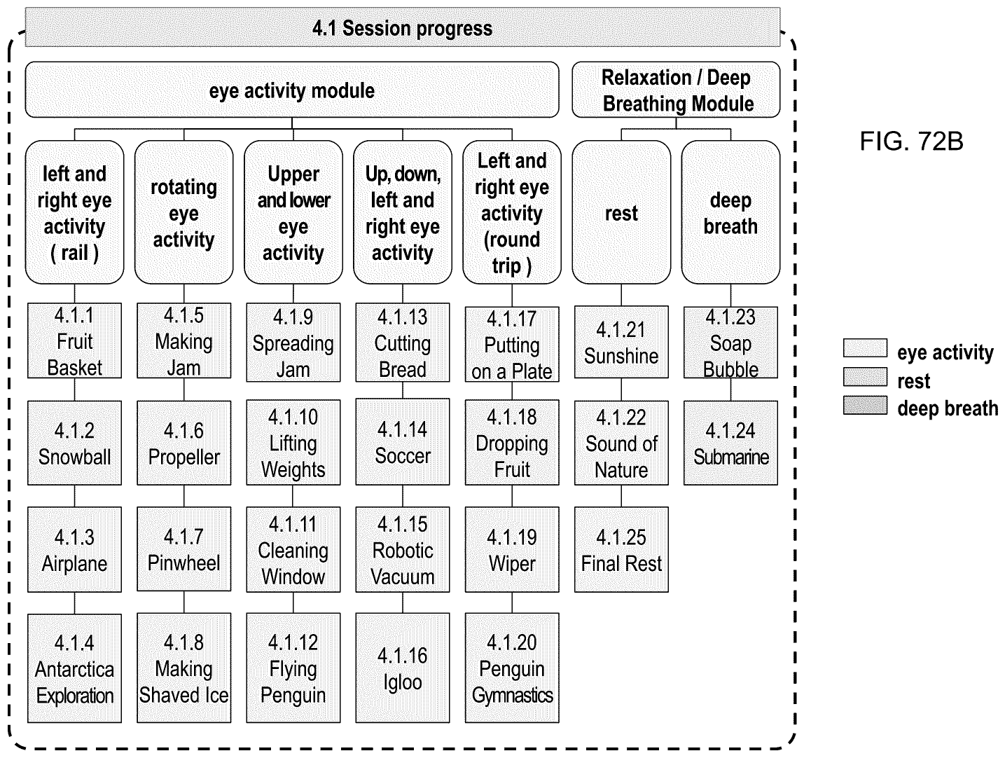

FIG. 72A depicts an example of a session provided in a digital application of the present disclosure. In some embodiments, the session comprises one or more digital therapeutic modules. The one or more digital therapeutic modules may include an eye exercise digital therapeutic module, a relaxation digital therapeutic module and a deep breathing module.

FIG. 72B depicts one or more digital therapeutic modules in a session. The one or more digital therapeutic modules may include an eye exercise digital therapeutic module, a relaxation digital therapeutic module and a deep breathing module, for improving the eyesight. The eye exercise digital therapeutic module includes one or more eyeball exercise instructions for a subject to follow. The one or more eyeball exercise instructions include: an eyeball exercise instruction for a subject to move at least one eyeball horizontally (or left and/or right), an eyeball exercise instruction for a subject to rotate at least one eyeball, an eyeball exercise instruction for a subject to move at least one eyeball vertically, an eyeball exercise instruction for a subject to move at least one eyeball vertically and horizontally and an eyeball exercise instruction for a subject to move at least one eyeball to the left and right repeatedly.

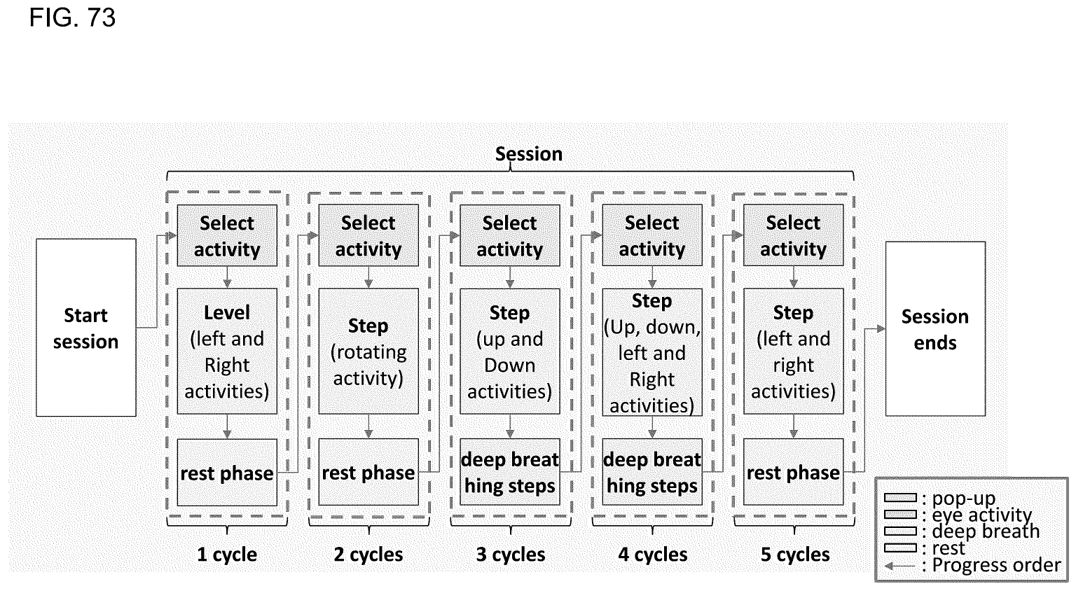

FIG. 73 depicts a flow chart illustrating an execution flow for a session in a digital application of the present disclosure. The session may be provided to a user (or patient) to follow a sequence of instructions in a predetermined time duration (e.g., 5 minutes). Thus, the session comprises one or more digital therapeutic modules to provide a user with instructions to follow in a predetermined order. In some embodiments, the predetermined order may change. The session may include at least 5 periods. At least one eye exercise module and one of a relaxation module and a deep breathing module may be provided, but not limited, in each period.

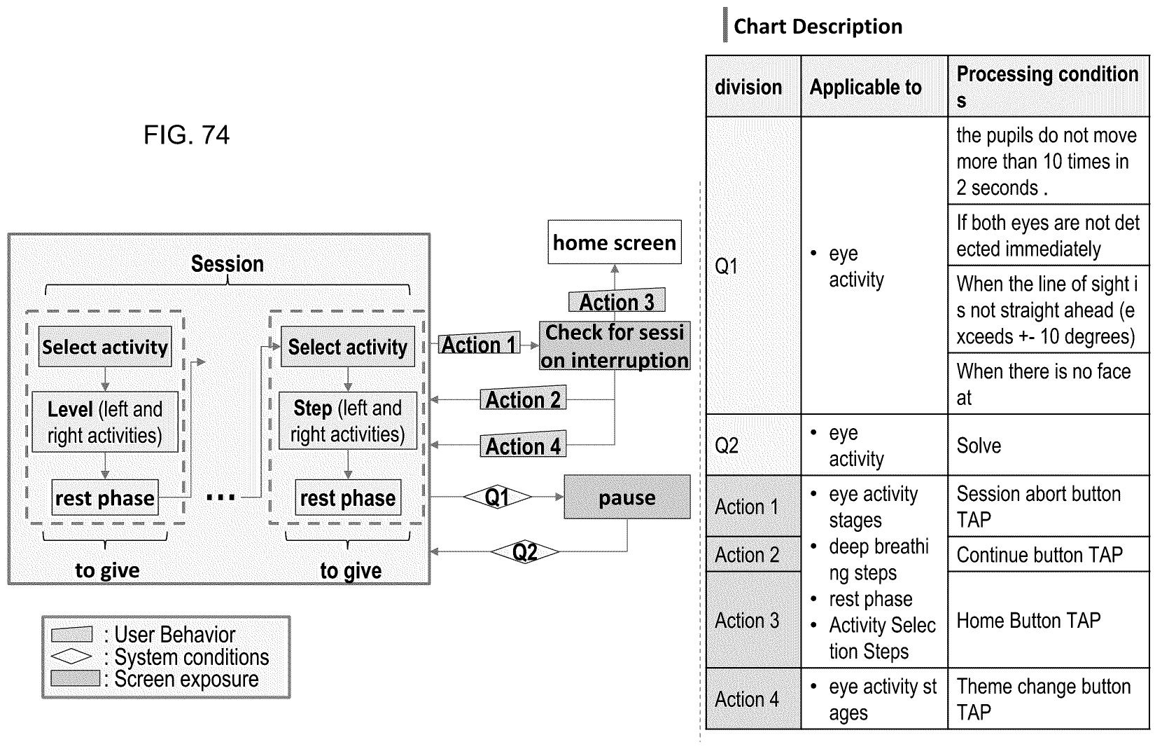

FIG. 74 depicts a flow chart illustrating an execution flow for a session in a digital application of the present disclosure. The execution flow includes a first flow to execute the session based on an interaction with a user and a second flow to determine whether or not to pause the session based on detection of at least one user eyeball status. In response to detection of a user input signal, the digital application may execute, stop or continue the session. In some embodiments, the digital application may detect or obtain at least one user status while the user following one or more instructions provided during the session (Q1) and may pause the session based on the user status. For example, the user status may include i) no movement of at least one user eyeball in a predetermined time duration, ii) failure of detection of two user eyeballs, iii) detection of a user gaze which does not correspond to the front, and iv) failure of detection of a user face in a predetermined distance. If any one of the user status is not detected any more, the digital application may continue the session (Q2).

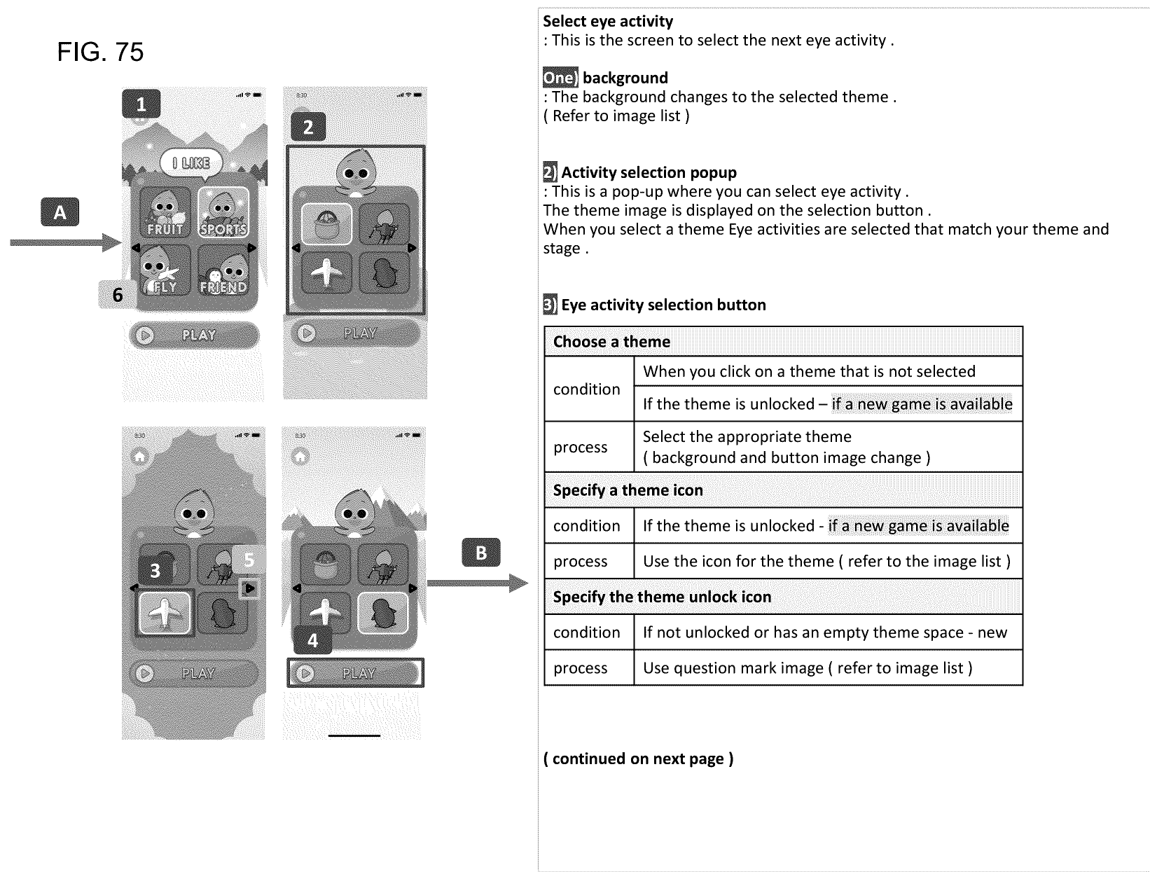

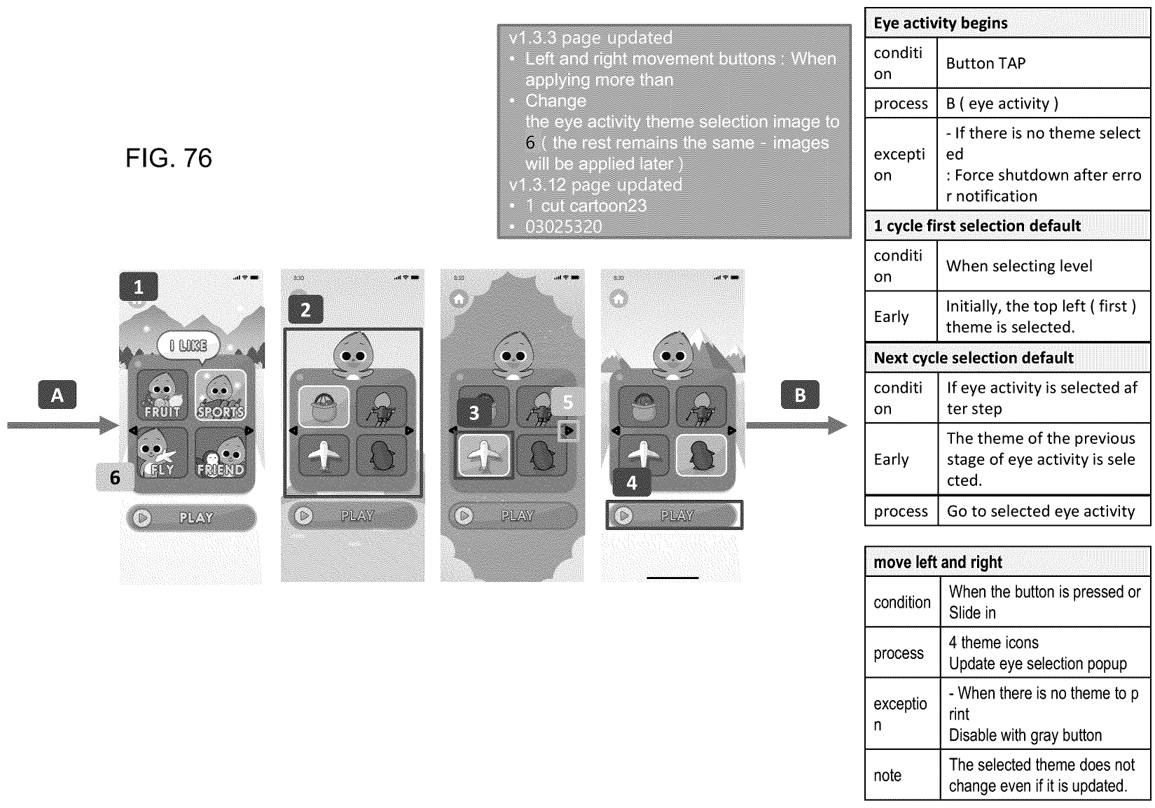

FIGS. 75-76 depict examples of user interfaces provided by a digital application of the present disclosure. The user interfaces may include visual images such as icons, images, characters and/or information representing one or more eyeball exercises. The user interfaces may receive a user input to select at least one eyeball exercise. In accordance with the user input, the digital application of the present disclosure instructs a processor of the digital apparatus to execute operations. The executed operations include digital therapeutic modules for improving eyesight.

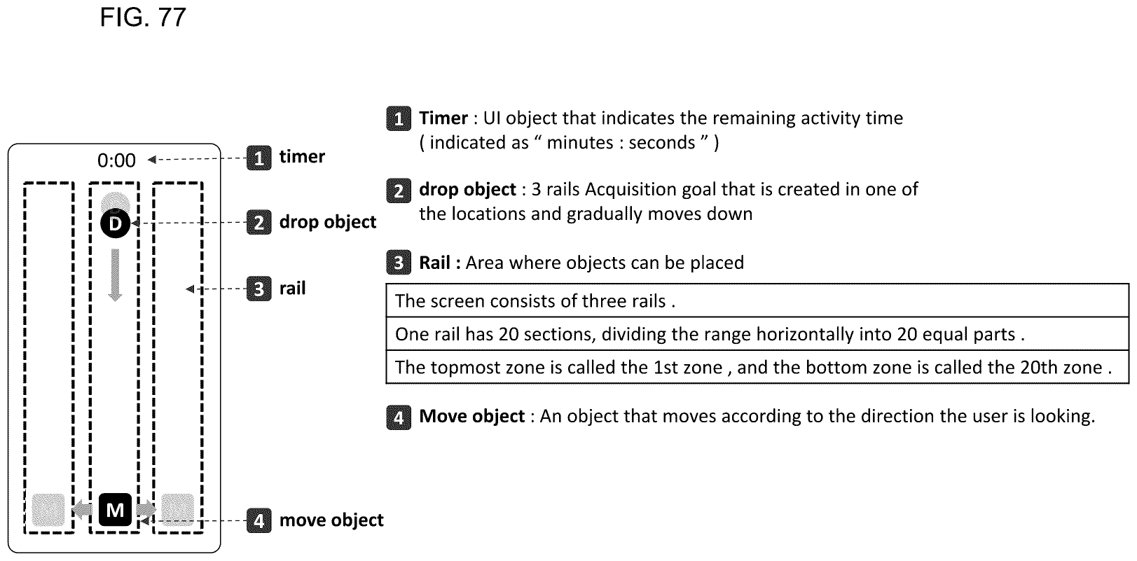

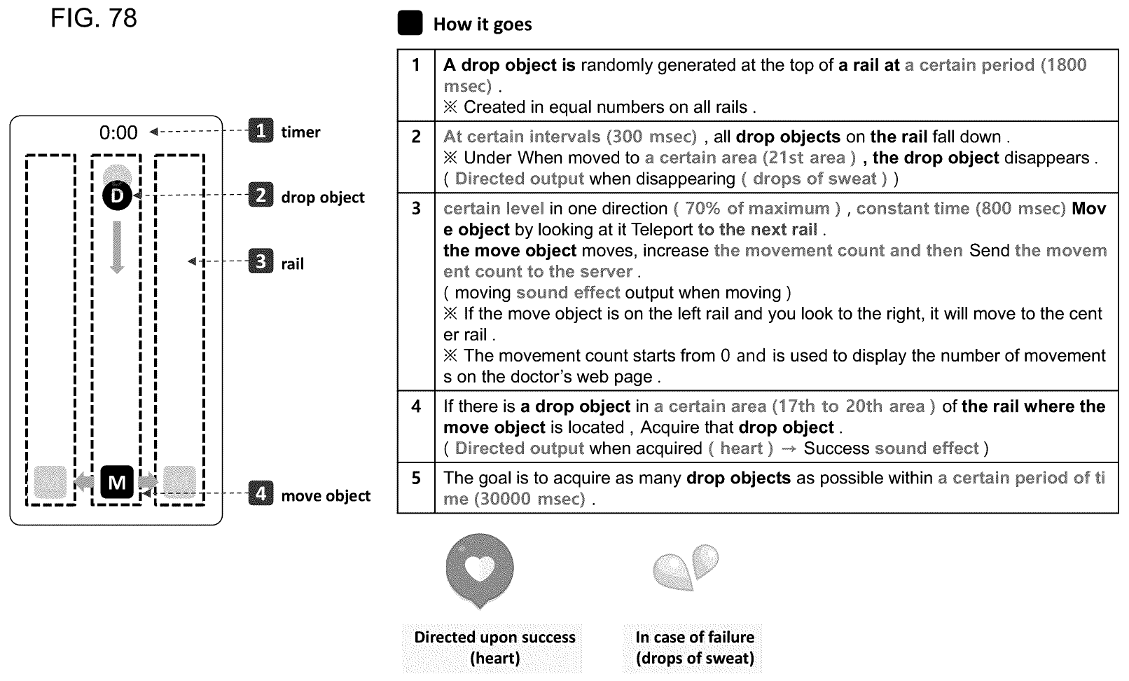

FIGS. 77-78 depict an example of a user interface for an eyeball exercise instruction displayed in the digital apparatus. The user interface may include visual images such as icons, images, characters and/or information and be displayed by the digital apparatus. The eye exercise digital therapeutic module includes an eyeball exercise instruction for a subject to move at least one eyeball horizontally (or left and/or right) following the object moving among rails displayed in the user interface. The eyeball movement is sensed by a sensor in the digital apparatus, adherence by the subject to the eyeball exercise instruction of the eye exercise digital therapeutic module.

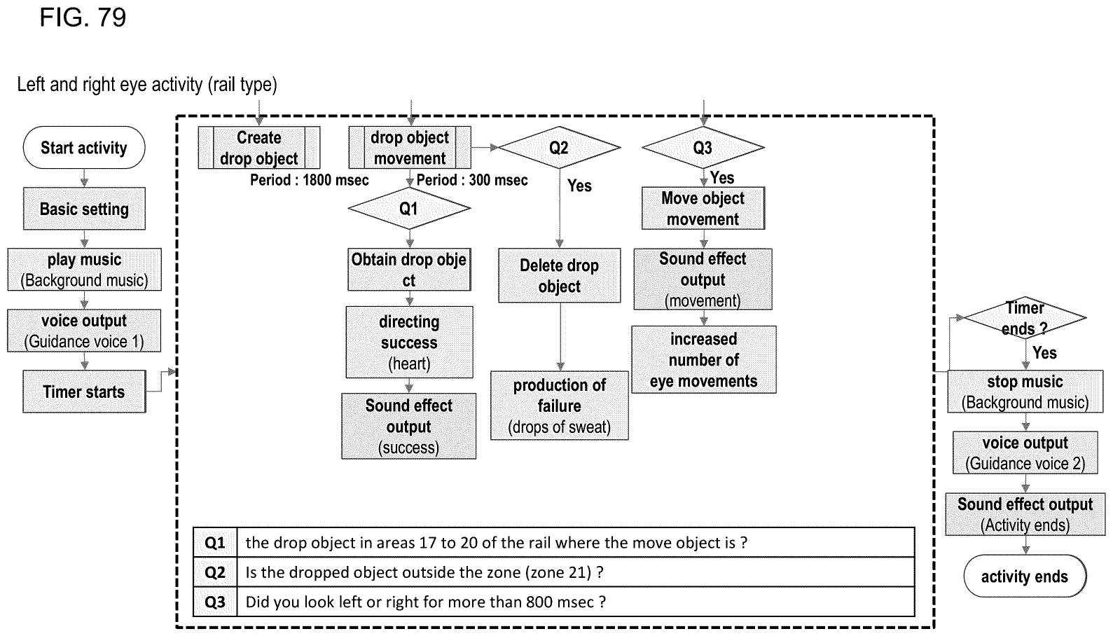

FIG. 79 is a flow chart illustrating an execution flow for the eyeball exercise instructions in FIGS. 77-78. The box depicted in FIG. 79 represents an execution flow of the user interface for the eyeball exercise instruction displayed by the digital apparatus.

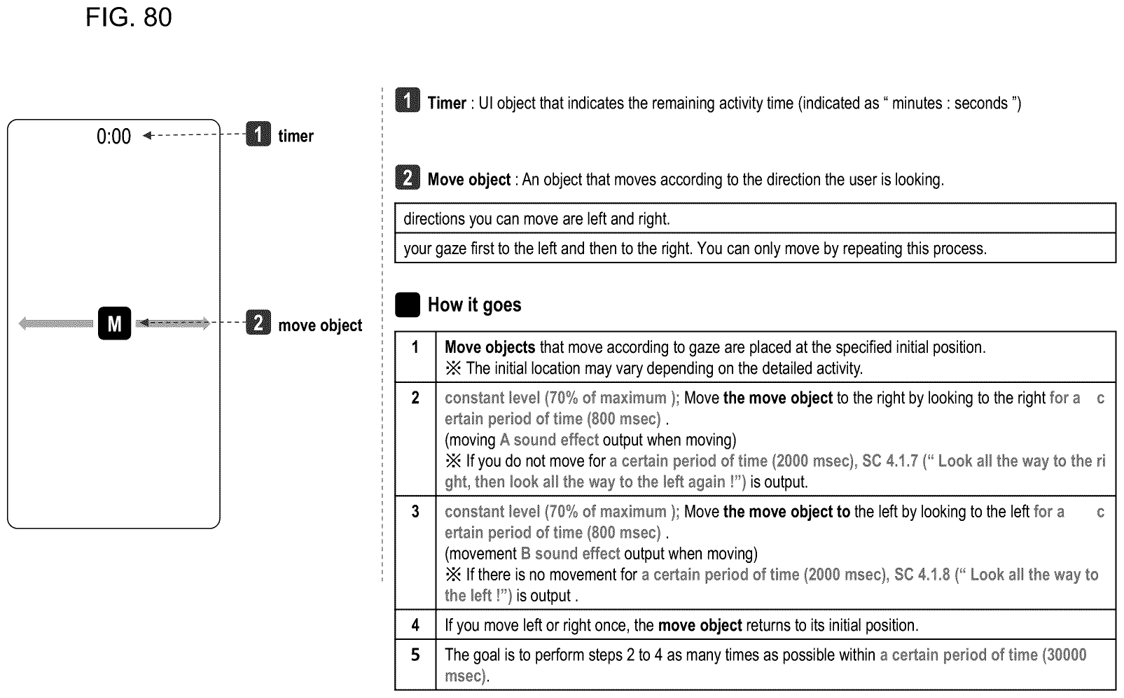

FIG. 80 depicts an example of a user interface for an eyeball exercise instruction displayed in the digital apparatus. The user interface may include visual images such as icons, images, characters and/or information and be displayed by the digital apparatus. The eye exercise digital therapeutic module includes an eyeball exercise instruction for a subject to move at least one eyeball to the left and right repeatedly. The eyeball movement is sensed by a sensor in the digital apparatus, adherence by the subject to the eyeball exercise instruction of the eye exercise digital therapeutic module.

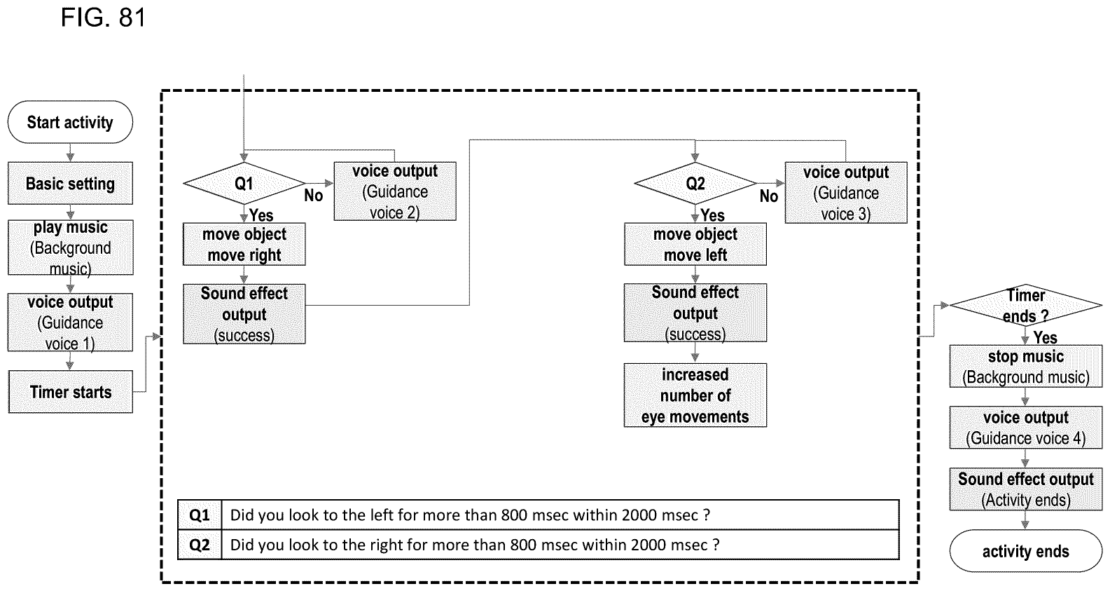

FIG. 81 is a flow chart illustrating an execution flow for the eyeball exercise instructions in FIG. 80.

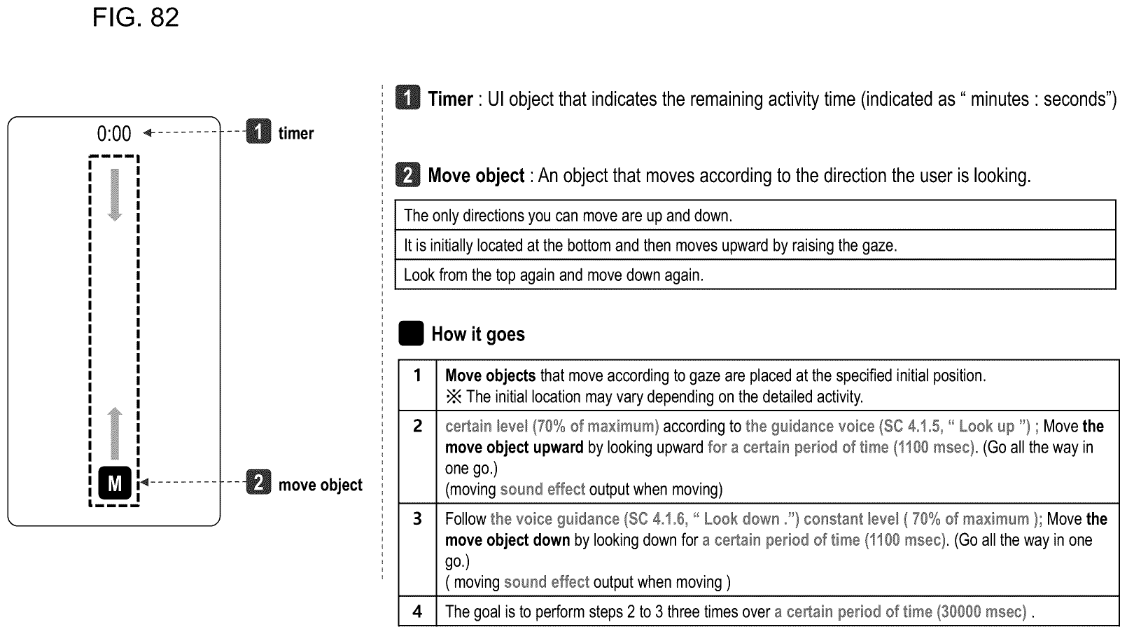

FIG. 82 depicts an example of a user interface for an eyeball exercise instruction displayed in the digital apparatus. The user interface may include visual images such as icons, images, characters and/or information and be displayed by the digital apparatus. The eye exercise digital therapeutic module includes an eyeball exercise instruction for a subject to move at least one eyeball vertically. The eyeball movement is sensed by a sensor in the digital apparatus, adherence by the subject to the eyeball exercise instruction of the eye exercise digital therapeutic module.

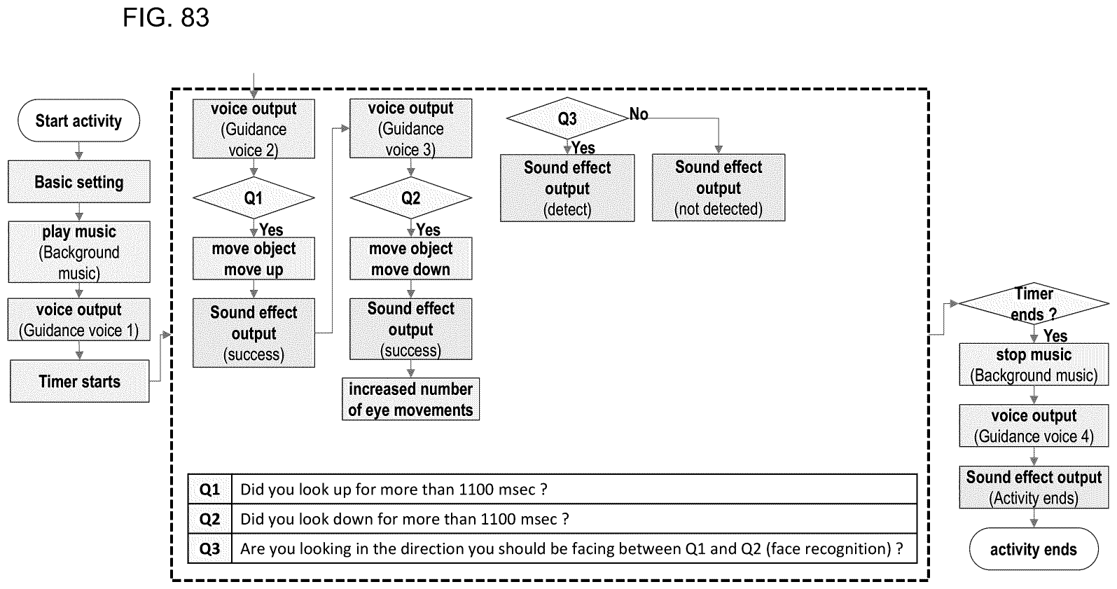

FIG. 83 is a flow chart illustrating an execution flow for the eyeball exercise instructions in FIG. 82.

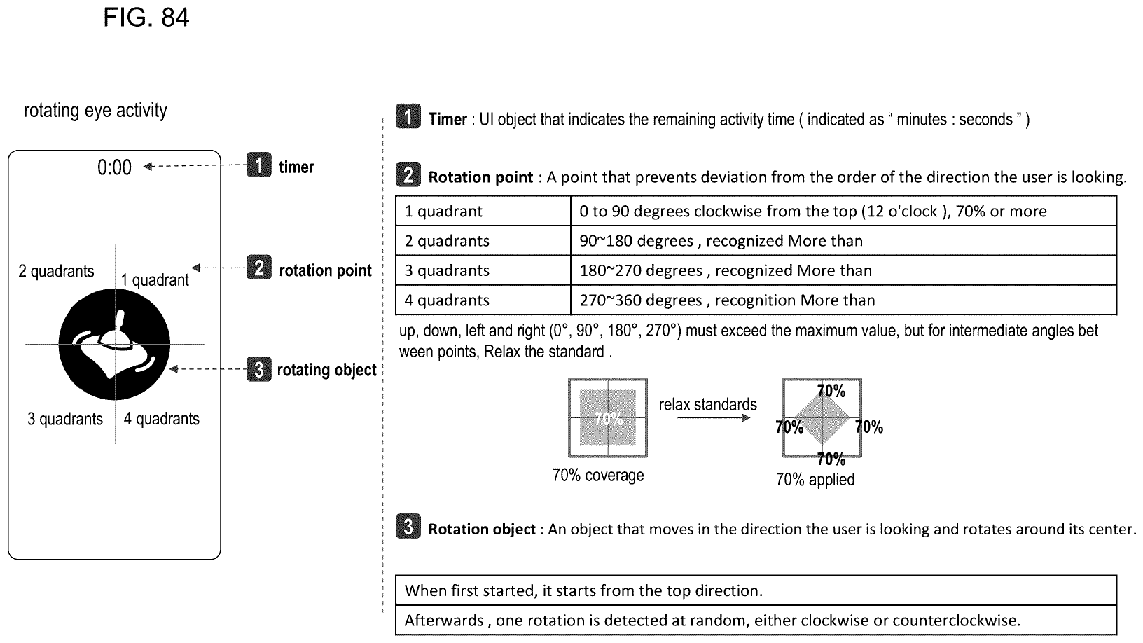

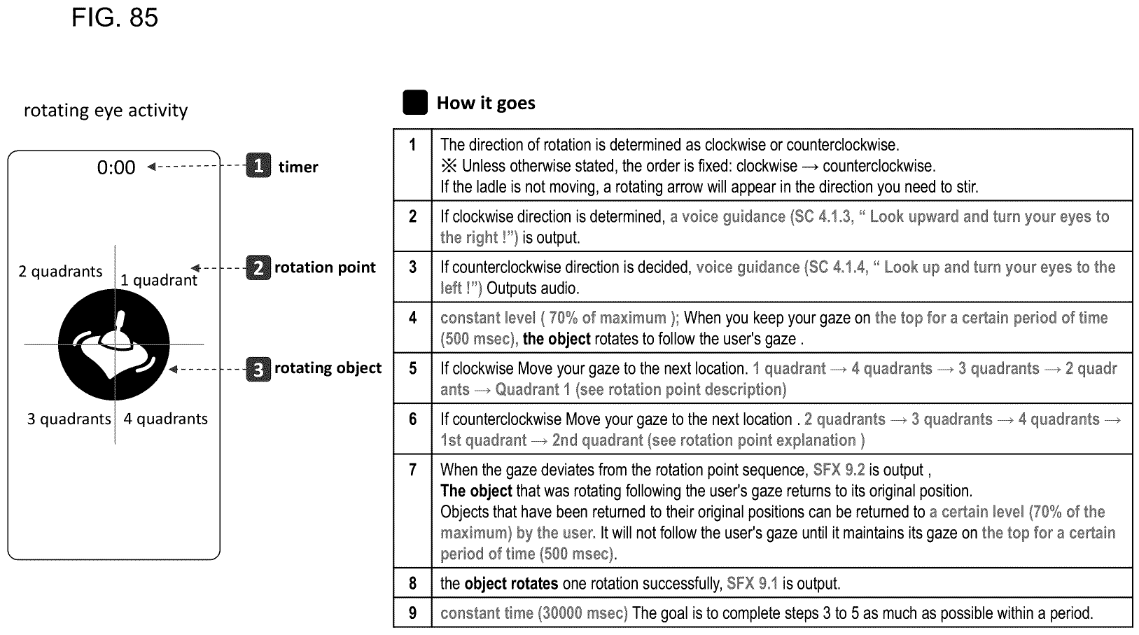

FIGS. 84-85 depict an example of a user interface for an eyeball exercise instruction displayed in the digital apparatus. The user interface may include visual images such as icons, images, characters and/or information and be displayed by the digital apparatus. The eye exercise digital therapeutic module includes an eyeball exercise instruction for a subject to rotate at least one eyeball. The eyeball movement is sensed by a sensor in the digital apparatus, adherence by the subject to the eyeball exercise instruction of the eye exercise digital therapeutic module.

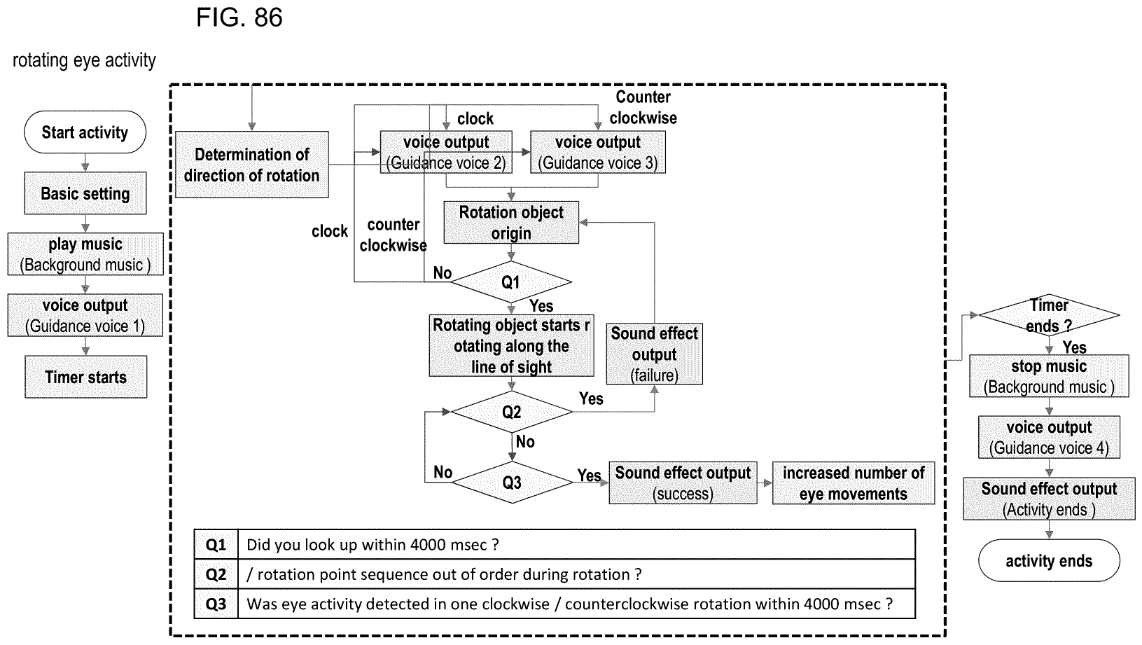

FIG. 86 is a flow chart illustrating an execution flow for the eyeball exercise instructions in FIGS. 84-85.

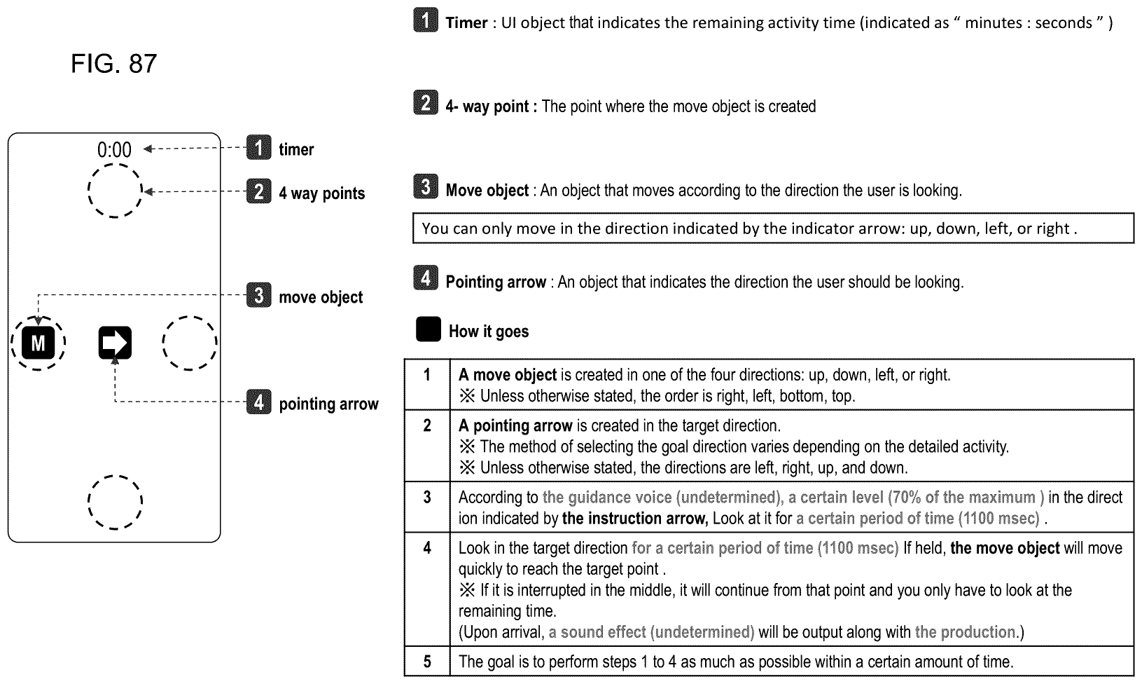

FIG. 87 depicts an example of a user interface for an eyeball exercise instruction displayed in the digital apparatus. The user interface may include visual images such as icons, images, characters and/or information and be displayed by a digital apparatus. The eye exercise digital therapeutic module includes an eyeball exercise instruction for a subject to move at least one eyeball vertically and horizontally. The eyeball movement is sensed by a sensor in the digital apparatus, adherence by the subject to the eyeball exercise instruction of the eye exercise digital therapeutic module.

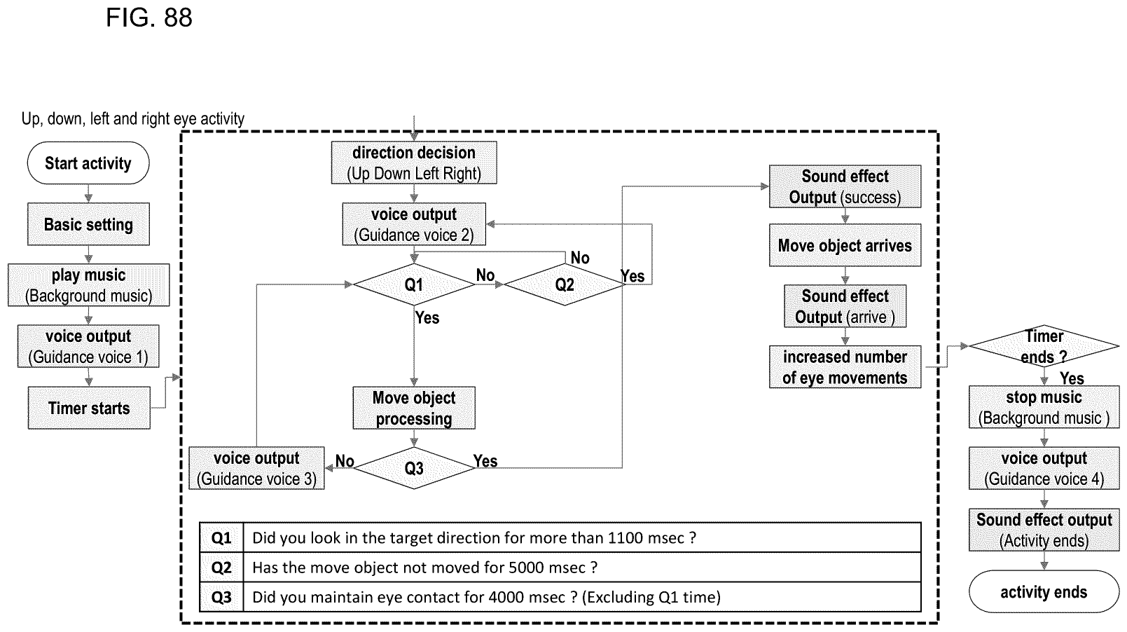

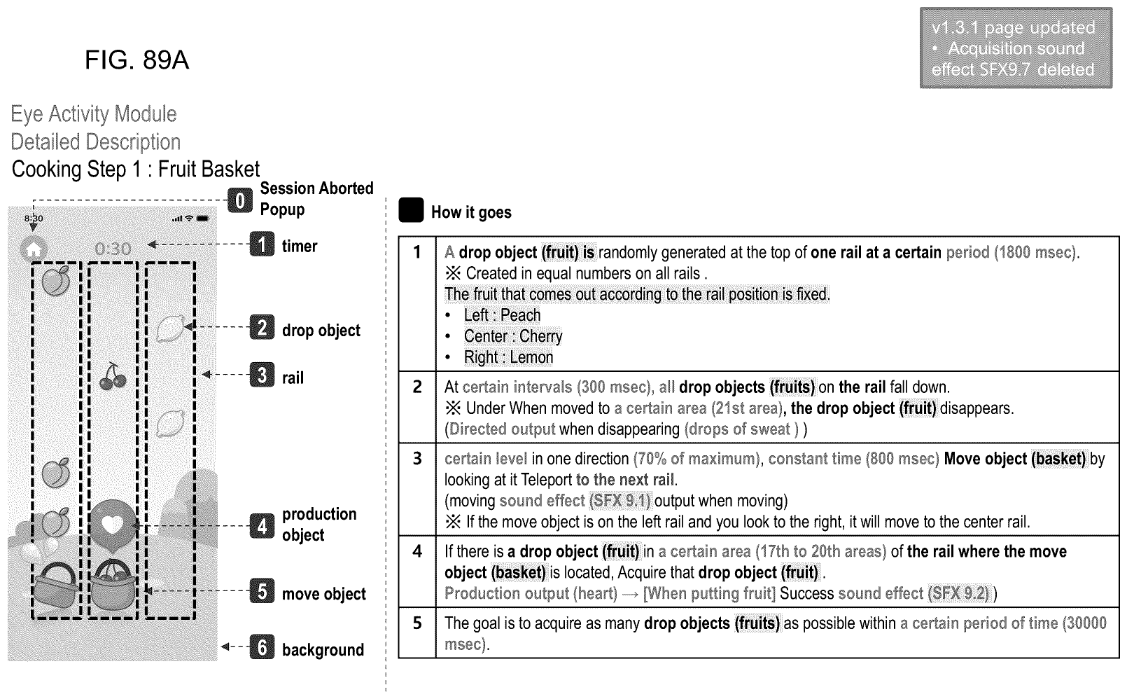

FIG. 88 is a flow chart illustrating an execution flow for the eyeball exercise instructions in FIG. 87.

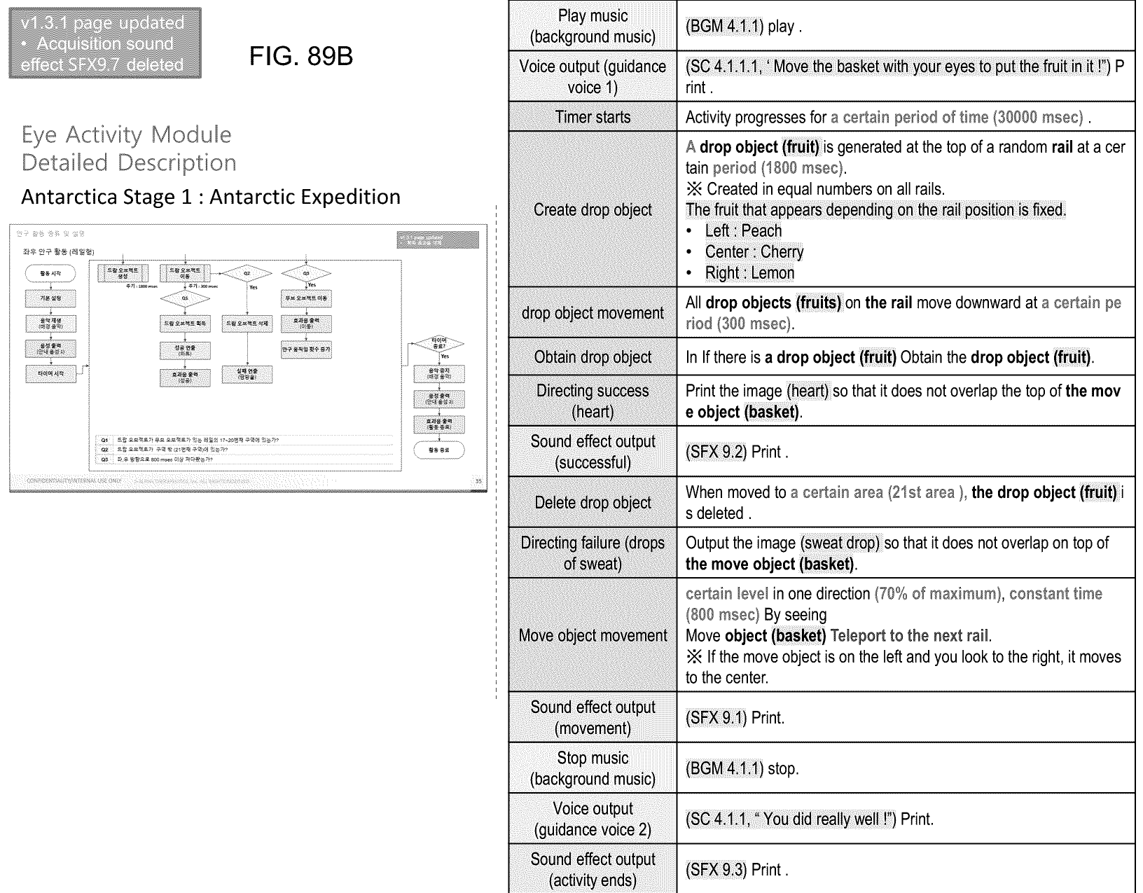

FIG. 89A depicts a screenshot of an eye exercise digital therapeutic module in FIGS. 77-78 of the present disclosure and FIG. 89B depicts a flow chart illustrating an execution flow for the eye exercise digital therapeutic module in FIGS. 77-78 of the present disclosure.

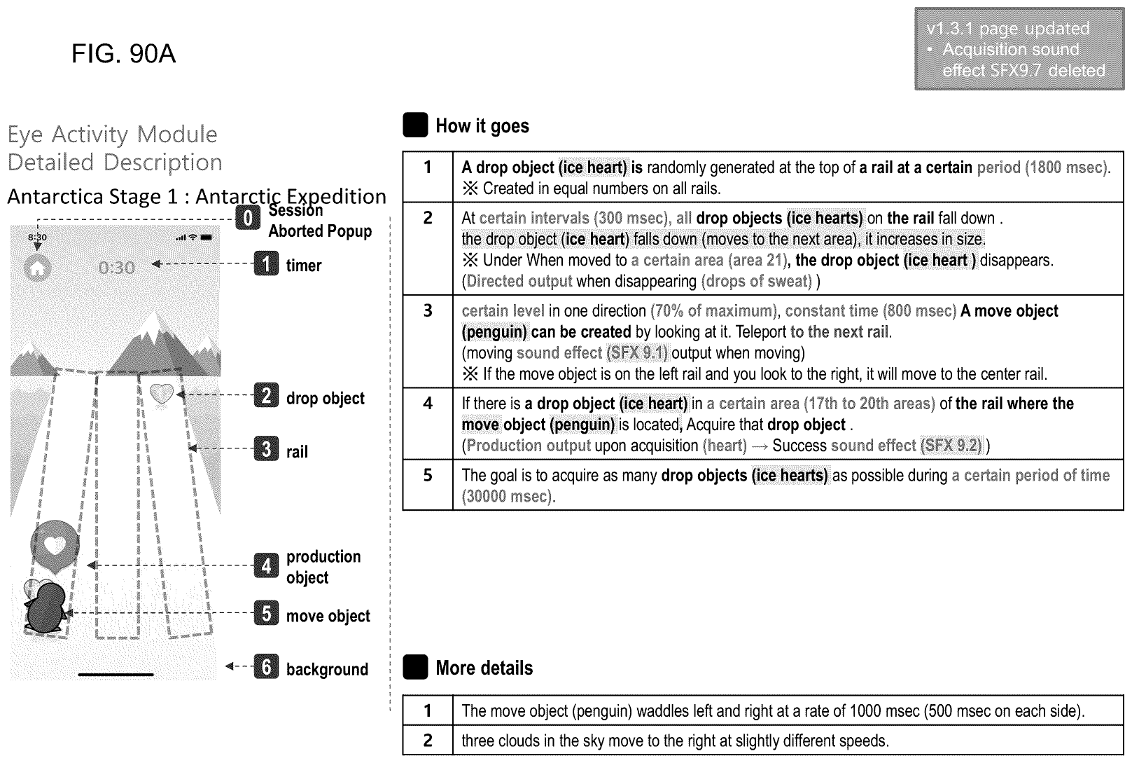

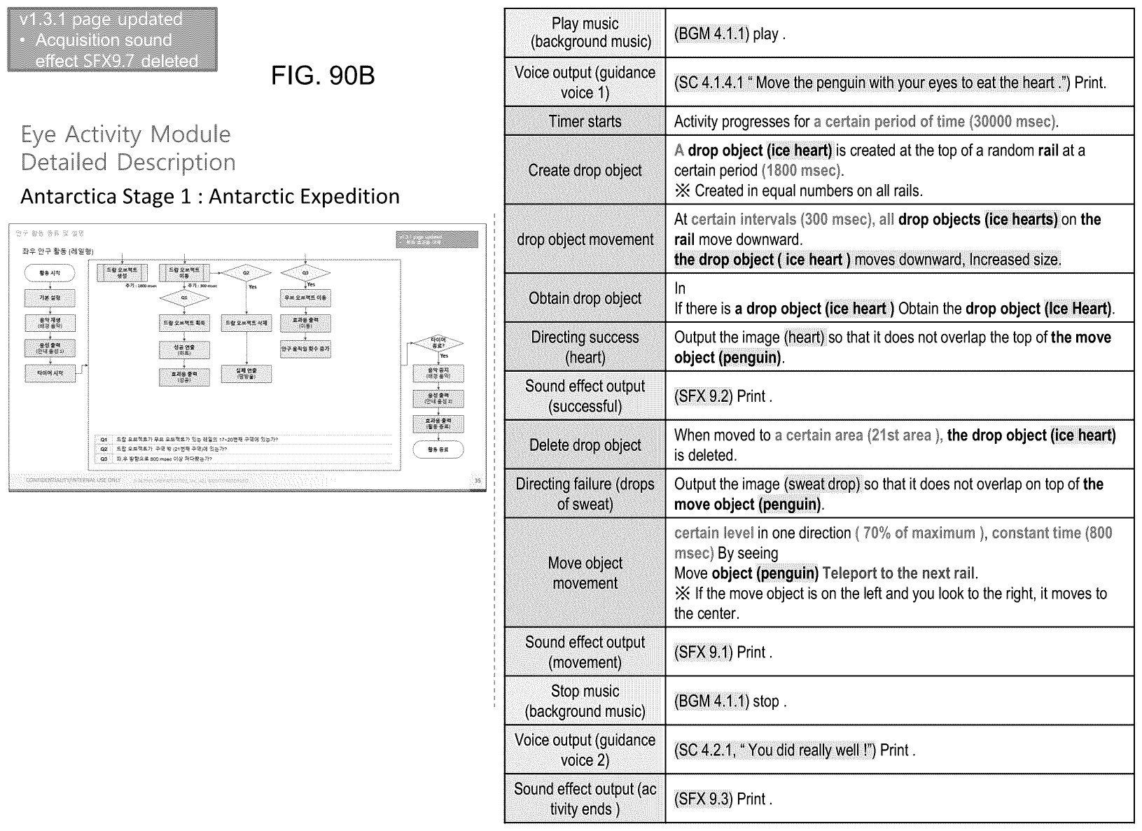

FIG. 90A depicts a screenshot of an eye exercise digital therapeutic module in FIGS. 77-78 of the present disclosure and FIG. 90B depicts a flow chart illustrating an execution flow for the eye exercise digital therapeutic module in FIGS. 77-78 of the present disclosure.

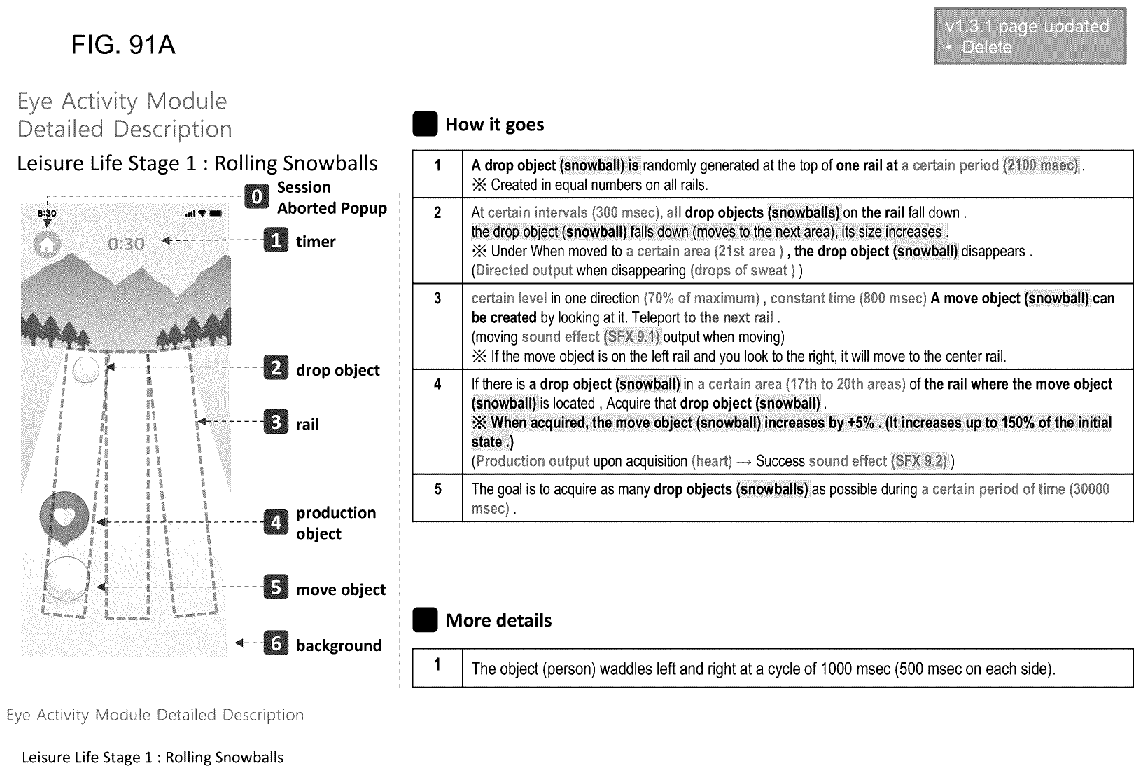

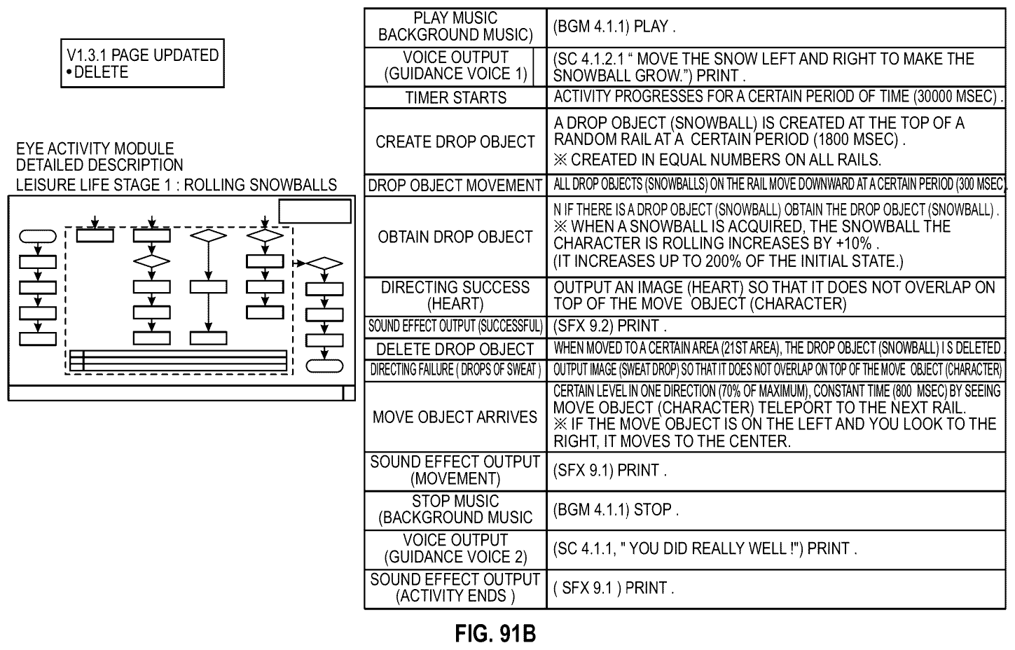

FIG. 91A depicts a screenshot of an eye exercise digital therapeutic module in FIGS. 77-78 of the present disclosure and FIG. 91B depicts a flow chart illustrating an execution flow for the eye exercise digital therapeutic module in FIGS. 77-78 of the present disclosure.

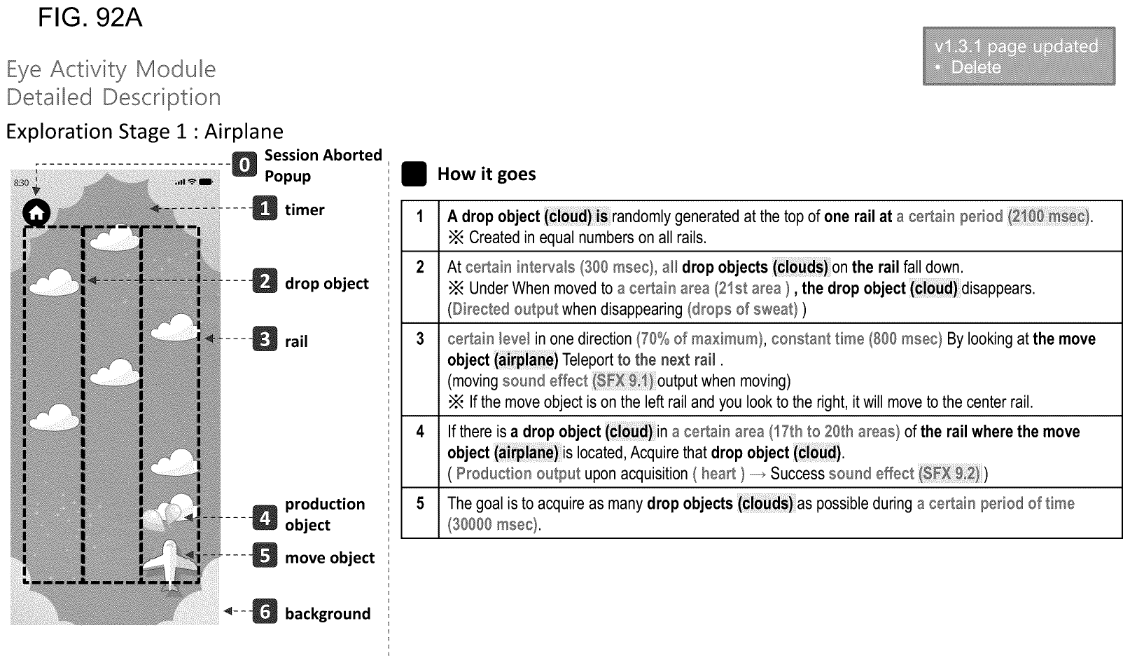

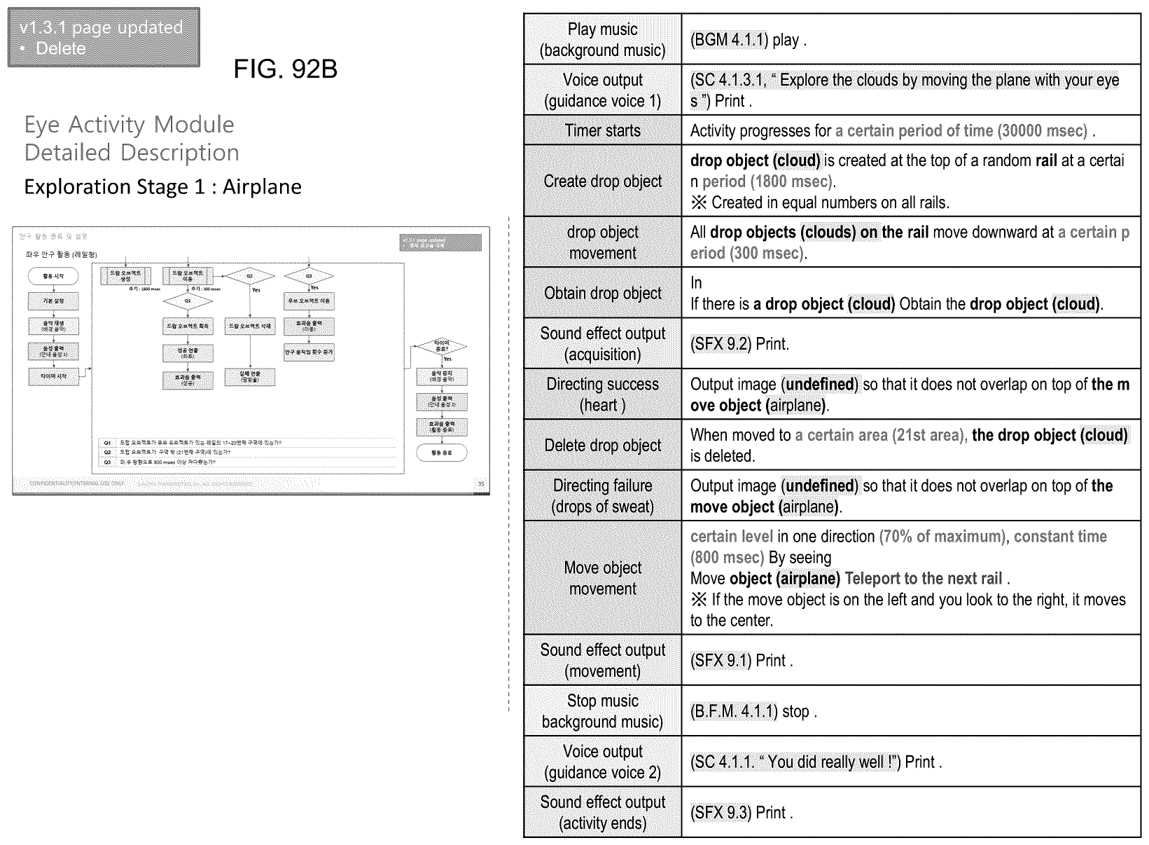

FIG. 92A depicts a screenshot of an eye exercise digital therapeutic module in FIGS. 77-78 of the present disclosure and FIG. 92B depicts a flow chart illustrating an execution flow for the eye exercise digital therapeutic module in FIGS. 77-78 of the present disclosure.

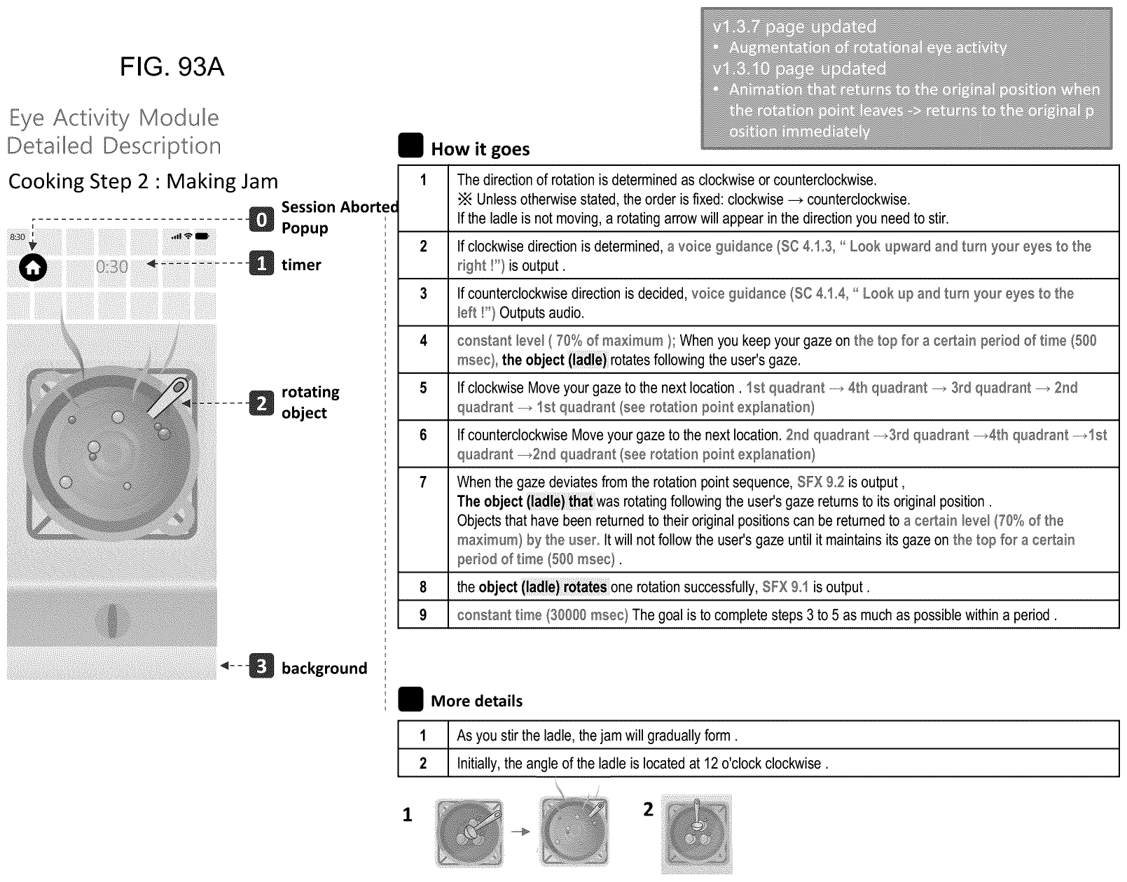

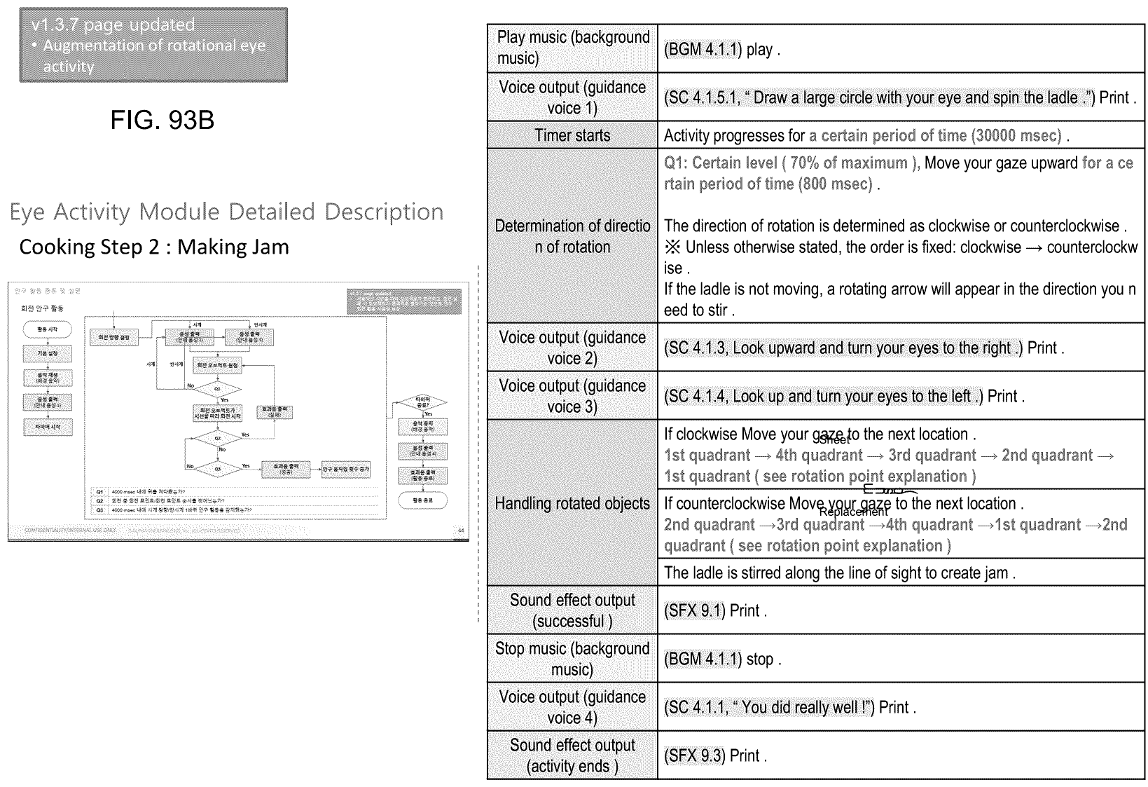

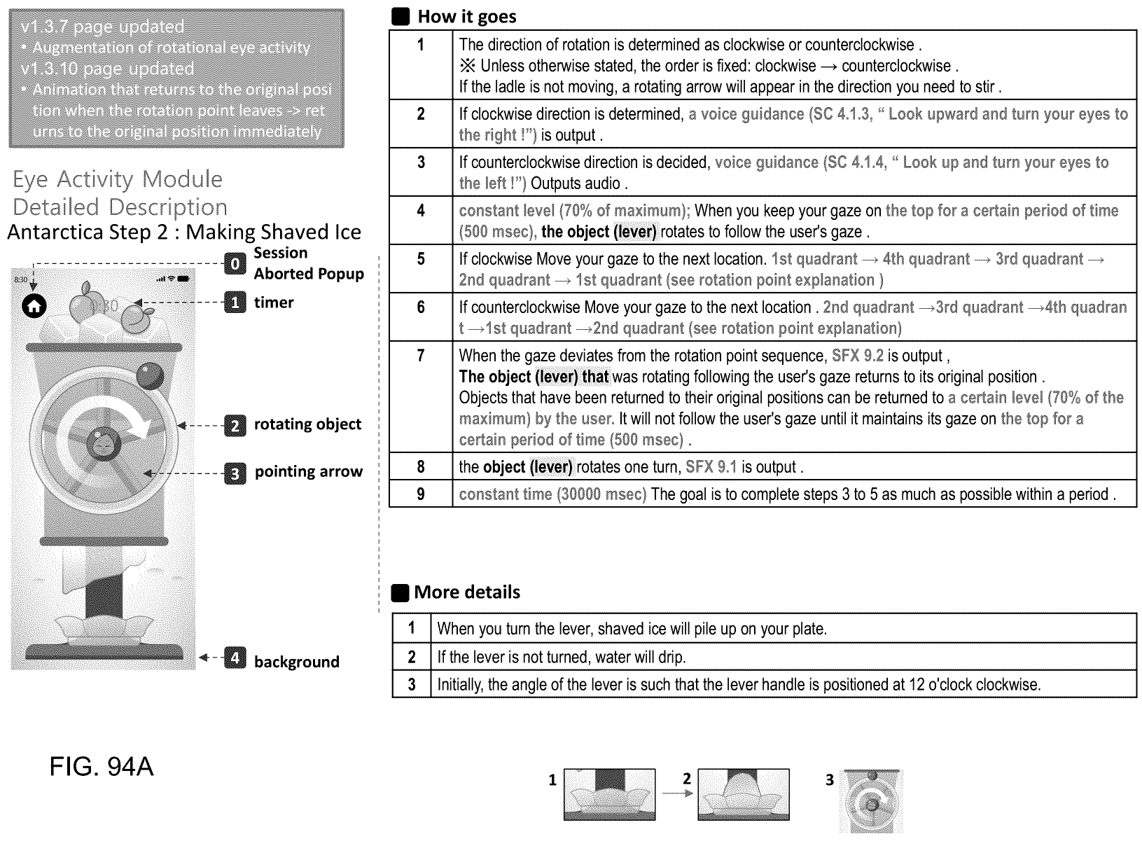

FIG. 93A depicts a screenshot of an eye exercise digital therapeutic module in FIGS. 84-85 of the present disclosure and FIG. 93B depicts a flow chart illustrating an execution flow for the eye exercise digital therapeutic module in FIGS. 84-85 of the present disclosure.

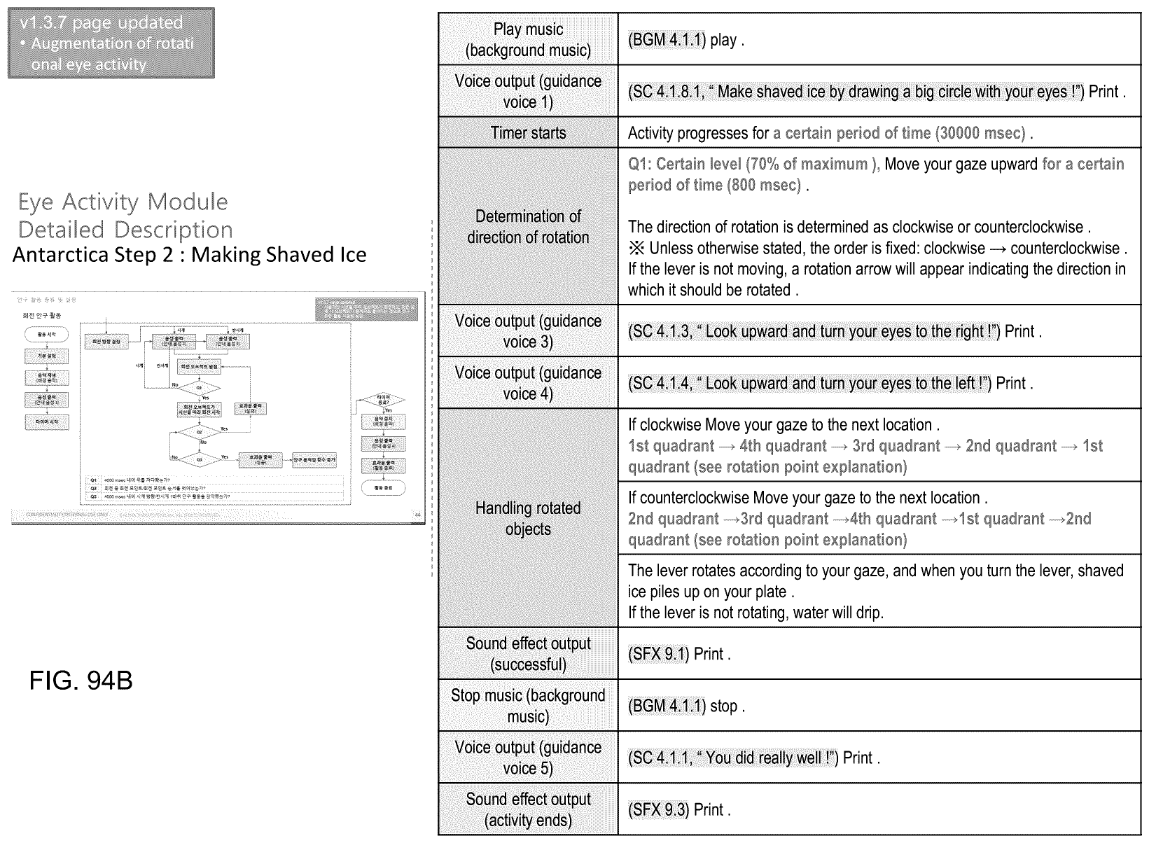

FIG. 94A depicts a screenshot of an eye exercise digital therapeutic module in FIGS. 84-85 of the present disclosure and FIG. 94B depicts a flow chart illustrating an execution flow for the eye exercise digital therapeutic module in FIGS. 84-85 of the present disclosure.

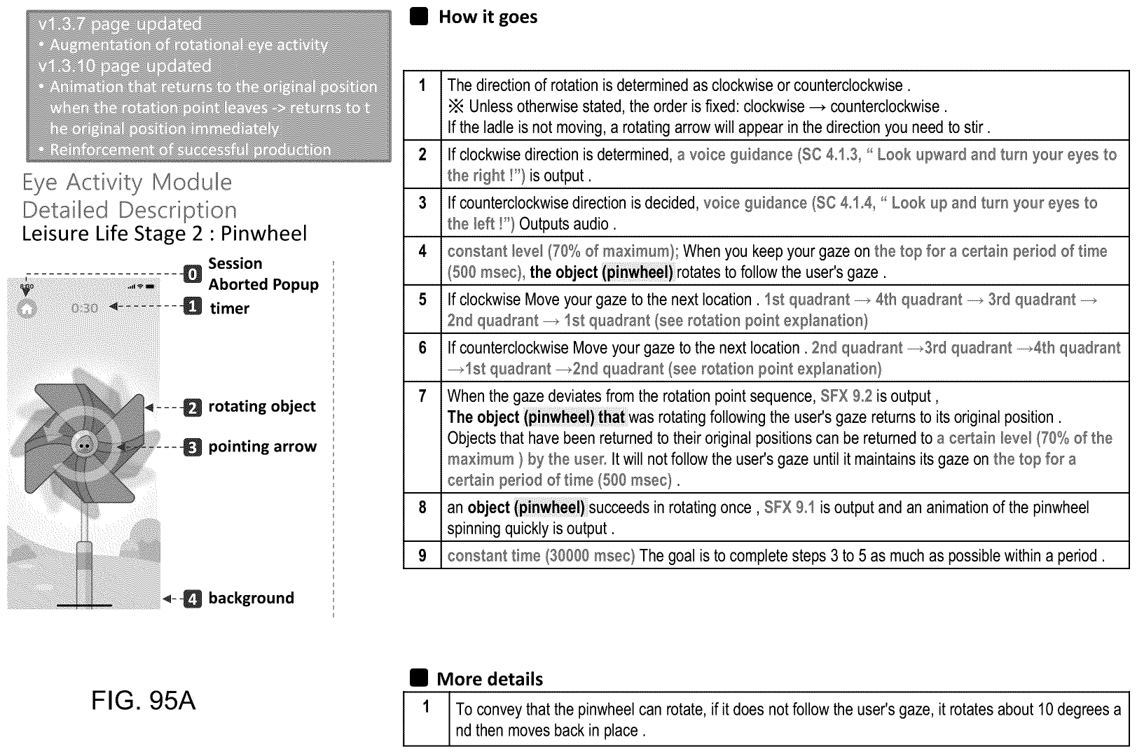

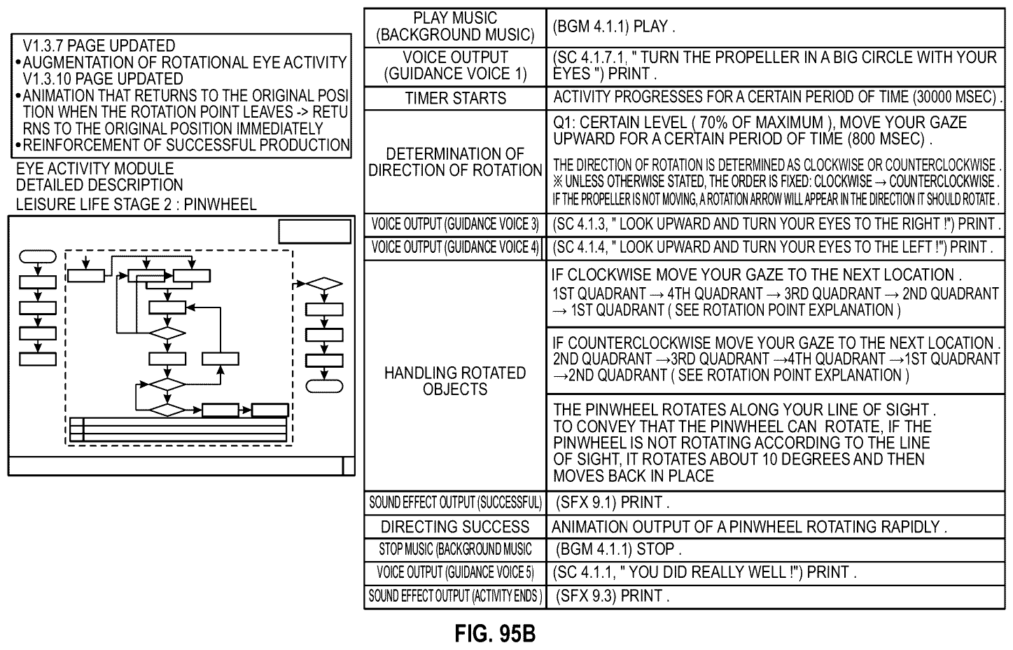

FIG. 95A depicts a screenshot of an eye exercise digital therapeutic module in FIGS. 84-85 of the present disclosure and FIG. 95B depicts a flow chart illustrating an execution flow for the eye exercise digital therapeutic module in FIGS. 84-85 of the present disclosure.

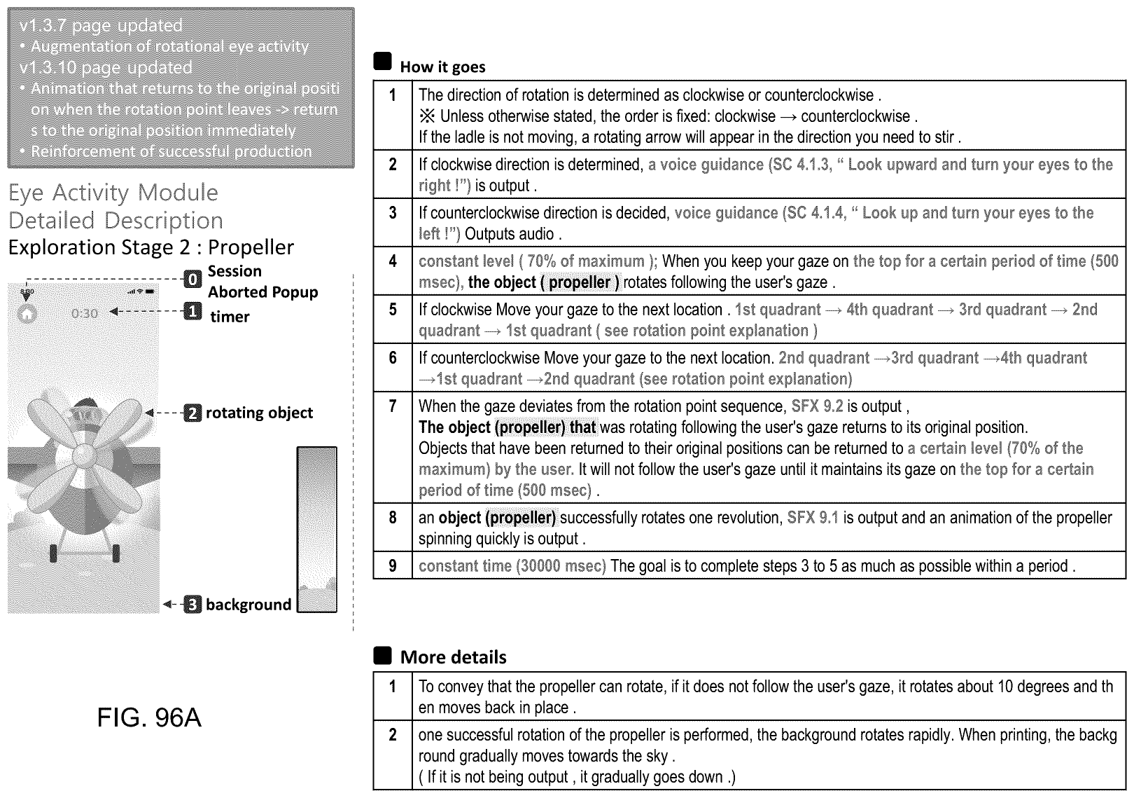

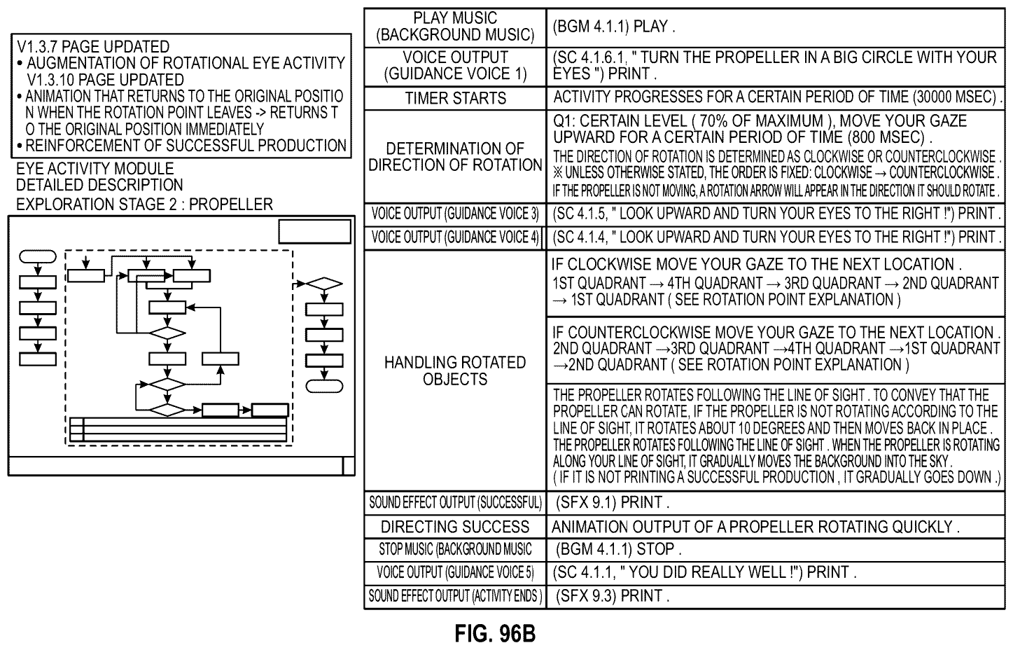

FIG. 96A depicts a screenshot of an eye exercise digital therapeutic module in FIGS. 84-85 of the present disclosure and FIG. 96B depicts a flow chart illustrating an execution flow for the eye exercise digital therapeutic module in FIGS. 84-85 of the present disclosure.

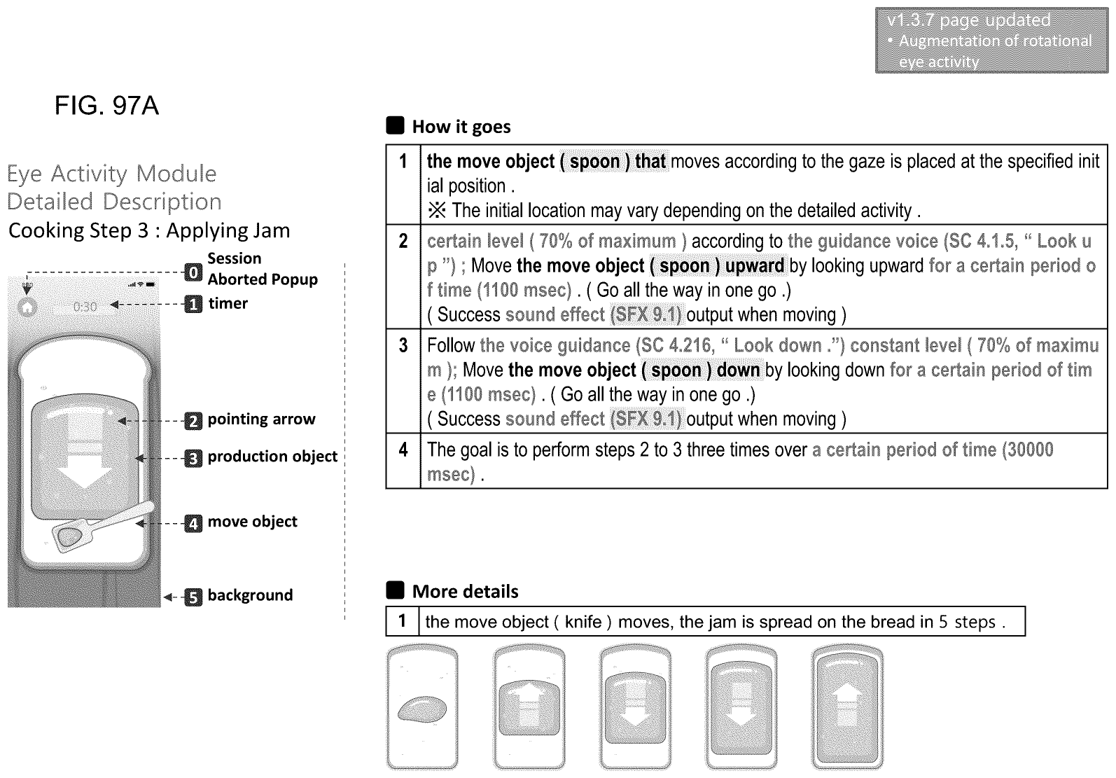

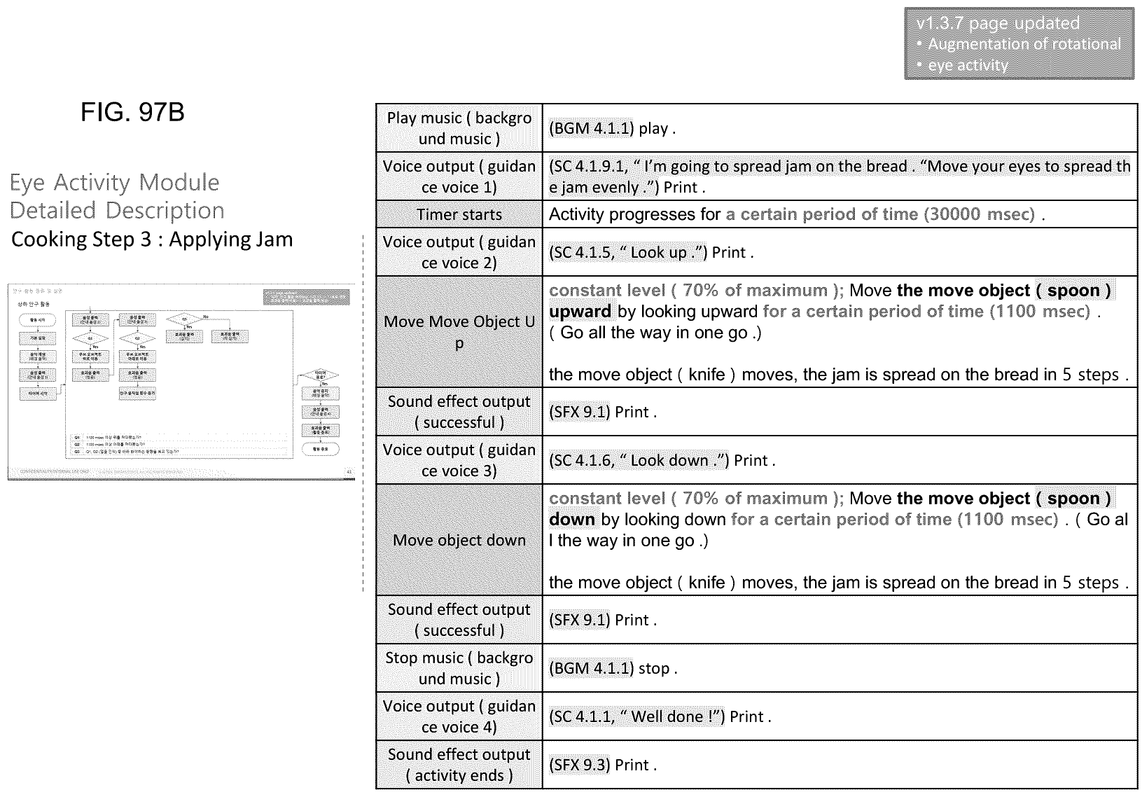

FIG. 97A depicts a screenshot of an eye exercise digital therapeutic module in FIG. 82 of the present disclosure and FIG. 97B depicts a flow chart illustrating an execution flow for the eye exercise digital therapeutic module in FIG. 82 of the present disclosure.

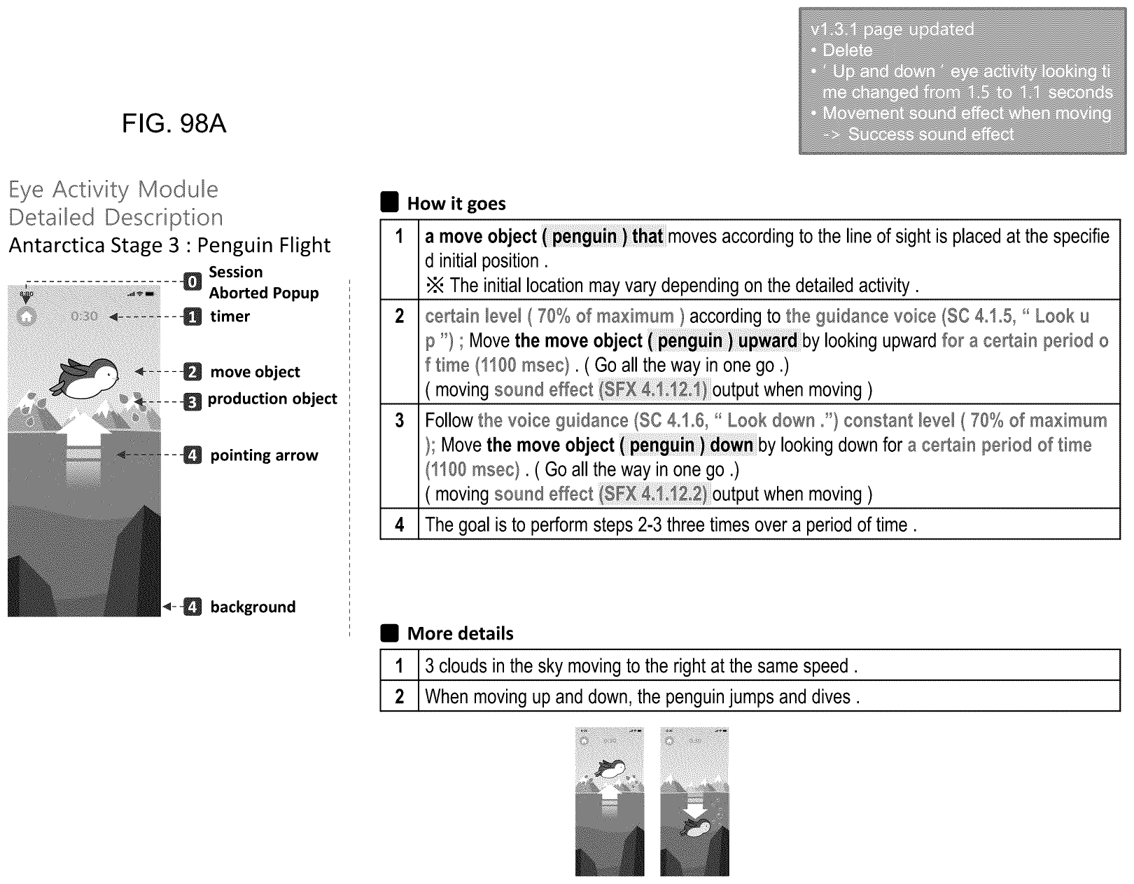

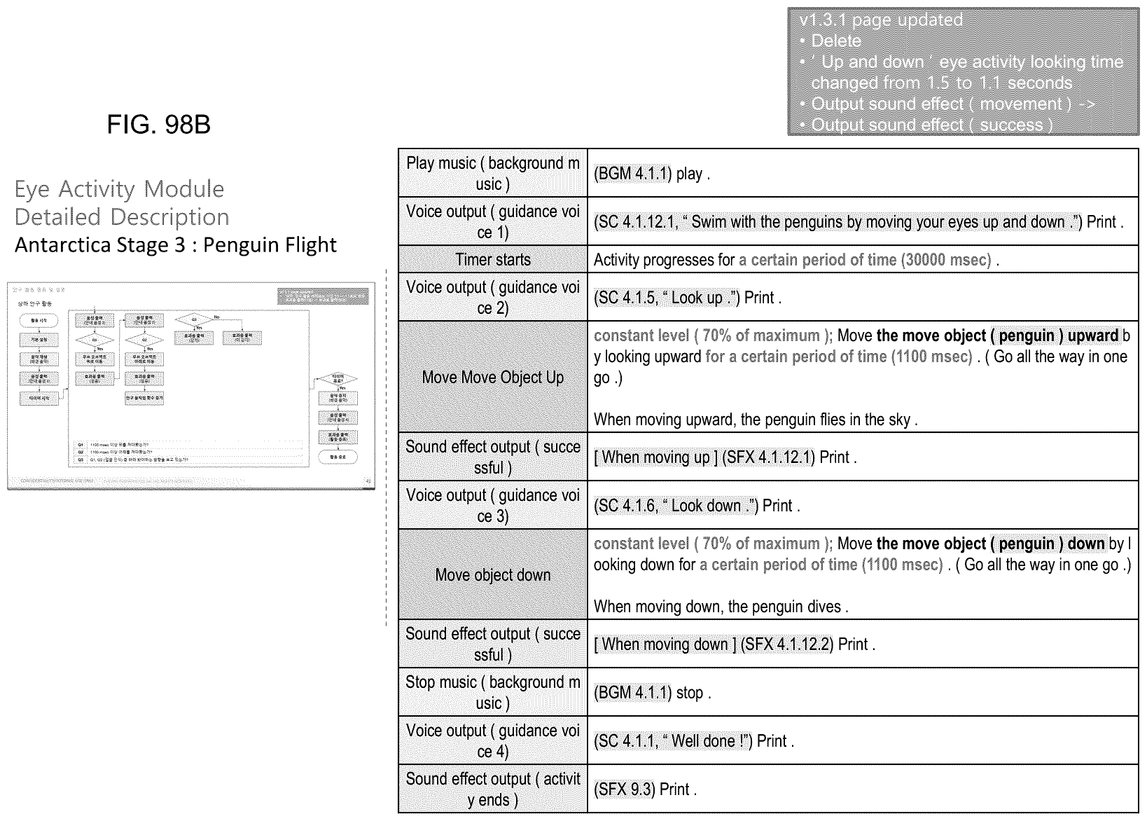

FIG. 98A depicts a screenshot of an eye exercise digital therapeutic module in FIG. 82 of the present disclosure and FIG. 98B depicts a flow chart illustrating an execution flow for the eye exercise digital therapeutic module in FIG. 82 of the present disclosure.

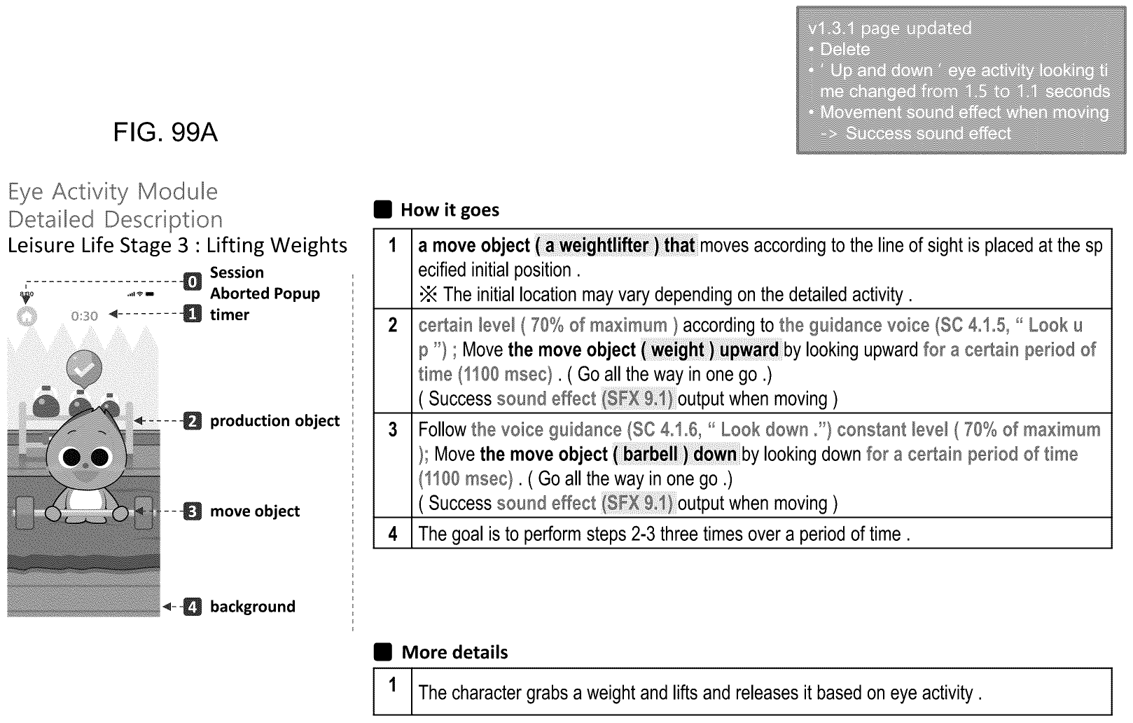

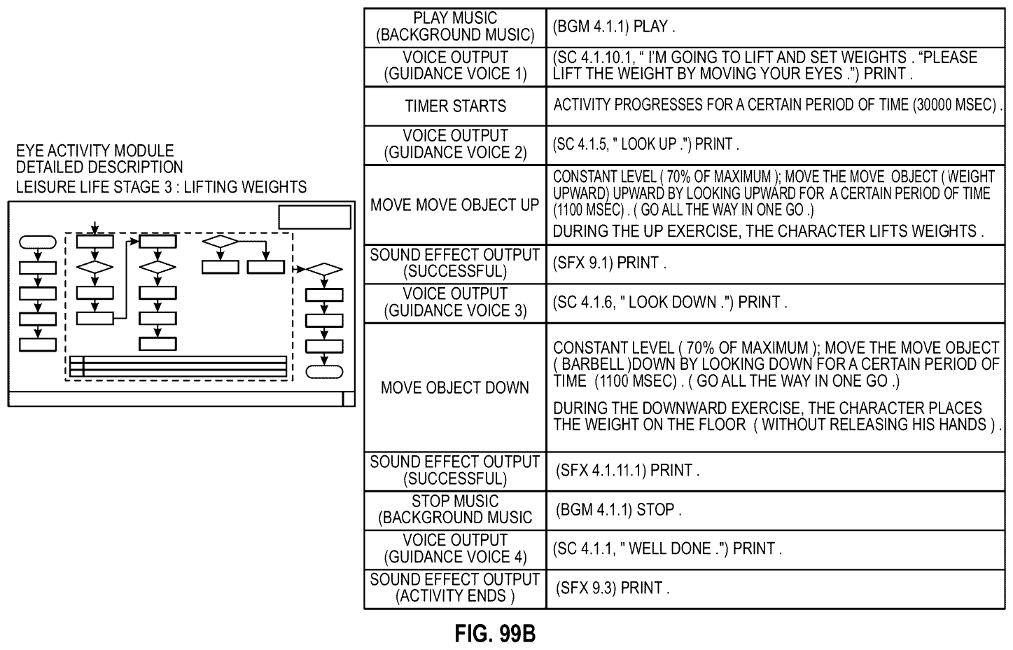

FIG. 99A depicts a screenshot of an eye exercise digital therapeutic module in FIG. 82 of the present disclosure and FIG. 99B depicts a flow chart illustrating an execution flow for the eye exercise digital therapeutic module in FIG. 82 of the present disclosure.

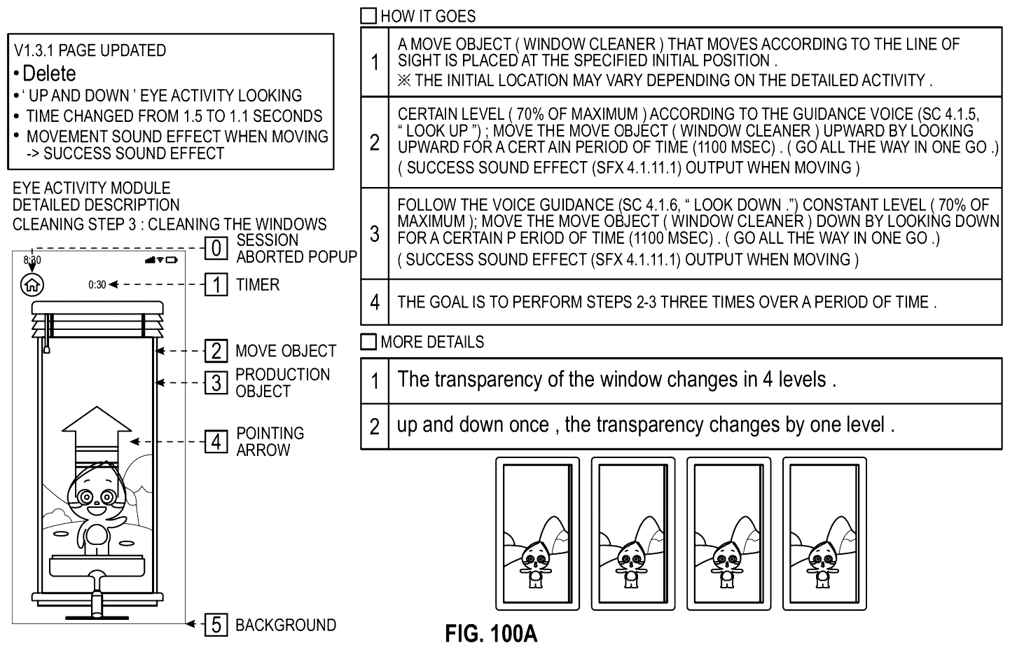

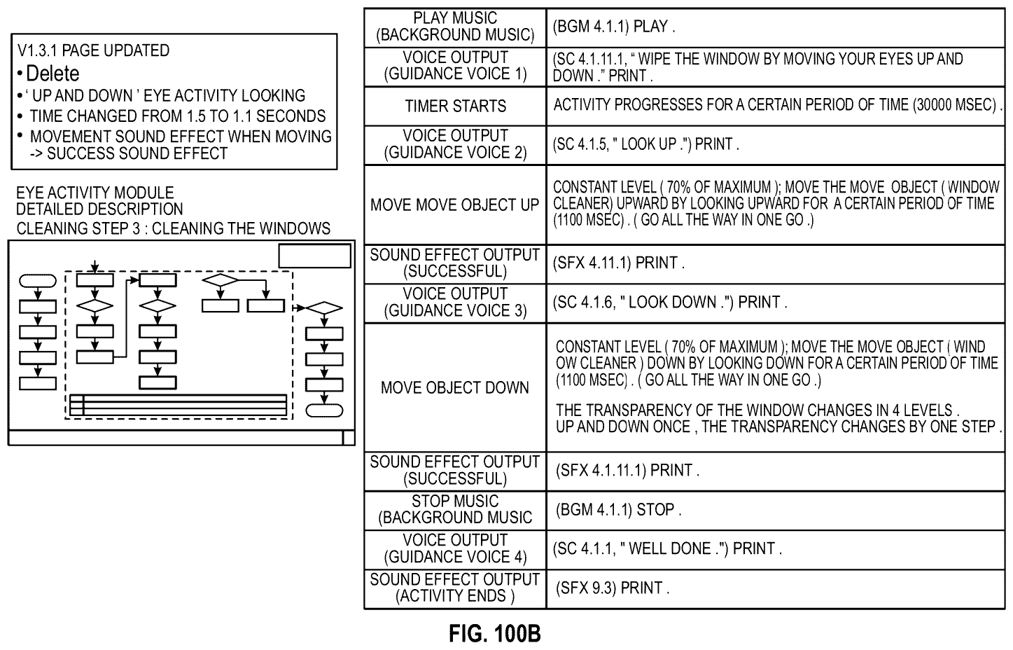

FIG. 100A depicts a screenshot of an eye exercise digital therapeutic module in FIG. 82 of the present disclosure and FIG. 100B depicts a flow chart illustrating an execution flow for the eye exercise digital therapeutic module in FIG. 82 of the present disclosure.

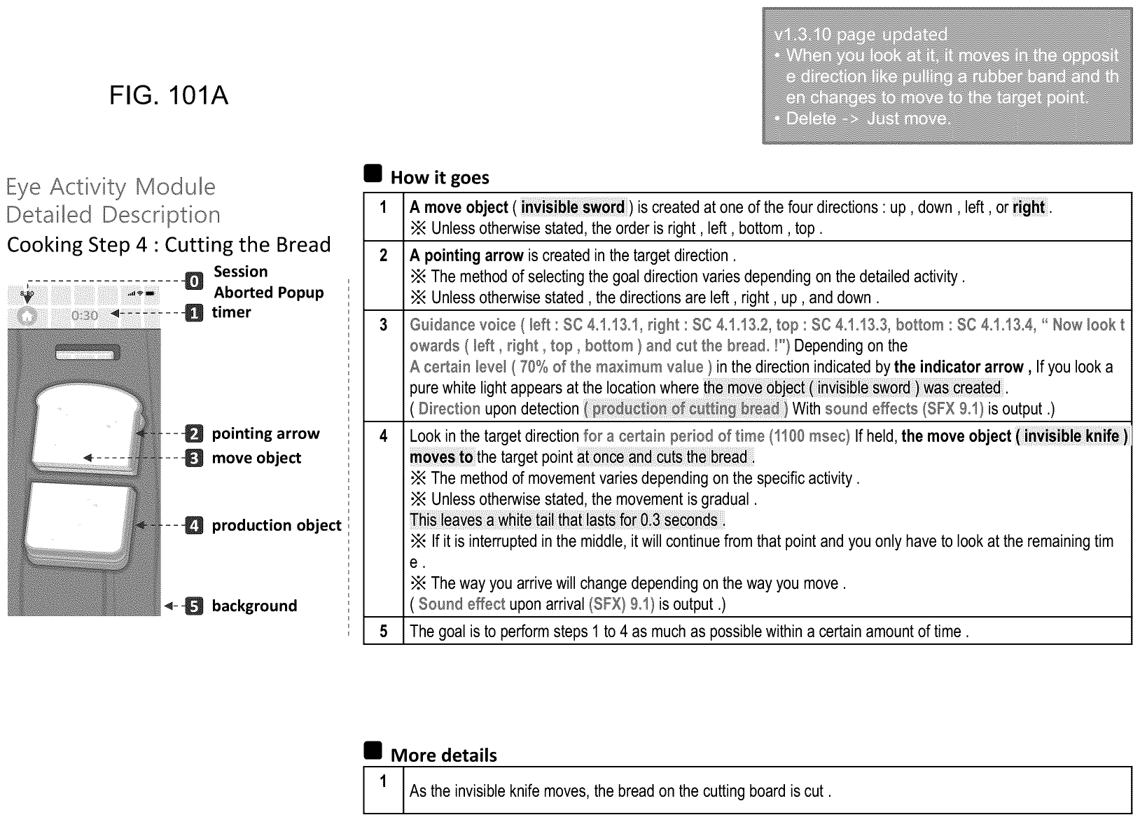

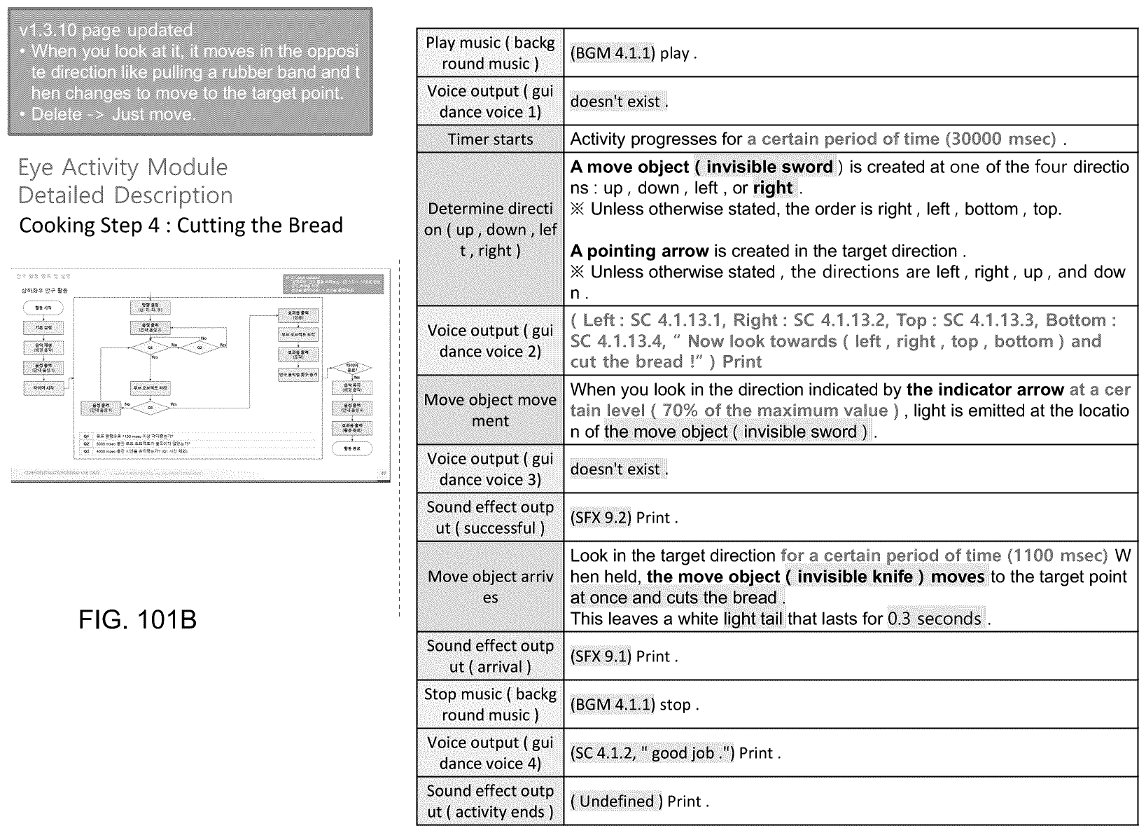

FIG. 101A depicts a screenshot of an eye exercise digital therapeutic module in FIG. 87 of the present disclosure and FIG. 101B depicts a flow chart illustrating an execution flow for the eye exercise digital therapeutic module in FIG. 87 of the present disclosure.

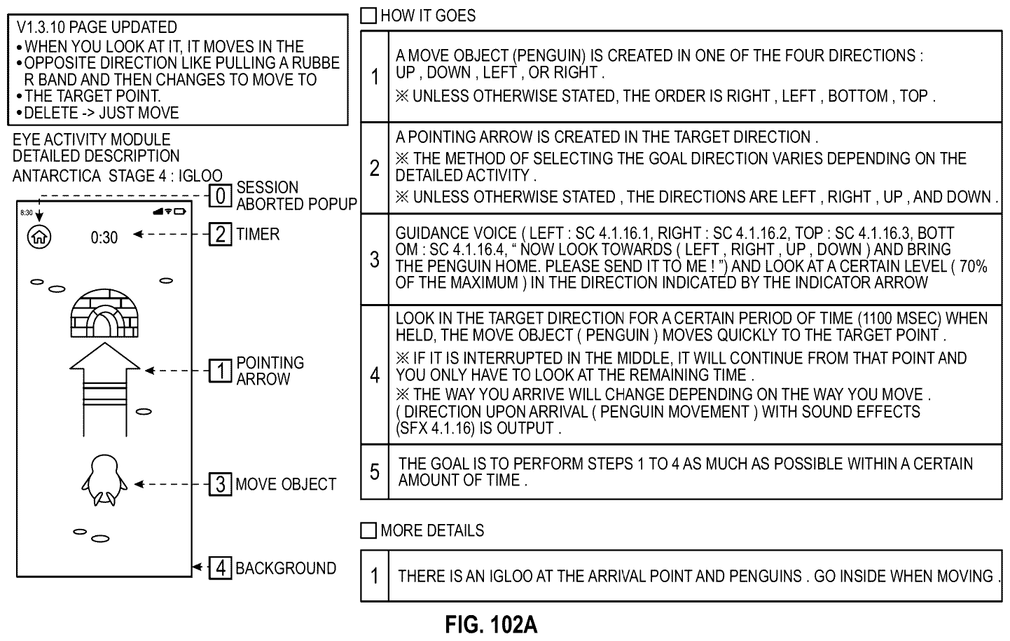

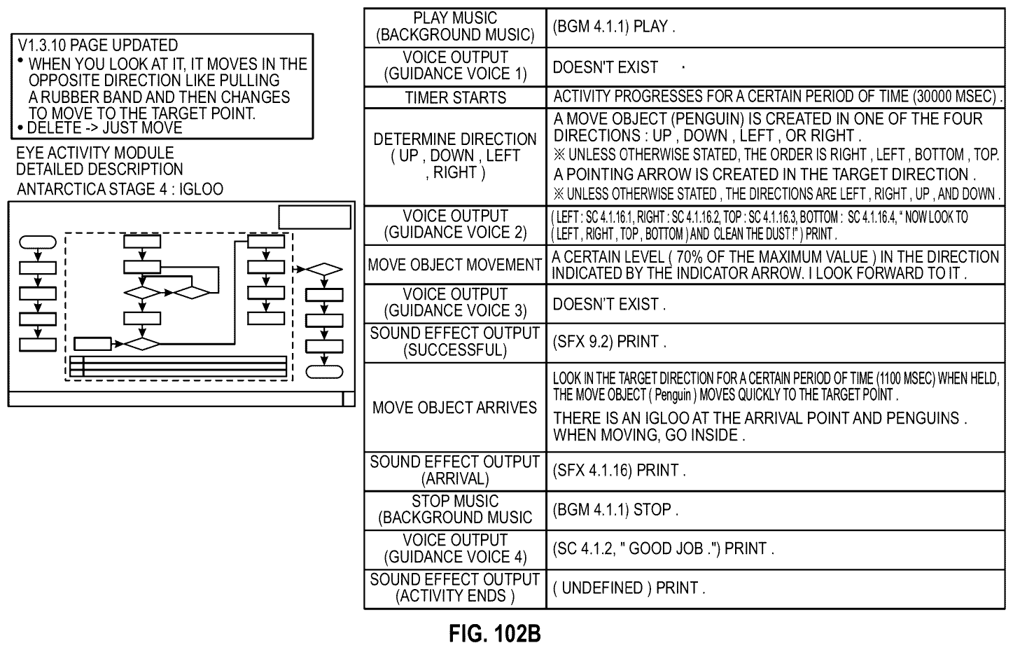

FIG. 102A depicts a screenshot of an eye exercise digital therapeutic module in FIG. 87 of the present disclosure and FIG. 102B depicts a flow chart illustrating an execution flow for the eye exercise digital therapeutic module in FIG. 87 of the present disclosure.

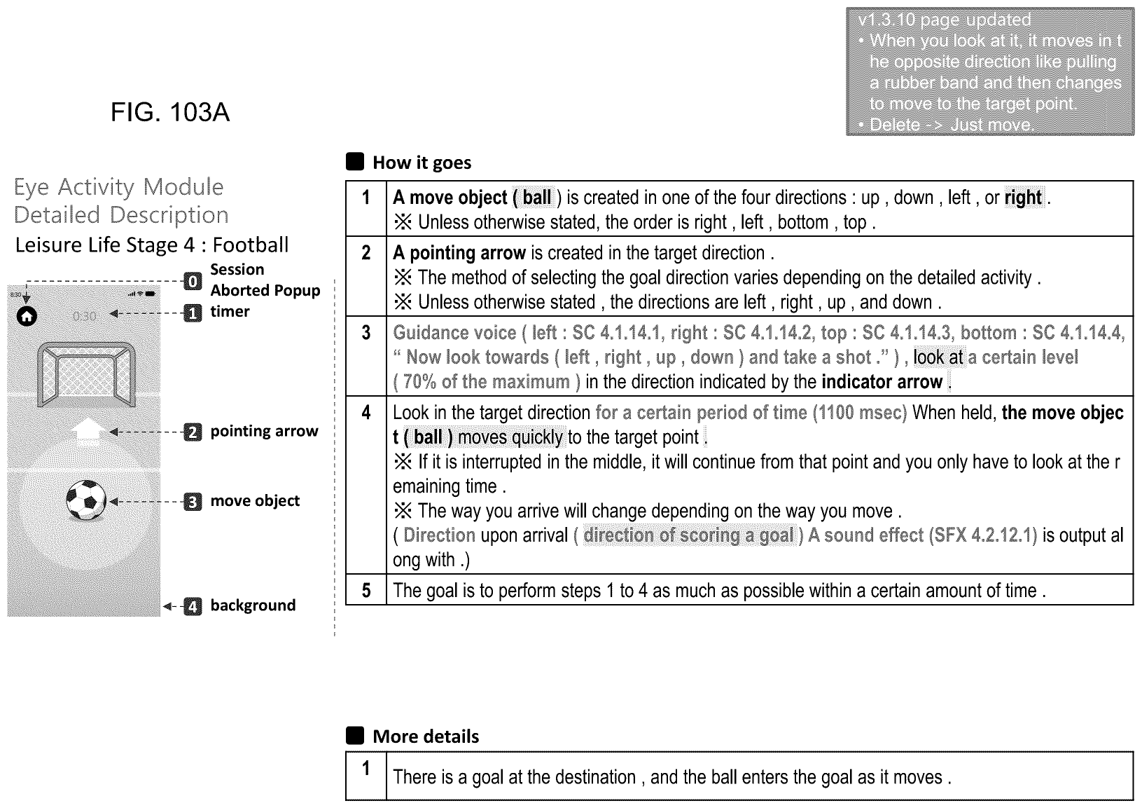

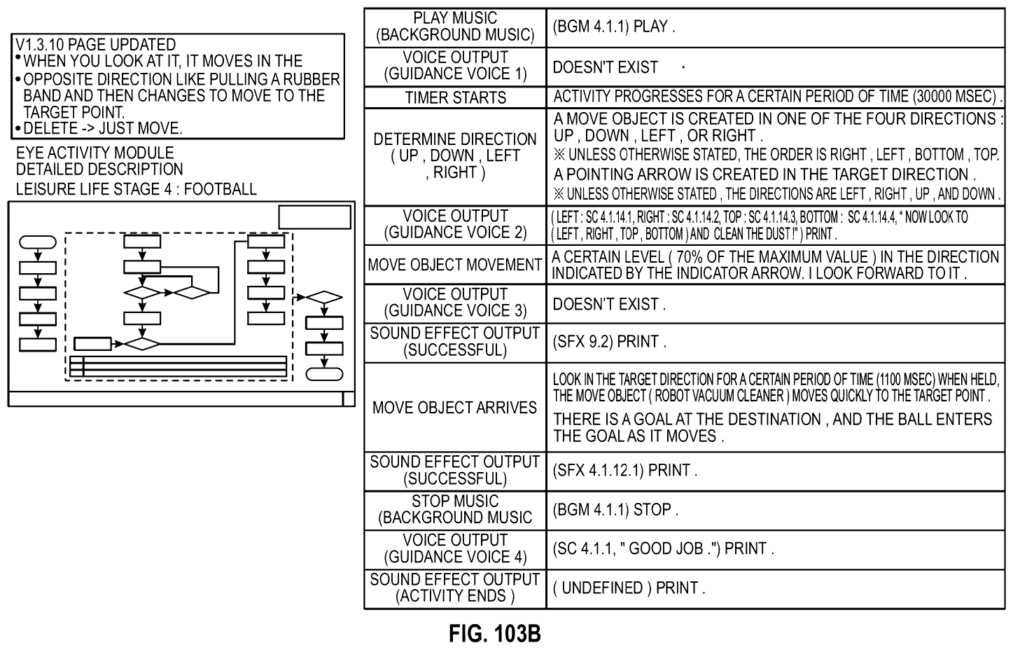

FIG. 103A depicts a screenshot of an eye exercise digital therapeutic module in FIG. 87 of the present disclosure and FIG. 103B depicts a flow chart illustrating an execution flow for the eye exercise digital therapeutic module in FIG. 87 of the present disclosure.

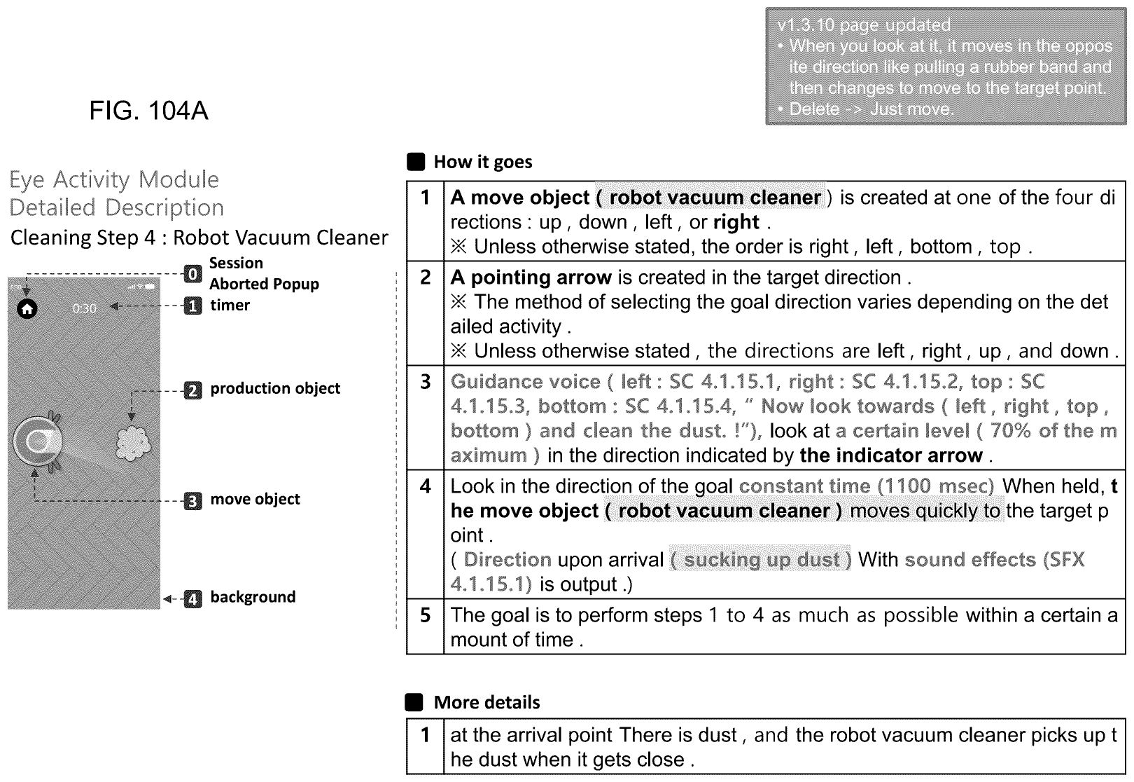

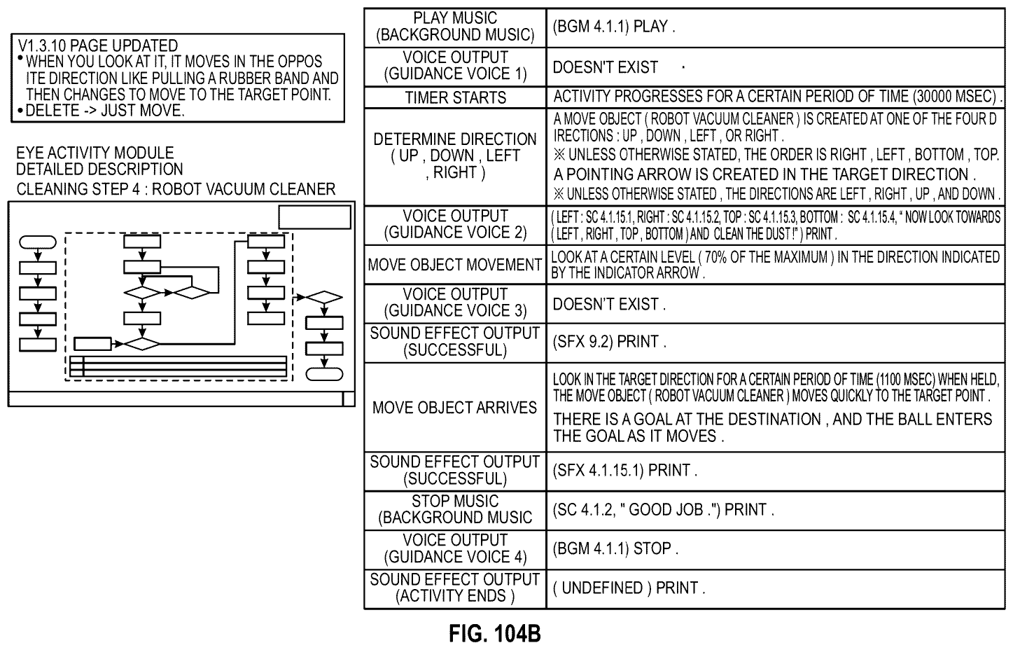

FIG. 104A depicts a screenshot of an eye exercise digital therapeutic module in FIG. 87 of the present disclosure and FIG. 104B depicts a flow chart illustrating an execution flow for the eye exercise digital therapeutic module in FIG. 87 of the present disclosure.

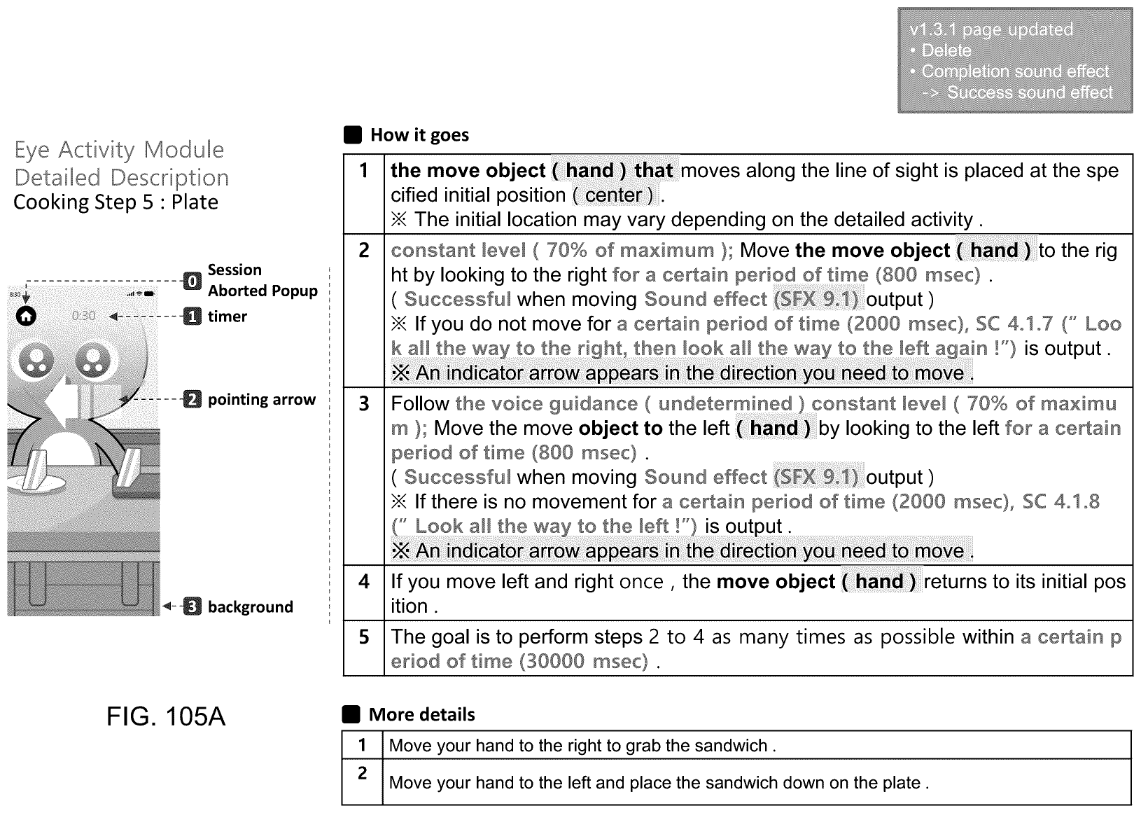

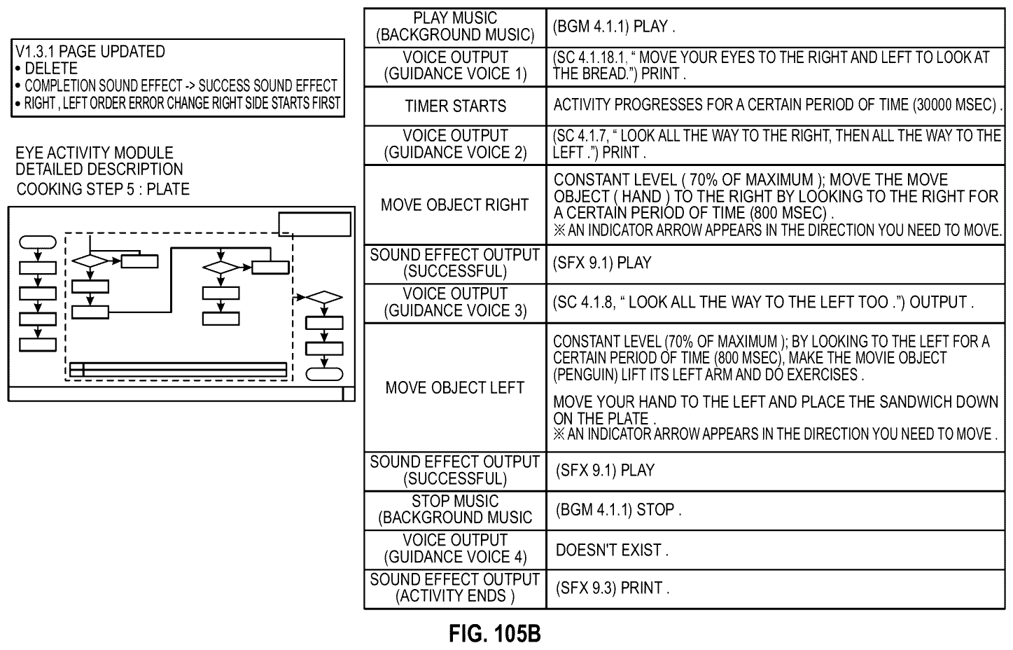

FIG. 105A depicts a screenshot of an eye exercise digital therapeutic module in FIG. 80 of the present disclosure and FIG. 105B depicts a flow chart illustrating an execution flow for the eye exercise digital therapeutic module in FIG. 80 of the present disclosure.

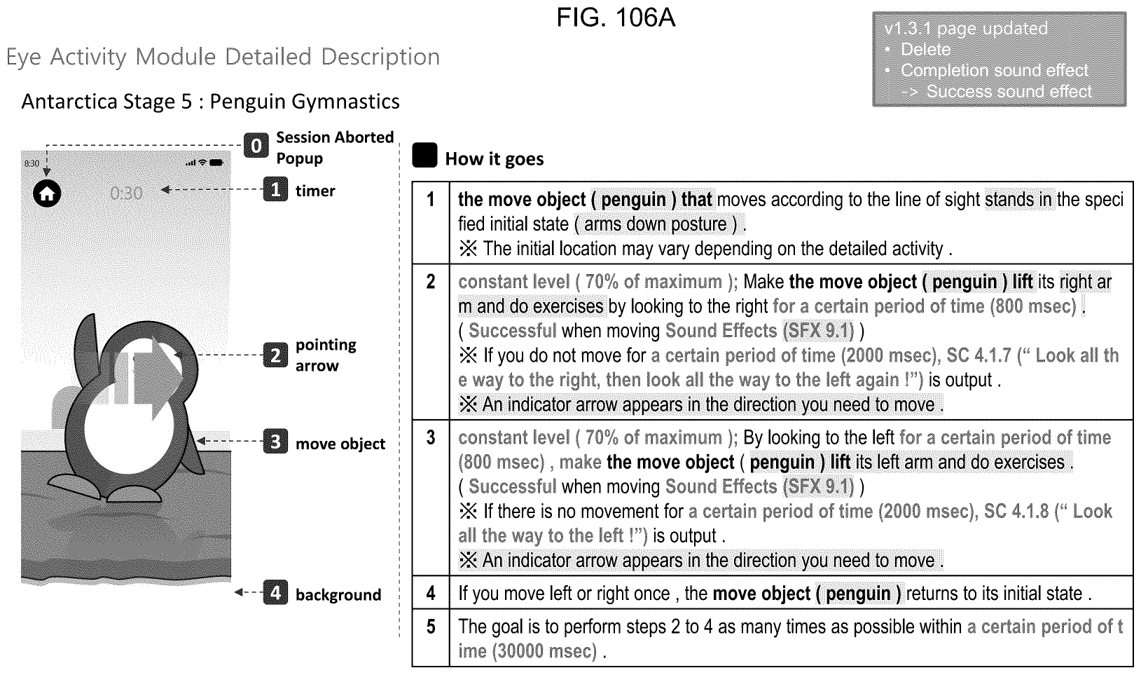

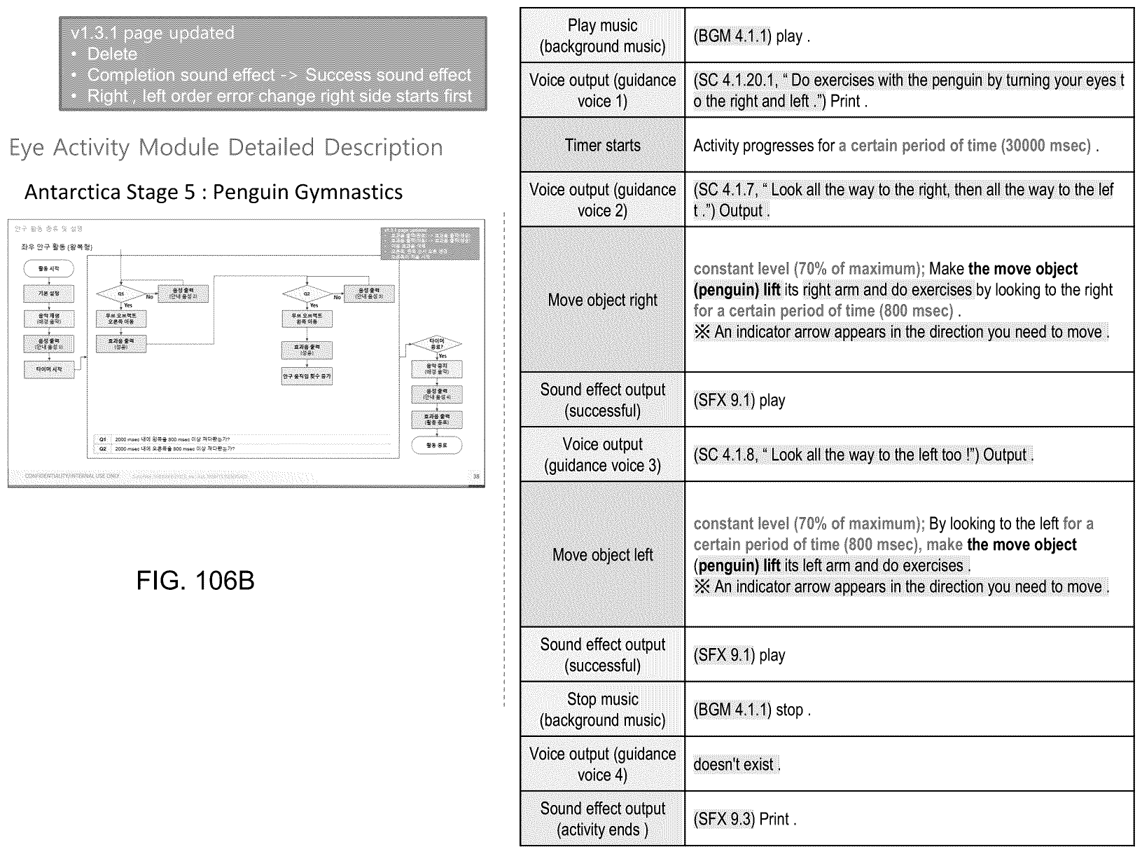

FIG. 106A depicts a screenshot of an eye exercise digital therapeutic module in FIG. 80 of the present disclosure and FIG. 106B depicts a flow chart illustrating an execution flow for the eye exercise digital therapeutic module in FIG. 80 of the present disclosure.

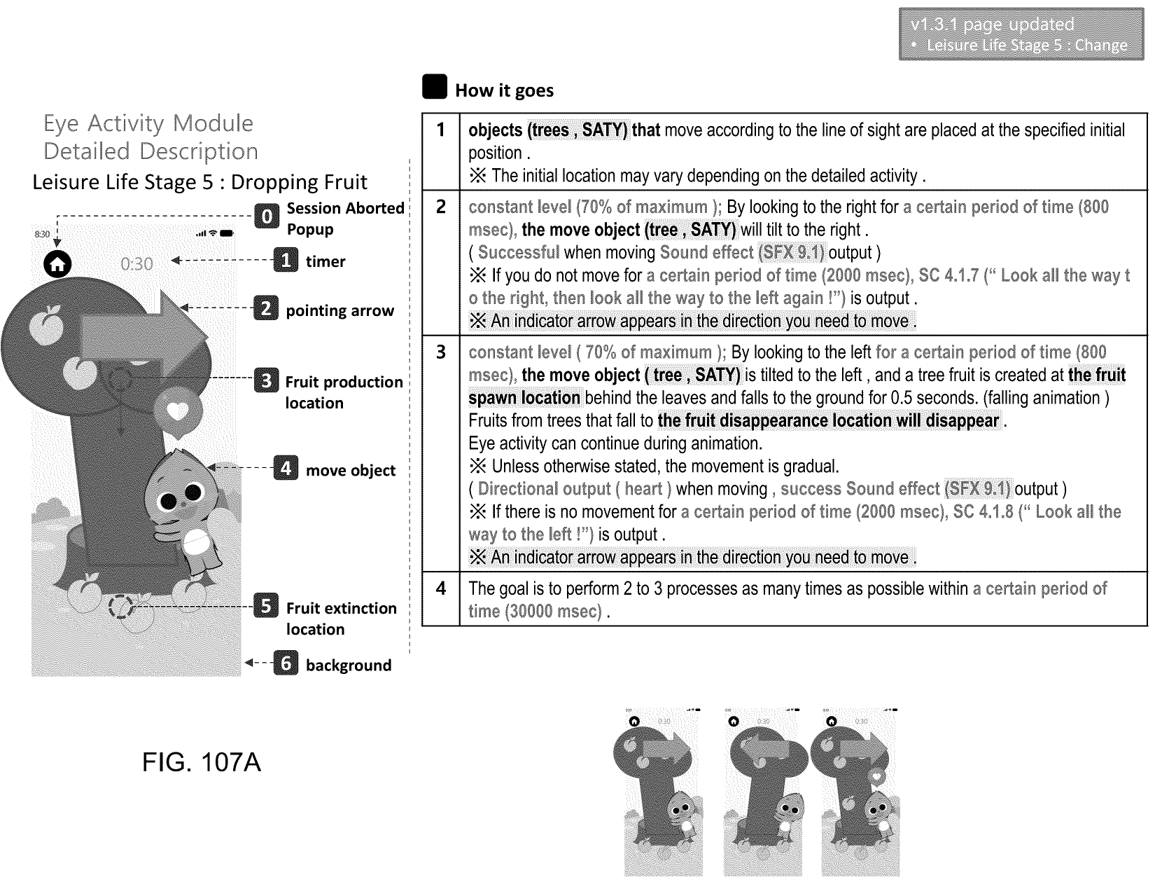

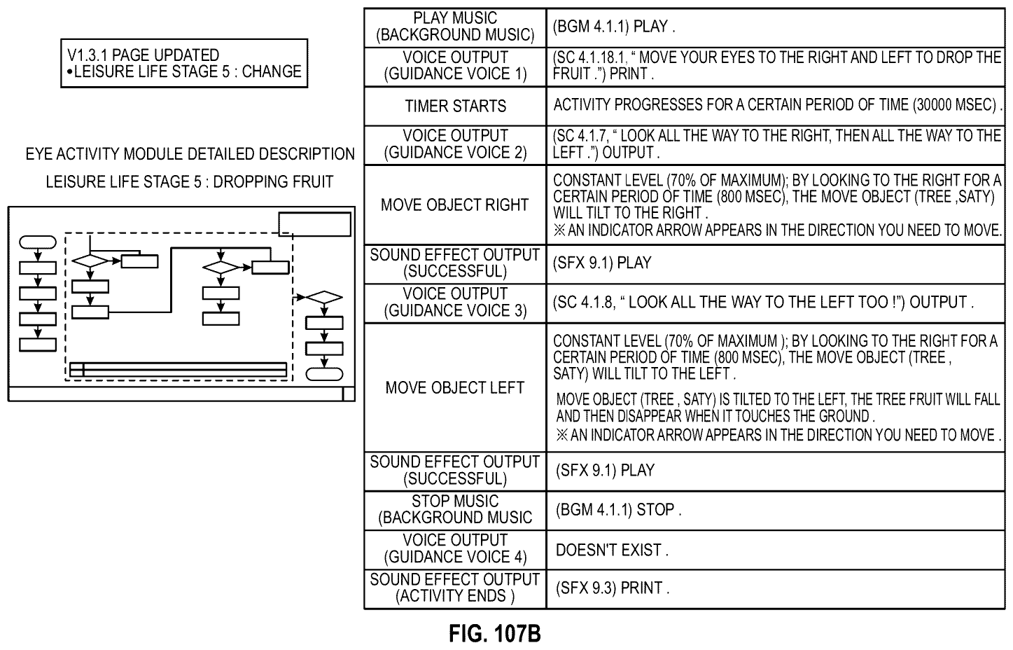

FIG. 107A depicts a screenshot of an eye exercise digital therapeutic module in FIG. 80 of the present disclosure and FIG. 107B depicts a flow chart illustrating an execution flow for the eye exercise digital therapeutic module in FIG. 80 of the present disclosure.

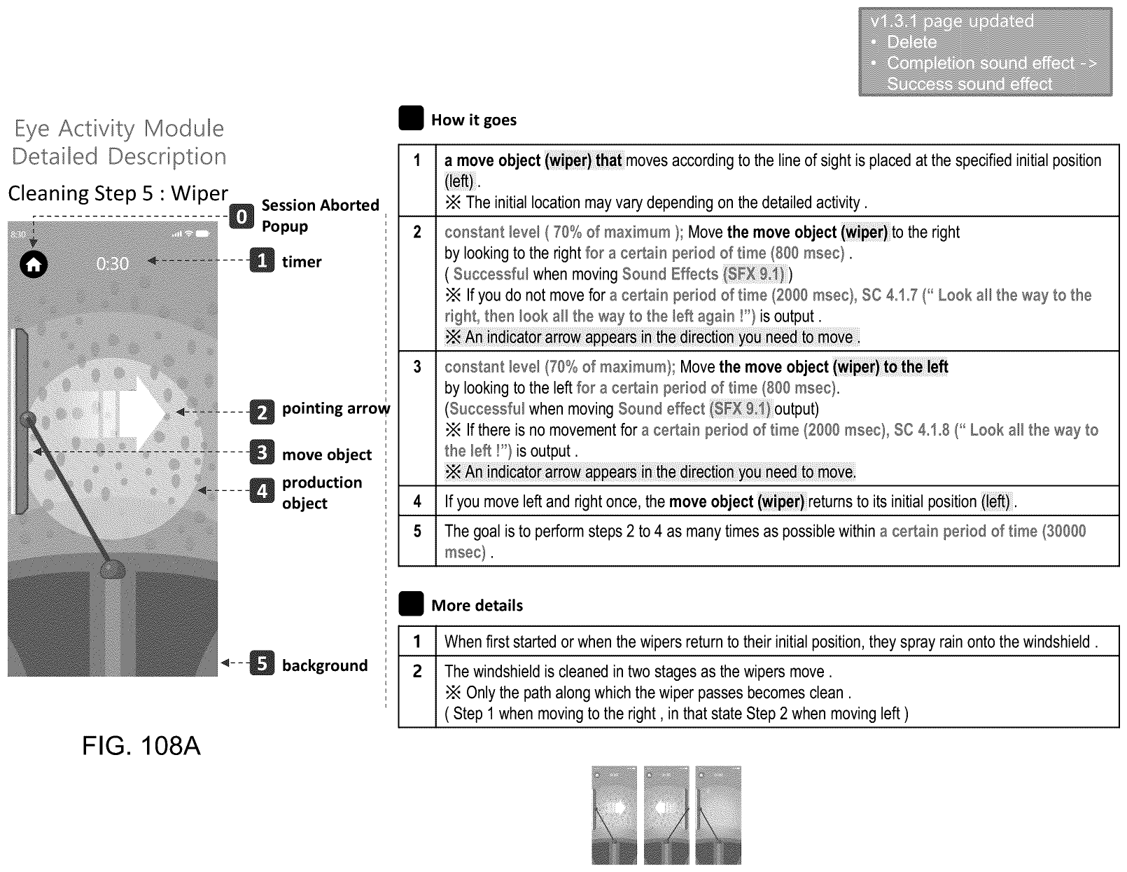

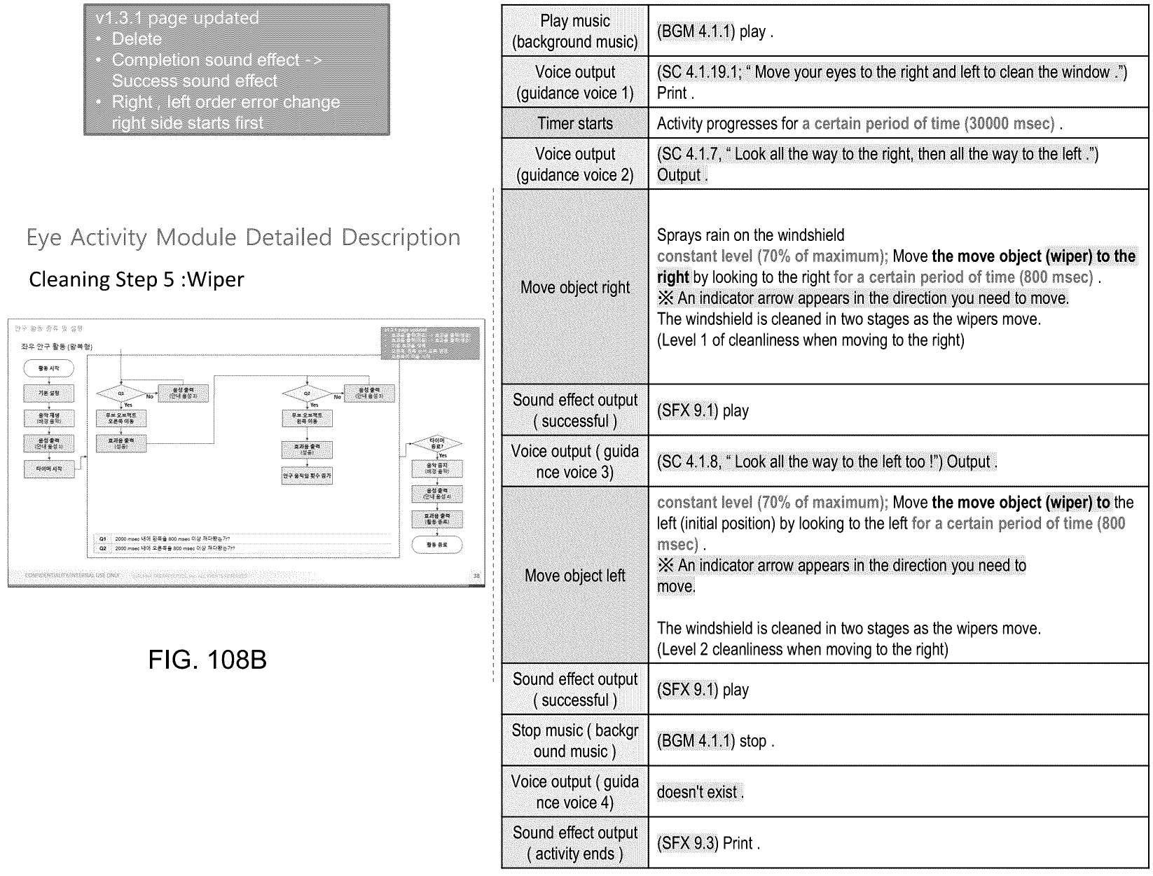

FIG. 108A depicts a screenshot of an eye exercise digital therapeutic module in FIG. 80 of the present disclosure and FIG. 108B depicts a flow chart illustrating an execution flow for the eye exercise digital therapeutic module in FIG. 80 of the present disclosure.

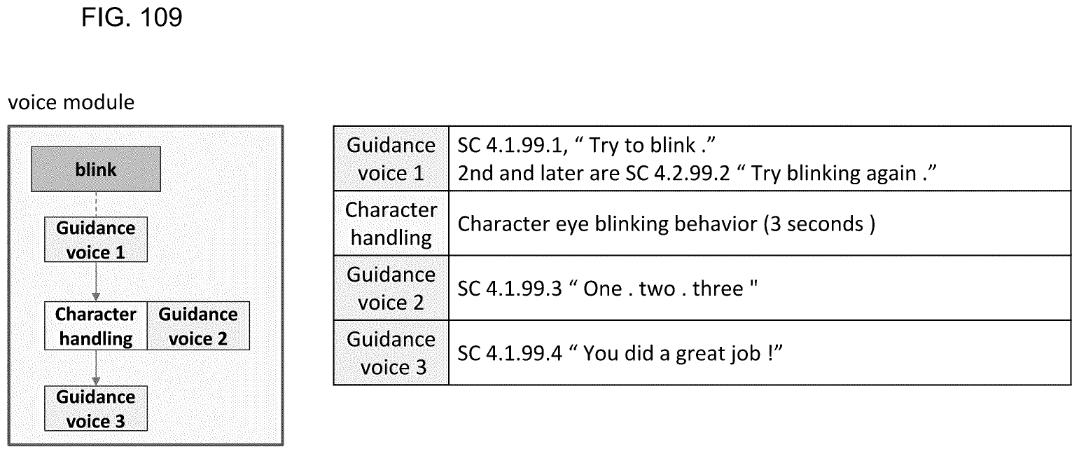

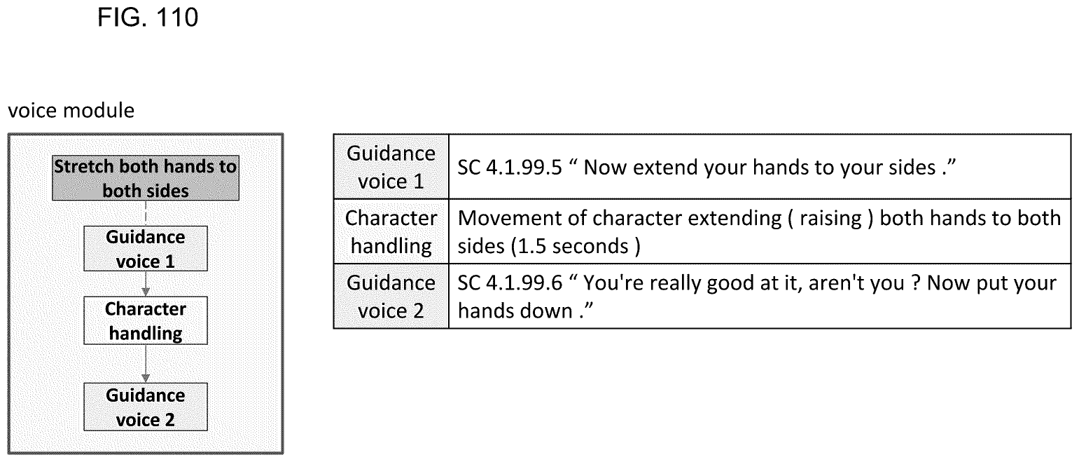

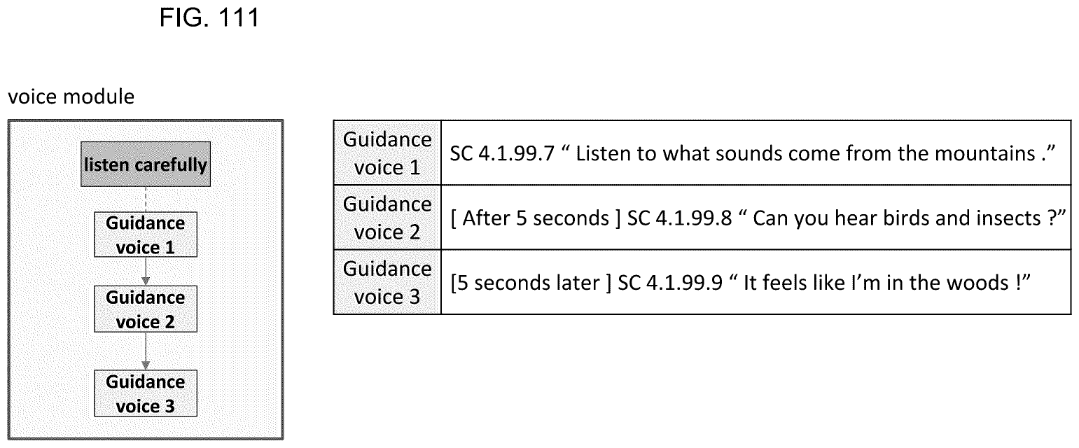

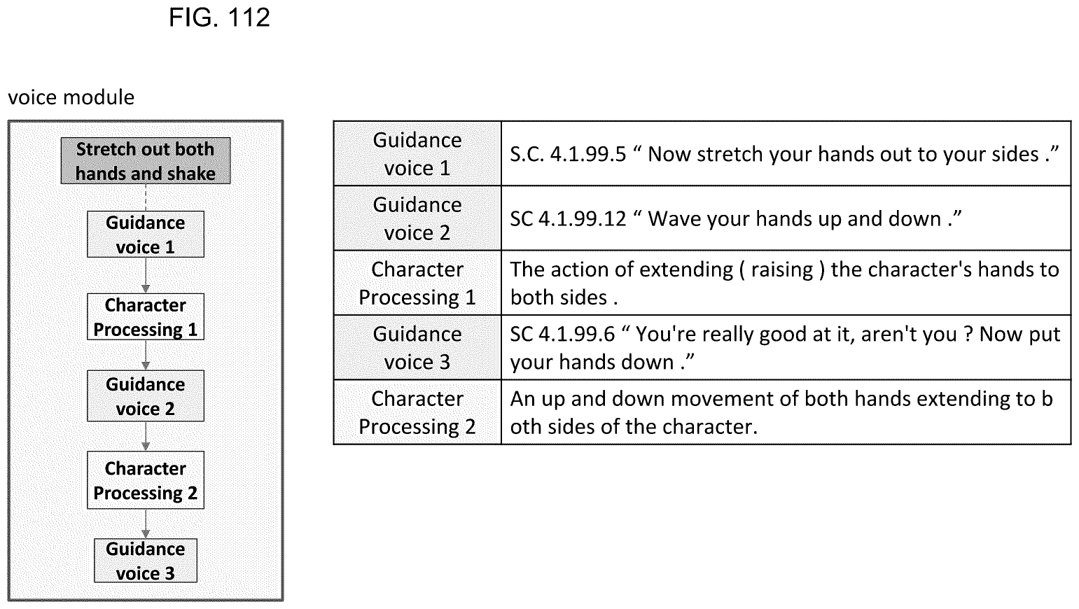

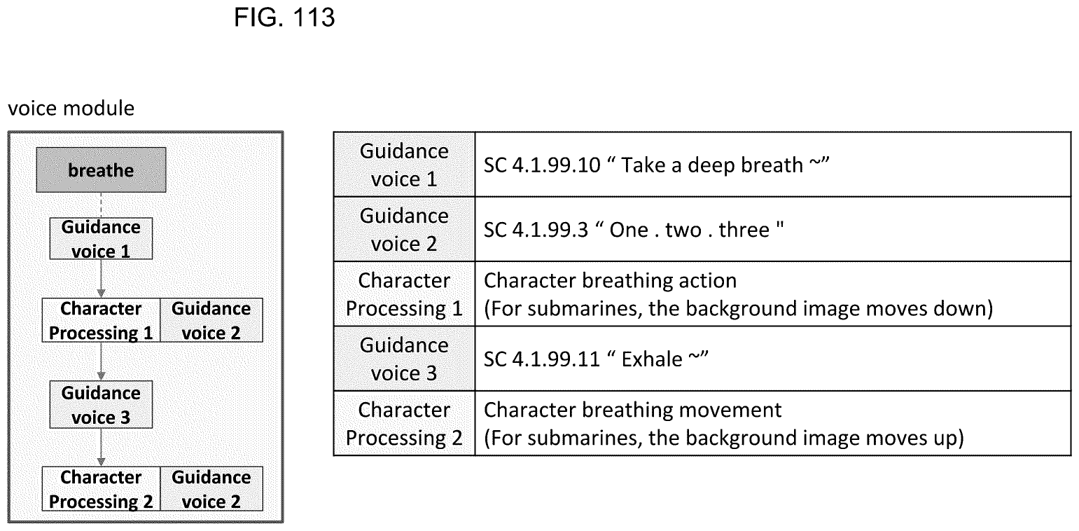

FIGS. 109-112 are flow charts illustrating an execution flow for a relaxation module and FIG. 113 is a flow chart illustrating an execution flow for a deep breathing module. The relaxation module may be composed of a series of behavioral instructions for a subject to follow in order to take a rest through blinking eyes, spreading arms to the side, listening to the music and the like. The deep breathing module may be composed of a series of behavioral instructions for a subject to follow to take a deep breath.

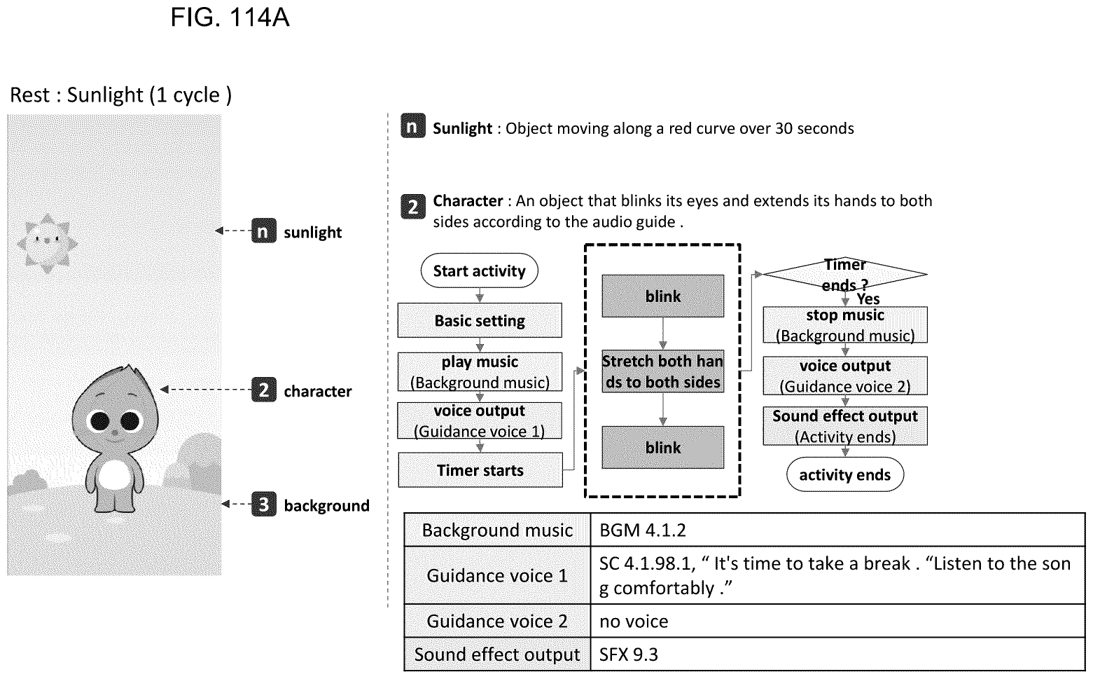

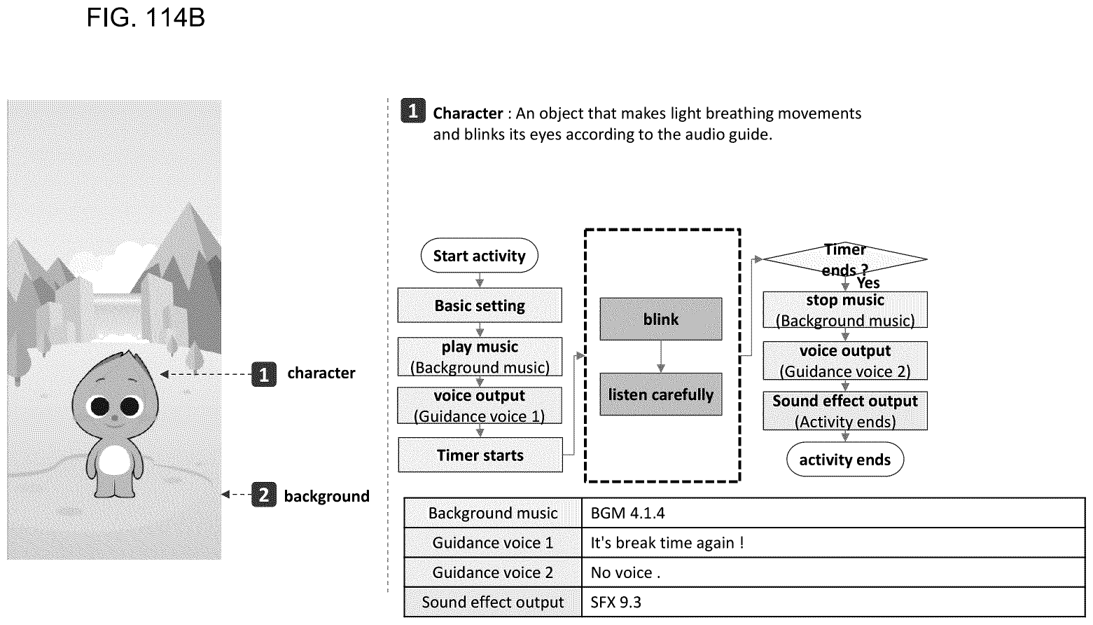

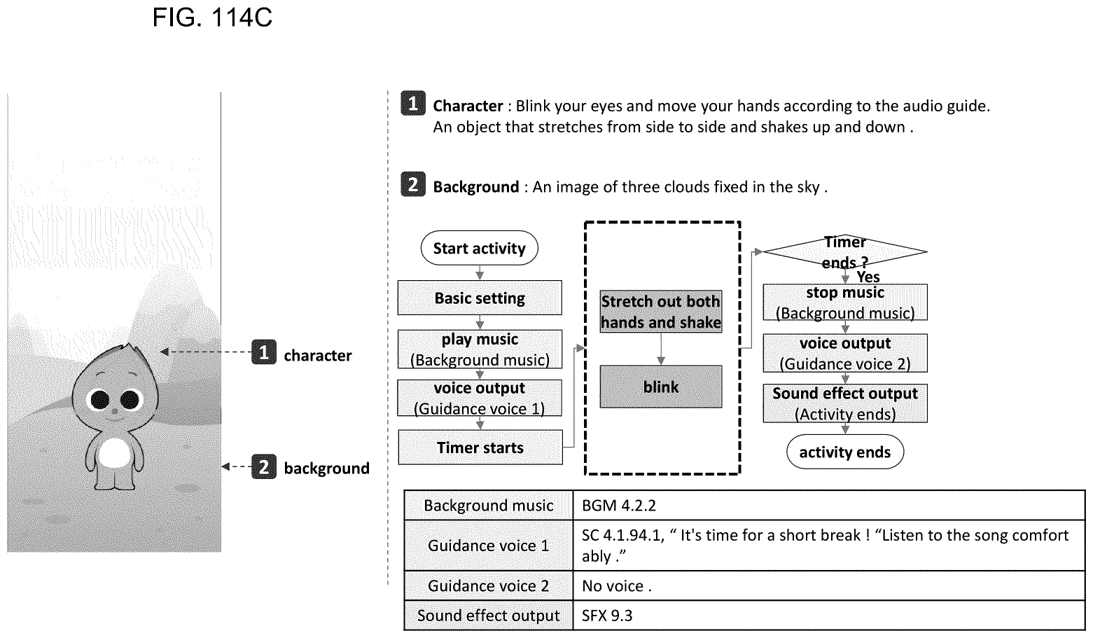

FIGS. 114A-C depict screenshots of a series of behavioral instructions of a relaxation module.

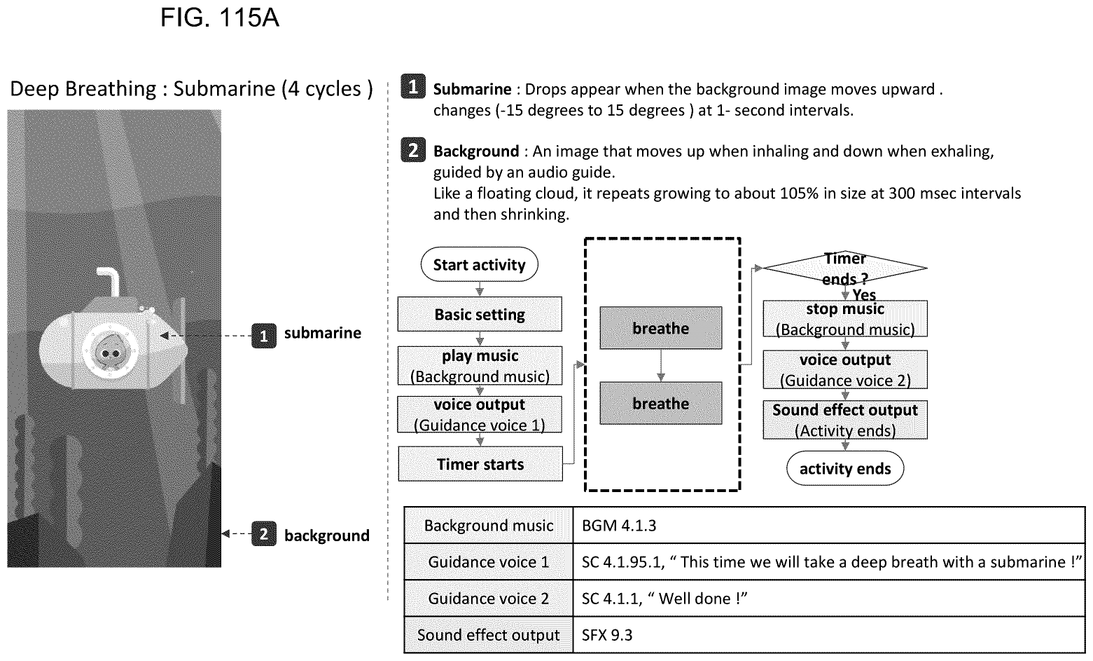

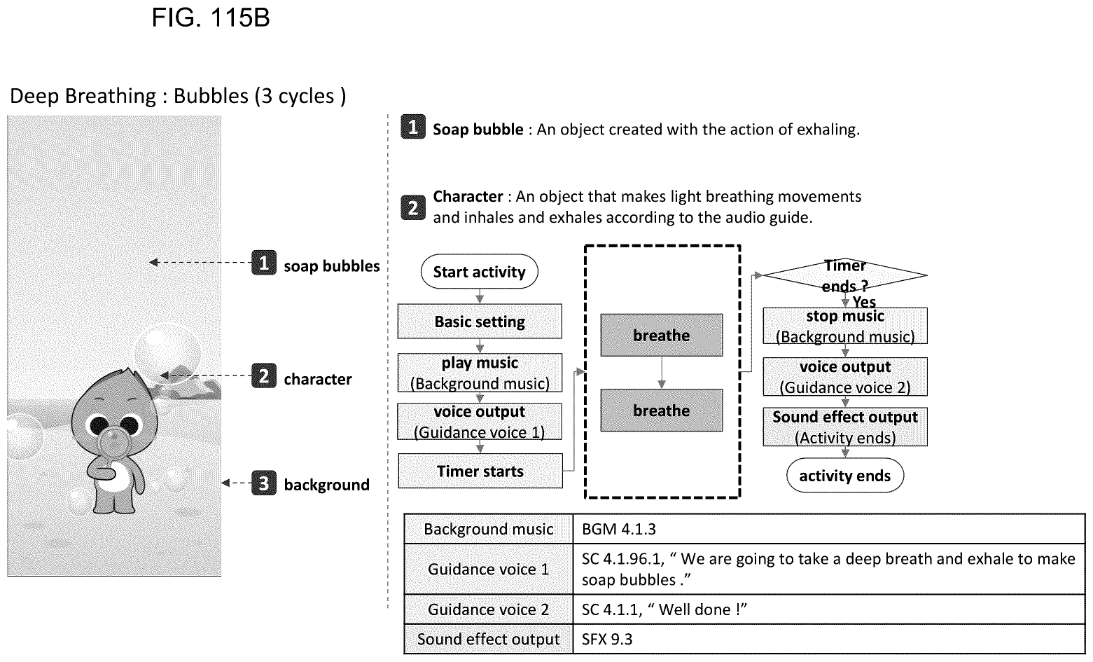

FIGS. 115A-B depict screenshots of a series of behavioral instructions of a deep breathing module.

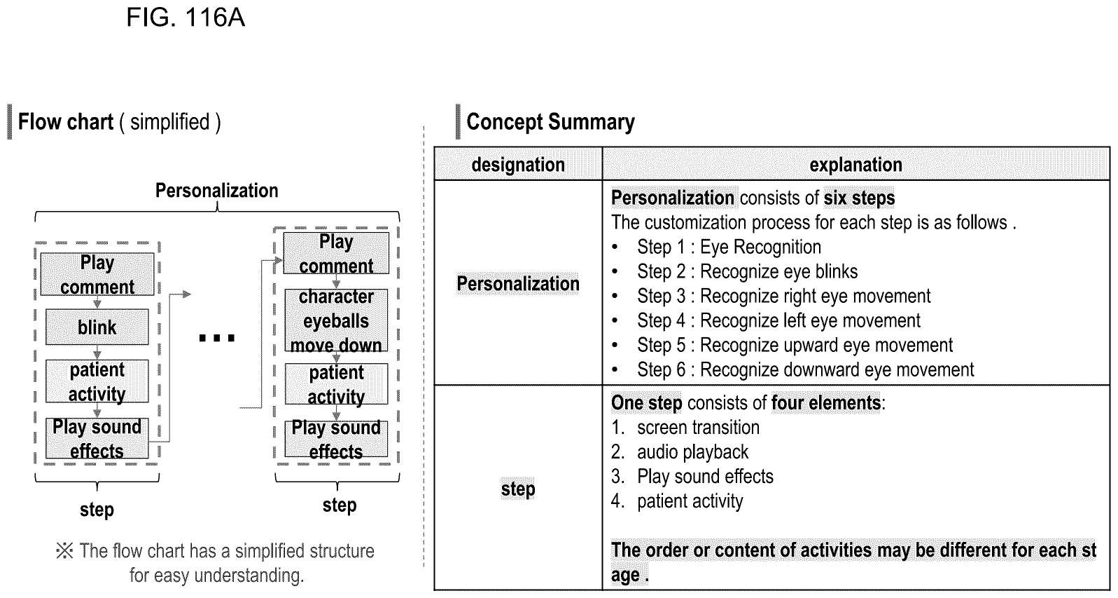

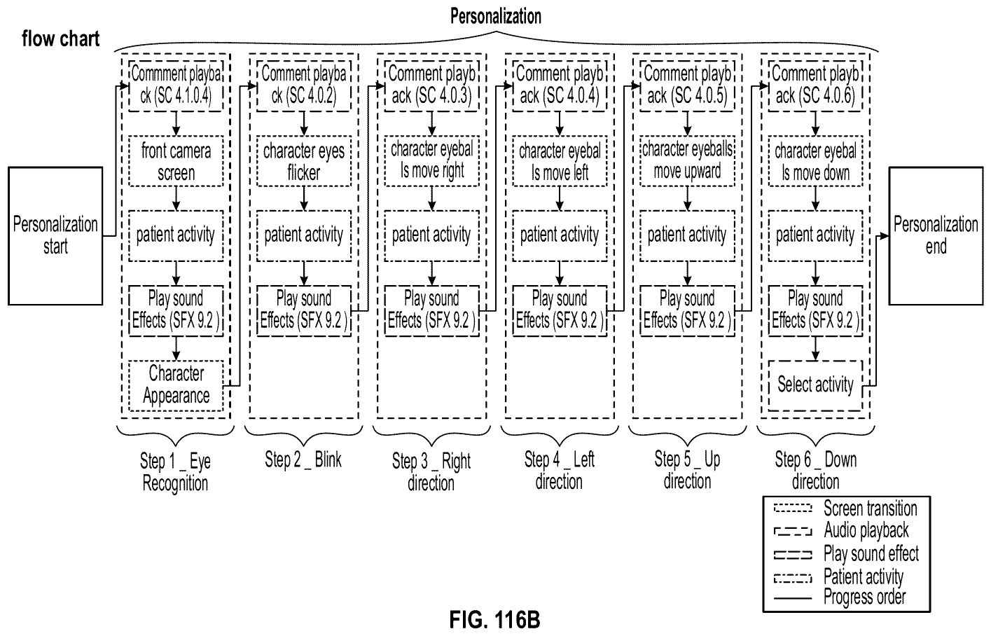

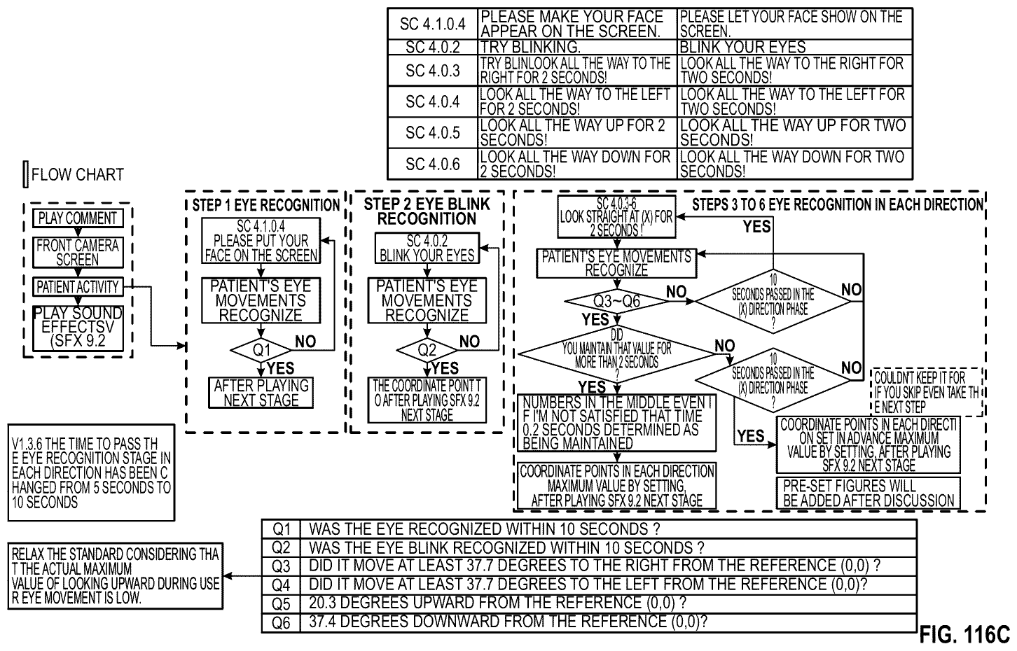

FIG. 116A depicts a flow chart illustrating an execution flow for a customization process of eyeball exercises, FIG. 116B depicts a flow chart illustrating six execution steps of the customization process of eyeball exercises, and FIG. 116C depicts a flow chart illustrating an execution flow for each execution step of the customization process of eyeball exercises. The customization process may recognize at least one eyeball of the subject or movement of the at least one eyeball of the subject to provide a series of eyeball exercise instructions for the subject based on the recognized eyeball movement.

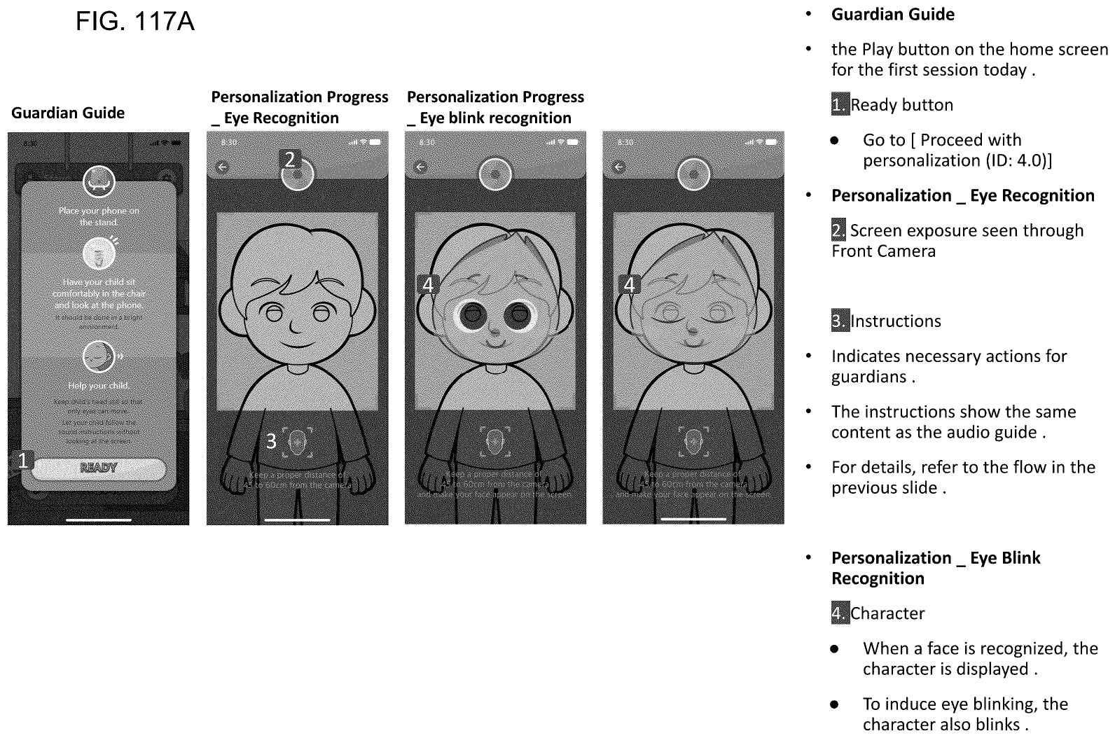

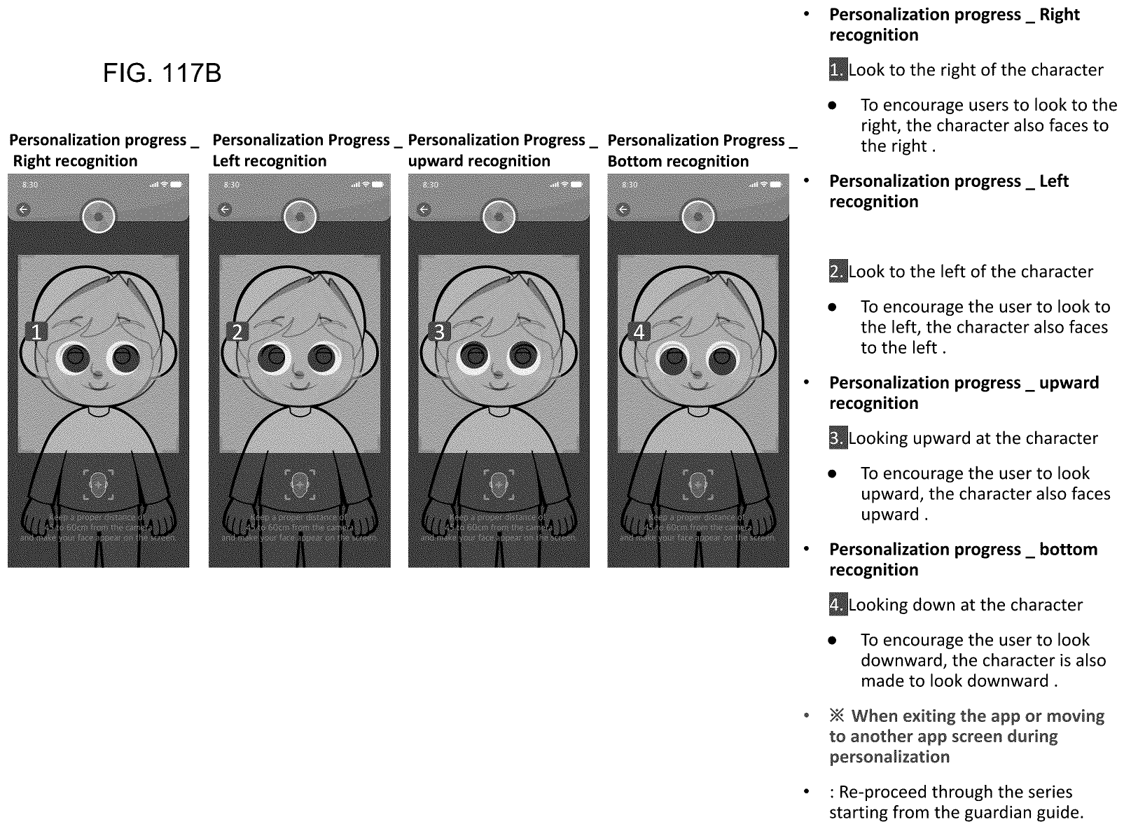

FIGS. 117A-B depict examples of user interfaces for a customization process of eyeball exercises provided by a digital application of the present disclosure. The user interfaces may include visual images such as icons, images, characters and/or information for a subject to move at least one eyeball. The sensor of the digital apparatus may detect at least one eyeball of the subject or movement of the at least one eyeball of the subject. In accordance with the detected result, the digital application of the present disclosure instructs a processor of the digital apparatus to execute operations for the customization process.

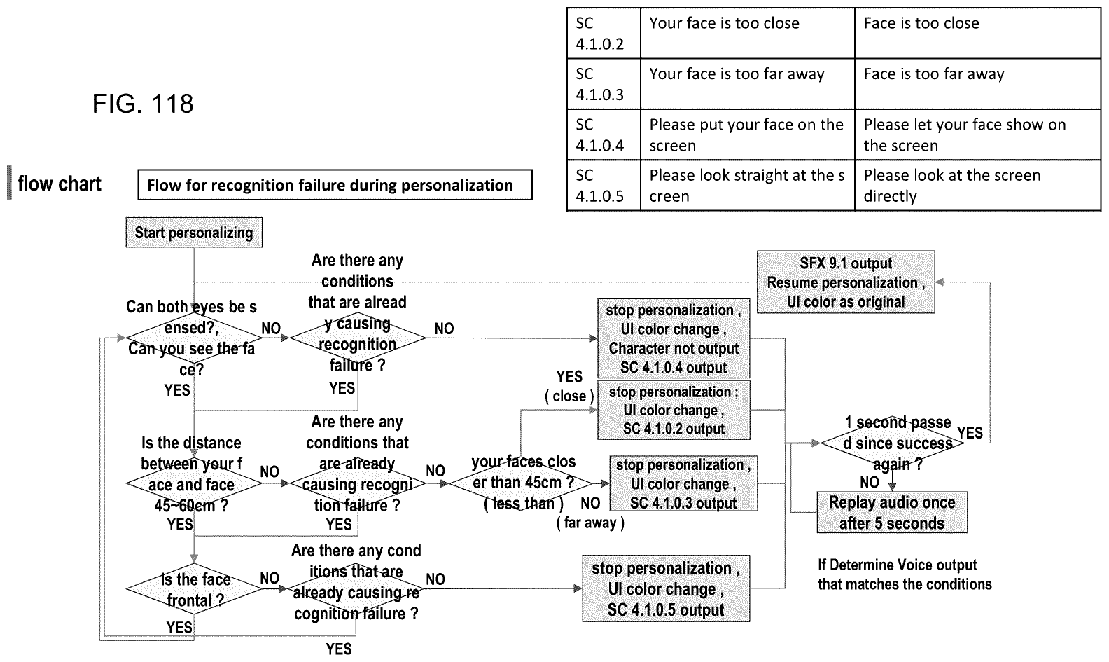

FIG. 118 is a flow chart illustrating an execution flow for a customization process of eyeball exercises when at least one eyeball movement is not recognized.

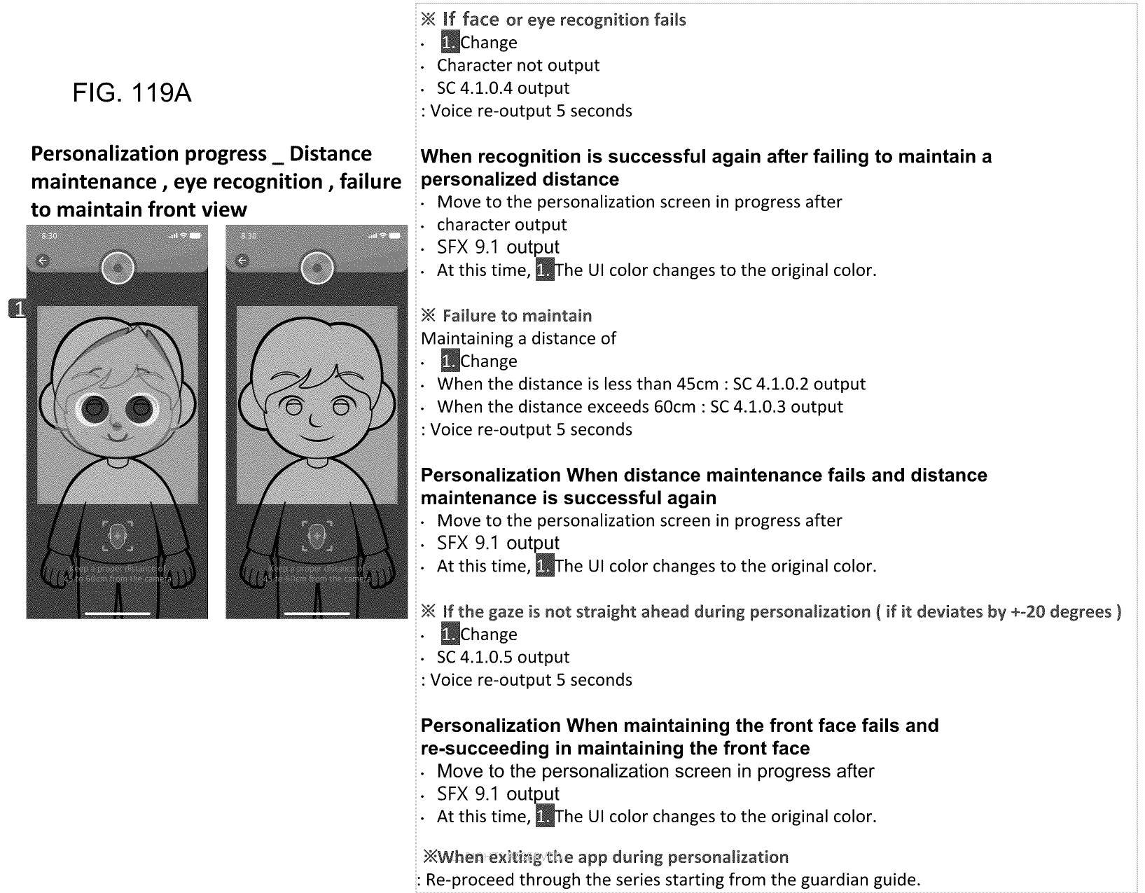

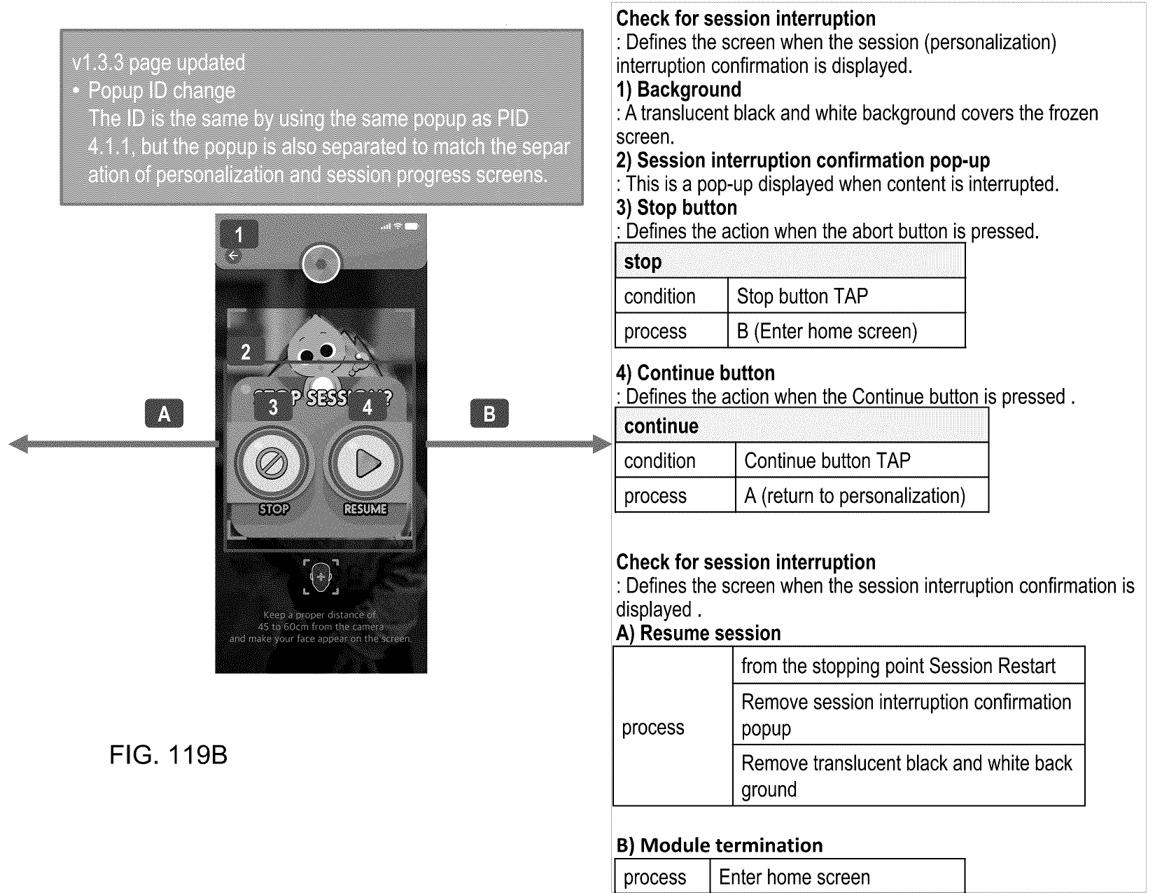

FIG. 119A-B depict examples of user interfaces for a customization process of eyeball exercises provided by a digital application of the present disclosure. The user interfaces may include visual images such as icons, images, characters and/or information representing the failure in detecting at least one eyeball of the subject or movement of the at least one eyeball of the subject or pause, reset and/or continuation of the session.

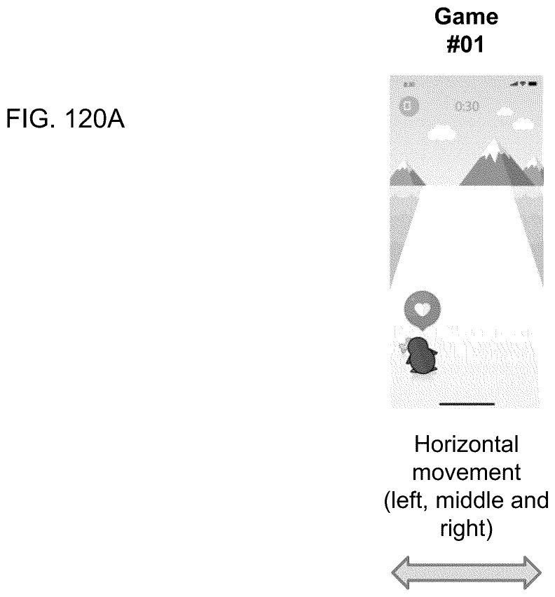

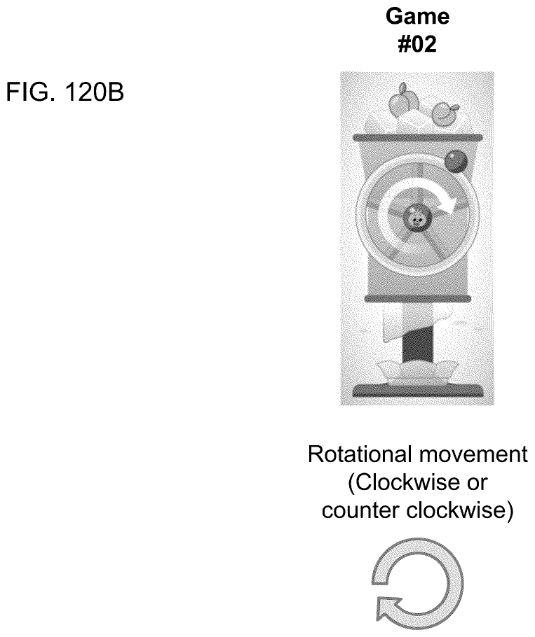

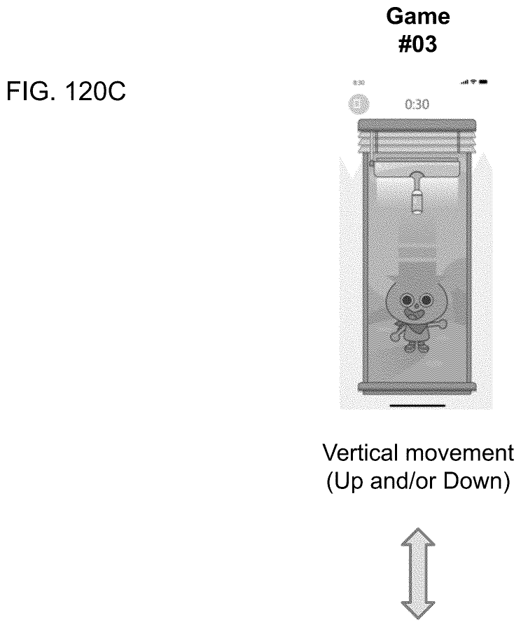

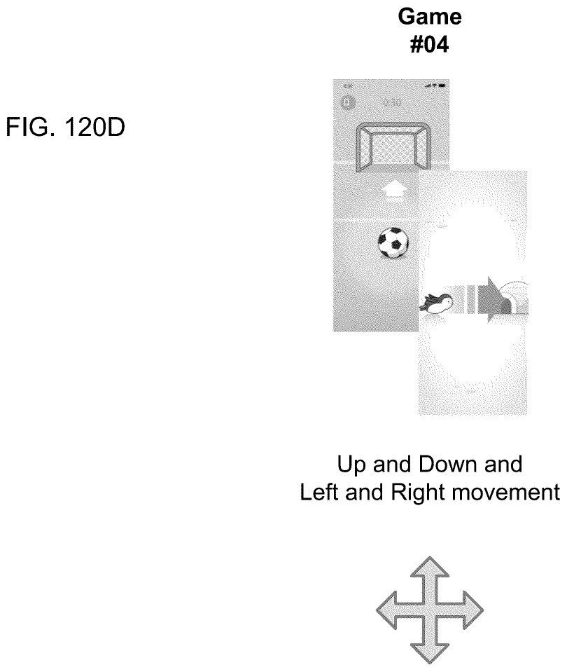

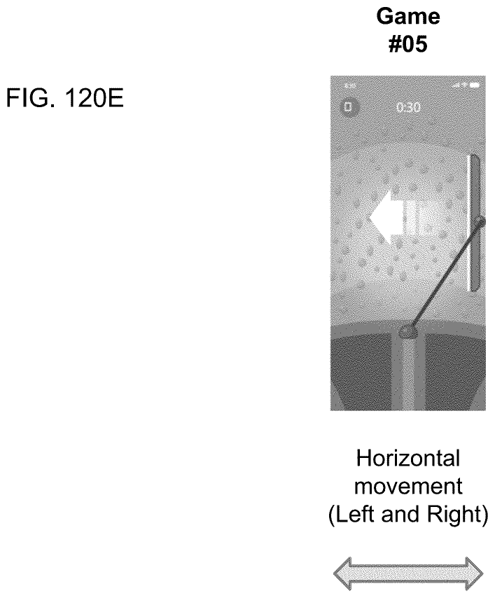

FIG. 120 to FIG. 125 are diagrams for explaining methods and results of a clinical trial to confirm the correlation between the exercise performance pattern and myopia progression of a subject or group of subjects of the digital therapy in accordance with the present disclosure.

FIG. 120 shows execution screens according to a different type of game instructing the subject to perform different eye movements.

FIG. 120A shows an execution screen of a game (Game 1) for inducing horizontal (left and right) movement of the eye of the subject. FIG. 120B shows an execution screen of a game (Game 2) for inducing rotational movement of the eye, and FIG. 120C shows an execution screen of a game (Game 3) for inducing vertical (up and down) movement, and FIG. 120D shows an execution screen of a game (Game 4) for inducing up and down and left and right movements of the eye, and FIG. 120E shows am execution screen of a game (e.g., Game 5) for inducing the horizontal (left and right) movement of the eye of the subject.

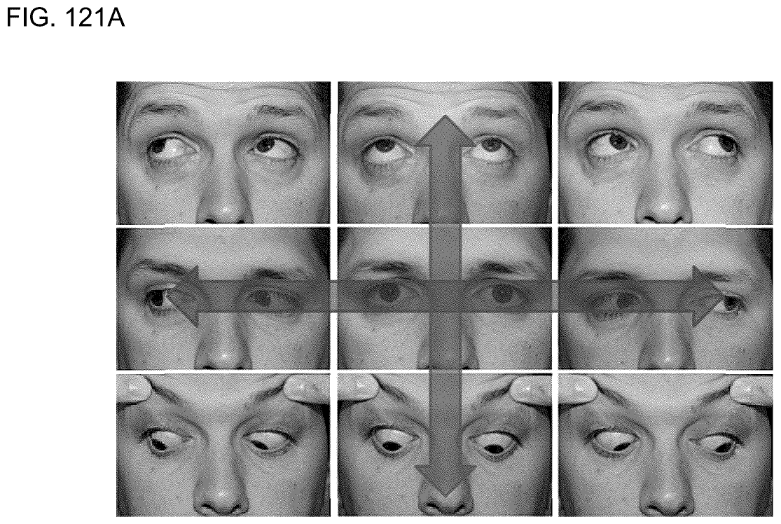

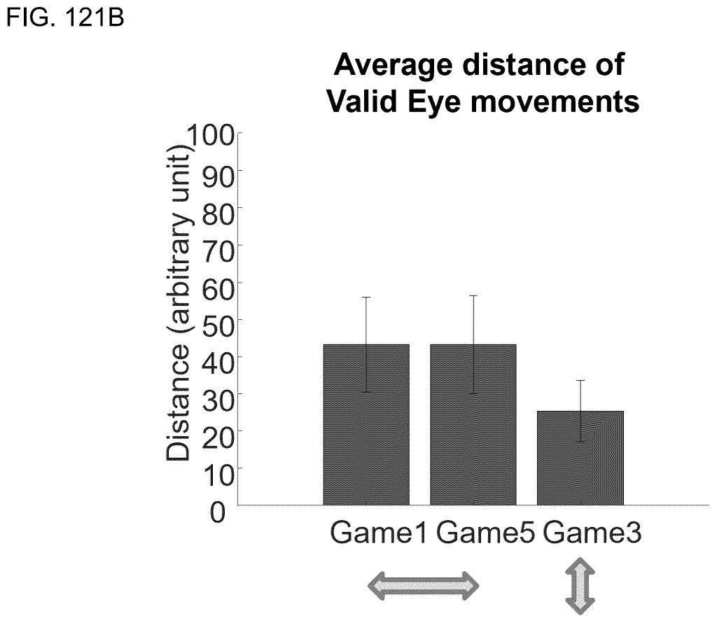

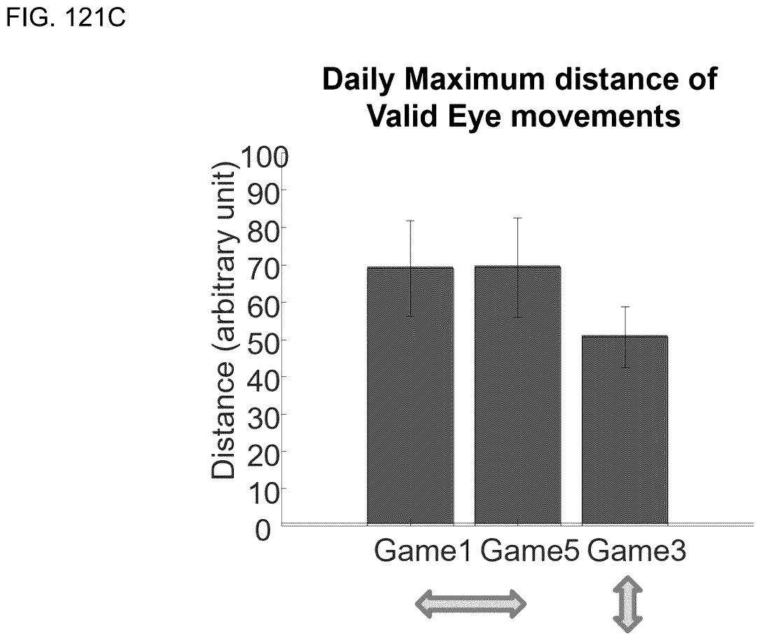

FIG. 121A is a photograph illustrating operating state of the subject's eyes due to a game that induces vertical or horizontal movement of the eyes. According to the guidance of one or more games illustrated in FIG. 120, FIG. 121A illustrates a photograph showing a position of the eyeball when the subject moves the eyeball up-down-left-right. As discussed above, the subject's eye position can be detected by the sensor of the digital device.

FIG. 121B and FIG. 121C illustrate graphs showing the average and maximum transactions of a subject's effective eye movements according to the guidance of the game illustrated in FIG. 120 (e.g., FIGS. 120A-E).

FIG. 121B illustrates a graph showing the average distance measured from the subject's effective eye movements according to Games 1 and 5 for inducing horizontal (left and right) movements of the eyes, and Game 3 for inducing vertical (up and down) movements of the eyes. Referring to FIG. 121B, the average distance of horizontal eye movement according to the game (Game 1, 5) for inducing horizontal (left and right) eye movement is longer than the average distance of vertical eye movements according to the game (Game 3) for inducing vertical (up and down) eye movement.

FIG. 121C illustrates a graph showing the maximum distance measured in the subject's effective eye movement according to Games 1 and 5, which are games for inducing horizontal (left and right) movements of the eyes, and Game 3, which is a game for inducing vertical (up and down) movements of the eyes.

Referring to FIG. 121C, the maximum distance of horizontal eye movement according to the game (Game 1, 5) for inducing horizontal (left and right) movement of the eye is longer than the maximum distance of vertical eye movement according to the game (Game 3) for inducing vertical (up and down) movement of the eye.

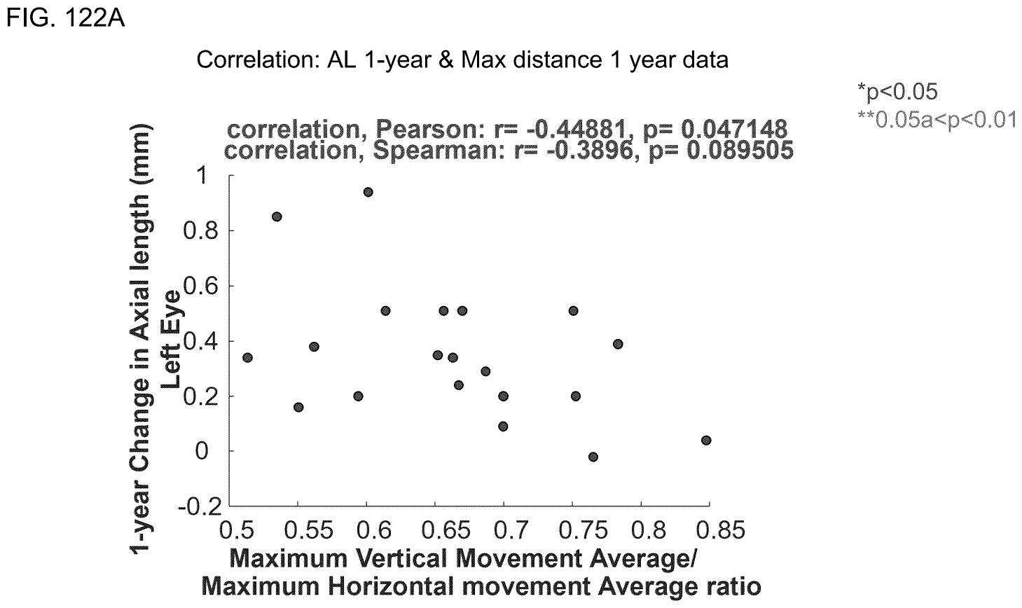

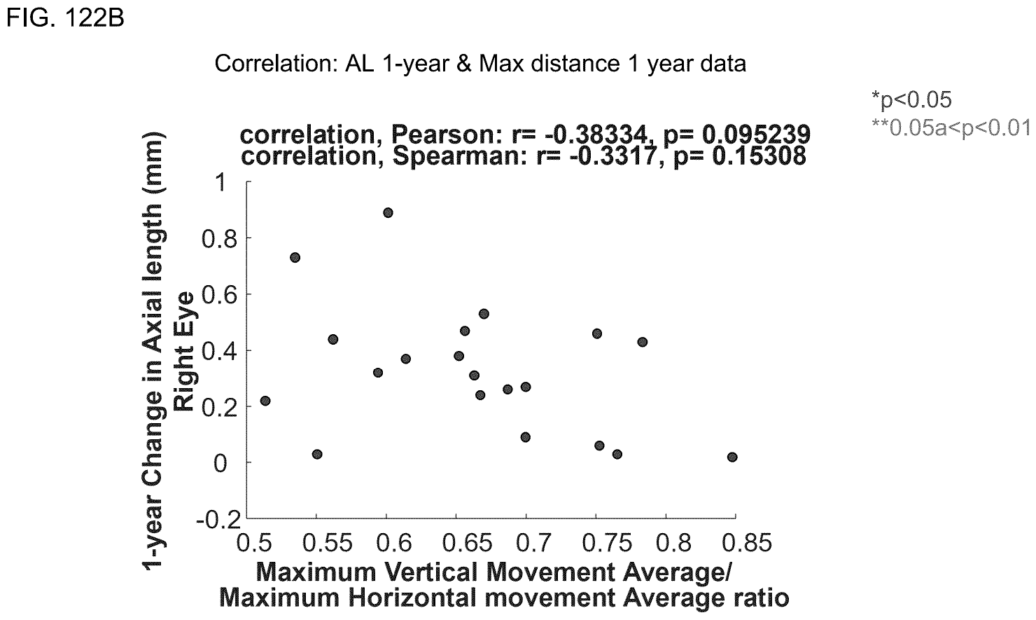

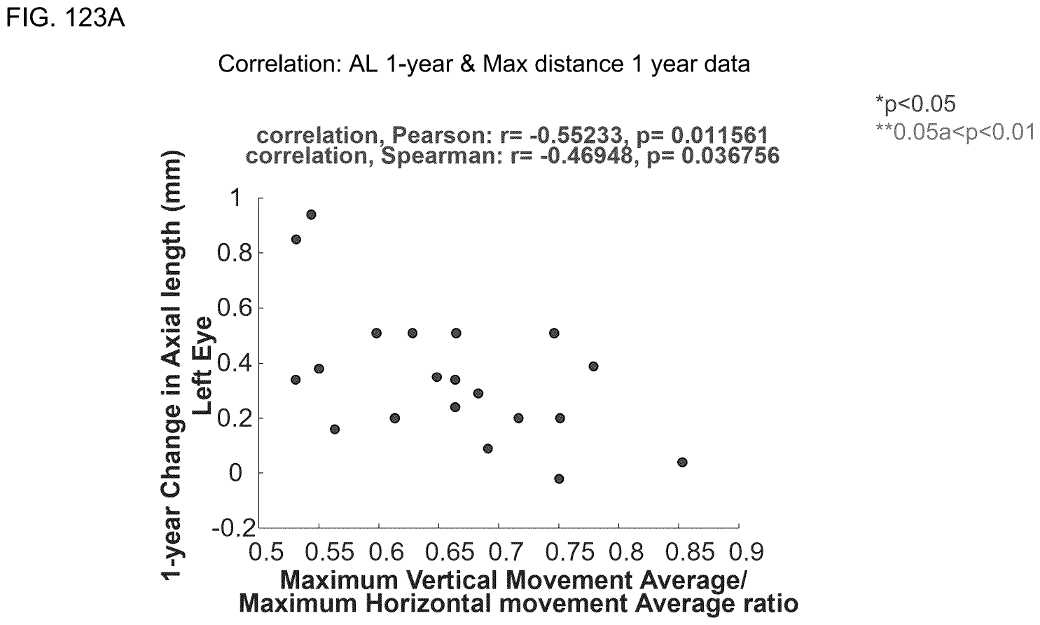

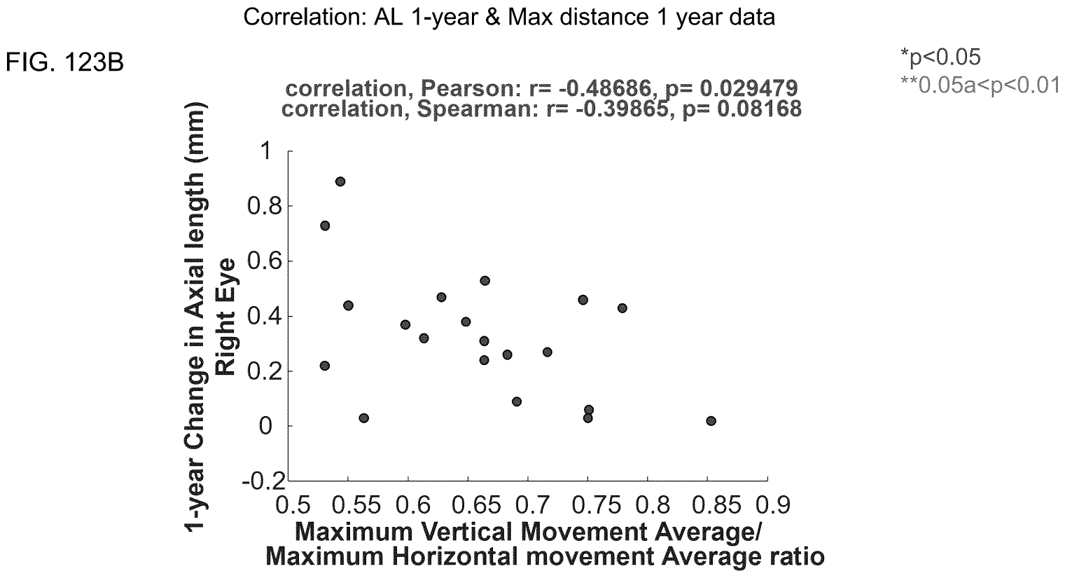

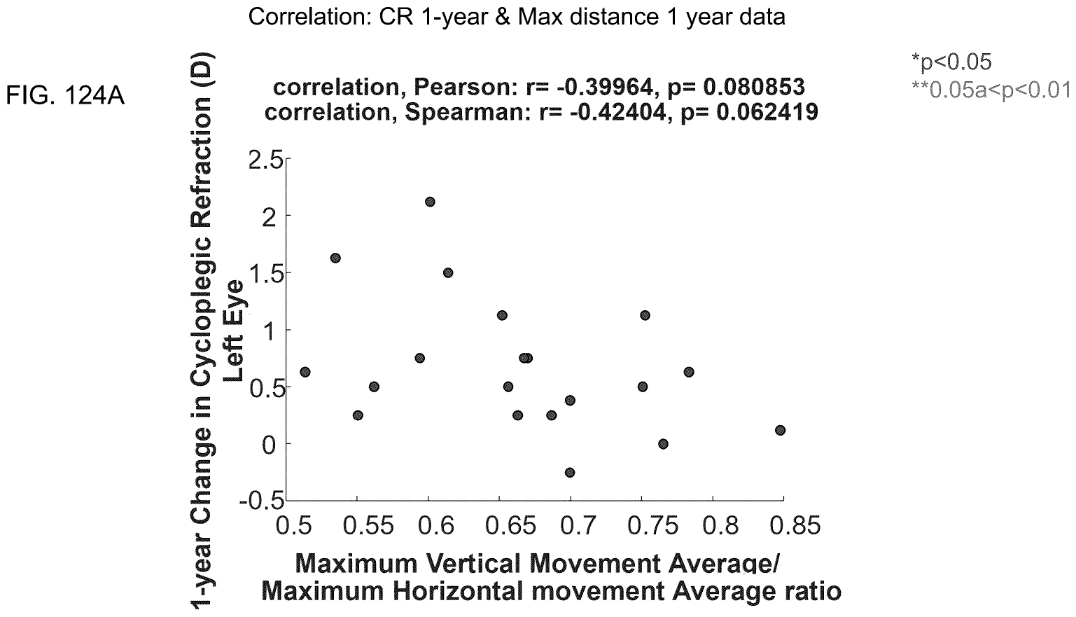

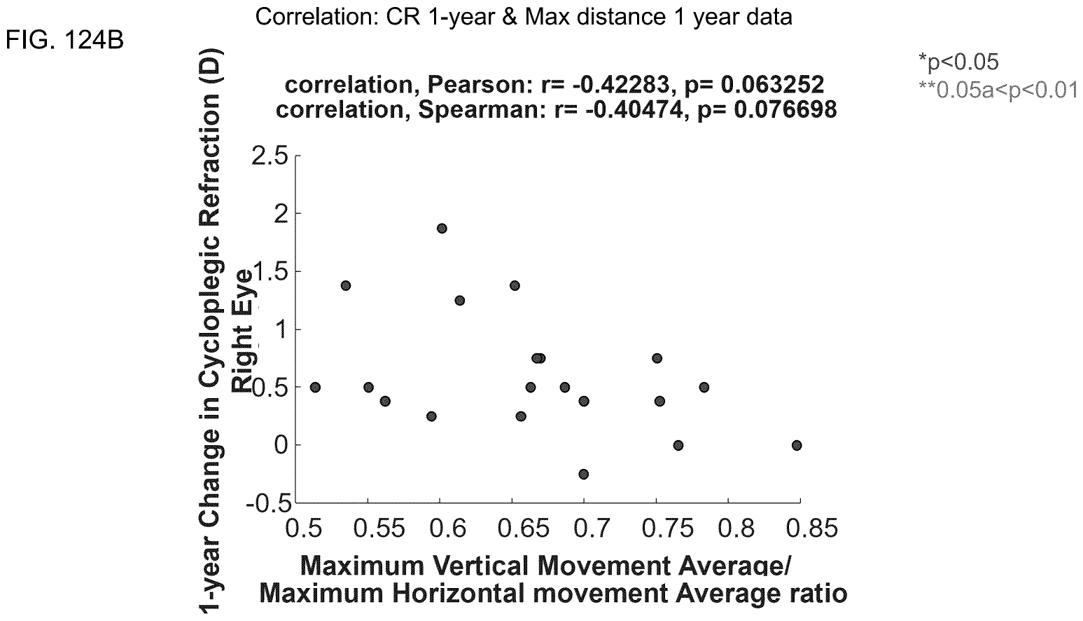

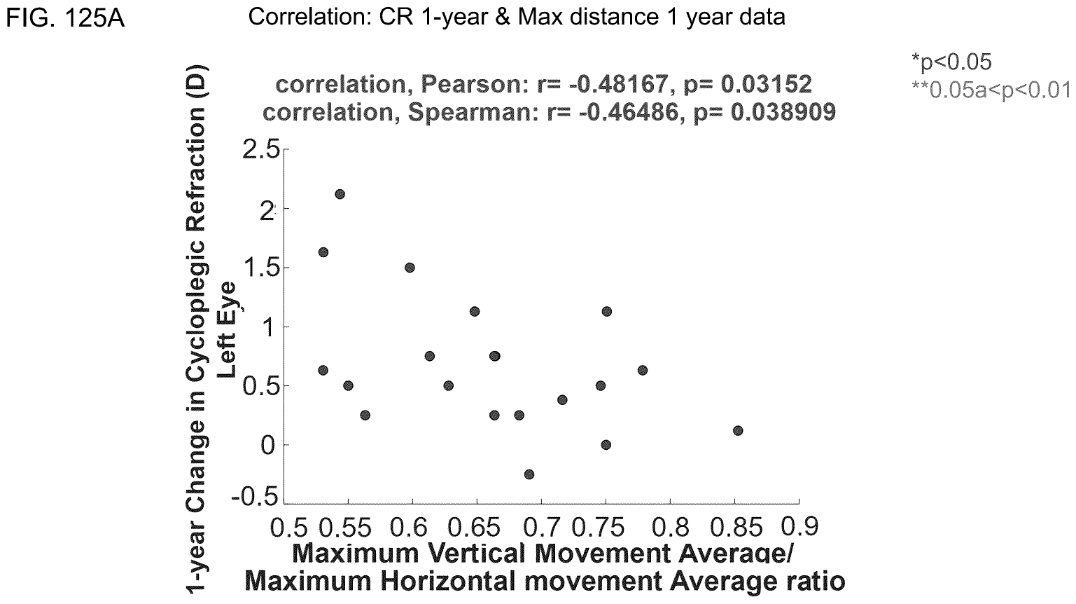

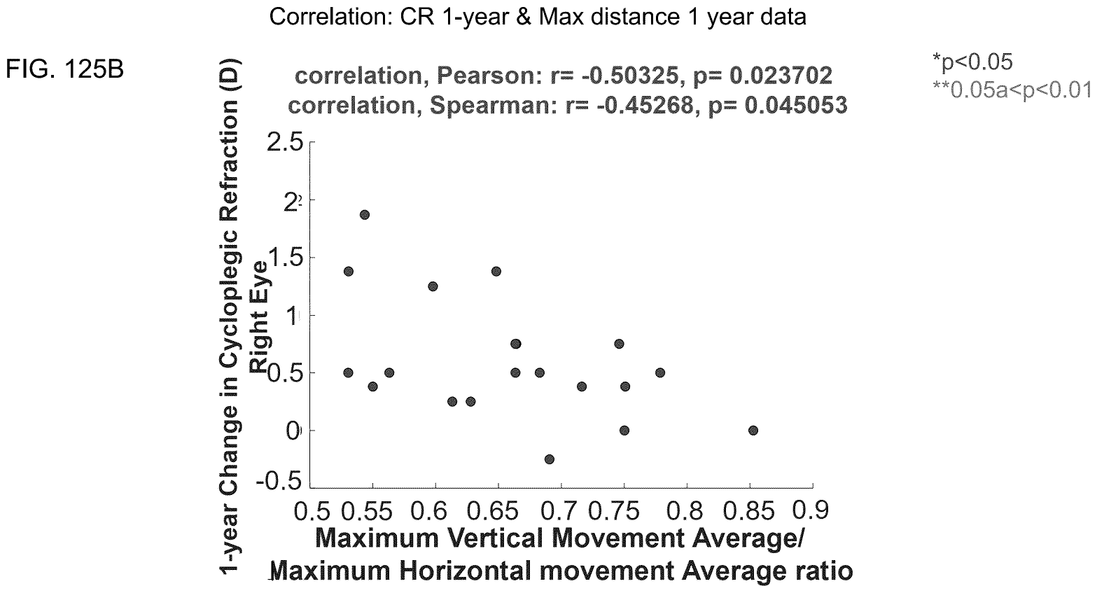

FIG. 122A to FIG. 125B are graphs showing the results of a clinical trial to confirm the correlation between the motor performance pattern and myopia progression of a subject or group of subjects of the digital therapy in accordance with the present disclosure.

This clinical trial has an exploratory purpose to evaluate the safety and effectiveness of digital therapy to improve vision, and the experimental group was comprised of pediatric myopia patients aged between 5 and 13 years old.

Pediatric myopia patients in the experimental group used the digital therapy for 30 minutes a day, 5 times a week for 48 weeks (about 1 year) in addition to conventional myopia treatment (wearing glasses).

In addition, this clinical trial aims to determine the relationship between exercise performance patterns and myopia progression over a specified period (1 year). All treatments for 1 year, including 4 prescriptions, were performed, and efficacy evaluation variables were evaluated. Only subjects who were fully collected were included.

This clinical trial is intended to test the hypothesis that the subjects' performance of vertical movements compared to horizontal movements is related to the progression of myopia.

In this clinical trial, i) the amount of change in axial length (AL) from the baseline to the 48th week [AL at 48 week-AL at baseline] and ii) the change in refractive power (CR: Cycloplegic refraction) from baseline to 48 weeks [CR at 48 week-CR at baseline] are used as the efficacy evaluation variables indicating the progression of myopia.

In addition, in this clinical trial, the performance ratio of subjects' vertical movements compared to horizontal movements was calculated as i) the one-year average of the daily maximum distance measured in the subjects' eye movements according to Game 3 (for horizontal movements) Game (for horizontal movements) divided by the one-year average of the maximum daily distance measured in the subject's eye movements according to Game 3 (for vertical movements), or ii) the one-year average of the maximum daily distances measured in the subject's eye movements.

TABLE 1
[Variables used in correlation analysis]

| Variables | Variable Description | Additional Description |
|---|---|---|
| Effectiveness | Refractive change over 48 weeks | Refractive at 48^{th} week- Refractive |
| evaluation | | at Baseline |
| variables | Change in axial length over 48 | axial length at 48^{th} week - axial |
| | weeks | length at Baseline |
| behavioral | Ratio of horizontal to vertical | The maximum exercise distance is |
| variables | maximum movement distance: | saved for each game every day, and |
| | Average of Maximum Distance of | the average for each game of data |
| | Game 3/Average of Maximum | collected over a year is used. |
| | Distance of Game 1 | |
| | Ratio of horizontal to vertical | |
| | maximum movement distance: | |
| | Average of Maximum Distance of | |
| | Game 3/Average of Maximum | |
| | Distance of Game 5 | |

In this clinical trial, correlation analysis was used to statistically confirm the relationship between [effectiveness evaluation variable] and [performance ratio of vertical movement compared to horizontal movement], both the parametric test, two sample t-test, and the non-parametric test method, Mann-Whitney test, were used.

FIGS. 122A-B illustrates a graph summarizing the results when the performance ratio of the subjects' vertical movements compared to the horizontal movements was measured as [1-year average of the daily maximum distance measured in the subject's eye movements according to Game 3/1-year average of the daily maximum distance measured in the subject's eye movements according to Game 1] and the efficacy evaluation variable was measured as the change in axial length (AL) from the baseline to the 48th week [AL at 48 week-AL at baseline].

Referring to FIGS. 122A-B, it shows that the higher the subjects' performance ratio of vertical movements compared to horizontal movements (i.e., the more vertical movements were possible as horizontal movements), the lower the progression of myopia (smaller one-year change in axial length). However, the left eye (FIG. 122A) shows a statistically significant correlation, but the right eye (FIG. 122B) shows a similar tendency, although it is not statistically significant.

FIGS. 123A-B illustrates a graph summarizing the results when the performance ratio of the subjects' vertical movements compared to the horizontal movements was measured as [1-year average of the daily maximum distance measured in the subject's eye movements according to Game 3/1-year average of the daily maximum distance measured in the subject's eye movements according to Game 5] and the efficacy evaluation variable was measured as the change in axial length (AL) from the baseline to the 48th week [AL at 48 week-AL at baseline].

Referring to FIGS. 123A-B, it shows that the higher the subjects' performance ratio of vertical movements compared to horizontal movements (i.e., the more vertical movements were possible as horizontal movements), the lower the progression of myopia (smaller 1-year change in axial length). This shows a statistically significant correlation (p<0.05) in both the left eye (FIG. 123A) and the right eye (FIG. 123B).

FIGS. 124A-B illustrates a graph summarizing the results when the performance ratio of the subjects' vertical movements compared to the horizontal movements was measured as [1-year average of the daily maximum distance measured in the subject's eye movements according to Game 3/1-year average of the daily maximum distance measured in the subject's eye movements according to Game 1] and the efficacy evaluation variable was measured as the change in refractive power (CR) from the baseline to the 48th week [CR at 48 week-CR at baseline].

Referring to FIGS. 124A-B, it shows that the higher the subjects' performance ratio of vertical movements compared to horizontal movements (i.e., the more vertical movements were possible as horizontal movements), the lower the progression of myopia (smaller one-year change in refractive power). However, both eyes (FIG. 124A and FIG. 124B) show a similar tendency, although not significant.

FIGS. 125A-B illustrates a graph summarizing the results when the performance ratio of the subjects' vertical movements compared to the horizontal movements was measured as [1-year average of the daily maximum distance measured in the subject's eye movements according to Game 3/1-year average of the daily maximum distance measured in the subject's eye movements according to Game 5] and the efficacy evaluation variable was measured as the change in refractive power (CR) from the baseline to the 48th week [CR at 48 week-CR at baseline].

Referring to FIGS. 125A-B, it shows that higher the subjects' performance ratio of vertical movements compared to horizontal movements (i.e., the more vertical movements were possible as horizontal movements), the lower the progression of myopia (smaller one-year change in refractive power). This shows a statistically significant correlation (p<0.05) in both the left eye (FIG. 125A) and the right eye (FIG. 125B).

In one aspect, the present disclosure relates to the following embodiments.

Embodiment 1. A method of improving an eyesight of a subject, the method comprising: providing, by a digital apparatus to the subject, a digital application comprising one or more digital therapeutic modules for improving the eyesight, each of the modules comprising one or more first instructions for the subject to follow, wherein the first instructions comprise a first eyeball exercise instruction for the subject to move at least one eyeball vertically.
Embodiment 2. A method of treating myopia in a subject in need thereof, the method comprising: providing, by a digital apparatus to the subject, a digital application comprising one or more digital therapeutic modules for treating myopia, each of the modules comprising one or more first instructions for the subject to follow, wherein the first instructions comprise a first eyeball exercise instruction for the subject to move at least one eyeball vertically.
Embodiment 3. The method of any one of the preceding embodiments, wherein the first eyeball exercise instruction is for the subject to move said at least one eyeball at least 50 out of 100 maximum vertical view of the subject.
Embodiment 4. The method of any one of the preceding embodiments, wherein the first eyeball exercise instruction is for the subject to move said at least one eyeball at least 70 out of 100 maximum vertical view of the subject.
Embodiment 5. The method of any one of the preceding embodiments, wherein the digital application comprises more instructions for vertical eye movements compared to instructions for horizontal eye movement.
Embodiment 6. The method of any one of the preceding embodiments, wherein the first eyeball exercise instruction is to move said at least one eyeball upward.
Embodiment 7. The method of any one of embodiments 1-5, wherein the first eyeball exercise instruction comprise more instruction to move said at least one eyeball upward compared to instruction to move said at least eyeball downward.
Embodiment 8. The method of any one of the preceding embodiments, wherein the first instructions exclude an instruction to move said at least one eyeball horizontally.
Embodiment 9. The method of any one of the preceding embodiments, wherein the method improves a growth rate of AL (Axial Length) of said at least one eyeball in the subject.
Embodiment 10. The method of any one of the preceding embodiments, wherein the method reduces a growth rate of AL (Axial Length) of said at least one eyeball in the subject.
Embodiment 11. The method of any one of the preceding embodiments, further comprising measuring a maximum vertical view of the subject.
Embodiment 12. The method of embodiment 10, wherein the measuring is performed by a sensor of the digital apparatus.
Embodiment 13. The method of any one of the preceding embodiments, wherein the subject is 10 years old or more.
Embodiment 14. The method of any one of the preceding embodiments, wherein the modules are selected based on a mechanism of action in and a therapeutic hypothesis, wherein the digital apparatus (i) comprises a sensor sensing adherence by the subject to the one or more first instructions of the modules, (ii) transmits adherence information, based on the adherence, to a server accessible by a healthcare provider through a healthcare provider portal, and (iii) receives one or more second instructions from the healthcare provider based on the adherence information.
Embodiment 15. The method of embodiment 14, wherein said one or more second instructions comprise a second eyeball exercise instructions for an eyeball movement at a speed adjusted based on the adherence information.
Embodiment 16. The method of any one of the preceding embodiments, wherein the digital application instructs a processor of the digital apparatus to execute operations comprising: generating digital therapeutic modules based on a mechanism of action and a therapeutic hypothesis.
Embodiment 17. The method of embodiment 14, wherein the generating of the digital therapeutic modules comprises generating the digital therapeutic modules based on neurohumoral factors.
Embodiment 18. The method of embodiment 14 or 15, wherein the operations further comprise generating a calibration module for calibrating one or more of an accuracy of measurement of the subject's eye position, and a light environment.
Embodiment 19. The method of embodiment 18, wherein the calibration module is generated prior to generating the digital therapeutic modules.
Embodiment 20. The method of embodiment 18 or 19, wherein the accuracy of measurement of the subject's eye position is calibrated, and said calibrating the accuracy of measurement of the subject's eye position comprises one or more of instructing the subject to position their face to appear on a screen of the digital apparatus, detecting the subject's eyes for a given period of time, instructing the subject to blink their eyes, detecting if the subject blinked their eyes, instructing the subject to stare at the screen, instructing the subject to move their eyes in a given direction or rotate their eyes, and determining a threshold for detecting the subject's eyes.
Embodiment 21. The method of any one of embodiments 18-20, wherein the accuracy of measurement of the light environment is calibrated, and said calibrating the light environment comprises one or more of detecting light in the subject's environment using a light sensor of the digital apparatus, and instructing the subject to turn on one or more lights in their environment.
Embodiment 22. The method of any one of the preceding embodiments, wherein the digital apparatus comprises one or more sensors for tracking movement of the subject's eyeball.
Embodiment 23. The method of any one of the preceding embodiments, wherein the digital application instructs a processor of the digital apparatus to execute operations comprising: generating digital therapeutic modules based on a mechanism of action in and a therapeutic hypothesis; generating digital instructions based on the digital therapeutic modules; providing the digital instruction to a subject; and collecting the subject's execution outcomes of the digital instructions.
Embodiment 24. The method of embodiment 23, wherein the generating of the digital instructions and the collecting of the subject's execution outcomes of the digital instructions are repeatedly executed several with multiple feedback loops, and the generating of the digital instructions comprises generating the subject's digital instructions for this cycle based on the subject's digital instructions in the previous cycle and the collected execution outcome data on the subject's digital instructions provided in the previous cycle.
Embodiment 25. The method of embodiment 23 or 24, wherein the collecting the subject's execution outcomes of the digital instructions comprises determining one or both of an exercise intensity (EI) and an average exercise intensity (AEI).
Embodiment 26. The method of embodiment 25, wherein AEI is determined as an averaged sum of differences between a final location of an eyeball of the subject and a starting location of the eyeball measured at a given interval.
Embodiment 27. The method of embodiment 26, wherein the interval is between about 10 milliseconds (ms) and about 500 ms.
Embodiment 28. The method of any one of embodiments 25-27, wherein the EI is determined according the formula:

$$EI = \frac{AEI \times 100}{145}$$

Embodiment 29. The method of any one of embodiments 25-28, wherein the AEI is determined as a sum of static AEI and dynamic AEI.
   Embodiment 30. The method of any one of embodiments 16-29, wherein the generating of the digital therapeutic modules comprises generating the digital therapeutic modules by applying imaginary parameters about the subject's environments, behaviors, emotions, and cognition to the mechanism of action in and the therapeutic hypothesis.
   Embodiment 31. The method of any one of the preceding embodiments, wherein the digital application instructs a processor of the digital apparatus to generate digital therapeutic modules comprising (i) an eye exercise module comprising the eyeball exercise instructions, and (ii) at least one of a relaxation module and a light therapy module.
   Embodiment 32. The method of embodiment 31, wherein
the eye exercise module further comprises one or more of biofeedback control instructions and eyeball-related behavior control instructions;
the relaxation module comprises one or more relaxation instructions for one or more of:
physical exercise instructions, ego enhancement instructions, safety feeling instructions, comfort feeling instructions, and fun instructions; and
the light therapy module comprises one or more light therapy instructions for controlling a light environment of the subject.
   Embodiment 33. The method of embodiment 32, wherein the one or more relaxation instructions comprise one or more of playing a sound or song, inducing blinking, and instructing the subject to perform gymnastics.
   Embodiment 34. The method of any one of embodiments 31-33, wherein the digital therapeutic modules further comprise an accomplishment module comprising one or more accomplishment instructions for task accomplishment and for providing compensation for the subject's adherence to the instructions of the two or more first modules.
   Embodiment 35. The method of any one of embodiments 31-34, wherein the digital therapeutic modules further comprise a fun module comprising one or more fun instructions for music, games, or videos.
   Embodiment 36. The method of any one of the embodiments 14-35, wherein the healthcare provider portal is configured to provide one or more options to the healthcare provider to perform one or more tasks to prescribe treatment in the subject based on the adherence information, wherein the one or more options provided to the healthcare provider are selected from the group consisting of adding or removing the subject, viewing or editing personal information for the subject, viewing adherence information for the subject, viewing a result of the subject for one or more at least partially completed digital therapeutic modules, prescribing one or more digital therapeutic modules to the subject, altering a prescription for one or more digital therapeutic modules, and communicating with the subject.
   Embodiment 37. The method of embodiment 36, wherein the one or more options comprise the viewing or editing personal information for the subject, and the personal information comprises one or more selected from the group consisting of an identification number for the subject, a name of the subject, a date of birth of the subject, an email of the subject, an email of the guardian of the subject, a contact phone number for the subject, a prescription for the subject, and one or more notes made by the healthcare provider about the subject.
   Embodiment 38. The method of embodiment 37, wherein the personal information comprises the prescription for the subject, and the prescription for the subject comprises one or more selected from the group consisting of a prescription identification number, a prescription type, a start date, a duration, a completion date, a number of scheduled or prescribed digital therapeutic modules to be performed by the subject, and a number of scheduled or prescribed digital therapeutic modules to be performed by the subject per day.
   Embodiment 39. The method of embodiment 36 or 37, wherein the one or more options comprise the viewing the adherence information, and the adherence information of the subject comprises one or more of a number of scheduled or prescribed digital therapeutic modules completed by the subject, and a calendar identifying one or more days on which the subject completed, partially completed, or did not complete one or more scheduled or prescribed digital therapeutic modules.
   Embodiment 40. The method of any one of embodiments 36-39, wherein the one or more options comprise the viewing the result of the subject, and the result of the subject for one or more at least partially completed digital therapeutic modules comprises one or more selected from the group consisting of a time at which the subject started a scheduled or prescribed digital therapeutic module, a time at which the subject ended a scheduled or prescribed digital therapeutic module, an indicator of whether the scheduled or prescribed digital therapeutic module was fully or partially completed, and an exercise intensity (EI).
   Embodiment 41. The method of any one of the embodiments 14-40, wherein the server is accessible by an administrator through an administrative portal configured to provide one or more options to an administrator of the system to perform one or more tasks to manage access to the system by the healthcare provider, and wherein the one or more options provided to the administrator of the method are selected from the group consisting of adding or removing the healthcare provider, viewing or editing personal information for the healthcare provider, viewing or editing de-identified information of the subject, viewing adherence information for the subject, viewing a result of the subject for one or more at least partially completed digital therapeutic modules, and communicating with the healthcare provider.
   Embodiment 42. The method of embodiment 41, wherein the one or more options comprise the viewing or editing the personal information, and the personal information of the healthcare provider comprises one or more selected from the group consisting of an identification number for the healthcare provider, a name of the healthcare provider, an email of the healthcare provider, and a contact phone number for the healthcare provider.
   Embodiment 43. The method of embodiment 41 or 42, wherein the one or more options comprise the viewing or editing the de-identified information of the subject, and the de-identified information of the subject comprises one or more selected from the group consisting of an identification number for the subject, and the healthcare provider for the subject.
   Embodiment 44. The method of any one of embodiments 41-43, wherein the one or more options comprise the viewing the adherence information for the subject, and the adherence information of the subject comprises one or more of a number of scheduled or prescribed digital therapeutic modules completed by the subject, and a calendar identifying one or more days on which the subject completed, partially completed, or did not complete one or more scheduled or prescribed digital therapeutic modules.
   Embodiment 45. The method of any one of embodiments 41-44, wherein the one or more options comprise the viewing the result of the subject, and the result of the subject for one or more at least partially completed digital therapeutic modules comprises one or more selected from the group consisting of a time at which the subject started a scheduled or prescribed digital therapeutic module, a time at which the subject ended a scheduled or prescribed digital therapeutic module, an indicator of whether the scheduled or prescribed digital therapeutic module was fully or partially completed, and an exercise intensity (EI).
   Embodiment 46. The method of any one of the preceding embodiments, wherein the digital application further comprises a push alarm for one or more of reminding the subject complete a digital therapeutic module and adjusting the light settings of the subject's environment.
   Embodiment 47. The method of embodiment 45, wherein the push alarm is activated to remind the subject to adjust the light settings such that the subject is exposed to sufficiently bright light at least 3 times per day.
   Embodiment 48. The method of any one of the preceding embodiments, wherein the subject is a child.
   Embodiment 49. The method of embodiment 48, wherein the subject is less than about 15 years old.
   Embodiment 50. The method of any one of the preceding embodiments, wherein the subject is assisted or supervised by an adult.
   Embodiment 51. The method of any one of the preceding embodiments, wherein the digital apparatus comprises: a digital instruction generation unit configured to generate digital therapeutic modules based on a mechanism of action (MOA) in and a therapeutic hypothesis, generate digital instructions based on the digital therapeutic modules, and provide the digital instructions to the subject; and an outcome collection unit configured to collect the subject's execution outcomes of the digital instructions.
   Embodiment 52. The method of embodiment 51, wherein the digital instruction generation unit generates the digital therapeutic modules based on neurohumoral factors.
   Embodiment 53. The method of embodiment 52, wherein the neurohumoral factors comprise insulin-like growth factor (IGF), cortisol, and dopamine.
   Embodiment 54. The method of any one of embodiments 51-53, wherein the digital instruction generation unit generates the digital therapeutic modules based on the inputs from the healthcare provider.
   Embodiment 55. The method of any one of embodiments 51-54, wherein the digital instruction generation unit generates the digital therapeutic modules based on information received from the subject.
   Embodiment 56. The method of embodiment 55, wherein the information is received from the subject comprises at least one of basal factors, medical information, and digital therapeutics literacy of the subject, the basal factors including the subject's activity, heart rate, sleep, and diet (including nutrition and calories), the medical information including the subject's electronic medical record (EMR), family history, genetic vulnerability, and genetic susceptibility, and the digital therapeutics literacy including the subject's accessibility, and technology adoption to the digital therapeutics and the apparatus.
   Embodiment 57. The method of any one of embodiments 51-56, wherein the digital instruction generation unit generates the digital therapeutic modules matching to imaginary parameters which correspond to the mechanism of action in and the therapeutic hypothesis.
   Embodiment 58. The method of embodiment 57, wherein the imaginary parameters are deduced in relation to the subject's environment, behaviors, emotions, and cognition.
   Embodiment 59. The method of any one of embodiments 51-58, wherein the outcome collection unit collects the execution outcomes of the digital instructions by monitoring the subject's adherence to the digital instructions or allowing the subject to directly input the subject's adherence to the digital instructions.
   Embodiment 60. The method of any one of embodiments 51-59, wherein the generation of the digital instructions at the digital instruction generation unit and the collection of the subject's execution outcomes of the digital instructions at the outcome collection unit are repeatedly executed several times with multiple feedback loops, and the digital instruction generation unit generates the subject's digital instructions for this cycle based on the subject's digital instructions in the previous cycle and the execution outcome data on the subject's digital instructions in the previous cycle collected at the outcome collection unit.
   Embodiment 61. A system for improving an eyesight in a subject, the system comprising: a digital apparatus configured to execute a digital application for improving an eyesight in the subject [depending method of embodiments 1-59]; a healthcare provider portal configured to provide one or more options to a healthcare provider to perform one or more tasks to prescribe treatment to improve the eyesight in the subject based on information received from the digital application; and an administrative portal configured to provide one or more options to an administrator of the system to perform one or more tasks to manage access to the system by the healthcare provider.
   Embodiment 62. A system for treating myopia in a subject in need thereof, the system comprising: a digital apparatus configured to execute a digital application for treating myopia in the subject [depending method of embodiments 1-59]; a healthcare provider portal configured to provide one or more options to a healthcare provider to perform one or more tasks to prescribe treatment to treat myopia in the subject based on information received from the digital application; and an administrative portal configured to provide one or more options to an administrator of the system to perform one or more tasks to manage access to the system by the healthcare provider.
   Embodiment 63. A non-transitory computer readable medium having stored thereon software instructions for improving an eyesight of a subject that, when executed by a processor, cause the processor to: display, by a digital apparatus to the subject, modules for improving an eyesight, each of the modules comprising one or more instructions for the subject to follow, wherein the first instructions comprise an eyeball exercise instruction for the subject to move at least one eyeball vertically; and sense, by a sensor in the digital apparatus, adherence by the subject to the instructions of the modules.
   Embodiment 64. A non-transitory computer readable medium having stored thereon software instructions for treating myopia in a subject in need thereof that, when executed by a processor, cause the processor to: display, by a digital apparatus to the subject, modules for treating myopia, each of the modules comprising one or more instructions for the subject to follow, wherein the first instructions comprise an eyeball exercise instruction for the subject to move at least one eyeball vertically; and sense, by a sensor in the digital apparatus, adherence by the subject to the instructions of the modules.
   Embodiment 65. The non-transitory computer readable medium of any one of the preceding embodiments, wherein the first eyeball exercise instruction is for the subject to move said at least one eyeball at least 50 out of 100 maximum vertical view of the subject.
   Embodiment 66. The non-transitory computer readable medium of any one of the preceding embodiments, wherein the first eyeball exercise instruction is for the subject to move said at least one eyeball at least 70 out of 100 maximum vertical view of the subject.
   Embodiment 67. The non-transitory computer readable medium of any one of the preceding embodiments, wherein the digital application comprises more instructions for vertical eye movements compared to instructions for horizontal eye movement.
   Embodiment 68. The non-transitory computer readable medium of any one of the preceding embodiments, wherein the first eyeball exercise instruction is to move said at least one eyeball upward.
   Embodiment 69. The non-transitory computer readable medium of any one of the embodiments 63-68, wherein the first eyeball exercise instruction comprise more instruction to move said at least one eyeball upward compared to instruction to move said at least eyeball downward.
   Embodiment 70. The non-transitory computer readable medium of any one of the preceding embodiments, wherein the first instructions exclude an instruction to move said at least one eyeball horizontally.
   Embodiment 71. The non-transitory computer readable medium of any one of the preceding embodiments, wherein the method improves a growth rate of AL (Axial Length) of said at least one eyeball in the subject.
   Embodiment 72. The non-transitory computer readable medium of any one of the preceding embodiments, wherein the method reduces a growth rate of AL (Axial Length) of said at least one eyeball in the subject.
   Embodiment 73. The non-transitory computer readable medium of any one of the preceding embodiments, further comprising measuring a maximum vertical view of the subject.
   Embodiment 74. The non-transitory computer readable medium of embodiment 73, wherein the measuring is performed by a sensor of the digital apparatus.
   Embodiment 75. The non-transitory computer readable medium of any one of the preceding embodiments, wherein the subject is 10 years old or more.
   Embodiment 76. The non-transitory computer readable medium of embodiment 63 or 64, wherein the modules are selected based on a mechanism of action in and a therapeutic hypothesis.
   Embodiment 77. The non-transitory computer readable medium of any one of embodiments 63 or 64, wherein the non-transitory computer readable medium further causes the processor to: transmit, by the digital apparatus, adherence information, based on the adherence, to a server accessible by a healthcare provider through a healthcare provider portal; and receive, from the server, one or more second instructions from the healthcare provider.
   Embodiment 78. The non-transitory computer readable medium of embodiment 77, wherein the digital application instructs a processor of the digital apparatus to execute operations comprising: generating digital therapeutic modules based on a mechanism of action in and a therapeutic hypothesis.
   Embodiment 79. The non-transitory computer readable medium of embodiment 78, wherein the generating of the digital therapeutic modules comprises generating the digital therapeutic modules based on neurohumoral factors.
   Embodiment 80. The non-transitory computer readable medium of embodiment 78 or 79, wherein the operations further comprise generating a calibration module for calibrating one or more of an accuracy of measurement of the subject's eye position, and a light environment.
   Embodiment 81. The non-transitory computer readable medium of embodiment 80, wherein the calibration module is generated prior to generating the digital therapeutic modules.
   Embodiment 82. The non-transitory computer readable medium of embodiment 80 or 81, wherein the accuracy of measurement of the subject's eye position is calibrated, and said calibrating the accuracy of measurement of the subject's eye position comprises one or more of instructing the subject to position their face to appear on a screen of the digital apparatus, detecting the subject's eyes for a given period of time, instructing the subject to blink their eyes, detecting if the subject blinked their eyes, instructing the subject to stare at the screen, instructing the subject to move their eyes in a given direction or rotate their eyes, and determining a threshold for detecting the subject's eyes.
   Embodiment 83. The non-transitory computer readable medium of embodiment 82, wherein the digital apparatus comprises one or more sensors for tracking movement of the subject's eyeball.
   Embodiment 84. The non-transitory computer readable medium of any one of embodiments 80-83, wherein the accuracy of measurement of the light environment is calibrated, and said calibrating the light environment comprises one or more of detecting light in the subject's environment using a light sensor of the digital apparatus, and instructing the subject to turn on one or more lights in their environment.
   Embodiment 85. The non-transitory computer readable medium of any one of embodiments 77-84, wherein the digital application instructs a processor of the digital apparatus to execute operations comprising: generating digital therapeutic modules based on a mechanism of action in and a therapeutic hypothesis; generating digital instructions based on the digital therapeutic modules; providing the digital instruction to a subject; and collecting the subject's execution outcomes of the digital instructions.
   Embodiment 86. The non-transitory computer readable medium of embodiment 85, wherein the generating of the digital instructions and the collecting of the subject's execution outcomes of the digital instructions are repeatedly executed several with multiple feedback loops, and the generating of the digital instructions comprises generating the subject's digital instructions for this cycle based on the subject's digital instructions in the previous cycle and the collected execution outcome data on the subject's digital instructions provided in the previous cycle.
   Embodiment 87. The non-transitory computer readable medium of embodiment 85 or 86, wherein the collecting the subject's execution outcomes of the digital instructions comprises determining one or both of an exercise intensity (EI) and an average exercise intensity (AEI).
   Embodiment 88. The non-transitory computer readable medium of embodiment 87, wherein AEI is determined as an averaged sum of differences between a final location of an eyeball of the subject and a starting location of the eyeball measured at a given interval.
   Embodiment 89. The non-transitory computer readable medium of embodiment 88, wherein the interval is between about 10 milliseconds (ms) and about 500 ms.
   Embodiment 90. The non-transitory computer readable medium of any one of embodiments 87-89, wherein the EI is determined according the formula:

$$EI = \frac{AEI \times 100}{145}$$

Embodiment 91. The non-transitory computer readable medium of any one of embodiments 87-90, wherein the AEI is determined as a sum of static AEI and dynamic AEI.
   Embodiment 92. The non-transitory computer readable medium of any one of embodiments 78-91, wherein the generating of the digital therapeutic modules comprises generating the digital therapeutic modules by applying imaginary parameters about the subject's environments, behaviors, emotions, and cognition to the mechanism of action in and the therapeutic hypothesis.
   Embodiment 93. The non-transitory computer readable medium of any one of embodiments 78-92, wherein the digital application instructs a processor of the digital apparatus to generate digital therapeutic modules comprising two or more modules selected from the group consisting of an eye exercise module, a relaxation module, and a light therapy module.
   Embodiment 94. The non-transitory computer readable medium of embodiment 93, wherein the eye exercise module comprises one or more exercise instructions for one or more of: eyeball exercise instructions, biofeedback control instructions, and eyeball-related behavior control instructions; the relaxation module comprises one or more relaxation instructions for one or more of: physical exercise instructions, ego enhancement instructions, safety feeling instructions, comfort feeling instructions, and fun instructions; and the light therapy module comprises one or more light therapy instructions for controlling a light environment of the subject.
   Embodiment 95. The non-transitory computer readable medium of embodiment 94, wherein the one or more relaxation instructions comprise one or more of playing a sound or song, inducing blinking, and instructing the subject to perform gymnastics.
   Embodiment 96. The non-transitory computer readable medium of any one of embodiments 93-95, wherein the digital therapeutic modules further comprise an accomplishment module comprising one or more accomplishment instructions for task accomplishment and for providing compensation for the subject's adherence to the instructions of the two or more first modules.
   Embodiment 97. The non-transitory computer readable medium of any one of embodiments 93-96, wherein the digital therapeutic modules further comprise a fun module comprising one or more fun instructions for music, games, or videos.
   Embodiment 98. The non-transitory computer readable medium of any one of embodiments 77-97, wherein the healthcare provider portal is configured to provide one or more options to the healthcare provider to perform one or more tasks to prescribe treatment in the subject based on the adherence information, wherein the one or more options provided to the healthcare provider are selected from the group consisting of adding or removing the subject, viewing or editing personal information for the subject, viewing adherence information for the subject, viewing a result of the subject for one or more at least partially completed digital therapeutic modules, prescribing one or more digital therapeutic modules to the subject, altering a prescription for one or more digital therapeutic modules, and communicating with the subject.
   Embodiment 99. The non-transitory computer readable medium of embodiment 98, wherein the one or more options comprise the viewing or editing personal information for the subject, and the personal information comprises one or more selected from the group consisting of an identification number for the subject, a name of the subject, a date of birth of the subject, an email of the subject, an email of the guardian of the subject, a contact phone number for the subject, a prescription for the subject, and one or more notes made by the healthcare provider about the subject.
   Embodiment 100. The non-transitory computer readable medium of embodiment 99, wherein the personal information comprises the prescription for the subject, and the prescription for the subject comprises one or more selected from the group consisting of a prescription identification number, a prescription type, a start date, a duration, a completion date, a number of scheduled or prescribed digital therapeutic modules to be performed by the subject, and a number of scheduled or prescribed digital therapeutic modules to be performed by the subject per day.
   Embodiment 101. The non-transitory computer readable medium of any one of embodiments 98-100, wherein the one or more options comprise the viewing the adherence information, and the adherence information of the subject comprises one or more of a number of scheduled or prescribed digital therapeutic modules completed by the subject, and a calendar identifying one or more days on which the subject completed, partially completed, or did not complete one or more scheduled or prescribed digital therapeutic modules.
   Embodiment 102. The non-transitory computer readable medium of any one of embodiments 98-101, wherein the one or more options comprise the viewing the result of the subject, and the result of the subject for one or more at least partially completed digital therapeutic modules comprises one or more selected from the group consisting of a time at which the subject started a scheduled or prescribed digital therapeutic module, a time at which the subject ended a scheduled or prescribed digital therapeutic module, an indicator of whether the scheduled or prescribed digital therapeutic module was fully or partially completed, and an exercise intensity (EI).
   Embodiment 103. The non-transitory computer readable medium of any one of embodiments 77-102, wherein the server is accessible by an administrator through an administrative portal configured to provide one or more options to an administrator of the system to perform one or more tasks to manage access to the system by the healthcare provider, and wherein the one or more options provided to the administrator of the method are selected from the group consisting of adding or removing the healthcare provider, viewing or editing personal information for the healthcare provider, viewing or editing de-identified information of the subject, viewing adherence information for the subject, viewing a result of the subject for one or more at least partially completed digital therapeutic modules, and communicating with the healthcare provider.
   Embodiment 104. The non-transitory computer readable medium of embodiment 103, wherein the one or more options comprise the viewing or editing the personal information, and the personal information of the healthcare provider comprises one or more selected from the group consisting of an identification number for the healthcare provider, a name of the healthcare provider, an email of the healthcare provider, and a contact phone number for the healthcare provider.
   Embodiment 105. The non-transitory computer readable medium of embodiment 103 or 104, wherein the one or more options comprise the viewing or editing the de-identified information of the subject, and the de-identified information of the subject comprises one or more selected from the group consisting of an identification number for the subject, and the healthcare provider for the subject.
   Embodiment 106. The non-transitory computer readable medium of any one of embodiments 103-105, wherein the one or more options comprise the viewing the adherence information for the subject, and the adherence information of the subject comprises one or more of a number of scheduled or prescribed digital therapeutic modules completed by the subject, and a calendar identifying one or more days on which the subject completed, partially completed, or did not complete one or more scheduled or prescribed digital therapeutic modules.
   Embodiment 107. The non-transitory computer readable medium of any one of embodiments 103-106, wherein the one or more options comprise the viewing the result of the subject, and the result of the subject for one or more at least partially completed digital therapeutic modules comprises one or more selected from the group consisting of a time at which the subject started a scheduled or prescribed digital therapeutic module, a time at which the subject ended a scheduled or prescribed digital therapeutic module, an indicator of whether the scheduled or prescribed digital therapeutic module was fully or partially completed, and an exercise intensity (EI).
   Embodiment 108. The non-transitory computer readable medium of any one of embodiments 77-107, wherein the digital application further comprises a push alarm for one or more of reminding the subject complete a digital therapeutic module and adjusting the light settings of the subject's environment.
   Embodiment 109. The non-transitory computer readable medium of any one of embodiments 77-108, wherein the push alarm is activated to remind the subject to adjust the light settings such that the subject is exposed to sufficiently bright light at least 3 times per day.
   Embodiment 110. The non-transitory computer readable medium of any one of embodiments 77-109, wherein the subject is a child.
   Embodiment 111. The non-transitory computer readable medium of embodiment 110, wherein the subject is less than about 15 years old, Embodiment 112. The non-transitory computer readable medium of any one of embodiments 77-111, wherein the subject is assisted or supervised by an adult.
   Embodiment 113. The non-transitory computer readable medium of any one of embodiments 77-112, wherein the digital apparatus comprises: a digital instruction generation unit configured to generate digital therapeutic modules based on a mechanism of action (MOA) in and a therapeutic hypothesis, generate digital instructions based on the digital therapeutic modules, and provide the digital instructions to the subject; and an outcome collection unit configured to collect the subject's execution outcomes of the digital instructions.
   Embodiment 114. The non-transitory computer readable medium of embodiment 113, wherein the digital instruction generation unit generates the digital therapeutic modules based on neurohumoral factors.
   Embodiment 115. The non-transitory computer readable medium of embodiment 114, wherein the neurohumoral factors comprise insulin-like growth factor (IGF), cortisol, and dopamine.
   Embodiment 116. The non-transitory computer readable medium of any one of embodiments 113-115, wherein the digital instruction generation unit generates the digital therapeutic modules based on the inputs from the healthcare provider.
   Embodiment 117. The non-transitory computer readable medium of any one of embodiments 113-116, wherein the digital instruction generation unit generates the digital therapeutic modules based on information received from the subject.
   Embodiment 118. The non-transitory computer readable medium of embodiment 117, wherein the information is received from the subject comprises at least one of basal factors, medical information, and digital therapeutics literacy of the subject, the basal factors including the subject's activity, heart rate, sleep, and diet (including nutrition and calories), the medical information including the subject's electronic medical record (EMR), family history, genetic vulnerability, and genetic susceptibility, and the digital therapeutics literacy including the subject's accessibility, and technology adoption to the digital therapeutics and the apparatus.
   Embodiment 119. The non-transitory computer readable medium of any one of embodiments 113-118, wherein the digital instruction generation unit generates the digital therapeutic modules matching to imaginary parameters which correspond to the mechanism of action in and the therapeutic hypothesis.
   Embodiment 120. The non-transitory computer readable medium of embodiment 119, wherein the imaginary parameters are deduced in relation to the subject's environment, behaviors, emotions, and cognition.
   Embodiment 121. The non-transitory computer readable medium of any one of embodiments 113-120, wherein the outcome collection unit collects the execution outcomes of the digital instructions by monitoring the subject's adherence to the digital instructions or allowing the subject to directly input the subject's adherence to the digital instructions.
   Embodiment 122. The non-transitory computer readable medium of any one of embodiments 113-121, wherein the generation of the digital instructions at the digital instruction generation unit and the collection of the subject's execution outcomes of the digital instructions at the outcome collection unit are repeatedly executed several times with multiple feedback loops, and the digital instruction generation unit generates the subject's digital instructions for this cycle based on the subject's digital instructions in the previous cycle and the execution outcome data on the subject's digital instructions in the previous cycle collected at the outcome collection unit.

What is claimed is:

1. A method of improving an eyesight of a subject, the method comprising:
providing, by a digital apparatus to the subject, a digital application including one or more digital therapeutic modules for improving the eyesight to the subject, each of the one or more digital therapeutic modules including one or more first instructions for the subject to follow, wherein the one or more first instructions include a first eyeball exercise instruction for the subject to move at least one eyeball vertically.

2. The method according to claim 1, wherein the first eyeball exercise instruction is for the subject to move said at least one eyeball at least 50 out of 100 maximum vertical view of the subject.

3. The method according to claim 1, wherein the first eyeball exercise instruction is for the subject to move said at least one eyeball at least 70 out of 100 maximum vertical view of the subject.

4. The method according to claim 1, wherein the digital application includes more instructions for vertical eye movements compared to instructions for horizontal eye movement.

5. The method according to claim 1, wherein the first eyeball exercise instruction is to move said at least one eyeball upward.

6. The method according to claim 1, wherein the first eyeball exercise instruction includes more instruction to move said at least one eyeball upward compared to instruction to move said at least eyeball downward.

7. The method according to claim 1, wherein the method reduces a growth rate of AL (Axial Length) of said at least one eyeball in the subject.

8. The method according to claim 1, further comprising measuring a maximum vertical view of the subject.

9. The method according to claim 1, further comprising calibrating one or more of an accuracy of measurement of the subject's eye position, and a light environment.

10. The method according to claim 9, wherein the accuracy of measurement of the light environment is calibrated, and the calibrating the light environment includes: one or more of detecting light in the subject's environment using a light sensor of the digital apparatus, and instructing the subject to turn on one or more lights in their environment.

11. The method according to claim 1, the method comprising:
generating the one or more digital therapeutic modules by applying imaginary parameters about the subject's environments, behaviors, emotions, and cognition to the mechanism of action in and the therapeutic hypothesis.

12. The method according to claim 1, comprising:
sensing adherence by the subject to the one or more first instructions of the one or more digital therapeutic modules.

13. The method according to claim 12, comprising:
transmitting adherence information, based on the adherence, to a server; and
receiving one or more second instructions from the server based on the adherence information.

14. The method according to claim 13, wherein the one or more second instructions comprise a second eyeball exercise instructions for an eyeball movement at a speed adjusted based on the adherence information.

15. The method according to claim 1, wherein the digital application instructs a processor of the digital apparatus to execute operations comprising: generating the one or more digital therapeutic modules based on a mechanism of action and a therapeutic hypothesis and collecting a subject's execution outcomes of the digital instructions.

16. The method according to claim 15, wherein the generating of the digital instructions and the collecting of the subject's execution outcomes of the digital instructions are repeatedly executed several with multiple feedback loops, and the generating of the digital instructions includes generating the subject's digital instructions for this cycle based on the subject's digital instructions in the previous cycle and the collected execution outcome data on the subject's digital instructions provided in the previous cycle.

17. The method according to claim 15, wherein the collecting the subject's execution outcomes of the digital instructions includes determining one or both of an exercise intensity (EI) and an average exercise intensity (AEI).

18. The method according to claim 17, wherein the AEI is determined as an averaged sum of differences between a final location of an eyeball of the subject and a starting location of the eyeball measured at a given interval.

19. The method according to claim 1, wherein the one or more digital therapeutic modules are generated based on neurohumoral factors.

20. A digital apparatus for improving an eyesight of a subject comprises:
a digital instruction generation unit configured to generate one or more digital therapeutic modules based on a mechanism of action (MOA) and a therapeutic hypothesis, generate digital instructions based on the digital therapeutic modules, and provide the digital instructions to the subject; and
an outcome collection unit configured to collect the subject's execution outcomes of the digital instructions, wherein each of the one or more digital therapeutic modules including one or more first instructions for the subject to follow, wherein the one or more first instructions include a first eyeball exercise instruction for the subject to move at least one eyeball vertically.

21. The digital apparatus according to claim 20, wherein the first eyeball exercise instruction is for the subject to move said at least one eyeball at least 50 out of 100 maximum vertical view of the subject.

22. The digital apparatus according to claim 20, wherein the first eyeball exercise instruction is for the subject to move said at least one eyeball at least 70 out of 100 maximum vertical view of the subject.

23. The digital apparatus according to claim 20, wherein the digital application includes more instructions for vertical eye movements compared to instructions for horizontal eye movement.

24. The digital apparatus according to claim 20, wherein the first eyeball exercise instruction is to move said at least one eyeball upward.

25. The digital apparatus according to claim 20, wherein the first eyeball exercise instruction includes more instruction to move said at least one eyeball upward compared to instruction to move said at least eyeball downward.

26. The digital apparatus according to claim 20, wherein the digital apparatus is further configured to provide a function for calibrating one or more of an accuracy of measurement of the subject's eye position, and a light environment.

27. The digital apparatus according to claim 26, wherein the accuracy of measurement of the light environment is calibrated, and the calibrating the light environment includes: one or more of detecting light in the subject's environment using a light sensor of the digital apparatus, and instructing the subject to turn on one or more lights in their environment.

28. The digital apparatus according to claim 20, wherein the digital apparatus is further configured to generate the one or more digital therapeutic modules by applying imaginary parameters about the subject's environments, behaviors, emotions, and cognition to the mechanism of action in and the therapeutic hypothesis.

29. The digital apparatus according to claim 20, comprising: a sensor configured to sense adherence by the subject to the one or more first instructions.

30. The digital apparatus according to claim 29, wherein the digital apparatus is further configured to transmit adherence information, based on the adherence, to a server and receive one or more second instructions from the server based on the adherence information.

31. The digital apparatus according to claim 30, wherein the one or more second instructions include a second eyeball exercise instructions for an eyeball movement at a speed adjusted based on the adherence information.

32. The digital apparatus according to claim 20, wherein the digital application instructs a processor of the digital apparatus to execute operations including: generating the one or more digital therapeutic modules based on a mechanism of action and a therapeutic hypothesis and collecting a subject's execution outcomes of the digital instructions.

33. The digital apparatus according to claim 32, wherein the generating of the digital instructions and the collecting of the subject's execution outcomes of the digital instructions are repeatedly executed several with multiple feedback loops, and the generating of the digital instructions includes generating the subject's digital instructions for this cycle based on the subject's digital instructions in the previous cycle and the collected execution outcome data on the subject's digital instructions provided in the previous cycle.

34. The digital apparatus according to claim 32, wherein the collecting the subject's execution outcomes of the digital instructions includes determining one or both of an exercise intensity (EI) and an average exercise intensity (AEI).

35. The digital apparatus according to claim 35, wherein the AEI is determined as an averaged sum of differences between a final location of an eyeball of the subject and a starting location of the eyeball measured at a given interval.

36. The digital apparatus according to claim 20, wherein the one or more digital therapeutic modules are generated based on neurohumoral factors.

37. Anon-transitory computer readable medium having stored thereon software instructions for improving an eyesight of a subject that, when executed by a processor of a digital apparatus, cause the processor to: provide, by a digital apparatus to the subject, a digital application including one or more digital therapeutic modules for improving the eyesight to the subject, each of the one or more digital therapeutic modules including one or more first instructions for the subject to follow, wherein the one or more first instructions include a first eyeball exercise instruction for the subject to move at least one eyeball vertically.
